(19) United States
(12) Patent Application Publication
(10) Pub. No.: US 2014/0288044 A1
(43) Pub. Date: Sep. 25, 2014

(54) COMPOSITIONS AND THERAPEUTIC USES OF IKK-RELATED KINASE EPSILON AND TANKBINDING KINASE 1 INHIBITORS

(71) Applicants: Ryan Holcomb, Salt Lake City, UT (US); Paul R. Sebahar, Sandy, UT (US); Kazuyuki Suzuki, Fukushima (JP); Donald A. McLeod, Sandy, UT (US); David M. Dastrup, Winchester, OH (US); Christophe Hoarau, Eugene, OR (US); Robert J. Halter, Salt Lake City, UT (US); Matthew Gregory Bursavich, Needham Heights, MA (US); Mark D. Shenderovich, Salt Lake City, UT (US); Burt Richards, Midway, UT (US); Paul L. Bartel, Salt Lake City, UT (US)

(72) Inventors: Ryan Holcomb, Salt Lake City, UT (US); Paul R. Sebahar, Sandy, UT (US); Kazuyuki Suzuki, Fukushima (JP); Donald A. McLeod, Sandy, UT (US); David M. Dastrup, Winchester, OH (US); Christophe Hoarau, Eugene, OR (US); Robert J. Halter, Salt Lake City, UT (US); Matthew Gregory Bursavich, Needham Heights, MA (US); Mark D. Shenderovich, Salt Lake City, UT (US); Burt Richards, Midway, UT (US); Paul L. Bartel, Salt Lake City, UT (US)

(73) Assignee: Alzheimer's Institute of America, Inc., Kansas City, KS (US)

(21) Appl. No.: 14/112,847

(22) PCT Filed: Apr. 12, 2012
(86) PCT No.: PCT/US12/33384

§ 371 Date: Feb. 5, 2014

Related U.S. Application Data
(60) Provisional application No. 61/474,366, filed on Apr. 12, 2011.

(51) Int. Cl. C07D 401/12; C07D 401/14; C07D 413/14; C07D 417/14; C07D 405/14; C07D 409/14

(52) U.S. Cl. CPC C07D 401/12; C07D 405/14; C07D 409/14; C07D 413/14; C07D 417/14; C07D 401/14
USPC 514/210.18; 544/331; 514/275; 544/122; 514/235.8; 544/296; 544/295; 514/252.18; 514/210.2; 514/236.5; 544/238; 514/252.02; 544/120; 435/375

(57) ABSTRACT

The invention relates to compounds, pharmaceutical compositions and medicaments comprising such compounds, and the use of these compounds, compositions, and medicaments in methods of treating diseases and disorders.

### CROSS-REFERENCE TO RELATED APPLICATIONS

This application claims the benefit of U.S. Provisional Application Ser. No. 61/474,366, filed Apr. 12, 2011, the contents of which are hereby incorporated by reference in their entirety.

### FIELD OF THE INVENTION

The present invention relates generally to the field of medicinal chemistry. Specifically, the present invention provides compounds that inhibit IKK-related kinase epsilon (IKKε), TANK-binding kinase 1 (TBK1), or both IKKε and TBK1. The invention also provides methods for making these compounds, pharmaceutical compositions comprising these compounds, and methods for treating diseases with these compounds and compositions.

### BACKGROUND OF THE INVENTION

The protein “I-kappa-B kinase epsilon” or “IKKε” (also known as “inducible IkappaB kinase” or “IKK-i”) is a member of the IκB family of kinases, and contains a kinase domain in its N-terminus, which shares substantial identity to that of I-kappa-B kinase alpha (IKKα) or I-kappa-B kinase beta (IKKβ), and even greater identity with the kinase domain of TANK-binding kinase 1 (TBK1). IKKε was first identified as a protein whose encoding messenger RNA is substantially induced by lipopolysaccharide (LPS). (Shimada, et al.; IKK-i, a novel lipopolysaccharide-inducible kinase that is related to IκB kinases; Int. Immunol., 11:1357-1362, 1999.) Subsequent studies revealed that the expression of IKKε is induced by activation of the inflammatory NF-κB signaling pathway. (Matsuda, et al.; Large-scale identification and characterization of human genes that activate NF-kappaB and MAPK signaling pathways; Oncogene, 22:3307-3318, 2003.) IKKε is expressed mainly in immune cells, and is induced in response to pro-inflammatory cytokines such as tumor necrosis factor-alpha, IL-1 and IL-6, in addition to lipopolysaccharide (LPS). Overexpression of wild-type IKKε results in the phosphorylation of IκB alpha, and stimulation of NF-kappaB activation. (Shimada, et al.; Int. Immunol., 11:1357-1362, 1999.)

While all of its functions are not completely understood, IKKε has been found to play many important roles in human cells. For example, it has been known for some time that IKKε plays a key role in integrating signals induced by pro-inflammatory stimuli. (Kravchenko et al., IKKi/IKKepsilon plays a key role in integrating signals induced by pro-inflammatory stimuli; J. Biol. Chem., 278:26612-26619, 2003.) Further, it is known that IKKε is involved in the antiviral interferon (IFN) response, and that, along with TBK1, IKKε forms a virus-activated kinase complex that phosphorylates interferon regulatory factors 3 and 7 (IRF3 & IRF7). (Sharma et al.; Triggering the interferon antiviral response through an IKK-related pathway; Science, 300:1148-1151, 2003.) Additionally, IKKε, along with TBK1, has been shown to play a role in maintaining macrophages in an activated, inflammatory state, following activation of the interferon response. (Solis, et al.; Involvement of TBK1 and IKKepsilon in lipopolysaccharide-induced activation of the interferon response in primary human macrophages; Eur. J. Immunol., 37:529-539, 2007.)

TBK1 is highly related to IKKε and is constitutively expressed in most cell types (Clement et al., The IKK-related kinases: from innate immunity to oncogenesis; Cell Res., 18:889-899, 2008). Similar to IKKε, TBK1 is responsible for phosphorylation of IRF3 & IRF7 and NF-kB transcription factors after activation of innate immune receptors leading to transcription of several proinflammatory proteins (Chau et al., Are the IKKs and IKK-related kinases TBK1 and IKK-epsilon similarly activated?; Trends Biochem Sci., 33:171-180, 2008). TBK1 and IKKε protein share redundant and possibly overlapping roles in innate immune signaling and possibly autoimmune diseases, therefore inhibition of both kinases may prove advantageous.

In view of the roles identified for IKKε in the interferon antiviral response, and in the maintenance of macrophages in an activated, inflammatory state, it is perhaps not surprising that IKKε, as part of the kinase complex, has also been found to play a role in the synovial inflammation, extracellular matrix destruction and activation of the viral program and innate immune response in rheumatoid arthritis (RA). (Sweeney et al., Regulation of c-Jun phosphorylation by the IκB kinase-ε complex in fibroblast-like synoviocytes; J. Immunol., 174:6424-6430, 2005.) Indeed, further studies of the role of IKKε and its downstream phosphorylation target IRF3 in RA, have demonstrated that IKKε and IRF3 protein levels are significantly elevated in RA synovium compared to osteoarthritic synovium, and that an IKKε-dependent mechanism results in the increased production of interferon beta, and RANTES in cultured synoviocytes. IKKε null mice demonstrated reduced inflammation and erosion as well as a decrease in clinical arthritis in the collagen-induced arthritis model (Con et al.; Synergistic benefit in inflammatory arthritis by targeting IκB kinase ε and interferon β; Ann. Rheum. Dis., 68:257-263, 2009). These results suggest that the IKKε-dependent pathway may be an important therapeutic target in the treatment of RA. (Sweeney et al.; Antiviral gene expression in rheumatoid arthritis; Arthritis Rheum., 56:743-752, 2007).

Systemic lupus erythematosus (SLE) is an autoimmune disease principally affecting women of child-bearing age. The disease is caused by an inappropriate immune response directed against intranuclear, self-antigens. It manifests systemically with involvement of many organs, including the kidneys, joints, skin and nervous system. The underlying inflammatory state predisposes patients to infections and cardiovascular disease, which are the major causes of mortality and morbidity in SLE. The current model for the molecular pathology of SLE is deregulation of T, B, and dendritic cell populations via an undetermined mechanism. This leads to imbalances of several cytokines and chemokines in T and B cell compartments eventually leading to organ damage (Crispin et al.; Pathogenesis of human systemic lupus erythematosus: recent advances; Trends Mol. Med., 16:47-57, 2010). In addition, the inability of dendritic cells to properly integrate signals from apoptotic cell debris or bacterial and viral infections leads to overproduction of the type I interferons (IFNα/β). In approximately half of all SLE patients a characteristic interferon gene signature has been identified (Baechler et al.; Interferon-inducible gene expression signature in peripheral blood cells of patients with severe lupus; Proc. Natl. Acad. Sci. U.S.A., 100:2610-2615, 2003). The expression of many of the interferon-regulated genes coincides with flares or periods of increased disease symptoms in SLE patients. While a single underlying cause has not been described to date, it is clear that adaptive and innate immune responses are compromised which leads to aberrant regulation of the entire immune system in SLE patients. The increase in IFNα/β production in SLE patients is due to activation of toll-like receptors (TLRs) and possibly intracellular nucleic acid receptors (Baccala et al.; TLR-dependent and TLR-independent pathways of type I interferon induction in systemic autoimmunity; Nat. Med., 13:543551, 2007). One of the downstream effects of receptor engagement is activation of the IKKε and TBK1 kinases leading to phosphorylation of transcription factors IRF3 and IRF7. Upon phosphorylation, the IRFs move into the nucleus and mediate upregulation of IFNα/β and associated interferon signature genes, including OAS1, OAS2, MX1, MX2, PKR, ISG54, ISG56, RANTES, CXCL-10, as well as others.

IKKε and TBK1 are involved in autoimmune diseases associated with accumulation of cytosolic nucleic acids. Several autoimmune diseases including; Sjögrens syndrome, Aicardi-Goutieres syndrome, subtypes of SLE, chilblain lupus, retinal vasculopathy and cerebral leukodystrophy (RVCL) appear to be caused by mutations in genes such as TREX1, SAMHD1, and RNASEH2A-C, which encode proteins involved in degrading viral nucleic acids or accumulated endogenous cytosolic nucleic acids (Crow and Rehwinkel; Aicardi-Goutières syndrome and related phenotypes: linking nucleic acid metabolism with autoimmunity; Hum. Mol. Genet., 18; 130-136, 2009; and Kavanagh, et al.; New roles for the major human 3′-5′ exonuclease TREX1 in human disease; Cell Cycle, 7:1718-1725, 2008). Patients carrying mutations that result in reduction or complete loss of protein activity have elevated expression of IFNβ and a set of “interferon signature” genes, and this elevated expression is dependent on IRF3 (Stetson et al.; Trex1 prevents cell-intrinsic initiation of autoimmunity; Cell, 134:587-598, 2008). IRF3 is phosphorylated by IKKε and/or TBK1 in response to signals from nucleic acid receptors, such as RIG-I, MDA5, DAI, IFI16, and others (Unterholzner et al.; IFI16 is an innate immune sensor for intracellular DNA; Nat. Immunol., E-pub Oct. 3, 2010), and phosphorylation of IFR3 leads to type I interferon production.

Systemic sclerosis, Sjögrens syndrome, dermatomyositis, polymyositis (Walsh et al.; Type I Interferon-Inducible Gene Expression in Blood Is Present and Reflects Disease Activity in Dermatomyositis and Polymyositis; Arthritis Rheum., 56:3784-3792, 2007) and plaque psoriasis (Delgado-Vega, et al.; Genetic associations in type I interferon related pathways with autoimmunity; Arthritis Res. Ther., April 14; 12 Suppl 1:S2, 2010) are autoimmune diseases characterized by elevated type I interferons and a characteristic interferon gene signature (Sozzani, et al.; Type I interferons in systemic autoimmunity; Autoimm., 43:196-203, 2010). Signaling pathways involving IKKε and TBK1 increase type I interferon expression following activation of upstream TLR3, TLR4, and cytosolic nucleic acid receptors (Honda et al.; Regulation of the type I IFN induction: a current view; Intern. Immunol, 17:1367-1378, 2005) consistent with a role in systemic sclerosis and myositis. Increased type I IFN signaling and the upregulation of viral dsRNA receptors including; TLR3, RIG1, and MDA5 in psoriatic skin support a role for IKKε and TBK1 in the pathogenesis of psoriasis (Prens et al.; IFN-alpha enhances poly-IC responses in human keratinocytes by inducing expression of cytosolic innate RNA receptors: relevance for psoriasis; J. Invest. Dermatol., 128: 932-938, 2008).

Chronic obstructive pulmonary disease (COPD) is characterized by inflammation of the lungs and narrowing of the airways. Exacerbation of COPD is caused by viral and bacterial infections that can prove fatal. Viral and bacterial pulmonary infections are recognized by toll-like receptors or cytosolic nucleic acid receptors (Takaoka and Taniguchi; Cytosolic DNA recognition for triggering innate immune response; Adv. Drug Delivery Rev., 60:847-857, 2008), which activate IKKε and TBK1 kinases and lead to proinflammatory response. The involvement of IKKε and TBK1 kinases in this response is supported by findings that several IRF3 and IRF7 responsive proinflammatory genes (e.g., IFNβ, IP-10 and IL-8) are induced during rhinovirus-induced COPD (Wang et al.; Role of double-stranded RNA pattern recognition receptors in rhinovirus-induced airway epithelial cell responses; J. Immunol., 183:6989-6997, 2009).

Inflammatory bowel disease (IBD) is an autoimmune-like disease characterized by an abnormal response to bacteria in the gut. TLRs have been implicated in IBD based on single-nucleotide polymorphisms in IBD patients (Cario; Toll-like receptors in inflammatory bowel diseases: a decade later; Inflamm. Bowel Dis., 16:1583-1597, 2010). The TLR4 protein is a bacterial lipopolysaccharide-recognizing receptor that activates the IRF3 pathway through IKKε and TBK1 kinases leading to RANTES and MCP-1 secretion. Elevation of both RANTES and MCP-1 protein levels are associated with IBD (McCormack et al.; Tissue cytokine and chemokine expression in inflammatory bowel disease; Inflamm. Res., 50:491-495, 2001).

It has been shown that a high-fat diet can increase NF-κB activation in mice, which leads to sustained elevation in the level of IKKε in liver, adipocytes, and adipose tissue macrophages. (See Chiang et al.; The protein kinase IKKε regulates energy balance in obese mice; Cell, 138:961-975, 2009) Further, mice in which the gene encoding IKKε was knocked out were found to be protected from high-fat diet-induced obesity, chronic inflammation in liver and fat, hepatic steatosis, and whole-body insulin resistance. These IKKε knockout mice were found to have increased energy expenditure and thermogenesis, and maintained insulin sensitivity in both liver and fat, without activation of the JNK pathway. Finally, these knockout mice were also found to have reduced expression of inflammatory cytokines, and altered expression of regulatory proteins and enzymes involved in glucose and lipid metabolism. In view of these observations, Chiang and coworkers concluded that IKKε may represent an attractive therapeutic target for obesity, insulin resistance, non-insulin-dependent diabetes mellitus (type 2 diabetes or NIDDM), metabolic syndrome, and other complications associated with these, and other, metabolic diseases and disorders. (Chiang et al.; Cell, 138:961-975, 2009.)

Additionally, TBK1 was implicated as a regulator of the insulin receptor in obese Zucker rats (an art-accepted model of insulin resistance/diabetes), suggesting TBK1 could be involved in mediating insulin resistance (Muñoz et al.; TANK-binding kinase 1 mediates phosphorylation of insulin receptor at serine residue 994: a potential link between inflammation and insulin resistance; J. Endocrinol., 201:185-197, 2009).

In addition to the above-described roles in macrophage activation, antiviral response, and inflammation, the gene encoding IKKε (i.e., IKBKE; Entrez Gene ID: 9641) has been identified as a breast cancer oncogene (Boehm, et al.; Integrative genomic approaches identify IKBKE as a breast cancer oncogene; Cell, 129:1065-1079, 2007). Further, IKKε has been found to directly phosphorylate the tumor suppressor CYLD in vivo, thereby decreasing the activity of CYLD, and leading to transformation and tumorigenesis (Hutti, et al.; Phosphorylation of the tumor suppressor CYLD by the breast cancer oncogene IKKepsilon promotes cell transformation; Mol. Cell, 34:461-472, 2009). In agreement with these observations, it has recently been discovered that overexpression of IKKε is a recurrent event in human ovarian cancer, and that this overexpression could play a role in both tumor progression and the development of cisplatin resistance (Guo, et al.; Deregulation of IKBKE is associated with tumor progression, poor prognosis, and cisplatin resistance in ovarian cancer; Am. J. Pathol., 175:324-333, 2009).

Another role for IKKε has recently been described in triggering an NF-kB antiapoptotic response in response to DNA damage. After genotoxic stress, IKKε translocates to the nucleus and phosphorylates PML to prevent cell death (Renner, et al.; SUMOylation-dependent localization of IKKε in PML nuclear bodies is essential for protection against DNA-damage-triggered cell death; Mol. Cell., 37:503-515, 2010). This newly described activity may contribute to IKKε's role as an oncogene and further support its role as a cancer target.

Additionally, TBK1 (Entrez Gene ID: 29110) has been identified as a proangiogenic gene that is induced under hypoxic conditions and is overexpressed in breast and colon cancers (Korherr, et al.; Identification of proangiogenic genes and pathways by high-throughput functional genomics: TBK1 and the IRF3 pathway; Proc. Natl. Acad. Sci. USA, 103:4240-4245, 2006). In cancer cells, TBK1 was found to restrict initiation of apoptotic programs typically engaged in the context of oncogenic stress (Chien et al.; RalB GTPase-mediated activation of the IκB family kinase TBK1 couples innate immune signaling to tumor cell survival; Cell, 127:157-170, 2006). TBK1 was also recently discovered to exhibit synthetic lethality with oncogenic Ras mutations in cancer cell lines. An RNA interference screen demonstrated potent reduction of cell viability when TBK1 protein was reduced in a Ras mutant background (Barbie, et al.; Systematic RNA interference reveals that oncogenic KRAS-driven cancers require TBK1; Nature, 462:108-112, 2009).

In view of the above, there is a clear need for compounds that selectively inhibit the kinase activities of IKKε, TBK1, or both IKKε and TBK1.

### BRIEF SUMMARY OF THE INVENTION

The present invention provides chemical compounds that selectively inhibit the kinase activities of IKKε, TBK1, or both IKKε and TBK1. Consequently, these compounds may be used in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

Specifically, the present invention provides compounds having structures according to Formula I (i.e., compounds according to Formula I):

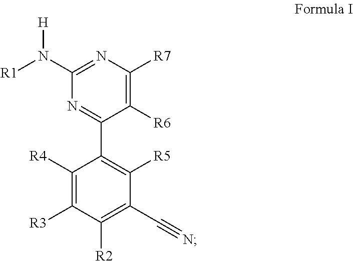

and pharmaceutically acceptable salts thereof, wherein R1, R2, R3, R4, R5, R6, and R7 are as defined below.

Specifically, the present invention provides compounds having structures according to Formula II (i.e., compounds according to Formula II):

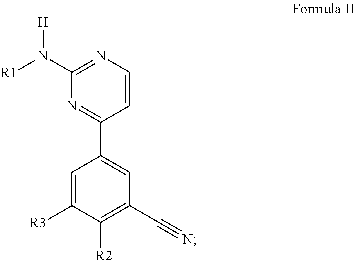

and pharmaceutically acceptable salts thereof; wherein R1, R2 and R3 are as defined herein below.

The compounds of the present invention include the compounds according to Formulae I and/or II as illustrated herein, as well as their geometric isomers, enantiomers, diastereomers, or racemates thereof. The compounds of the present invention also include the pharmaceutically acceptable salts of such compounds.

As noted above, the present invention provides chemical compounds that selectively inhibit the kinase activities of IKKε, TBK1, or both IKKε and TBK1, and therefore can be used in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. Thus, the present invention also provides methods for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, by administering to a patient in need of such treatment a therapeutically effective amount of a compound of the present invention, particularly a compound according to Formulae I and/or II, or a pharmaceutically acceptable salt thereof.

Also provided is the use of at least one of the compounds according to Formulae I and/or II for the manufacture of a medicament useful for therapy, including therapy for the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. In addition, the present invention also provides pharmaceutical compositions having at least one compound according to Formulae I and/or II and one or more pharmaceutically acceptable excipients. Further, methods for the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, by administering to a patient in need of such treatment, a pharmaceutical composition of the invention, are also encompassed.

In addition, the present invention also provides methods for treating or delaying the onset of the symptoms associated with inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. These methods comprise administering an effective amount of a compound of the present invention, generally in the form of a pharmaceutical composition or medicament, to an individual having, or at risk of having, inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

The compounds according to Formulae I and/or II may also be used in combination therapies. Thus, combination therapy methods are also provided for treating or delaying the onset of the symptoms associated with inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. Such methods comprise administering to a patient in need thereof a compound of the present invention and, together or separately, at least one other anti-cancer, anti-inflammation, anti-rheumatoid arthritis, anti-obesity, anti-insulin resistance, anti-metabolic syndrome, anti-type 2 diabetes, anti-SLE, or anti-psoriasis therapy.

For the convenience of combination therapy, the compound of the present invention may be administered together in the same formulation with another agent or therapeutic compound used for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer. Thus, the present invention also provides pharmaceutical compositions or medicaments for combination therapy, comprising an effective amount of at least one compound according to the present invention, and an effective amount of at least one other therapeutic agent or compound, which is different from the compounds according to Formulae I and/or II.

The foregoing and other advantages and features of the invention, and the manner in which they are accomplished, will become more readily apparent upon consideration of the following detailed description of the invention taken in conjunction with the accompanying examples, which illustrate embodiments of the present invention.

Unless otherwise defined, the technical and scientific terms used herein have the same meaning as commonly understood by one of ordinary skill in the art to which the present invention pertains. Although methods and materials similar or equivalent to those described herein may be used in the practice or testing of the present invention, suitable methods and materials are described below. In case of conflict, the present specification, including definitions, will control. In addition, the materials, methods, and examples are illustrative and not intended to be limiting.

Other features and advantages of the invention will be apparent to one of skill in the art from the following detailed description, and from the claims.

### DETAILED DESCRIPTION OF THE INVENTION

### 1. Definitions

As used herein, the terms “alkyl” or “alkyl group,” as employed herein alone or as part of another group refers to a saturated aliphatic hydrocarbon straight chain group having, unless otherwise specified, 1 to 20 carbon atoms (whenever it appears herein, a numerical range such as “1 to 20” refers to each integer in the given range; e.g., “1 to 20 carbon atoms” means that the alkyl group may consist of 1, 2, 3, 4, 5, 6, 7, 8, 9, 10, 11, 12, 13, 14, 15, 16, 17, 18, 19 or 20 carbon atoms), or a saturated aliphatic hydrocarbon branched chain group having 3, 4, 5, 6, 7, 8, 9, 10, 11, 12, 13, 14, 15, 16, 17, 18, 19 or 20 carbon atoms. An alkyl group may be optionally substituted with one or more substituents as valencies allow (generally one to three substitutents except in the case of halogen substituents, e.g., perchloro). As used herein, a C₁₋₆ alkyl group refers to an alkyl having 1, 2, 3, 4, 5, or 6 carbon atoms (e.g., including methyl, ethyl, propyl, isopropyl, butyl, sec-butyl, tert-butyl, 3-pentyl, and hexyl), which may be optionally substituted.

The term “lower alkyl” as used herein, refers to an alkyl group, as defined above, but containing 1, 2, 3, 4, 5, or 6 carbon atoms (i.e., a C₁₋₆ alkyl group).

The term “alkylene,” or “alkylene group,” as used herein means a saturated aliphatic hydrocarbon straight chain group having 1, 2, 3, 4, 5, 6, 7, 8, 9, 10, 11, 12, 13, 14, 15, 16, 17, 18, 19 or 20 carbon atoms or a saturated aliphatic hydrocarbon branched chain group having 3, 4, 5, 6, 7, 8, 9, 10, 11, 12, 13, 14, 15, 16, 17, 18, 19 or 20 carbon atoms having two connecting points. For example, an “ethylene” group represents the group —CH₂—CH₂—. Alkylene groups may also be optionally substituted with one or more substituents.

The term “alkenyl” as employed herein by itself or as part of another group means a straight chain radical of 2, 3, 4, 5, 6, 7, 8, 9, or 10 carbon atoms or a branched chain radical of 3, 4, 5, 6, 7, 8, 9, or 10 carbon atoms, unless the chain length is limited thereto, including at least one double bond between two of the carbon atoms in the chain. The alkenyl group may be optionally substituted with one or more substituents (generally one to three substitutents except in the case of halogen substituents, e.g., perchloro or perfluoroalkyls). For example, a C₃₋₆ alkenyl group refers to a straight or branched chain radical containing 3, 4, 5 or 6 carbon atoms and having at least one double bond between two of the carbon atoms in the chain (e.g., ethenyl, 1-propenyl, 2-propenyl, 2-methyl-1-propenyl, 1-butenyl and 2-butenyl, which may be optionally substituted).

The term “alkenylene” as used herein means an alkenyl group having two connecting points. For example, “ethenylene” represents the group —CH═CH—. Alkenylene groups may also be optionally substituted with one or more substituents.

The term “alkynyl” as used herein by itself or as part of another group means a straight chain radical of 2, 3, 4, 5, 6, 7, 8, 9, or 10 carbon atoms or branched chain radical of 4, 5, 6, 7, 8, 9, or 10 carbon atoms, unless the chain length is limited thereto, wherein there is at least one triple bond between two of the carbon atoms in the chain. The alkynyl group may be optionally substituted with one or more substituents as valencies allow (generally one to three substitutents except in the case of halogen substituents, e.g., perchloro or perfluoroalkyls). For example, a C₄₋₆ alkynyl group refers to a straight or branched chain radical containing 4, 5, or 6 carbon atoms and having at least one triple bond between two of the carbon atoms in the chain (e.g., ethynyl, 1-propynyl, 1-methyl-2-propynyl, 2-propynyl, 1-butynyl and 2-butynyl), which may be optionally substituted.

The term “alkynylene” as used herein means an alkynyl having two connecting points. For example, “ethynylene” represents the group —C≡C—. Alkynylene groups may also be optionally substituted with one or more substituents.

The term “carbocycle” as used herein by itself or as part of another group means cycloalkyl and non-aromatic partially saturated carbocyclic groups such as cycloalkenyl and cycloalkynyl. A carbocycle may be optionally substituted with one or more substituents so long as the resulting compound is sufficiently stable and suitable for the uses of the present invention.

The term “cycloalkyl” as used herein by itself or as part of another group refers to a fully saturated 3, 4, 5, 6, 7, or 8-membered cyclic hydrocarbon ring (i.e., a cyclic form of an alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkyl”). Thus, a cycloalkyl may exist as a monocyclic ring, bicyclic ring, or a spiral ring. When a cycloalkyl is referred to as a Cₓ cycloalkyl, this means a cycloalkyl in which the fully saturated cyclic hydrocarbon ring (which may or may not be fused to another ring) has x number of carbon atoms. When a cycloalkyl is recited as a substituent on a chemical entity, it is intended that the cycloalkyl moiety is attached to the entity through a carbon atom within the fully saturated cyclic hydrocarbon ring of the cycloalkyl. In contrast, a substituent on a cycloalkyl can be attached to any carbon atom of the cycloalkyl. A cycloalkyl group may be optionally substituted with one or more substitutents so long as the resulting compound is sufficiently stable and suitable for the uses of the present invention. Examples of cycloalkyl groups include cyclopropyl, cyclobutyl, cyclopentyl, cyclohexyl and cycloheptyl.

The term “cycloalkenyl” as used herein by itself or as part of another group refers to a non-aromatic partially saturated 3, 4, 5, 6, 7, or 8-membered cyclic hydrocarbon ring having at least one double bond therein (i.e., a cyclic form of an alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl may exist as a monocyclic ring, bicyclic ring, polycyclic or a spiral ring. When a cycloalkenyl is referred to as a Cₓ cycloalkenyl, this means a cycloalkenyl in which the non-aromatic partially saturated cyclic hydrocarbon ring (which may or may not be fused to another ring) has x number of carbon atoms. When a cycloalkenyl is recited as a substituent on a chemical entity, it is intended that the cycloalkenyl moiety is attached to the entity through a carbon atom within the non-aromatic partially saturated ring (having a double bond therein) of the cycloalkenyl. In contrast, a substituent on a cycloalkenyl can be attached to any carbon atom of the cycloalkenyl. A cycloalkenyl group may be optionally substituted with one or more substitutents. Examples of cycloalkenyl groups include cyclopentenyl, cycloheptenyl and cyclooctenyl.

The term “heterocycle” (or “heterocyclyl” or “heterocyclic”) as used herein by itself or as part of another group means a saturated or partially saturated 3, 4, 5, 6, or 7-membered non-aromatic cyclic ring formed with carbon atoms and from one to four heteroatoms independently chosen from O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of atoms with such other rings) (“polycyclic heterocycle”). Thus, a heterocycle may exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle may be optionally substituted with one or more substituents so long as the resulting compound is sufficiently stable and suitable for the uses of the present invention.

Some examples of saturated or partially saturated heterocyclic groups include tetrahydrofuranyl, pyranyl, tetrahydropyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with 6 or 8 carbon atoms in the ring (“monocylic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is referred to as a Cₓ aryl, this means an aryl in which the all-carbon aromatic ring (which may or may not be fused to another ring) has x number of carbon atoms. When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl may be optionally substituted with one or more substituents so long as the resulting compound is sufficiently stable and suitable for the uses of the present invention.

The term “heteroaryl” as employed herein refers to a stable aromatic ring having 5, 6 or 7 ring atoms with 1, 2, 3 or 4 hetero ring atoms in the ring which are oxygen, nitrogen or sulfur or a combination thereof (“monocylic heteroaryl”). In addition to monocyclic hetero aromatic rings, the term “heteroaryl” also encompasses a group having the monocyclic hetero aromatic ring above fused to another cycloalkyl, cycloalkynyl, cycloalkenyl, heterocycle, aryl or heteroaryl ring (i.e., sharing an adjacent pair of atoms with such other rings) (“polycyclic heteroaryl”). When a heteroaryl is recited as a substituent on a chemical entity, it is intended that the heteroaryl moiety is attached to the entity through an atom within the hetero aromatic ring of the heteroaryl. In contrast, a substituent on a heteroaryl can be attached to any suitable atom of the heteroaryl. A heteroaryl may be optionally substituted with one or more substituents so long as the resulting compound is sufficiently stable and suitable for the uses of the present invention.

Heteroaryl groups include, for example, thienyl (thiophenyl), including without limitation 2-thienyl, benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, including without limitation imidazol-4-yl, and imidazol-5-yl, pyrazolyl, including without limitation pyrazol-4-yl, and pyrazol-5-yl, pyridyl (pyridinyl), including without limitation 2-pyridyl, 3-pyridyl, and 4-pyridyl, pyrazinyl, including without limitation pyrazin-3-yl, pyrimidinyl, including without limitation pyrimidin-2-yl, pyrimidin-4-yl, and pyrimidin-5-yl, pyridazinyl, including without limitation pyridazinyl-3-yl, and pyridazinyl-4-yl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, oxazolyl, including without limitation oxazol-2-yl, isoxazolyl, including without limitation isoxazol-5-yl, thiazolyl, including without limitation thiazol-2-yl, triazolyl, including without limitation 1,2,4-triazol-3-yl furazanyl, phenoxazinyl, 1,4-dihydroquinoxaline-2,3-dione, 7-amino-isocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

As used herein, the term “halo” refers to fluoro, chloro, bromo, or iodo substitutents.

As used herein, the term “hydro” refers to a bound hydrogen (i.e., an —H group).

As used herein, the term “hydroxyl” refers to an —OH group.

As used herein, the term “alkoxy” refers to an —O-(alkyl). Lower alkoxy refers to —O— (lower alkyl) groups.

As used herein, the term “alkenyloxy” refers to an —O-(alkenyl).

As used herein, the term “alkynyloxy” refers to an —O-(alkenyl).

As used herein, the term “cycloalkyloxy” refers to an —O-cycloakyl group.

As used herein, the term “heterocycloxy” refers to an —O-heterocycle group.

As used herein, the term “mercapto” group refers to an —SH group.

The term “alkylthio” group refers to an —S-alkyl group.

The term “arylthio” group refers to an —S-aryl group.

The term “arylalkyl” is used herein to mean an alkyl group, as defined above, substituted with an aryl group, as defined above. Examples of arylalkyl groups include benzyl, phenethyl and naphthylmethyl, etc. An arylalkyl group may be optionally substituted with one or more substituents so long as the resulting compound is sufficiently stable and suitable for the uses of the present invention.

The term “heteroarylalkyl” is used herein to mean an alkyl group, as defined above, substituted with a heteroaryl group, as defined above. A heteroarylalkyl may be optionally substituted with one or more substituents so long as the resulting compound is sufficiently stable and suitable for the uses of the present invention.

The term “arylalkynyl” is used herein to mean any of the above-defined alkynyl groups substituted with any of the above-defined aryl groups.

The term “heteroarylalkenyl” is used herein to mean any of the above-defined alkenyl groups substituted with any of the above-defined heteroaryl groups.

The term “aryloxy” is used herein to mean aryl-O— or —O-aryl wherein aryl is as defined above. Aryloxy groups include phenoxy and 4-methylphenoxy.

The term “heteroaryloxy” is used herein to mean heteroaryl-O— or —O-heteroaryl wherein heteroaryl is as defined above.

The term “arylalkoxy” is used herein to mean an alkoxy group substituted with an aryl group as defined above. Arylalkoxy groups include benzyloxy and phenethyloxy.

“Heteroarylalkoxy” is used herein to mean any of the above-defined alkoxy groups substituted with any of the above-defined heteroaryl groups.

“Haloalkyl” means an alkyl group that is substituted with one or more fluorine, chlorine, bromine or iodine atoms. Haloalkyl groups include, for example, fluoromethyl, difluoromethyl, trifluoromethyl, pentafluoroethyl, 1,1-difluoroethyl, chloromethyl, chlorofluoromethyl and trichloromethyl groups.

As used herein, the term “oxo” refers to an oxygen atom double bonded to another atom (i.e., “═O”).

As used herein, the term “carbonyl” group refers to a —C(═O)R″ group, where R″ is chosen from hydro, alkyl, cycloalkyl, aryl, heteroaryl (bonded through a ring carbon) and heterocyclyl (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)-group.

As used herein, the term “thiocarbonyl” group refers to a —C(═S)R″ group, with R″ as defined herein. “Alkylthiocarbonyl” refers to an alkyl-C(═S)— group.

“Alkanoyl,” as used herein, refers to an alkyl-C(═O)— group.

As used herein the term “acetyl” group refers to a —C(═O)CH₃ group.

As used herein term “heterocyclonoyl” group refers to a heterocyclocarbonyl, or heterocyclo-C(═O)— group.

The term “heterocycloketone,” as used herein refers to a heterocycle group in which one of the carbon atoms which form the ring has an oxygen double-bonded to it—i.e., one of the ring carbon atoms is a —C(═O)— group.

As used herein the term “O-carboxy” group refers to a R″C(═O)O— group, where R″ is as defined herein.

The term “C-carboxy” group, as used herein, refers to a —C(═O)OR″ groups where R″ is as defined herein.

As used herein, the term “carboxylic acid” refers to a C-carboxy group in which R″ is hydro. In other words, the term “carboxylic acid” refers to —COOH.

As used herein, the term “ester” is a C-carboxy group, as defined herein, wherein R″ is as defined above, except that it is not hydro. Example ester groups include, methyl ester, ethyl ester, propyl ester, and lower alkyl ester).

As used herein, the term “C-carboxy salt” refers to a —C(═O)O⁻M⁺ group wherein M⁺ is chosen from lithium, sodium, magnesium, calcium, potassium, barium, iron, zinc and quaternary ammonium.

The term “carboxyalkyl,” as used herein, refers to —C₁₋₆ alkylene-C(═O)OR″ (that is, a C₁₋₆ alkyl group connected to the core structure wherein the alkyl group is substituted with —C(═O)OR″ with R″ being defined herein). Examples of carboxyalkyl include, but are not limited to, —CH₂COOH, —(CH₂)₂COOH, —(CH₂)₃COOH, —(CH₂)₄COOH, and —(CH₂)₅COOH.

“Carboxyalkenyl” refers to -alkenylene-C(═O)OR″ with R″ being defined herein.

The term “carboxyalkyl salt” refers to a —(CH₂)ᵣC(═O)O⁻M⁺ wherein M⁺ is chosen from lithium, sodium, potassium, calcium, magnesium, barium, iron, zinc and quaternary ammonium, wherein r is 1, 2, 3, 4, 5, or 6.

The term “carboxyalkoxy” refers to —O—(CH₂)ᵣC(═O)OR″ wherein r is 1, 2, 3, 4, 5, or 6, and R″ is as defined herein.

“Cₓ carboxyalkanoyl” means a carbonyl group (—C(═O)—) attached to an alkyl or cycloalkylalkyl group that is substituted with a carboxylic acid or carboxyalkyl group, wherein the total number of carbon atom is x (an integer of 2 or greater).

“Cₓ carboxyalkenoyl” means a carbonyl group (—C(═O)—) attached to an alkenyl or alkyl or cycloalkylalkyl group that is substituted with a carboxylic acid or carboxyalkyl or carboxyalkenyl group, wherein at least one double bond (—CH═CH—) is present and wherein the total number of carbon atom is x (an integer of 2 or greater).

“Carboxyalkoxyalkanoyl” means refers to R″OC(═O)—C₁₋₆ alkylene-O—C₁₋₆ alkylene-C(═O)—, R″ is as defined herein.

As used herein, the term “heterocycloyl”, by itself or as part of another group, means a radical of formula heterocycle-C(═O)—.

“Amino” refers to an —NR^{x}R^{y} group, with R^{x} and R^{y} as defined herein.

“Alkylamino,” as used herein, means an amino group with at least one alkyl substituent.

“Aminoalkyl” means an alkyl group connected to the core structure of a molecule and having at least one amino substituent.

“Quaternary ammonium” refers to a —⁺N(R^{x})(R^{y})(R^{z}) group wherein R^{x}, R^{y}, and R^{z} are as defined herein.

The term “nitro” refers to a —NO₂ group.

As used herein the term “O-carbamyl” refers to a —OC(═O)N(R^{x})(R^{y}) group with R^{x} and R^{y} as defined herein.

The term “N-carbamyl,” as used herein, refers to a R^{y}OC(═O)N(R^{x})— group, with R^{x} and R^{y} as defined herein.

As used herein the term “O-thiocarbamyl” refers to a —OC(═S)N(R^{x})(R^{y}) group with R^{x} and R^{y} as defined herein.

The term “N-thiocarbamyl,” as used herein, refers to a R^{x}OC(═S)NR^{y}— group, with R^{x} and R^{y} as defined herein.

As used herein the term “C-amido” refers to a —C(═O)N(R^{x})(R^{y}) group with R^{x} and R^{y} as defined herein.

“N-amido,” as used herein, refers to a R^{x}C(═O)N(R^{y})— group with R^{x} and R^{y} as defined herein.

“Carbamoylamino” or “carbamide linker” are used alternatively herein to refer to a R″N(R^{y})C(═O)N(R^{x})— group with R^{x}, R^{y} and R″ as defined herein.

“Aminothiocarbonyl” refers to a —C(═S)N(R^{x})(R^{y}) group with R^{x} and R^{y} as defined herein.

“Hydroxyaminocarbonyl” means a —C(═O)N(R^{x})(OH) group with R^{x} as defined herein.

“Alkoxyaminocarbonyl” means a —C(═O)N(R^{x})(alkoxy) group with R^{x} as defined herein.

The terms “cyano,” “cyanyl,” and “nitrile” group, as used interchangably herein, refer to a —C≡N group.

The term “cyanato” refers to a —CNO group.

The term “isocyanato” refers to a —NCO group.

The term “thiocyanato” refers to a —CNS group.

The term “isothiocyanato” refers to a —NCS group.

The term “sulfinyl” refers to a —S(═O)R″ group, where R″ is as defined herein.

The term “sulfonyl” refers to a —S(═O)₂R″ group, where R″ is as defined herein.

The term “sulfonamide” or “sulfamoyl” are used interchangeably herein to refer to an —N(R^{x})—S(═O)₂R″ group, with R″ and R^{x} as defined herein.

“Aminosulfonyl” means (R^{x})(R^{y})N—S(═O)₂— with R^{x} and R^{y} as defined herein.

“Aminosulfonyloxy” means a (R^{x})(R^{y})N—S(═O)₂—O— group with R^{x} and R^{y} as defined herein.

“Sulfonamidecarbonyl” means R″—S(═O)₂—N(R^{x})—C(═O)— with R″ and R^{x} as defined herein.

“Alkanoylaminosulfonyl” refers to an alkyl-C(═O)—N(R^{x})—S(═O)₂— group with R^{x} as defined herein.

The term “trihalomethylsulfonyl” refers to a X₃CS(═O)₂— group with X being halo.

The term “trihalomethylsulfonamide” refers to a X₃CS(═O)₂N(R^{x})— group with X being halo and R^{x} as defined herein.

R″ is chosen from hydro, alkyl, cycloalkyl, aryl, heteroaryl and heterocycle, each being optionally substituted.

R^{x}, R^{y}, and R^{z} are independently chosen from hydro and optionally substituted alkyl.

The term “bioisostere”, as used herein, generally refers to compounds or moieties that have chemical and physical properties producing broadly similar biological properties. Examples of carboxylic acid bioisosteres include, but are not limited to, carboxyalkyl, carboxylic acid ester, tetrazole, oxadiazole, isoxazole, hydroxythiadiazole, thiazolidinedione, oxazolidinedione, sulfonamide, aminosulfonyl, sulfonamidecarbonyl, C-amido, sulfonylcarboxamide, phosphonic acid, phosphonamide, phosphinic acid, sulfonic acid, alkanoylaminosulfonyl, mercaptoazole, trifluoromethylcarbonyl, and cyanamide.

“Pharmaceutical composition” refers to at least one compound and a pharmaceutically acceptable vehicle, with which the compound is administered to a patient.

“Pharmaceutically acceptable vehicle” refers to a diluent, adjuvant, excipient or carrier with which a compound is administered.

“Patient” includes humans. The terms “human” and “patient” are used interchangeably herein.

“Preventing” or “prevention” refers to a reduction in risk of acquiring a disease or disorder (i.e., causing at least one of the clinical symptoms of the disease not to develop in a patient that may be predisposed to the disease but does not yet experience or display symptoms of the disease).

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

Unless specifically stated otherwise or indicated by a bond symbol (dash, double dash, or triple dash, etc.), the point at which a recited substituent group connects to the remainder of the molecule will be via the right-most stated moiety. Further, the names of chemical moieties, as defined above, can simply be linked together to identify more complex substituent groups. In such instances, the point at which the recited complex substituent is connected to the remainder of the molecule will be through the right-most stated moiety. Thus, for example, a “hydroxyalkyl” group is connected to the remainder of the molecule through the alkyl moiety while the hydroxyl is a substituent on the alkyl. Similarly, for example, a “heterocycloalkyl” group is connected to the remainder of the molecule through the alkyl moiety while the heterocycle is a substituent on the alkyl.

In most instances names for the compounds disclosed were generated in accordance with International Union of Pure and Applied Chemistry (IUPAC) conventions using Advanced Chemistry Development, Inc., (ACD/Labs) (Toronto, Ontario, Canada) ACD/Name IUPAC nomenclature software release 12.00, version 12.01. In some cases, however, names for compounds and synthetic intermediates were generated using the IUPAC naming feature supplied with either the Symyx® Draw package, version 3.2 or 3.3, available from Symyx Technologies, Inc. (Santa Clara, Calif.), or the Autonom 2000 plug-in for the Isis™/Draw 2.5 SP1 chemical drawing program, formerly available from MDL Information Systems, a division of Symyx Technologies, Inc. (Santa Clara, Calif.). In all cases, if there is a conflict between a name and a structure when a structure is provided along with a name, the structure is to be taken as ultimately defining the compound being described.

### 2. Compounds of the Present Invention

The present invention provides chemical compounds that selectively inhibit the kinase activities of IKKε and/or TBK1, and particularly compounds that selectively inhibit the kinase activities of IKKε and/or TBK1 over the kinase activities of IKKα and IKKIβ. Consequently, these compounds may be used in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

Specifically, the present invention provides compounds having structures according to Formula I (i.e., compounds according to Formula I):

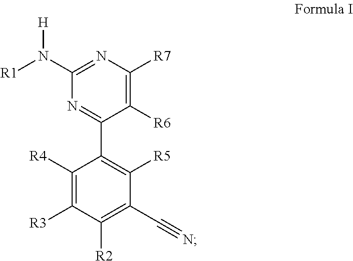

and pharmaceutically acceptable salts thereof, wherein:

R1 is optionally-substituted heteroaryl, optionally-substituted heterocyclyl; optionally-substituted heteroarylalkylene, optionally-substituted heterocycloalkylene, optionally-substituted heteroarylalkenylene, optionally-substituted heterocycloalkenylene, optionally-substituted heteroarylalkynylene, or optionally-substituted heterocycloalkynylene;

R2 is chosen from alkyl, alkenyl, alkynyl, carbocycle, cycloalkyl, cycloalkenyl, heterocyclyl, aryl, heteroaryl, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, alkyl-N-amido, cycloalkyl-N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide, wherein any of the foregoing groups are optionally substituted one or more times with alkyl, alkenyl, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide; and

R3, R4, R5, R6, and R7 are each independently chosen from alkyl, alkenyl, alkynyl, carbocycle, cycloalkyl, cyclo alkenyl, heterocyclyl, aryl, heteroaryl, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, alkyl-N-amido, cycloalkyl-N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide, wherein any of the foregoing groups are optionally substituted one or more times with alkyl, alkenyl, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide;

with the proviso that when R3, R4, R5, R6, and R7, are all hydro, then R2 is not heterocyclyl bonded to the phenyl ring through a nitrogen atom of the heterocyclyl; and

with the proviso that the compound is NOT:
Benzonitrile, 5-[2-(1H-benzimidazol-6-ylamino)-4-pyrimidinyl]-2-[(tetrahydro-2H-pyran-4-yl)oxy]-;
Benzonitrile, 5-[2-(1,3-benzodioxol-5-ylamino)-4-pyrimidinyl]-2-[(tetrahydro-2H-pyran-4-yl)oxy]-;
Benzonitrile, 5-[2-(6-benzothiazolylamino)-4-pyrimidinyl]-2-[(tetrahydro-2H-pyran-4-yl)oxy]-; or
Benzonitrile, 5-[2-(5-benzothiazolylamino)-4-pyrimidinyl]-2-[(tetrahydro-2H-pyran-4-yl)oxy]-.

In some embodiments of the compounds of Formula I, R1 is selected from heteroaryl, heterocyclo; heteroarylalkylene, heterocycloalkylene, heteroarylalkenylene, heterocycloalkenylene, heteroarylalkynylene, and heterocycloalkynylene, wherein any of the foregoing groups are optionally substituted one or more times with alkyl, alkenyl, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide.

In some embodiments of the compounds of Formula I, R1 is selected from heteroaryl and heterocyclyl; wherein either of the foregoing groups is optionally substituted one or more times with alkyl, alkenyl, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide.

In some embodiments of the compounds of Formula I, R3, R4, R5, R6, and R7 are each independently selected from hydro, halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, and sulfinyl.

In some embodiments of the compounds of Formula I, R4, R5, R6, and R7 are each hydro.

In some embodiments of the compounds of Formula I, R3 is hydro or methoxy.

Specifically, the present invention provides compounds having structures according to Formula II (i.e., compounds according to Formula II):

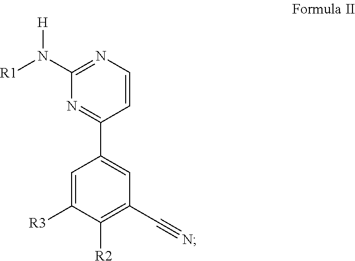

and pharmaceutically acceptable salts thereof,

wherein R1 is an optionally-substituted 5 or 6-membered heteroaryl group comprising from one to three heteroatoms independently chosen from nitrogen (N), oxygen (O), and sulfur (S);

R2 is chosen from an optionally substituted C₁₋₄ alkoxyl, optionally substituted heterocycloxyl, optionally substituted cycloalkylalkoxyl, optionally substituted heterocycloalkoxyl, optionally substituted C₁₋₄ alkyl-N-amido, and optionally substituted cycloalkyl-N-amido; and

R3 is hydro or methoxy.

In particular embodiments of Formulae I and/or II, R1 is an optionally substituted six-membered heteroaryl group comprising one or two nitrogens. In some of such embodiments R1 can be an optionally substituted pyridyl, pyridazinyl, pyrimidinyl, or pyrazinyl, group. Specifically, in some of such embodiments R1 is an optionally substituted 2-pyridyl, 3-pyridyl, 4-pyridyl, 3-pyridazinyl, 4-pyridazinyl, 2-pyrimidinyl, 4-pyrimidinyl, 5-pyrimidinyl, or 2-pyrazinyl group.

In other embodiments of Formulae I and/or II, R1 is an optionally substituted five-membered heteroaryl group comprising one, two, or three nitrogens. In some of such embodiments R1 can be an optionally substituted pyrazolyl, imidazolyl, thienyl, oxazolyl, isoxazolyl, thiozolyl or triazolyl group. Specifically, in some of such embodiments R1 is an optionally substituted 4-pyrazolyl, 5-pyrazolyl, 4-imidazolyl, 5-imidazolyl, or 3-triazolyl group.

In other embodiments of Formulae I and/or II, R1 is an optionally substituted five-membered heteroaryl group comprising one, two, or three heteroatoms independently chosen from N, O and S. In some of such embodiments R1 can be an optionally substituted thienyl, oxazolyl, isoxazolyl, or thiozolyl group. Specifically, in some of such embodiments R1 is an optionally substituted 2-thienyl, 2-oxazolyl, 5-isoxazolyl, or 2-thiozolyl group.

In some embodiments of Formulae I and/or II the heteroaryl group or heterocyclyl group of R1 is substituted and the substituent is attached to a ring carbon of the heteroaryl or heterocyclyl group. In some of such embodiments, the substituent is chosen from: halo, methoxyl, ethoxyl, trihalomethyl, hydroxyl, hydroxylalkyl, C₁-C₄ alkyl, C-carboxyl, carbocyclyl, 4-6 membered heterocyclyl, heterocyclylalkyl, heterocyclylalkenyl, heterocyclonoyl, heterocyclonoylalkyl, heteroaryl, amino, aminoalkyl, N-amido, N-amidoalkyl, sulfamoylalkyl, C-amido, and N-amidoalkyl. In some of such embodiments, the substituent is optionally further substituted with halo, hydroxyl, hydroxylalkyl, methoxyl, ethoxyl, alkoxyalkoxyl, C₁-C₄ alkyl, hydroylated C₁-C₄ alkyl, amino, alkoxyamino, heterocyclyl, sulfonyl, hydroylated heterocyclyl, or aminated heterocyclyl group.

In particular embodiments Formulae I and/or II, R1 is chosen from

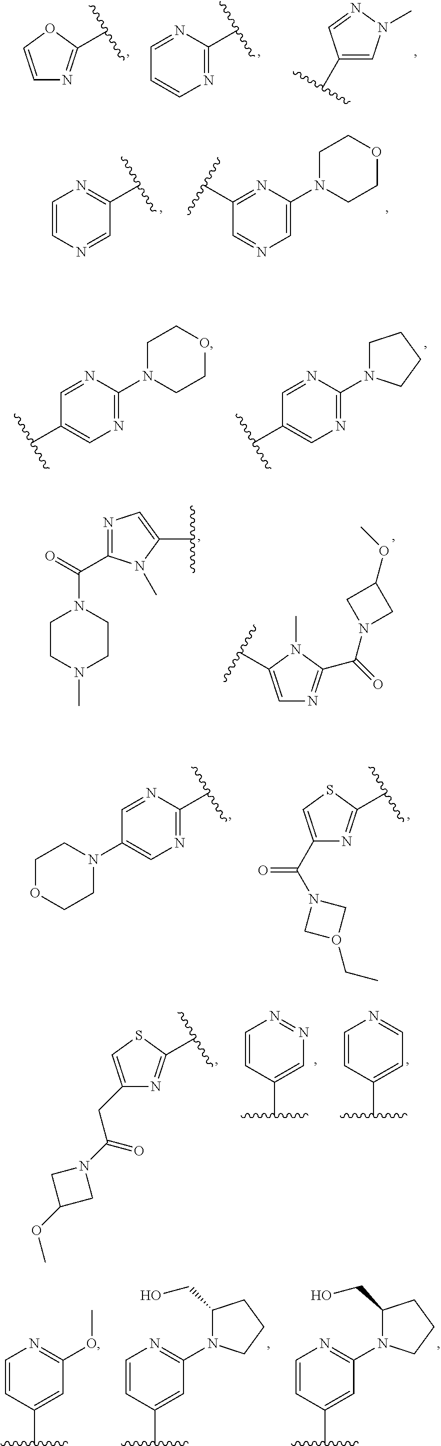

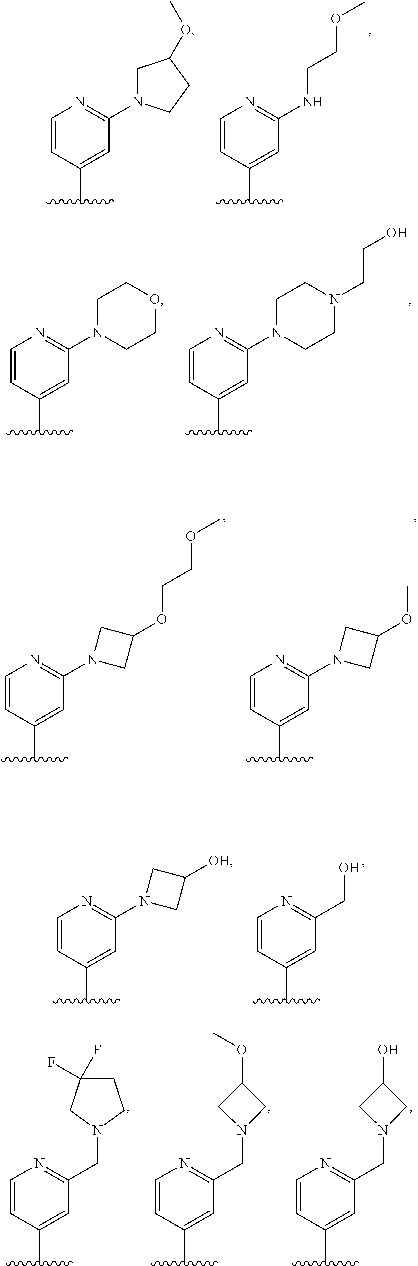

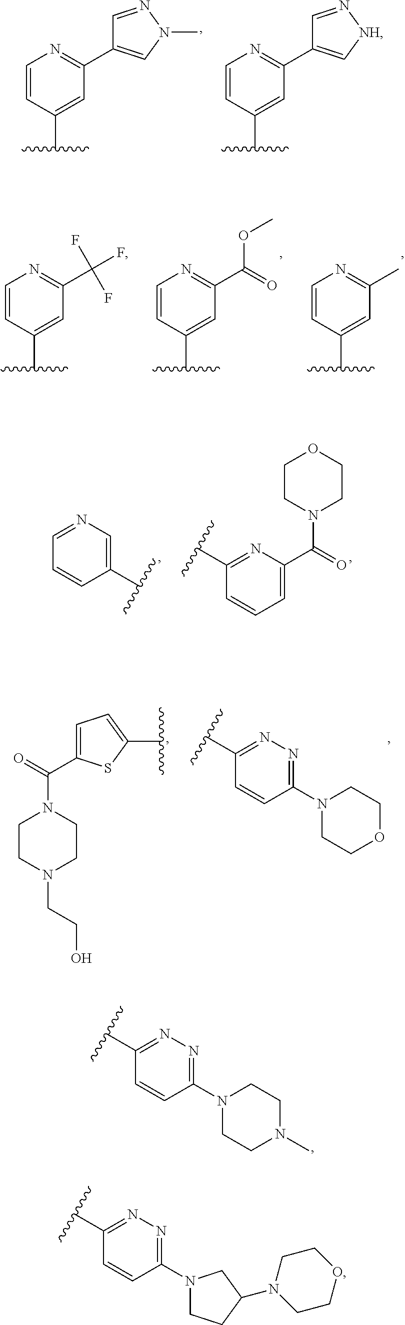

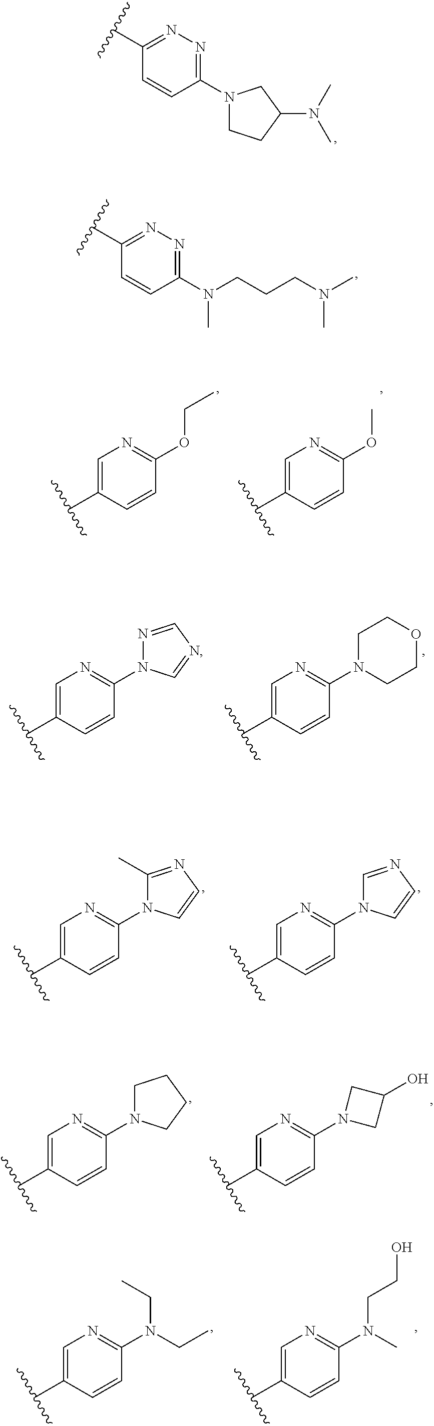

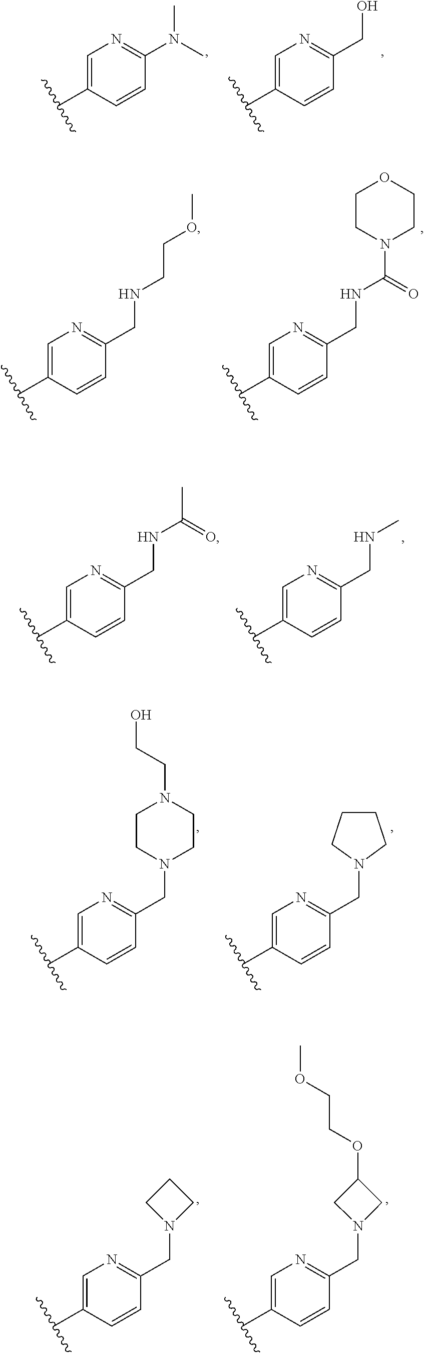

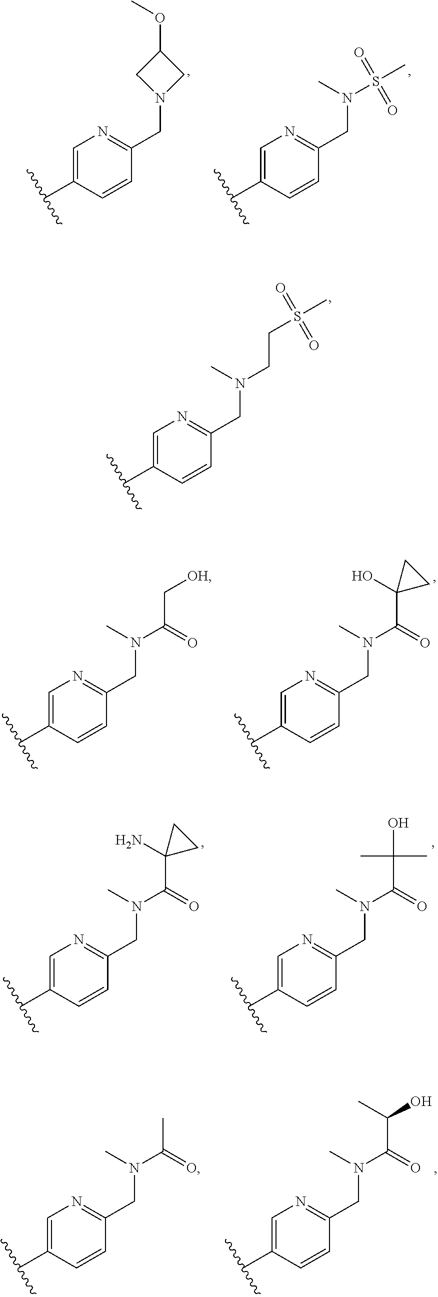

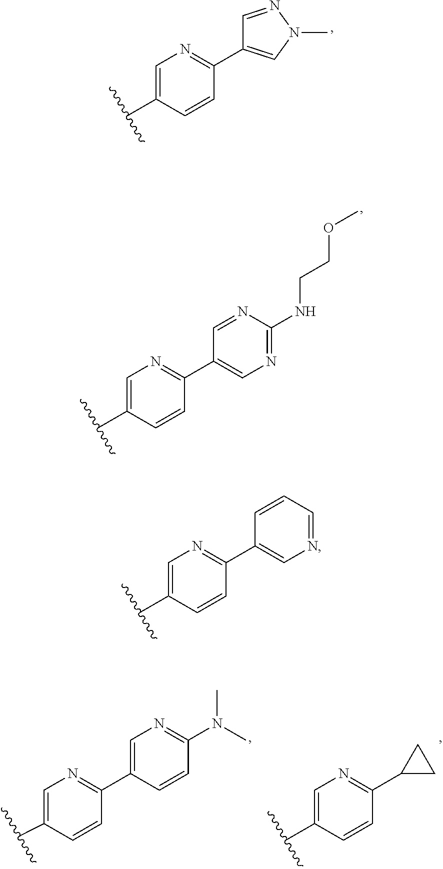 [SMILES: *c1ccc(-c2cnn(C)c2)nc1 ; *c1ccc(-c2cnc(NCCOC)nc2)nc1 ; *c1ccc(-c2cccnc2)nc1 ; *c1ccc(-c2ccc(N(C)C)nc2)nc1 ; *c1ccc(C2CC2)nc1]

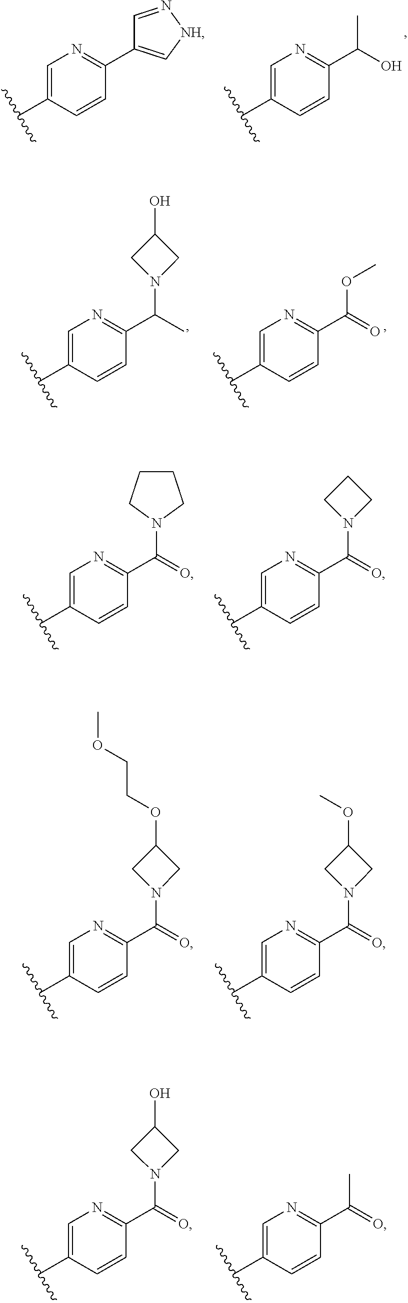

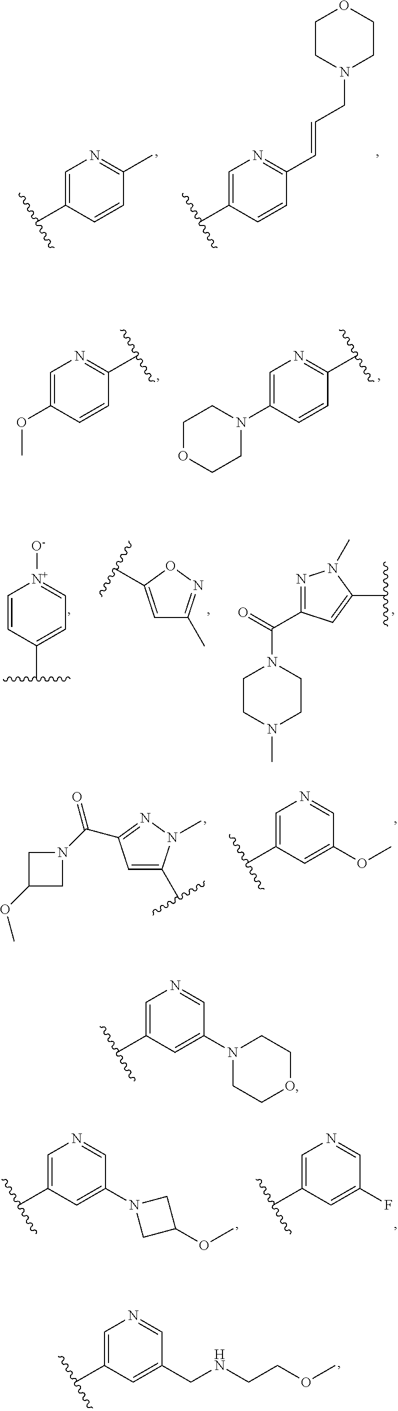

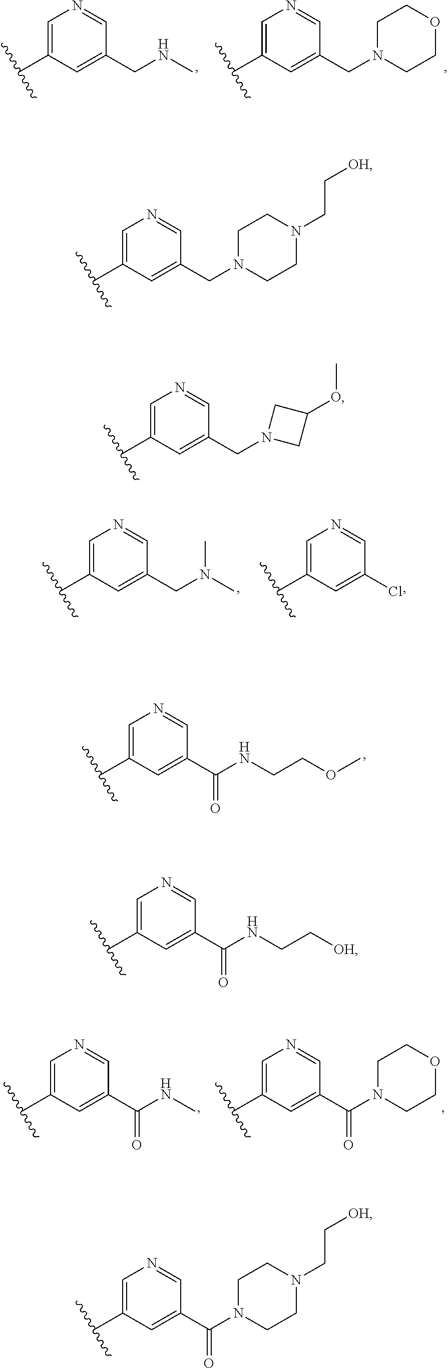

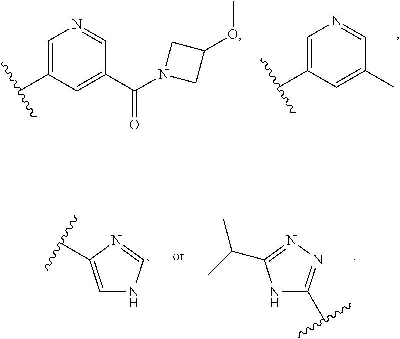

In particular embodiments of Formulae I and/or II, R2 is an optionally substituted tetrahydropyran-4-yloxyl, cyclopropanecarbonylamino, pyrrolidin-3-yloxyl, 2-methylpropanoylamino, 4-piperidyloxyl, cyclopropylmethoxyl, methoxyl, (3-methyloxetan-3-yl)methoxyl, isobutoxyl, or methyl group.

In those embodiments of Formulae I and/or II in which R2 is substituted, the substituent is a hydroxy-C₁-C₄ alkanoyl.

In specific embodiments of Formulae I and/or II in which R2 is substituted pyrrolidin-3-yloxyl or 4-piperidyloxyl, the substitutent is chosen from 2-hydroxyethanoyl (2-hydroxyacetyl) or 2-hydroxypropanoyl, including stereoisomers (2R)-2-hydroxypropanoyl, and (2S)-2-hydroxypropanoyl.

In particular embodiments of Formulae I and/or 11, R2 is chosen from

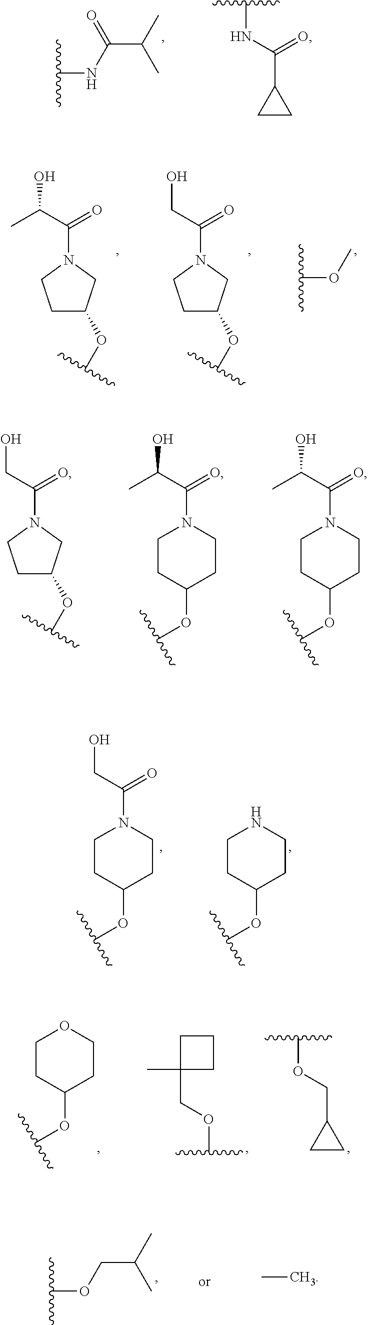

In particular embodiments of Formulae I and/or II, R3 is either hydro or methoxy group. In some embodiments of Formulae I and/or II R3 is hydro.

In particular embodiments a compound according to Formulae I and/or II is chosen from:
5-[2-({6-[(2-Hydroxyethyl)(methyl)amino]pyridin-3-yl}amino)pyrimidin-4-yl]-2-(tetrahydro-2H-pyran-4-yloxy)benzonitrile;
5-(2-{[2-(Morpholin-4-yl)pyrimidin-5-yl]amino}pyrimidin-4-yl)-2-(tetrahydro-2H-pyran-4-yloxy)benzonitrile;
5-(2-{[2-(Pyrrolidin-1-yl)pyrimidin-5-yl]amino}pyrimidin-4-yl)-2-(tetrahydro-2H-pyran-4-yloxy)benzonitrile;
5-{2-[(6-Cyclopropylpyridin-3-yl)amino]pyrimidin-4-yl}-2-(tetrahydro-2H-pyran-4-yloxy)benzonitrile;
5-(2-{[6-(Pyrrolidin-1-yl)pyridin-3-yl]amino}pyrimidin-4-yl)-2-(tetrahydro-2H-pyran-4-yloxy)benzonitrile;
5-(2-{[6-(Morpholin-4-yl)pyridin-3-yl]amino}pyrimidin-4-yl)-2-(tetrahydro-2H-pyran-4-yloxy)benzonitrile;
5-{2-[(6-Methylpyridin-3-yl)amino]pyrimidin-4-yl}-2-(tetrahydro-2H-pyran-4-yloxy)benzonitrile;
5-{2-[(6-Ethoxypyridin-3-yl)amino]pyrimidin-4-yl}-2-(tetrahydro-2H-pyran-4-yloxy)benzonitrile;
5-(2-{[6-(Diethylamino)pyridin-3-yl]amino}pyrimidin-4-yl)-2-(tetrahydro-2H-pyran-4-yloxy)benzonitrile;
5-(2-{[6-(Dimethylamino)pyridin-3-yl]amino}pyrimidin-4-yl)-2-(tetrahydro-2H-pyran-4-yloxy)benzonitrile;
5-[2-(Pyridin-3-ylamino)pyrimidin-4-yl]-2-(tetrahydro-2H-pyran-4-yloxy)benzonitrile;
5-({4-[3-Cyano-4-(tetrahydro-2H-pyran-4-yloxy)phenyl]pyrimidin-2-yl}amino)-N-methylpyridine-3-carboxamide;
5-({4-[3-Cyano-4-(tetrahydro-2H-pyran-4-yloxy)phenyl]pyrimidin-2-yl}amino)-N-(2-hydroxyethyl)pyridine-3-carboxamide;
5-{2-[(5-{[4-(2-Hydroxyethyl)piperazin-1-yl]carbonyl}pyridin-3-yl)amino]pyrimidin-4-yl}-2-(tetrahydro-2H-pyran-4-yloxy)benzonitrile;
5-({4-[3-Cyano-4-(tetrahydro-2H-pyran-4-yloxy)phenyl]pyrimidin-2-yl}amino)-N-(2-methoxyethyl)pyridine-3-carboxamide;
5-(2-{[5-(Morpholin-4-ylcarbonyl)pyridin-3-yl]amino}pyrimidin-4-yl)-2-(tetrahydro-2H-pyran-4-yloxy)benzonitrile;
5-[2-({5-[(3-Methoxyazetidin-1-yl)carbonyl]pyridin-3-yl}amino)pyrimidin-4-yl]-2-(tetrahydro-2H-pyran-4-yloxy)benzonitrile;
5-[2-({5-[(Methylamino)methyl]pyridin-3-yl}amino)pyrimidin-4-yl]-2-(tetrahydro-2H-pyran-4-yloxy)benzonitrile;
5-[2-({5-[(Dimethylamino)methyl]pyridin-3-yl}amino)pyrimidin-4-yl]-2-(tetrahydro-2H-pyran-4-yloxy)benzonitrile;
5-{2-[(5-{[4-(2-Hydroxyethyl)piperazin-1-yl]methyl}pyridin-3-yl)amino]pyrimidin-4-yl}-2-(tetrahydro-2H-pyran-4-yloxy)benzonitrile;
5-(2-{[5-(Morpholin-4-ylmethyl)pyridin-3-yl]amino}pyrimidin-4-yl)-2-(tetrahydro-2H-pyran-4-yloxy)benzonitrile;
5-{2-[(5-{[(2-Methoxyethyl)amino]methyl}pyridin-3-yl)amino]pyrimidin-4-yl}-2-(tetrahydro-2H-pyran-4-yloxy)benzonitrile;
5-[2-({5-[(3-Methoxyazetidin-1-yl)methyl]pyridin-3-yl}amino)pyrimidin-4-yl]-2-(tetrahydro-2H-pyran-4-yloxy)benzonitrile;
5-{2-[(5-Methylpyridin-3-yl)amino]pyrimidin-4-yl}-2-(tetrahydro-2H-pyran-4-yloxy)benzonitrile;
5-{2-[(5-Fluoropyridin-3-yl)amino]pyrimidin-4-yl}-2-(tetrahydro-2H-pyran-4-yloxy)benzonitrile;
5-{2-[(5-Chloropyridin-3-yl)amino]pyrimidin-4-yl}-2-(tetrahydro-2H-pyran-4-yloxy)benzonitrile;
5-{2-[(5-Methoxypyridin-3-yl)amino]pyrimidin-4-yl}-2-(tetrahydro-2H-pyran-4-yloxy)benzonitrile;
5-{2-[(6-{[3-(2-Methoxyethoxy)azetidin-1-yl]carbonyl}pyridin-3-yl)amino]pyrimidin-4-yl}-2-(tetrahydro-2H-pyran-4-yloxy)benzonitrile;
5-(2-{[6-(Pyrrolidin-1-ylcarbonyl)pyridin-3-yl]amino}pyrimidin-4-yl)-2-(tetrahydro-2H-pyran-4-yloxy)benzonitrile;
5-(2-{[6-(Azetidin-1-ylcarbonyl)pyridin-3-yl]amino}pyrimidin-4-yl)-2-(tetrahydro-2H-pyran-4-yloxy)benzonitrile;
5-[2-({6-[(3-Methoxyazetidin-1-yl)carbonyl]pyridin-3-yl}amino)pyrimidin-4-yl]-2-(tetrahydro-2H-pyran-4-yloxy)benzonitrile;
5-[2-({6-[(3-Hydroxyazetidin-1-yl)carbonyl]pyridin-3-yl}amino)pyrimidin-4-yl]-2-(tetrahydro-2H-pyran-4-yloxy)benzonitrile;
Methyl 5-({-4-[3-cyano-4-(tetrahydro-2H-pyran-4-yloxy)phenyl]pyrimidin-2-yl}amino)pyridine-2-carboxylate;
5-[2-(Pyridin-4-ylamino)pyrimidin-4-yl]-2-(tetrahydro-2H-pyran-4-yloxy)benzonitrile;
5-{2-[(1-Oxidopyridin-4-yl)amino]pyrimidin-4-yl}-2-(tetrahydro-2H-pyran-4-yloxy)benzonitrile;
Methyl 4-({4-[3-cyano-4-(tetrahydro-2H-pyran-4-yloxy)phenyl]pyrimidin-2-yl}amino)pyridine-2-carboxylate;
5-[2-({2-[(2R)-2-(Hydroxymethyl)pyrrolidin-1-yl]pyridin-4-yl}amino)pyrimidin-4-yl]-2-(tetrahydro-2H-pyran-4-yloxy)benzonitrile;
5-[2-({2-[(2-Methoxyethyl)amino]pyridin-4-yl}amino)pyrimidin-4-yl]-2-(tetrahydro-2H-pyran-4-yloxy)benzonitrile;
5-(2-{[2-(3-Methoxypyrrolidin-1-yl)pyridin-4-yl]amino}pyrimidin-4-yl)-2-(tetrahydro-2H-pyran-4-yloxy)benzonitrile;
5-[2-({2-[(2S)-2-(Hydroxymethyl)pyrrolidin-1-yl]pyridin-4-yl}amino)pyrimidin-4-yl]-2-(tetrahydro-2H-pyran-4-yloxy)benzonitrile;
5-(2-{[2-(3-Hydroxyazetidin-1-yl)pyridin-4-yl]amino}pyrimidin-4-yl)-2-(tetrahydro-2H-pyran-4-yloxy)benzonitrile;
5-[2-({2-[4-(2-Hydroxyethyl)piperazin-1-yl]pyridin-4-yl}amino)pyrimidin-4-yl]-2-(tetrahydro-2H-pyran-4-yloxy)benzonitrile;
5-(2-{[2-(3-Methoxyazetidin-1-yl)pyridin-4-yl]amino}pyrimidin-4-yl)-2-(tetrahydro-2H-pyran-4-yloxy)benzonitrile;
5-(2-{[2-(Morpholin-4-yl)pyridin-4-yl]amino}pyrimidin-4-yl)-2-(tetrahydro-2H-pyran-4-yloxy)benzonitrile;
5-[2-({2-[3-(2-Methoxyethoxy)azetidin-1-yl]pyridin-4-yl}amino)pyrimidin-4-yl]-2-(tetrahydro-2H-pyran-4-yloxy)benzonitrile;
5-(2-{[6-(3-Hydroxyazetidin-1-yl)pyridin-3-yl]amino}pyrimidin-4-yl)-2-(tetrahydro-2H-pyran-4-yloxy)benzonitrile;
5-(2-{[6-(Azetidin-1-ylmethyl)pyridin-3-yl]amino}pyrimidin-4-yl)-2-(tetrahydro-2H-pyran-4-yloxy)benzonitrile;
5-[2-({6-[(3-Methoxyazetidin-1-yl)methyl]pyridin-3-yl}amino)pyrimidin-4-yl]-2-(tetrahydro-2H-pyran-4-yloxy)benzonitrile;
5-{2-[(6-{[3-(2-Methoxyethoxy)azetidin-1-yl]methyl}pyridin-3-yl)amino]pyrimidin-4-yl}-2-(tetrahydro-2H-pyran-4-yloxy)benzonitrile;
5-{2-[(6-{[(2-Methoxyethyl)amino]methyl}pyridin-3-yl)amino]pyrimidin-4-yl}-2-(tetrahydro-2H-pyran-4-yloxy)benzonitrile;
5-(2-{[6-(Pyrrolidin-1-ylmethyl)pyridin-3-yl]amino}pyrimidin-4-yl)-2-(tetrahydro-2H-pyran-4-yloxy)benzonitrile;
5-[2-({6-[(Methylamino)methyl]pyridin-3-yl}amino)pyrimidin-4-yl]-2-(tetrahydro-2H-pyran-4-yloxy)benzonitrile;
N-{[5-({4-[3-Cyano-4-(tetrahydro-2H-pyran-4-yloxy)phenyl]pyrimidin-2-yl}amino)pyridin-2-yl]methyl}-N-methylacetamide;
5-(2-{[6-({Methyl[2-(methylsulfonyl)ethyl]amino}methyl)pyridin-3-yl]amino}pyrimidin-4-yl)-2-(tetrahydro-2H-pyran-4-yloxy)benzonitrile;
N-{[5-({4-[3-Cyano-4-(tetrahydro-2H-pyran-4-yloxy)phenyl]pyrimidin-2-yl}amino)pyridin-2-yl]methyl}-N-methylmethanesulfonamide;
N-{[5-({4-[3-Cyano-4-(tetrahydro-2H-pyran-4-yloxy)phenyl]pyrimidin-2-yl}amino)pyridin-2-yl]methyl}-2-hydroxy-N,2-dimethylpropanamide;
(2R)—N-{[5-({4-[3-Cyano-4-(tetrahydro-2H-pyran-4-yloxy)phenyl]pyrimidin-2-yl}amino)pyridin-2-yl]methyl}-2-hydroxy-N-methylpropanamide;
N-{[5-({4-[3-Cyano-4-(tetrahydro-2H-pyran-4-yloxy)phenyl]pyrimidin-2-yl}amino)pyridin-2-yl]methyl}-2-hydroxy-N-methylacetamide;
N-{[5-({4-[3-Cyano-4-(tetrahydro-2H-pyran-4-yloxy)phenyl]pyrimidin-2-yl}amino)pyridin-2-yl]methyl}-1-hydroxy-N-methylcyclopropanecarboxamide;
1-Amino-N-{[5-({4-[3-cyano-4-(tetrahydro-2H-pyran-4-yloxy)phenyl]pyrimidin-2-yl}amino)pyridin-2-yl]methyl}-N-methylcyclopropanecarboxamide;
5-(2-{[6-(1-Hydroxyethyl)pyridin-3-yl]amino}pyrimidin-4-yl)-2-(tetrahydro-2H-pyran-4-yloxy)benzonitrile;
5-{2-[(6-Acetylpyridin-3-yl)amino]pyrimidin-4-yl}-2-(tetrahydro-2H-pyran-4-yloxy)benzonitrile;
5-[2-({6-[1-(3-Hydroxyazetidin-1-yl)ethyl]pyridin-3-yl}amino)pyrimidin-4-yl]-2-(tetrahydro-2H-pyran-4-yloxy)benzonitrile;
5-(2-{[5-(3-Methoxyazetidin-1-yl)pyridin-3-yl]amino}pyrimidin-4-yl)-2-(tetrahydro-2H-pyran-4-yloxy)benzonitrile;
5-(2-{[5-(Morpholin-4-yl)pyridin-3-yl]amino}pyrimidin-4-yl)-2-(tetrahydro-2H-pyran-4-yloxy)benzonitrile;
5-(2-{[2-(Hydroxymethyl)pyridin-4-yl]amino}pyrimidin-4-yl)-2-(tetrahydro-2H-pyran-4-yloxy)benzonitrile;
N-{[5-({4-[3-Cyano-4-(tetrahydro-2H-pyran-4-yloxy)phenyl]pyrimidin-2-yl}amino)pyridin-2-yl]methyl}morpholine-4-carboxamide;
N-{[5-({4-[3-Cyano-4-(tetrahydro-2H-pyran-4-yloxy)phenyl]pyrimidin-2-yl}amino)pyridin-2-yl]methyl}acetamide;
5-[2-({2-[(3-Methoxyazetidin-1-yl)methyl]pyridin-4-yl}amino)pyrimidin-4-yl]-2-(tetrahydro-2H-pyran-4-yloxy)benzonitrile;
5-[2-({2-[(3-Hydroxyazetidin-1-yl)methyl]pyridin-4-yl}amino)pyrimidin-4-yl]-2-(tetrahydro-2H-pyran-4-yloxy)benzonitrile;
5-[2-({2-[(3,3-Difluoropyrrolidin-1-yl)methyl]pyridin-4-yl}amino)pyrimidin-4-yl]-2-(tetrahydro-2H-pyran-4-yloxy)benzonitrile;
5-(2-{[6-(Morpholin-4-ylcarbonyl)pyridin-2-yl]amino}pyrimidin-4-yl)-2-(tetrahydro-2H-pyran-4-yloxy)benzonitrile;
5-(2-{[6-(2-Methyl-1H-imidazol-1-yl)pyridin-3-yl]amino}pyrimidin-4-yl)-2-(tetrahydro-2H-pyran-4-yloxy)benzonitrile;
5-(2-{[5-(Morpholin-4-yl)pyrimidin-2-yl]amino}pyrimidin-4-yl)-2-(tetrahydro-2H-pyran-4-yloxy)benzonitrile;
5-(2-{[6-(1H-Imidazol-1-yl)pyridin-3-yl]amino}pyrimidin-4-yl)-2-(tetrahydro-2H-pyran-4-yloxy)benzonitrile;
2-(Tetrahydro-2H-pyran-4-yloxy)-5-(2-{[6-(1H-1,2,4-triazol-1-yl)pyridin-3-yl]amino}pyrimidin-4-yl)benzonitrile;
5-(2-{[6-(1-Methyl-1H-pyrazol-4-yl)pyridin-3-yl]amino}pyrimidin-4-yl)-2-(tetrahydro-2H-pyran-4-yloxy)benzonitrile;
5-(2-{[6-(1H-Pyrazol-4-yl)pyridin-3-yl]amino}pyrimidin-4-yl)-2-(tetrahydro-2H-pyran-4-yloxy)benzonitrile;
5-[2-(2,3′-Bipyridin-5-ylamino)pyrimidin-4-yl]-2-(tetrahydro-2H-pyran-4-yloxy)benzonitrile;
5-{2-[(6-{2-[(2-Methoxyethyl)amino]pyrimidin-5-yl}pyridin-3-yl)amino]pyrimidin-4-yl}-2-(tetrahydro-2H-pyran-4-yloxy)benzonitrile;
5-(2-{[6′-(Dimethylamino)-2,3′-bipyridin-5-yl]amino}pyrimidin-4-yl)-2-(tetrahydro-2H-pyran-4-yloxy)benzonitrile;
5-(2-{[2-(1-Methyl-1H-pyrazol-4-yl)pyridin-4-yl]amino}pyrimidin-4-yl)-2-(tetrahydro-2H-pyran-4-yloxy)benzonitrile;
5-(2-{[2-(1H-Pyrazol-4-yl)pyridin-4-yl]amino}pyrimidin-4-yl)-2-(tetrahydro-2H-pyran-4-yloxy)benzonitrile;
5-[2-({6-[3-(Morpholin-4-yl)pyrrolidin-1-yl]pyridazin-3-yl}amino)pyrimidin-4-yl]-2-(tetrahydro-2H-pyran-4-yloxy)benzonitrile;
5-(2-{[6-(4-Methylpiperazin-1-yl)pyridazin-3-yl]amino}pyrimidin-4-yl)-2-(tetrahydro-2H-pyran-4-yloxy)benzonitrile;
5-{2-[(6-{[3-(Dimethylamino)propyl](methyl)amino}pyridazin-3-yl)amino]pyrimidin-4-yl}-2-(tetrahydro-2H-pyran-4-yloxy)benzonitrile;
5-[2-({6-[3-(Dimethylamino)pyrrolidin-1-yl]pyridazin-3-yl}amino)pyrimidin-4-yl]-2-(tetrahydro-2H-pyran-4-yloxy)benzonitrile;
5-(2-{[6-(Morpholin-4-yl)pyridazin-3-yl]amino}pyrimidin-4-yl)-2-(tetrahydro-2H-pyran-4-yloxy)benzonitrile;
5-(2-{[6-(Morpholin-4-yl)pyrazin-2-yl]amino}pyrimidin-4-yl)-2-(tetrahydro-2H-pyran-4-yloxy)benzonitrile;
5-[2-(Pyrimidin-2-ylamino)pyrimidin-4-yl]-2-(tetrahydro-2H-pyran-4-yloxy)benzonitrile;
5-[2-(Pyridazin-4-ylamino)pyrimidin-4-yl]-2-(tetrahydro-2H-pyran-4-yloxy)benzonitrile;
5-[2-(Pyrazin-2-ylamino)pyrimidin-4-yl]-2-(tetrahydro-2H-pyran-4-yloxy)benzonitrile;
5-(2-{[5-(Morpholin-4-yl)pyridin-2-yl]amino}pyrimidin-4-yl)-2-(tetrahydro-2H-pyran-4-yloxy)benzonitrile;
5-[2-({6-[(1E)-3-(Morpholin-4-yl)prop-1-en-1-yl]pyridin-3-yl}amino)pyrimidin-4-yl]-2-(tetrahydro-2H-pyran-4-yloxy)benzonitrile;
5-{2-[(5-{[4-(2-Hydroxyethyl)piperazin-1-yl]carbonyl}thiophen-2-yl)amino]pyrimidin-4-yl}-2-(tetrahydro-2H-pyran-4-yloxy)benzonitrile;
5-{2-[(3-Methyl-1,2-oxazol-5-yl)amino]pyrimidin-4-yl}-2-(tetrahydro-2H-pyran-4-yloxy)benzonitrile;
5-{2-[(1-Methyl-1H-pyrazol-4-yl)amino]pyrimidin-4-yl}-2-(tetrahydro-2H-pyran-4-yloxy)benzonitrile;
5-[2-(1,3-oxazol-2-ylamino)pyrimidin-4-yl]-2-(tetrahydro-2H-pyran-4-yloxy)benzonitrile;
5-[2-(1H-Imidazol-4-ylamino)pyrimidin-4-yl]-2-(tetrahydro-2H-pyran-4-yloxy)benzonitrile;
5-[2-({3-[(3-Methoxyazetidin-1-yl)carbonyl]-1-methyl-1H-pyrazol-5-yl}amino)pyrimidin-4-yl]-2-(tetrahydro-2H-pyran-4-yloxy)benzonitrile;
5-[2-({1-Methyl-3-[(4-methylpiperazin-1-yl)carbonyl]-1H-pyrazol-5-yl}amino)pyrimidin-4-yl]-2-(tetrahydro-2H-pyran-4-yloxy)benzonitrile;
5-[2-({2-[(3-Methoxyazetidin-1-yl)carbonyl]-1-methyl-1H-imidazol-5-yl}amino)pyrimidin-4-yl]-2-(tetrahydro-2H-pyran-4-yloxy)benzonitrile;
5-[2-({1-Methyl-2-[(4-methylpiperazin-1-yl)carbonyl]-1H-imidazol-5-yl}amino)pyrimidin-4-yl]-2-(tetrahydro-2H-pyran-4-yloxy)benzonitrile;
5-[2-({4-[(3-Methoxyazetidin-1-yl)carbonyl]-1,3-thiazol-2-yl}amino)pyrimidin-4-yl]-2-(tetrahydro-2H-pyran-4-yloxy)benzonitrile;
5-[2-({4-[2-(3-Methoxyazetidin-1-yl)-2-oxoethyl]-1,3-thiazol-2-yl}amino)pyrimidin-4-yl]-2-(tetrahydro-2H-pyran-4-yloxy)benzonitrile;
2-Methoxy-5-{2-[(2-methoxypyridin-4-yl)amino]pyrimidin-4-yl}benzonitrile;
2-Methoxy-5-{2-[(5-methoxypyridin-2-yl)amino]pyrimidin-4-yl}benzonitrile;
2-Methoxy-5-{2-[(6-methoxypyridin-3-yl)amino]pyrimidin-4-yl}benzonitrile;
5-[2-({6-[(2-Hydroxyethyl)(methyl)amino]pyridin-3-yl}amino)pyrimidin-4-yl]-2-(2-methylpropoxy)benzonitrile;
2-(Cyclopropylmethoxy)-5-{2-[(6-ethoxypyridin-3-yl)amino]pyrimidin-4-yl}benzonitrile;
2-(Cyclopropylmethoxy)-5-[2-({6-[(2-hydroxyethyl)(methyl)amino]pyridin-3-yl}amino)pyrimidin-4-yl]benzonitrile;
2-(Cyclopropylmethoxy)-5-{2-[(6-methylpyridin-3-yl)amino]pyrimidin-4-yl}benzonitrile;
2-[(3-Methyloxetan-3-yl)methoxy]-5-{2-[(6-methylpyridin-3-yl)amino]pyrimidin-4-yl}benzonitrile;
3-Methoxy-5-{2-[(6-methylpyridin-3-yl)amino]pyrimidin-4-yl}-2-(tetrahydro-2H-pyran-4-yloxy)benzonitrile;
5-(2-{[6-(Azetidin-1-ylmethyl)pyridin-3-yl]amino}pyrimidin-4-yl)-3-methoxy-2-(tetrahydro-2H-pyran-4-yloxy)benzonitrile;
3-Methoxy-5-[2-({6-[(3-methoxyazetidin-1-yl)methyl]pyridin-3-yl}amino)pyrimidin-4-yl]-2-(tetrahydro-2H-pyran-4-yloxy)benzonitrile;
5-(2-{[6-(Diethylamino)pyridin-3-yl]amino}pyrimidin-4-yl)-2-{[(3R)-1-(hydroxyacetyl)pyrrolidin-3-yl]oxy}benzonitrile;
2-{[(3R)-1-(Hydroxyacetyl)pyrrolidin-3-yl]oxy}-5-{2-[(6-methylpyridin-3-yl)amino]pyrimidin-4-yl}benzonitrile;
2-{[(3R)-1-(Hydroxyacetyl)pyrrolidin-3-yl]oxy}-5-(2-{[6-(morpholin-4-yl)pyridin-3-yl]amino}pyrimidin-4-yl)benzonitrile;
2-({(3R)-1-[(2S)-2-Hydroxypropanoyl]pyrrolidin-3-yl}oxy)-5-{2-[(6-methylpyridin-3-yl)amino]pyrimidin-4-yl}benzonitrile;
2-{[(3R)-1-(Hydroxyacetyl)pyrrolidin-3-yl]oxy}-5-{2-[(2-methoxypyridin-4-yl)amino]pyrimidin-4-yl}benzonitrile;
2-{[(3R)-1-(Hydroxyacetyl)pyrrolidin-3-yl]oxy}-5-(2-{[2-(trifluoromethyl)pyridin-4-yl]amino}pyrimidin-4-yl)benzonitrile;
2-{[(3R)-1-(Hydroxyacetyl)pyrrolidin-3-yl]oxy}-5-{2-[(2-methylpyridin-4-yl)amino]pyrimidin-4-yl}benzonitrile;
5-(2-{[6-(Dimethylamino)pyridin-3-yl]amino}pyrimidin-4-yl)-2-{[1-(hydroxyacetyl)pyrrolidin-3-yl]oxy}benzonitrile;
2-{[(3R)-1-(Hydroxyacetyl)pyrrolidin-3-yl]oxy}-5-[2-({6-[(3-methoxyazetidin-1-yl)methyl]pyridin-3-yl}amino)pyrimidin-4-yl]benzonitrile;
2-{[(3R)-1-(Hydroxyacetyl)pyrrolidin-3-yl]oxy}-5-(2-{[2-(1-methyl-1H-pyrazol-4-yl)pyridin-4-yl]amino}pyrimidin-4-yl)benzonitrile;
2-({1-[(2S)-2-Hydroxypropanoyl]piperidin-4-yl}oxy)-5-(2-{[6-(morpholin-4-yl)pyridin-3-yl]amino}pyrimidin-4-yl)benzonitrile;
2-({1-[(2S)-2-Hydroxypropanoyl]piperidin-4-yl}oxy)-5-{2-[(6-methylpyridin-3-yl)amino]pyrimidin-4-yl}benzonitrile;
2-({1-[(2R)-2-Hydroxypropanoyl]piperidin-4-yl}oxy)-5-{2-[(6-methylpyridin-3-yl)amino]pyrimidin-4-yl}benzonitrile;
2-{[1-(Hydroxyacetyl)piperidin-4-yl]oxy}-5-{2-[(6-methylpyridin-3-yl)amino]pyrimidin-4-yl}benzonitrile;
2-{[1-(Hydroxyacetyl)piperidin-4-yl]oxy}-5-{2-[(2-methoxypyridin-4-yl)amino]pyrimidin-4-yl}benzonitrile;
N-[2-Cyano-4-(2-{[2-(pyrrolidin-1-yl)pyrimidin-5-yl]amino}pyrimidin-4-yl)phenyl]-2-methylpropanamide;
N-{2-Cyano-4-[2-({6-[(2-hydroxyethyl)(methyl)amino]pyridin-3-yl}amino)pyrimidin-4-yl]phenyl}-2-methylpropanamide;
N-[2-Cyano-4-(2-{[6-(morpholin-4-yl)pyridin-3-yl]amino}pyrimidin-4-yl)phenyl]-2-methylpropanamide;
N-[2-Cyano-4-(2-{[6-(pyrrolidin-1-yl)pyridin-3-yl]amino}pyrimidin-4-yl)phenyl]-2-methylpropanamide;
N-(2-Cyano-4-{2-[(6-methylpyridin-3-yl)amino]pyrimidin-4-yl}phenyl)-2-methylpropanamide;
N-(2-Cyano-4-{2-[(6-ethoxypyridin-3-yl)amino]pyrimidin-4-yl}phenyl)-2-methylpropanamide;
N-[2-Cyano-4-(2-{[6-(dimethylamino)pyridin-3-yl]amino}pyrimidin-4-yl)phenyl]cyclopropanecarboxamide;
N-[2-Cyano-4-(2-{[6-(diethylamino)pyridin-3-yl]amino}pyrimidin-4-yl)phenyl]cyclopropanecarboxamide;
N-(2-Cyano-4-{2-[(6-cyclopropylpyridin-3-yl)amino]pyrimidin-4-yl}phenyl)cyclopropanecarboxamide;
N-[2-Cyano-4-(2-{[6-(morpholin-4-yl)pyridin-3-yl]amino}pyrimidin-4-yl)phenyl]cyclopropanecarboxamide;
N-[2-Cyano-4-(2-{[2-(3-methoxyazetidin-1-yl)pyridin-4-yl]amino}pyrimidin-4-yl)phenyl]cyclopropanecarboxamide;
N-{2-Cyano-4-[2-({2-[4-(2-hydroxyethyl)piperazin-1-yl]pyridin-4-yl}amino)pyrimidin-4-yl]phenyl}cyclopropanecarboxamide;
N-[2-Cyano-4-(2-{[6-(hydroxymethyl)pyridin-3-yl]amino}pyrimidin-4-yl)phenyl]cyclopropanecarboxamide;
N-(2-Cyano-4-{2-[(6-{[4-(2-hydroxyethyl)piperazin-1-yl]methyl}pyridin-3-yl)amino]pyrimidin-4-yl}phenyl)cyclopropanecarboxamide;
N-{2-Cyano-4-[2-({6-[(3-methoxyazetidin-1-yl)methyl]pyridin-3-yl}amino)pyrimidin-4-yl]phenyl}cyclopropanecarboxamide;
N-(2-Cyano-4-{2-[(6-{[3-(2-methoxyethoxy)azetidin-1-yl]methyl}pyridin-3-yl)amino]pyrimidin-4-yl}phenyl)cyclopropanecarboxamide;
N-(2-Cyano-4-{2-[(6-{[(2-methoxyethyl)amino]methyl}pyridin-3-yl)amino]pyrimidin-4-yl}phenyl)cyclopropanecarboxamide;
N-(2-Cyano-4-{2-[(6-methylpyridin-3-yl)amino]pyrimidin-4-yl}phenyl)cyclopropanecarboxamide;
5-(2-{[3-(Propan-2-yl)-1H-1,2,4-triazol-5-yl]amino}pyrimidin-4-yl)-2-(tetrahydro-2H-pyran-4-yloxy)benzonitrile; or
   a compound selected from Table 2.

Further description of exemplary compounds according to Formulae I and/or II are provided in the Examples section below, in the form of over two hundred specific example compounds made.

For therapeutic use, salts of the compounds according to Formulae I and/or II are those wherein the counterion is pharmaceutically acceptable. However, salts of acids and bases which are non-pharmaceutically acceptable may also find use, for example, in the preparation or purification of a pharmaceutically acceptable compound.

The pharmaceutically acceptable addition salts as mentioned herein are meant to comprise the therapeutically active non-toxic acid addition salt forms which the compounds according to Formulae I and/or II are able to form. The latter can be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds according to Formulae I and/or II containing acidic protons may be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanediol, hydrabamine salts, and salts with amino acids such as, for example, arginine, lysine and the like. Conversely, the salt form can be converted by treatment with acid into the free acid form.

The term addition salt also comprises the hydrates and solvent addition forms which the compounds according to Formulae I and/or II are able to form. Examples of such forms are e.g. hydrates, alcoholates and the like.

The term “quaternary amine” as used herein defines the quaternary ammonium salts which the compounds according to Formulae I and/or II are able to form by reaction between a basic nitrogen of a compound according to Formulae I and/or II and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate, among others. The counterion of choice can be introduced using ion exchange resins.

Pharmaceutically acceptable salts of the compound of the present invention include all salts and are exemplified by alkaline salts with an inorganic acid or a salt with an organic acid that are known in the art. In addition, pharmaceutically acceptable salts include acid salts of inorganic bases, as well as acid salts of organic bases. Their hydrates, solvates, and the like are also encompassed in the present invention. In addition, N-oxide compounds are also encompassed in the present invention.

It will be appreciated that some of the compounds according to Formulae I and/or II and their N-oxides, addition salts, quaternary amines and stereochemically isomeric forms may contain one or more centers of chirality and exist as stereochemically isomeric forms.

The term “stereochemically isomeric forms” as used hereinbefore defines all possible stereoisomeric forms which the compounds according to Formulae I and/or II, and their N-oxides, addition salts, quaternary amines or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of the compounds according to Formulae I and/or II and their N-oxides, salts, solvates or quaternary amines substantially free, i.e. associated with less than about 10%, less than about 5%, less than about 2% and less than about 1% of the other isomers. Stereogenic centers may have the R- or S-configuration; substituents on bivalent cyclic (partially) saturated radicals may have either the cis- or trans-configuration. Compounds encompassing double bonds can have an E- or Z-stereochemistry at said double bond. Stereochemically isomeric forms of the compounds according to Formulae I and/or II are fully intended to be embraced within the scope of the present invention.

The N-oxide forms of the compounds according to Formulae I and/or II are meant to comprise the compounds according to Formulae I and/or II wherein one or several nitrogen atoms are oxidized to the so-called N-oxide.

Some of the compounds according to Formulae I and/or II may also exist in their tautomeric form. Such forms, although not explicitly indicated in the above formulae, are intended to be included within the scope of the present invention.

Whenever used hereinafter, the term “compounds according to Formulae I and/or II” is meant to also include the N-oxide forms, salts, and quaternary amines, as well as the stereochemically isomeric forms of the compound according to Formulae I and/or II. Of particular interest are those compounds according to Formulae I and/or II that are stereochemically pure.

Some compounds according to Formulae I and/or II are provided having an IC₅₀, as determined in the in-vitro IKKε kinase inhibition assays as described below (i.e., In-Vitro IKKε and TBK1 Kinase Assays), ranging from about 490 nM to about 50 nM. Other compounds according to Formulae I and/or II are provided having an IC₅₀, as determined in the in-vitro IKKε kinase inhibition assays as described below, ranging from about 50 nM to about 5 nM. Other compounds according to Formulae I and/or II are provided having an IC₅₀, as determined in the in-vitro IKKε kinase inhibition assays as described below, of less than about 5 nM.

It is believed that compounds according to Formulae I and/or II and having an IKKε kinase inhibitory activity (IC₅₀ value) of less than about 0.005 μM (5 nM), as determined in the in-vitro IKKε kinase inhibition assays as described below, are sufficiently active for the uses disclosed hereinafter. These compounds include, for example, Example Compounds 4, 5, 7, 8, 9, 10, 11, 12, 13, 15, 21, 22, 23, 27, 29, 30, 32, 33, 34, 36, 37, 38, 39, 40, 41, 42, 43, 44, 45, 46, 48, 52, 53, 54, 55, 56, 57, 58, 59, 60, 61, 62, 63, 66, 67, 68, 69, 70, 71, 75, 76, 77, 78, 79, 80, 81, 82, 83, 87, 88, 89, 94, 95, 102, 104, 109, 111, 117, 119, 121, 123, 126, 127, 128, 130, 131, 138, 139, 141, 142, 143, and 149, as identified below.

It should also be understood that in the compounds according to Formulae I and/or II, reference to any bound hydrogen atom may also encompass a deuterium atom bound at the same position. Substitution of hydrogen atoms with deuterium atoms is conventional in the art. See, e.g., U.S. Pat. Nos. 5,149,820 & 7,317,039. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms dramatically slows the catabolism of the deuterated compound, relative to the non-deuterated compound, such that the deuterated compound exhibits a longer half-life in the bodies of individuals administered such compounds. This is particularly the case when the catabolism of the hydrogenated compound is mediated by cytochrome P450 systems. See Kushner et al., Can. J. Physiol. Pharmacol. (1999) 77:79-88.

### 3. Pharmaceutical Compositions and Formulations

The present invention also provides medicaments or pharmaceutical compositions comprising a therapeutically or prophylactically effective amount of at least one compound according to the present invention (i.e., at least one compound according to Formulae I and/or II). Particularly, the present invention also provides medicaments or pharmaceutical compositions comprising a therapeutically or prophylactically effective amount of at least one compound according to the present invention having an IKKε kinase inhibitory activity (IC50 value) of less than about 0.005 μM (5 nM), as determined in the in-vitro IKKε kinase inhibition assays as described below. These compounds include, for example, Example Compounds 4, 5, 7, 8, 9, 10, 11, 12, 13, 15, 21, 22, 23, 27, 29, 30, 32, 33, 34, 36, 37, 38, 39, 40, 41, 42, 43, 44, 45, 46, 48, 52, 53, 54, 55, 56, 57, 58, 59, 60, 61, 62, 63, 66, 67, 68, 69, 70, 71, 75, 76, 77, 78, 79, 80, 81, 82, 83, 87, 88, 89, 94, 95, 102, 104, 109, 111, 117, 119, 121, 123, 126, 127, 128, 130, 131, 138, 139, 141, 142, 143, and 149, as identified below.

Typically, therapeutic compounds, such as the compounds according to Formulae I and/or II, may be effective at an amount ranging from about 0.01 μg/kg to about 100 mg/kg per day based on total body weight of a human patient. The effective amount of a therapeutic compound in such a medicament or pharmaceutical formulation may be administered all at once and at one time, or may be divided into a number of smaller doses that are administered at predetermined intervals of time, or predetermined times of the day, for a specific duration of time or a specified number of days. The suitable dosage unit containing the effective amount of a therapeutic compound may, for each administration, range in total mass from about 1 μg to about 2000 mg, or may range from about 5 μg to about 1000 mg.

In the case of combination therapy, a therapeutically effective amount of one or more other therapeutically effective compounds can be administered in a separate pharmaceutical composition, or alternatively can be included in the pharmaceutical composition according to the present invention along with at least one compound according to Formulae I and/or II. The pharmacology and toxicology of many of such other therapeutically effective compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such other therapeutically effective compounds used in art can be equally applicable in the present invention.

It should be understood that the dosage ranges set forth above are exemplary and are not intended to limit the scope of the present invention. The therapeutically effective amount for each therapeutically effective compound may vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration of therapeutically effective compounds may be adjusted as the various factors change over time.

In the pharmaceutical compositions of the present invention, the one or more compounds according to Formulae I and/or II can be in any pharmaceutically acceptable salt form, as described above.

For oral administration, the one or more compounds according to Formulae I and/or II may be incorporated into a pharmaceutical formulation that includes one or more pharmaceutically acceptable vehicles, excipients or carriers such as binders, lubricants, disintegrating agents, and sweetening or flavoring agents, as known in the art. The formulation can be incorporated into enclosed gelatin capsules or compressed tablets. Capsules and tablets can be prepared using conventional techniques. The capsules and tablets may also be coated with various coatings known in the art to modify the flavors, tastes, colors, and shapes of the capsules and tablets. In addition, liquid carriers such as fatty oil may also be included in capsules.

Suitable oral formulations can also be in the form of suspensions, syrups, chewing gum, wafers, elixirs, and the like. If desired, conventional agents for modifying flavors, tastes, colors, and shapes of the various forms may also be included.

The compounds according to Formulae I and/or II can also be administered parenterally in the form of a preformed solution or suspension, or a solution or suspension prepared from a lyophilized form before use. In such formulations, pharmaceutically acceptable diluents or pharmaceutically acceptable carriers such as sterile water, saline and buffered saline can be used. Other conventional and pharmaceutically acceptable solvents, pH buffers, stabilizers, anti-bacterial agents, surfactants, and antioxidants can be included. The parenteral formulations may be stored in conventional containers such as vials and ampoules that may be sized for preparing or delivering single doses of the formulation.

Routes of topical administration include, but are not limited to, dermal, nasal, bucal, mucosal, ocular, rectal, or vaginal applications. For topical administration, the active compounds may be formulated into lotions, creams, ointments, gels, powders, pastes, sprays, suspensions, drops and aerosols. Thus, one or more thickening agents, humectants, and stabilizing agents may be included in the formulations. One form of topical administration is delivery by a transdermal patch. Methods for preparing transdermal patches are disclosed, e.g., in Brown, et al; Annual Review of Medicine, 39:221-229, 1988.

Subcutaneous implantation for sustained release of the one or more compounds according to Formulae I and/or II may also be a suitable route of administration. This entails surgical procedures for implanting an active compound in any suitable formulation into a subcutaneous space, e.g., beneath the anterior abdominal wall. See, e.g., Wilson et al.; J. Clin. Psych., 45:242-247, 1984. Hydrogels may be used as a carrier for the sustained release of the active compounds. Hydrogels are generally known in the art. They are typically made by crosslinking high molecular weight biocompatible polymers into a network, which swells in water to form a gel like material. For the therapeutic methods of the present invention, hydrogels that are biodegradable or biosorbable are preferred. See, e.g., Phillips et al.; J. Pharmaceut. Sci., 73:1718-1720, 1984.

The compounds according to Formulae I and/or II may also be conjugated to a water soluble non-immunogenic, non-peptidic, high molecular weight polymer to form a polymer conjugate. For example, one or more compounds according to Formulae I and/or II may be covalently linked to polyethylene glycol to form a conjugate. Typically, such a conjugate exhibits improved solubility, stability, and reduced toxicity and immunogenicity. Thus, when administered to a patient, the one or more compounds according to Formulae I and/or II in the conjugate can have a longer half-life in the body, and exhibit better efficacy. See generally, Burnham; Am. J. Hosp. Pharm., 15:210-218, 1994. PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). In some embodiments of the present invention the covalent linkage between the polymer and the therapeutic compound or the polymer itself is hydrolytically degradable under physiological conditions. Such conjugates represent a type of “prodrug” that may readily release the active compound inside the body. Controlled release of an active compound may also be achieved by incorporating the active ingredient into microcapsules, nanocapsules, or hydrogels, as generally known in the art.

Liposomes may also be used as carriers for the compounds according to Formulae I and/or II. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y., 1976.

The one or more compounds according to Formulae I and/or II may also be administered in combination with one or more other therapeutic compounds that synergistically treats or prevents the same symptoms or is effective for another disease or symptom for which the patient is being treated, so long as the one or more other therapeutic compounds does not interfere with, or adversely affect, the effects of the compounds according to Formulae I and/or II. Such other therapeutic compounds include, but are not limited to, anti-inflammation agents, antiviral agents, antibiotics, antifungal agents, antithrombotic agents, cardiovascular drugs, cholesterol-lowering agents, anti-cancer drugs, hypertension drugs, and the like.

### 4. Therapeutic Methods

a. Treating Inflammation

In view of the discovery that IKKε plays a central role in integrating signals induced by pro-inflammatory stimuli (Kravchenko et al.; J. Biol. Chem., 278:26612-26619, 2003); and that IKKε, along with TBK1, has been shown to be involved in maintaining macrophages in an activated inflammatory state following activation of the interferon response (Solis, et al.; Eur. J. Immunol.; 37:529-539, 2007); it is believed that inhibition of IKKε kinase activity, TBK1 kinase activity, or the kinase activities of both IKKε and TBK1 would be effective in treating inflammation resulting from a wide range of causes, including both systemic and chronic inflammation. Hence, the present invention provides methods of treating inflammation, and complications associated with inflammation, comprising administering a therapeutically effective amount of one or more IKKε and/or TBK1-inhibiting compounds according to Formulae I and/or II to a patient in need of such treatment.

b. Treating Rheumatoid Arthritis (RA)

In view of the discovery that IKKε, as part of a complex kinases, has been found to play a role in the synovial inflammation, extracellular matrix destruction and activation of the anti-viral program and innate immune response in RA (Sweeney et al.; J. Immunol., 174:6424-6430, 2005), it is believed that inhibition of IKKε and/or TBK1 kinase activity would be effective in treating RA. Consequently, the present invention provides methods of treating RA, and complications associated with RA, comprising administering a therapeutically effective amount of one or more IKKε and/or TBK1-inhibiting compounds according to Formulae I and/or II to a patient in need of such treatment.

c. Treating Systemic Lupus Erythematosus (SLE)

In view of the role of phosphorylated transcription factors IRF3 and IRF7 in mediating the upregulation of IFNα/β and associated type I interferon signature genes that is a hallmark of flare-ups of SLE symptoms in SLE patients, and further view of the roles of IKKε and TBK in respectively phosphorylating IFR3 and IRF7, it is believed that inhibition of IKKε and/or TBK activity might be provide an effective means to reduce the intensity and longevity of such flare-ups in patients suffering from SLE. Consequently, the present invention provides methods of treating SLE, and complications associated with SLE flare-ups, comprising administering a therapeutically effective amount of one or more IKKε and/or TBK1-inhibiting compounds according to Formulae I and/or II to a patient in need of such treatment.

d. Treating Diseases Associated with Aberrant Accumulation of Cytosolic Nucleic Acids: Sjögrens Syndrome, Aicardi-Goutières Syndrome, Certain Forms of Systemic Lupus Erythematosus, Chilblain Lupus, Retinal Vasculopathy and Cerebral Leukodystrophy (RVCL)

Sjögrens syndrome, Aicardi-Goutieres syndrome, certain forms of systemic lupus erythematosus, chilblain lupus, RVCL are commonly associated with mutations in at least one of the following genes: TREX1; RNASEH2B; RNASEH2C; RNASEH2A; and SAMHD1 (Crow and Rehwinkel; Aicardi-Goutières syndrome and related phenotypes: linking nucleic acid metabolism with autoimmunity; Hum. Mol. Genet., 18:130-136, 2009; Kavanagh, et al.; New roles for the major human 3′-5′ exonuclease TREX1 in human disease; Cell Cycle, 7:1718-1725, 2008). These proteins are involved in degrading nucleic acids that are aberrantly located in the cytosolic compartment. If nucleic acids accumulate in the cytosol and are recognized by DNA or RNA receptors (i.e., RIG-I, MDA5, DAI, and others) this recognition leads to type I interferon production and autoimmune disease. The TBK1 and IKKε kinases are part of the signal cascade that leads to type I interferon production through phosphorylation of IRF3 and/or IRF7, and NFκB transcription factors (Hornung and Latz; Intracellular DNA Recognition; Nat. Rev. Immunol., 10:123-130, 2010). As such, small molecule inhibitors of IKKε and/or TBK1 kinases are expected to block type I interferon expression and provide therapeutic benefits to patients who are unable to properly degrade aberrantly localized cytosolic nucleic acids. Consequently, the present invention provides methods of treating diseases associated with the abberent accumulation of cytosolic nucleic acids, including Sjögrens syndrome, Aicardi-Goutieres syndrome, certain forms of systemic lupus erythematosus, chilblain lupus, RVCL, and complications associated with these diseases, comprising administering a therapeutically effective amount of one or more IKKε and/or TBK1-inhibiting compounds according to Formulae I and/or II to a patient in need of such treatment.

e. Treating Systemic Sclerosis

Systemic sclerosis is an autoimmune disease that targets connective tissue. The immune abnormalities cause increased production of extracellular matrix proteins in skin and vascular tissues through the interactions of several cell types, including endothelial cells, lymphocytes, macrophages, and fibroblast cells. A recognized feature of this disease is an abnormal type I interferon-gene expression signature (Assassi, et al.; Systemic sclerosis and lupus: points in an interferon-mediated continuum; Arthritis Rheum., 62:589-598, 2010). As with other autoimmune diseases, the exact cause of systemic sclerosis is not completely understood, but inhibition of type I interferons and fibrogenic cytokines (e.g. TGF-β) through TLR3 pathway inhibition may be therapeutically useful (Farina, et al.; Poly(I:C) Drives Type I IFN- and TGFbeta-Mediated Inflammation and Dermal Fibrosis Simulating Altered Gene Expression in Systemic Sclerosis; J. Invest. Dermato., epub, Jul. 8, 2010). The IKKε and/or TBK1 kinases are essential for production of type I interferon and for TGF-β signaling through TLR3 receptor activation. Small molecule inhibitors of the IKKε & TBK1 kinases, such as the compounds according to Formulae I and/or II, may benefit patients suffering from systemic sclerosis. Consequently, the present invention provides methods of treating systemic sclerosis, and complications associated with systemic sclerosis, comprising administering a therapeutically effective amount of one or more IKKε and/or TBK1-inhibiting compounds according to Formulae I and/or II to a patient in need of such treatment.

f. Treating Dermatomyositis and Polymyositis—Subtypes of Myositis

Myositis describes a collection of several poorly defined autoimmune diseases represented by the most common subtypes; dermatomyositis, polymyocitis, and inclusion-body myositis. Production of autoantibodies that target unknown muscle tissue antigens result in muscle weakness and skin abnormalities (Dalakas; Immunotherapy of Myositis: Issues, Concerns and Future Prospects; Nat. Rev. Rheum., 6:129-137, 2010). A recently identified feature of dermatomyositis and polymyositis is an aberrent type I interferon-gene expression signature profile in both muscle and PBMC samples from diseased patients (Baechler, et al.; An Interferon Signature in the Peripheral Blood of Dermatomyositis Patients is Associated with Disease Activity; Mol. Med., 13:59-68, 2007). The interferon-gene signature results from elevated IFN-α/β cytokines that are aberrantly produced. The IKKε/TBK1 pathway is essential for the production of IFN-α/β proteins upon activation of TLR3, TLR4, and cytosolic nucleic acid receptors; RIG-I, MDA5, DAI, and others. It is expected that patients suffering from dermatomyositis and polymyocitis would benefit from treatment with small molecule IKKε and/or TBK1 inhibitors such as the compounds according to Formulae I and/or II. Consequently, the present invention provides methods of treating dermatomyositis and polymyocitis, and complications associated with these diseases, comprising administering a therapeutically effective amount of one or more IKKε and/or TBK1-inhibiting compounds according to Formulae I and/or II to a patient in need of such treatment.

g. Treating Psoriasis

In view of the fact that psoriasis is a chronic inflammatory skin disorder involving upregulation of interleukins IL-23, IL-17A and IL-22, and in further view of the discovery that IKKε plays a role in integrating signals induced by pro-inflammatory stimuli (Kravchenko et al.; J. Biol. Chem.; 278:26612-26619, 2003.); and that IKKε, along with TBK1, has been shown to play a role in maintaining macrophages in an activated, inflammatory state, following activation of the interferon response (Solis, et al.; Eur. J. Immunol.; 37:529-539, 2007); it is believed that inhibition of IKKε and TBK activity might provide an effective means to treating psoriasis. Consequently, the present invention provides methods of treating psoriasis, and complications associated with psoriasis, comprising administering a therapeutically effective amount of one or more IKKε and/or TBK1-inhibiting compounds according to Formulae I and/or II to a patient in need of such treatment.

h. Treating Chronic Obstructive Pulmonary Disease (COPD)

COPD is characterized by chronic inflammation of the lungs and narrowing of the airways often caused by cigarette smoke (Churg, et al.; Mechanisms of cigarette smoke-induced COPD: Insights from animal models; Am. J. Physiol. Lung Cell. Mol. Physiol., 294:612-631, 2008). Viral and bacterial infections exacerbate the chronic inflammation in patients with COPD and result in approximately 120,000 deaths each year. Pulmonary infections can be recognized by nucleic acid receptors that activate IKKε/TBK1 signaling, leading to proinflammatory chemokine secretion of RANTES, IP-10 and IL-8. These chemokines recruit a variety of proinflammatory cells, including T-cells, eosinophils, basophils, neutrophils, natural killer and dendritic cells, to lungs. Recruitment of proinflammatory cells to the lungs results in lung tissue damage. Eosinophils and T cells play a primary role in causing tissue damage due to their release of cytotoxic proteins and proteases. Inhibition of the IKKε/TBK1 pathway is likely to have therapeutic benefits in Asthma and COPD patients. Consequently, the present invention provides methods of treating COPD, and complications associated with COPD, comprising administering a therapeutically effective amount of one or more IKKε and/or TBK1-inhibiting compounds according to Formulae I and/or II to a patient in need of such treatment.

i. Treating Inflammatory Bowel Disease (IBD)

IBD is an autoimmune-like disorder characterized by chronic inflammation of the intestinal mucosal tissue. The gut is an immunologically unique organ, which must protect the host from pathogens while being tolerant to dietary antigens and essential commensal bacteria. The intestinal wall is therefore an actively regulated barrier. IBD is characterized by a dysregulated immune response to commensal bacteria in genetically susceptible patients. Toll-like receptor (TLR) transmembrane proteins are a central component of the intestinal bacterial surveillance system expressed by intestinal epithelial cells, T cells, antigen-presenting macrophages, and dendritic cells. TLRs have been genetically implicated in IBD based on the identification of single-nucleotide polymorphisms in a number of TLRs (TLR1, 2, 4, 6, and 9) that are associated with increase disease susceptibility or extent of disease in IBD patients (Cario; Toll-like Receptors in Inflammatory Bowel Diseases: A Decade Later; Inflamm. Bowel Dis., 16:1583-1597, 2010). TLR4 is upregulated in IBD, whereas in normal intraepithelial cells it is expressed at such low levels as to be undetectable. TLR4 is a bacterial lipopolysaccharide-recognizing receptor, and one of the outputs from the TLR4 receptor signaling complex involves IKKε and/or TBK1 kinases. This pathway directs the activation of the transcription factor IRF3 via phosphorylation by IKKε and/or TBK1 kinase, which induces expression of proinflammatory chemokines RANTES and MCP1. Modulation of overactive TLR4 signaling, via inhibition of the IKKε/TBK1 signaling pathway by a compound of the present invention may have therapeutic benefit to IBD patients. Consequently, the present invention provides methods of treating IBD, and complications associated with IBD, comprising administering a therapeutically effective amount of one or more IKKε and/or TBK1-inhibiting compounds according to Formulae I and/or II to a patient in need of such treatment.

j. Treating Obesity, Insulin Resistance, Type 2 Diabetes (NIDDM), and Metabolic Syndrome

In view of the discovery that IKKε knockout mice were protected from high-fat diet-induced obesity, chronic inflammation in liver and fat, hepatic steatosis, and whole-body insulin resistance; and in further view of the fact that these IKKε knockout mice were found to have increased energy expenditure and thermogenesis, maintained insulin sensitivity in both liver and fat, reduced expression of inflammatory cytokines, and altered expression of regulatory proteins and enzymes involved in glucose and lipid metabolism (Chiang et al.; Cell, 138:961-975, 2009); it is believed that inhibition of IKKε kinase activity would be effective in treating obesity, insulin resistance, NIDDM, and metabolic syndrome, and complications associated with these and other metabolic diseases and disorders. Consequently, the present invention provides methods of treating obesity, insulin resistance, metabolic syndrome, type 2 diabetes, and complications associated with these diseases, and other metabolic diseases and disorders, comprising administering a therapeutically effective amount of one or more IKKε and/or TBK1-inhibiting compounds according to Formulae I and/or II to a patient in need of such treatment.

In further view of the discovery that TBK1 mediates phosphorylation of insulin receptor at serine residue 994, and thereby provides a potential link between inflammation and insulin resistance (Muñoz et al; J. Endocrinol., 201:185-197, 2009), it is believed that inhibition of TBK1 kinase activity might be effective in treating insulin resistance. Consequently, the present invention provides methods of treating insulin resistance, and complications associated with insulin resistance, comprising administering a therapeutically effective amount of one or more IKKε and/or TBK1-inhibiting compounds according to Formulae I and/or II to a patient in need of such treatment.

k. Treating Cancer:

In view of the discovery that the gene encoding IKKε (i.e., IKBKE; Entrez Gene ID: 9641) has been identified as a breast cancer oncogene (Boehm, et al.; Cell; 129:1065-1079, 2007); that IKKε directly phosphorylates the tumor suppressor CYLD in vivo, thereby decreasing the activity of CYLD, and leading to transformation and tumorigenesis (Hutti, et al.; Mol. Cell; 34:461-472, 2009); and that overexpression of IKKε is a recurrent event in human ovarian cancer, and that this overexpression could play a pivotal role in both tumor progression and the development of cisplatin resistance (Guo, et al.; Am. J. Pathol.; 175:324-333, 2009); it is believed that inhibition of IKKε kinase activity would be effective in treating of a wide range of cancers. Consequently, the present invention provides methods of treating a wide range of cancers comprising administering a therapeutically effective amount of one or more IKKε-inhibiting compounds according to Formulae I and/or II to a patient in need of such treatment.

In further view of the discovery that GTPase-mediated activation of TBK1 couples innate immune signaling to tumor cell survival (Chien et al.; Cell; 127:157-170, 2006), it is believed that inhibition of TBK1 kinase activity would be effective in treating of a wide range of cancers. Consequently, the present invention provides methods of treating a wide range of cancers comprising administering a therapeutically effective amount of one or more TBK1-inhibiting compounds according to Formulae I and/or II to a patient in need of such treatment.

As used herein, the term “cancer” has its conventional meaning in the art. Cancer includes any condition of the animal or human body characterized by abnormal cellular proliferation. The cancers to be treated comprise a group of diseases characterized by the uncontrolled growth and spread of abnormal cells. Compounds of the invention have been shown to be effective in cell-based cancer models, and are thus thought to have utility in treating a broad range of cancers. However, therapeutic methods of the present invention would best be directed towards cancers that are found to respond favorably to treatment with an IKKε and/or TBK1 kinase inhibitor. Further, “treating cancer” should be understood as encompassing treating a patient who is at any one of the several stages of cancer, including diagnosed but as yet asymptomatic cancer. A patient having cancer can be identified by conventional diagnostic techniques known in the art, and the identified patient may be treated with a compound of the present invention, once their cancer has been found to be susceptible to treatment with an IKKε and/or TBK1 kinase inhibitor.

As noted, cancers that may be treated by the methods of the invention are those cancers that respond favorably to treatment with an IKKε and/or TBK1 kinase inhibitor. Such cancers may include, but are not limited to, Hodgkin's disease, non-Hodgkin's lymphoma, acute lymphocytic leukemia, chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

The present invention further provides methods for combination therapy for treating cancer by treating a patient (either a human or another animal) in need of such treatment with a compound of the present invention together with one or more other anti-cancer therapies. Such other anti-cancer therapies include traditional chemotherapy agents, targeted agents, radiation therapy, surgery, hormone therapy, etc. In the combination therapy, the compound of the present invention may be administered separately from, or together with the one or more other anti-cancer therapies.

As noted above, it is believed that inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer are disease and disorders that will respond favorably to therapy with an IKKε or TBK1 kinase inhibitor. Consequently, the present invention provides therapeutic methods for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders. These therapeutic methods involve treating a patient (either a human or another animal) in need of such treatment, with a therapeutically effective amount of at least one compound according to Formulae I and/or II, or a pharmaceutical composition comprising a therapeutically effective amount of at least one compound according to Formulae I and/or II. These therapeutic methods also administering to a patient (either a human or another animal) in need of such treatment, a therapeutically effective amount of at least one compound according to Formulae I and/or II, or a pharmaceutical composition comprising a therapeutically effective amount of at least one compound according to Formulae I and/or II.

It is believed that compounds according to Formulae I and/or II and having an IKKε kinase inhibitory activity (IC50 value) of less than about 0.005 μM (5 nM), as determined in the in-vitro IKKε kinase inhibition assays as described below, are sufficiently active for the therapeutic methods proposed. These compounds include, for example, Example Compounds 4, 5, 7, 8, 9, 10, 11, 12, 13, 15, 21, 22, 23, 27, 29, 30, 32, 33, 34, 36, 37, 38, 39, 40, 41, 42, 43, 44, 45, 46, 48, 52, 53, 54, 55, 56, 57, 58, 59, 60, 61, 62, 63, 66, 67, 68, 69, 70, 71, 75, 76, 77, 78, 79, 80, 81, 82, 83, 87, 88, 89, 94, 95, 102, 104, 109, 111, 117, 119, 121, 123, 126, 127, 128, 130, 131, 138, 139, 141, 142, 143, and 149, as identified below.

The present invention also comprises treating isolated cells with a therapeutically effective amount of at least one compound according to Formulae I and/or II, or a pharmaceutical composition comprising a therapeutically effective amount of at least one compound according to Formulae I and/or II.

As used herein, the phrase “treating . . . with . . . a compound” means either administering a compound according to Formulae I and/or II, or a pharmaceutical compositions comprising a compound according to Formulae I and/or II, directly to isolated cells or to an animal, or administering to cells or an animal another agent to cause the presence or formation of a compound according to Formulae I and/or II inside the cells or the animal. Consequently, the methods of the present invention comprise administering to cells in vitro or to a warm-blood animal, particularly a mammal, and more particularly a human, a pharmaceutical composition comprising an effective amount of at least one compound according to Formulae I and/or II, or causing the presence or formation of at least one compound according Formulae I and/or II inside the cells or the animal.

As would be appreciated by the skilled artisan, at least one therapeutic compound according to Formulae I and/or II may be administered in one dose at one time, or may be divided into a number of smaller doses to be administered at predetermined intervals of time. The suitable dosage unit for each administration may be determined based on the effective daily amount and the pharmacokinetics of the compounds. In the case of combination therapy, a therapeutically effective amount of one or more other therapeutically effective compound can be administered in a separate pharmaceutical composition, or alternatively included in the pharmaceutical composition according to the present invention which contains a compound according to the present invention. The pharmacology and toxicology of many therapeutically effective compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage range set forth herein is exemplary and is not intended to limit the scope of the present invention. The therapeutically effective amount for each active compound of the invention may vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration may be adjusted as the various factors change over time.

The present invention also provides methods for methods for combination therapy for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, by treating a patient in need thereof, with a therapeutically effective amount of at least one compound according to Formulae I and/or II, together with a therapeutically effective amount of one or more other compounds that have been shown to be effective in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

For the convenience of combination therapy, at least one compound according to Formulae I and/or II can be administered together in the same formulation with the one or more other compounds that have been shown to be effective in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, in the same formulation or dosage form. Thus, the present invention also provides pharmaceutical compositions or medicaments for combination therapy, comprising an effective amount of at least one compound according to Formulae I and/or II, and an effective amount of at least one other compound that has been shown to be effective in the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders.

### 5. Methods of Making the Compounds According to Formulae I and/or II

Methods of making the compounds according to Formulae I and/or II, and intermediates used in their synthesis, are provided in the Examples section below. Apprised of the general synthetic schemes, specific intermediates, and detailed example of specific syntheses disclosed in the following section, the skilled artisan would be readily enabled to make the remaining compounds disclosed herein. In all cases, the syntheses were begun using commercially-available starting materials.

### EXAMPLES

### Chemical Examples

### Preparation of Intermediates Standard Methods

### Standard Method A; Nitro Reduction

The nitro compound was hydrogenated for 4-18 h in MeOH with a catalytic amount of Pd/C. The suspension was filtered through Celite and concentrated to provide the amine. If necessary, purification was performed by MPLC (SiO₂, EtOAc/Hexanes, 0-100%, optionally followed by a gradient from 100% EtOAc to 100% of 1:1 CH₂Cl₂/MeOH).

### Standard Method B; BOC Deprotection

A solution of the BOC protected amine in THF was treated with TFA (1-20%) for 1-18 h. The reaction was concentrated onto Celite and purified by RP-MPLC(C₁₈, MeOH/H₂O, 0-100% w/0.1% TFA) to provide the desired compounds as the TFA salts. Alternatively, the reaction was neutralized in the course of an aqueous workup to give the product as a free base.

### Standard Method C; HATU Coupling

To a solution of the carboxylic acid (1.0 eq), the amine (1.0-1.5 eq), DIPEA (1.0-1.5 eq) in DMF was added HATU (1.0-1.5 eq). The reaction mixture was stirred at rt for 8-16 h. The solvent was evaporated and the residue purified by RP-MPLC (C₁₈, MeOH/H₂O, 0-100% w/0.1% TFA) to provide the desired compounds. The desired fractions were collected and the solvent evaporated under reduced pressure. Alternatively, the resulting solid was recrystallized from EtOAc/Hexanes to afford the desired compound.

### Standard Method D; LAH Reduction of Amides

A solution of the amide in THF (0.25 M) was treated with LAH (10 eq) and the solution heated to reflux. The reaction mixture was stirred at reflux for 18 h, and then cooled to rt. The reaction was quenched by carefully pouring the mixture onto ice. Alternatively, the reaction was quenched by carefully adding n mL of 1 M NaOH (aq) solution, 3 times n mL of H₂O, and n mL of 1 M NaOH (aq) solution, where n is the number of moles of LAH used, then filtered. Concentration under vacuum afforded a semi-solid slurry. Acetonitrile is added to dissolve the product. Concentration of the organic solution afforded the pure amine.

### Standard Method E; LAH Reduction of Carboxylic Acids

A solution of the amide in THF (0.25 M) was treated with LAH (5-10 eq) and the solution heated (from 40° C. to reflux). The reaction mixture was stirred for 4-18 h, then cooled in an ice bath. The reaction was quenched by carefully adding n mL of 1 M NaOH (aq) solution, 3 times n mL of H₂O, and n mL of 1 M NaOH (aq) solution, where n is the number of moles of LAH used. Stirring continued at room temperature for 1 h. The mixture is filtered and the filtrate concentrated under reduced pressure to give the product alcohol.

### Standard Method F; Acylation or Sulfonylation

A solution of an amine (1.0 eq) and DIPEA (1.0-1.5 eq) in DCM was treated with an acid chloride, an acid anhydride, or a sulfonyl chloride (1.0-1.5 eq). DMAP was sometimes added to catalyze the reaction. The reaction mixture was stirred at rt for 1-16 h. The reaction was quenched by adding an aqueous solution of K₂CO₃ and stirring continued at rt until the reactants are consumed. An organic solvent such as EtOAc was added and the layers were separated. The organic layer was dried (Na₂SO₄) and concentrated under vacuum. The residue was purified by RP-MPLC (C₁₈, MeOH/H₂O, 0-100% w/0.1% TFA) to provide the desired compounds.

### Standard Method G; EDCI Coupling

To a solution of the carboxylic acid (1.0 eq), the amine (1.0-1.5 eq), DIPEA (1.0-1.5 eq) in DMF was added HOBt (1.0 eq) and EDCI (1.0 eq). The reaction mixture was stirred at rt or with heating to 65° C. for several hours as required for reaction completion. The solvent was evaporated and the residue purified by RP-MPLC (_{C18}, MeOH/H₂O, 0-100% w/0.1% TFA) to provide the desired compounds. The desired fractions were collected and the solvent evaporated under reduced pressure. The resulting solid was recrystallized from EtOAc/Hexanes to afford the desired compound.

### Synthesis of Intermediates

### Preparation of Intermediate I-1; 5-(2-Chloropyrimidin-4-yl)-2-hydroxy-benzonitrile

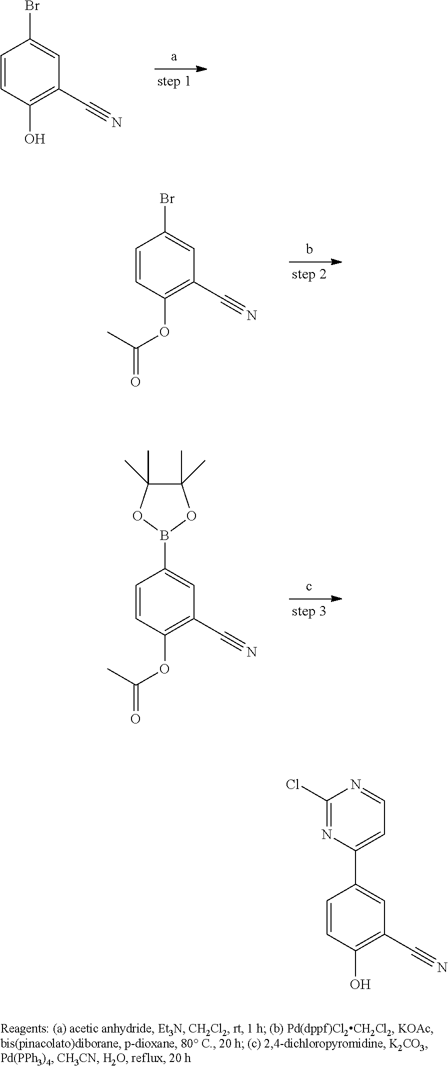 [SMILES: N#Cc1cc(Br)ccc1O ; CC(=O)Oc1ccc(Br)cc1C#N ; CC(=O)Oc1ccc(B2OC(C)(C)C(C)(C)O2)cc1C#N ; N#Cc1cc(-c2ccnc(Cl)n2)ccc1O]

Step 1. 4-Bromo-2-cyanophenyl acetate: To a solution of 5-bromo-2-hydroxy-benzonitrile (3.96 g, 20.0 mmol) and Et₃N (6 mL) in CH₂Cl₂ (60 mL) was added Ac₂O (4 mL, 42.4 mmol) at rt. After stirring for 1 h at rt, the mixture was diluted with CH₂Cl₂ (100 mL), washed with H₂O (100 mL) and brine (100 mL), dried (MgSO₄), and concentrated under vacuum. The residue (4.7 g, 19.6 mmol) was used without further purification.

Step 2. 2-Cyano-4-(4,4,5,5-tetramethyl-1,3,2-dioxaborolan-2-yl)phenyl acetate: To a solution of 4-bromo-2-cyanophenyl acetate (4.7 g, 19.6 mmol) in p-dioxane (100 mL) was added Pd(dppf)Cl₂.CH₂Cl₂ (0.80 g, 0.98 mmol), bis(pinacolato)diborane (7.46 g, 29.4 mmol) and KOAc (5.86 g, 60 mmol). After stirring at 80° C. for 20 h, the mixture was filtered to remove salts and the filtrate was concentrated under reduced pressure. The residue was purified by column chromatography (SiO₂, EtOAc/Hexanes, 0-50%) to afford the title compound (4.2 g, 75%).

Step 3. 5-(2-Chloropyrimidin-4-yl)-2-hydroxybenzonitrile: To a solution of 2-cyano-4-(4,4,5,5-tetramethyl-1,3,2-dioxaborolan-2-yl)phenyl acetate (4.2 g, 14.6 mmol) and 2,4-dichloropyrimidine (2.18 g, 14.6 mmol) in CH₃CN (100 mL) was added H₂O (40 mL), K₂CO₃ (6.04 g, 43.8 mmol), and Pd(PPh₃)₄ (0.84 g, 0.73 mmol). After refluxing for 20 h, the mixture was concentrated to remove CH₃CN and the product was extracted with a solution of i-PrOH/CHCl₃ (1:3) (200 mL). The organic solution was washed with brine (100 mL), dried (MgSO₄), and concentrated under reduced pressure. The residue was purified by column chromatography (SiO₂, MeOH 20% in CH₂Cl₂ with 0.1% NH₄OH), to give the title compound; LC-MS [M−H]⁻ 229.

### Preparation of Intermediate I-2; 5-(2-Chloropyrimidin-4-yl)-2-methoxybenzonitrile

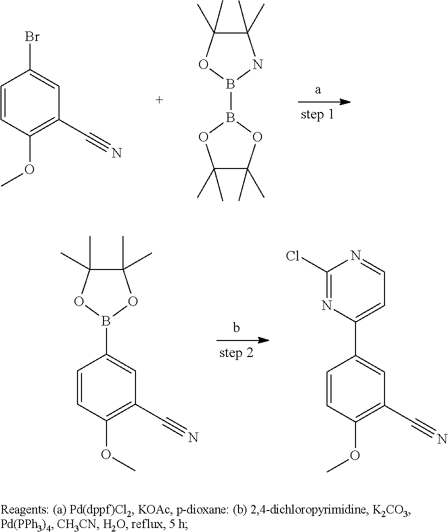

Step 1. 2-Methoxy-5-(4,4,5,5-tetramethyl-1,3,2-dioxaborolan-2-yl)benzonitrile: To a solution of 2-methoxy-5-bromobenzonitrile (5.0 g, 23.6 mmol) in p-dioxane (125 mL), bis(pinacolato)diborane (9.0 g, 35.4 mmol), KOAc (7.0 g, 71.3 mmol), and Pd(dppf)Cl₂ (0.863 g, 1.17 mmol) were added. The resulting mixture was stirred for 18 h at 80° C. The cooled reaction crude was diluted with 120 mL EtOAc, washed with H₂O and brine, dried (Na₂SO₄), filtered, and concentrated in vacuo. The residue was purified by column chromatography on SiO₂ (Hexanes/EtOAc) to afford the title compound (5.6 g, 92%). GC/MS (EI, M⁺) 245

Step 2. 5-(2-Chloropyrimidin-4-yl)-2-methoxybenzonitrile: To a solution of 2-methoxy-5-(4,4,5,5-tetramethyl-1,3,2-dioxaborolan-2-yl)benzonitrile (5.6 g, 21.6 mmol) in CH₃CN (100 mL) and H₂O (35 mL), 2,4-dichloropyrimidine (3.22 g, 21.6 mmol), K₂CO₃ (9.0 g, 65 mmol), and Pd(PPh₃)₄ (1.25 g, 1.06 mmol) were added. The resulting mixture was stirred for 5 h at 90° C. Upon cooling, the product precipitated from solution and was filtered and washed with a 3:1 CH₃CN/H₂O mixture, and dried in vacuo to afford the title compound (4.04 g, 76%). ¹H NMR (CDCl₃) δ 8.66 (d, 1H), 8.36-8.33 (m, 2H), 7.59 (d, 1H), 7.13-7.11 (m, 1H), 4.04 (s, 3H). LC-MS [M+H]⁺ 245.9

### Preparation of Intermediate I-3; 5-(2-Chloropyrimidin-4-yl)-2-(tetrahydro-2H-pyran-4-yloxy)benzonitrile

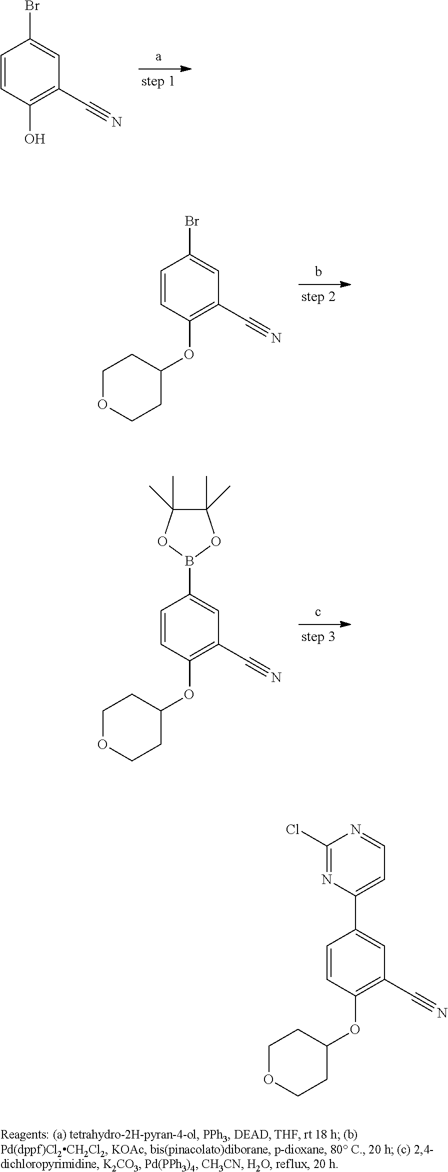 [SMILES: N#Cc1cc(Br)ccc1O ; N#Cc1cc(Br)ccc1OC1CCOCC1 ; CC1(C)OB(c2ccc(OC3CCOCC3)c(C#N)c2)OC1(C)C ; N#Cc1cc(-c2ccnc(Cl)n2)ccc1OC1CCOCC1]

Step 1. 5-Bromo-2-(tetrahydro-2H-pyran-4-yloxy)benzonitrile: To a solution of 5-bromo-2-hydroxy-benzonitrile (1.98 g, 10.0 mmol) in dry THF (40 mL) was added tetrahydro-2H-pyran-4-ol (1.02 g, 10 mmol), and PPh₃ (3.15 g, 12 mmol), followed by the addition of DEAD (1.89 mL, 12 mmol) at rt. After stirring for 18 h, the reaction mixture was concentrated under reduced pressure. The residue was purified by column chromatography (SiO₂, EtOAc/Hexanes, 0-80%) to afford the title compound (2.7 g, 96%). ¹H NMR (DMSO-d₆) δ 8.02 (d, 1H), 7.81 (dd, 1H), 7.35 (d, 1H), 4.85-4.78 (m, 1H), 3.86-3.80 (m, 2H), 3.55-3.47 (m, 2H), 2.01-1.96 (m, 2H), 1.67-1.58 (m, 2H).

Step 2. 2-(Tetrahydro-2H-pyran-4-yloxy)-5-(4,4,5,5-tetramethyl-1,3,2-dioxaborolan-2-yl)benzonitrile: To a solution of 5-bromo-2-(tetrahydro-2H-pyran-4-yloxy)benzonitrile (2.7 g, 9.6 mmol) and bis(pinacolato)diborane (2.4 g, 9.6 mmol) in p-dioxane (50 mL) was added Pd(dppf)Cl₂.CH₂Cl₂ (0.408 g, 0.50 mmol), and KOAc (2.94 g, 30 mmol). After stirring at 80° C. for 20 h, the mixture was filtered to remove KOAc and the filtrate was concentrated under reduced pressure. The residue was purified by column chromatography (SiO₂, EtOAc/Hexanes, 0-60%) to afford the title compound (3.1 g, 98%).

Step 3. 5-(2-Chloropyrimidin-4-yl)-2-(tetrahydro-2H-pyran-4-yloxy)benzonitrile: To a solution of 2-(tetrahydro-2H-pyran-4-yloxy)-5-(4,4,5,5-tetramethyl-1,3,2-dioxaborolan-2-yl)benzonitrile (3.1 g, 9.4 mmol) and 2,4-dichloropyrimidine (1.40 g, 9.4 mmol) in CH₃CN (40 mL) and H₂0 (15 mL) was added K₂CO₃ (4.14 g, 30 mmol) and Pd(PPh₃)₄ (0.58 g, 0.5 mmol). After refluxing for 20 h, the mixture was concentrated to remove CH₃CN and the residue was extracted with EtOAc (200 mL). The organic solution was washed with brine (100 mL), dried (MgSO₄), and concentrated under reduced pressure. The residue was purified by column chromatography (SiO₂, EtOAc/Hexanes, 0-100%) to give the title compound (1.3 g, 41%). ¹H NMR (DMSO-d₆) δ 8.83 (d, 1H), 8.60 (d, 1H), 8.46 (dd, 1H), 8.21 (d, H), 7.57 (d, 1H), 5.00-4.94 (m, 1H), 3.90-3.84 (m, 2H), 3.58-3.53 (m, 2H), 2.06-1.99 (m, 2H), 1.73-1.65 (m, 2H).

### Alternative Preparation of Intermediate I-3; 5-(2-Chloropyrimidin-4-yl)-2-tetrahydropyran-4-yloxy-benzonitrile

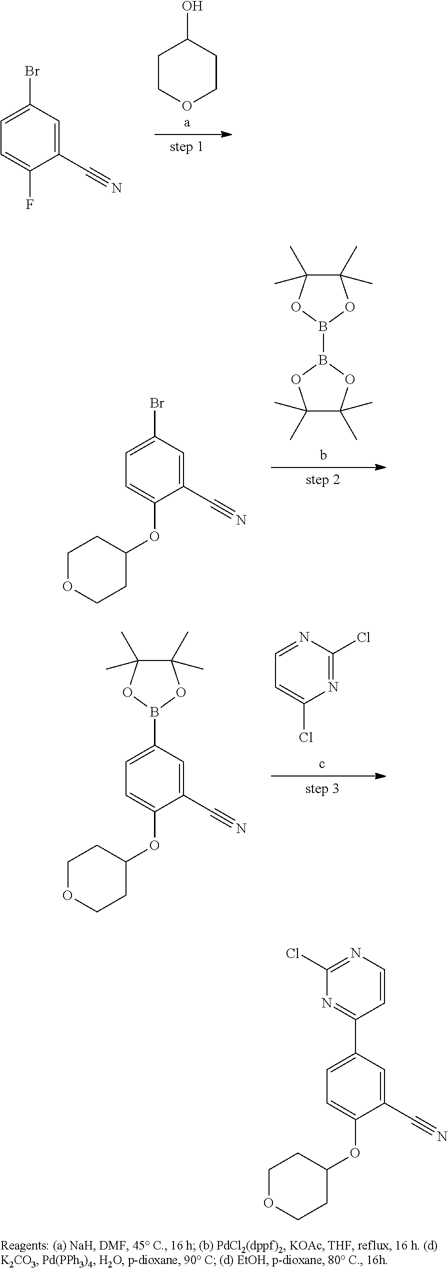

Step 1: 5-Bromo-2-tetrahydropyran-4-yloxy-benzonitrile: To tetrahydropyranol (7.1 g, 69.5 mmol) in DMF (130 mL) at 0° C. was added NaH (2.78 g, 69.5 mmol). 5-bromo-2-fluorobenzonitrile (11.6 g, 57.9 mmol) was added dropwise as a solution in DMF (63 mL). The reaction was stirred at 45° C. for 16 h. The reaction was cooled to room temperature and quenched by pouring the reaction into H₂O (1.5 L). The precipitate was filtered and dried under vacuum to provide 16.8 g of material (88%). The product was used without further purification. ¹H NMR (DMSO-d₆) δ 8.02 (s, 1H), 7.82 (d, 1H), 7.35 (d, 1H), 4.85-4.76 (m, 1H), 3.90-3.80 (m, 2H), 3.58-3.49 (m, 2H), 2.04-1.95 (m, 2H), 1.70-1.60 (m, 2H).

Step 2: 2-Tetrahydropyran-4-yloxy-5-(4,4,5,5-tetramethyl-1,3,2-dioxaborolan-2-yl)benzonitrile: To 5-Bromo-2-tetrahydropyran-4-yloxy-benzonitrile (7.8 g, 23.5 mmol) in p-dioxane (78 mL) was added bis(pinacolato)diboron (8.9 g, 35.3 mmol), KOAc (6.9 g, 70.5 mmol), and Pd(dppf)Cl₂ (0.86 g, 1.2 mmol). The reaction was heated to 90° C. for 16 h. The reaction was quenched with H₂O (50 mL), followed by extraction with EtOAc (3×25 mL). The aqueous and organic layers were separated. The organic layer was washed with saturated NaCl (aq) solution and dried (Na₂SO₄). Purification by medium pressure liquid chromatography (0-100% EtOAc in Hexanes) provided 7.6 g (98%) material. ¹H NMR (CDCl₃) δ 8.04 (s, 1H), 7.90 (d, 1H), 6.95 (d, 1H), 4.77-4.70 (m, 1H), 4.10-4.00 (m, 2H), 3.67-3.60 (m, 2H), 2.10-2.00 (m, 2H), 1.90-1.81 (m, 2H), 1.15 (s, 12H).

Step 3: 5-(2-Chloropyrimidin-4-yl)-2-tetrahydropyran-4-yloxy-benzonitrile: To 2-tetrahydropyran-4-yloxy-5-(4,4,5,5-tetramethyl-1,3,2-dioxaborolan-2-yl)benzonitrile (8.0 g, 24.3 mmol) in p-dioxane (60 mL) and H₂O (20 mL) was added 2,4-dichloropyrimidine (3.6 g, 24.3 mmol), K₂CO₃ (6.7 g, 48.6 mmol), and Pd(PPh₃)₄ (1.4 g, 1.2 mmol). The reaction was heated to 90° C. for 16 h. The reaction was quenched with H₂O (50 mL) followed by extraction with EtOAc (3×25 mL). The aqueous and organic layers were separated. The organic layer was washed with saturated NaCl (aq) and dried (Na₂SO₄), filtered and concentrated. Purification by MPLC (0-100% EtOAc in Hexanes) provided 7.5 g (98%) material. ¹H NMR (CDCl₃) δ 8.66 (d, 1H), 8.35-8.29 (m, 2H), 7.65 (d, 1H), 7.05 (d, 1H), 4.82-4.85 (m, 1H), 4.10-4.00 (m, 2H), 3.71-3.62 (m, 2H), 2.15-2.05 (m, 2H), 1.99-1.89 (m, 2H).

### Preparation of Intermediate I-4; 5-(2-Chloropyrimidin-4-yl)-3-methoxy-2-tetrahydropyran-4-yloxy-benzonitrile

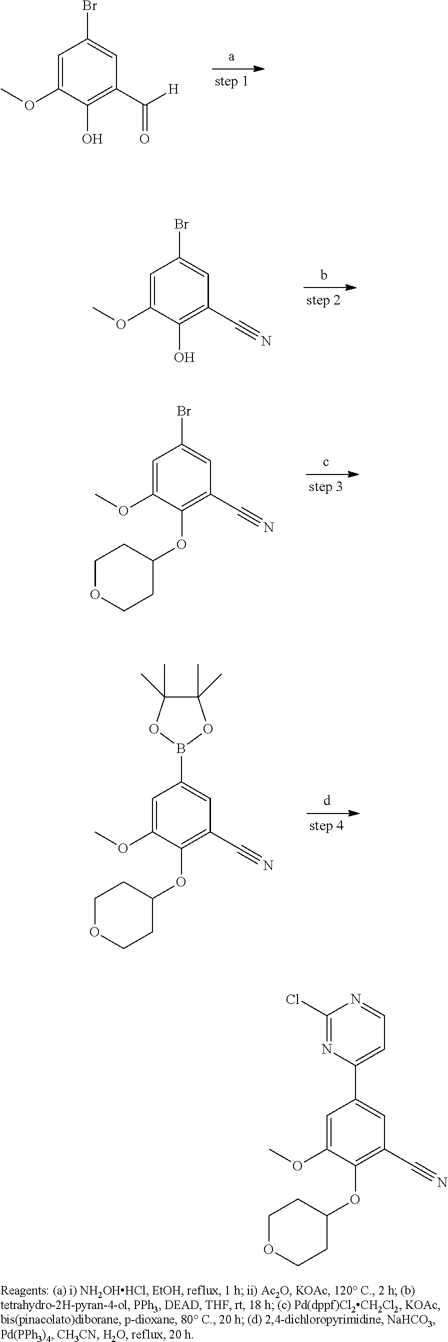 [SMILES: COc1cc(Br)cc(C=O)c1O ; COc1cc(Br)cc(C#N)c1O ; COc1cc(Br)cc(C#N)c1OC1CCOCC1 ; COc1cc(B2OC(C)(C)C(C)(C)O2)cc(C#N)c1OC1CCOCC1 ; COc1cc(-c2ccnc(Cl)n2)cc(C#N)c1OC1CCOCC1]

Step 1. 5-Bromo-2-hydroxy-3-methoxy-benzonitrile: A mixture of 5-bromo-2-hydroxy-3-methoxy-benzaldehyde (2.31 g, 10.0 mmol) and hydroxylamine hydrogen chloride (0.834 g, 12.0 mmol) in EtOH (10 mL) was stirred at reflux for 1 h. After removal of EtOH and drying in vacuo, the residue was added to Ac₂O (10 mL) and KOAc (2.0 g) and the solution was stirred at 120° C. for 2 h. After cooling to rt, the reaction mixture was added H₂O (100 mL) and MeOH (10 mL), and basified with solid K₂CO₃ to about pH 10. After stirring for 24 h, the mixture was acidified with conc. HCl (aq) to a pH of 4.5. The resulting precipitate was collected and dried in vacuo to give 2.1 g of the title compound as an off-white powder. GC/MS (EI, M+) 227.

Step 2. 5-Bromo-3-methoxy-2-tetrahydropyran-4-yloxy-benzonitrile: To a solution of 5-bromo-2-hydroxy-3-methoxy-benzonitrile (1.14 g, 5.0 mmol) in dry THF (20 mL) was added tetrahydropyran-4-ol (0.56 g, 5.5 mmol), PPh₃ (1.57 g, 6.0 mmol), followed by addition of DEAD (1.0 mL, 6.0 mmol) at 0° C. After stirring at rt for 18 h, the reaction mixture was concentrated under reduced pressure. The residue was purified by column chromatography (SiO₂, EtOAc/Hexanes, 0-100%) to afford the title compound (1.45 g, 78.0%). GC/MS (EI, M+) 313.

Step 3. 3-Methoxy-2-tetrahydropyran-4-yloxy-5-(4,4,5,5-tetramethyl-1,3,2-dioxaborolan-2-yl)benzonitrile: To a solution of 5-bromo-3-methoxy-2-tetrahydropyran-4-yloxy-benzonitrile (1.45 g, 4.66 mmol)) in p-dioxane (30 mL) was added Pd(dppf)Cl₂.CH₂Cl₂ (0.204 g, 0.25 mmol), bis(pinacolato)diborane (1.18 g, 4.66 mmol) and KOAc (1.47 g, 15 mmol). After stirring at 80° C. for 20 h, the mixture was filtered to remove KOAc, and the filtrate was concentrated under reduced pressure. The residue was purified by column chromatography (SiO₂, EtOAc/Hexanes, 0-100%) to afford the title compound. GC/MS (EI, M+) 359.

Step 4. 5-(2-Chloropyrimidin-4-yl)-3-methoxy-2-tetrahydropyran-4-yloxy-benzonitrile: To a solution of 3-methoxy-2-tetrahydropyran-4-yloxy-5-(4,4,5,5-tetramethyl-1,3,2-dioxaborolan-2-yl)benzonitrile (1.67 g, 4.66 mmol) and 2,4-dichloropyrimidine (0.69 g, 4.66 mmol) in CH₃CN (30 mL) and H₂O (10 mL) was added Na₂CO₃ (1.26 g, 15 mmol) and Pd(PPh₃)₄ (0.29 g, 0.25 mmol). After refluxing for 20 h, the mixture was concentrated to remove CH₃CN, and the residue was extracted with EtOAc (200 mL). The organic solution was washed with brine (100 mL), dried (MgSO₄), and concentrated under reduced pressure. The residue was purified by column chromatography (SiO₂, EtOAc/Hexanes, 0-85%) to give the title compound (1.2 g, 75.0%). LC-MS [M+H]⁺ 346.1023.

### Preparation of Intermediate I-5; 5-(2-Chloropyrimidin-4-yl)-2-isobutoxy-benzonitrile

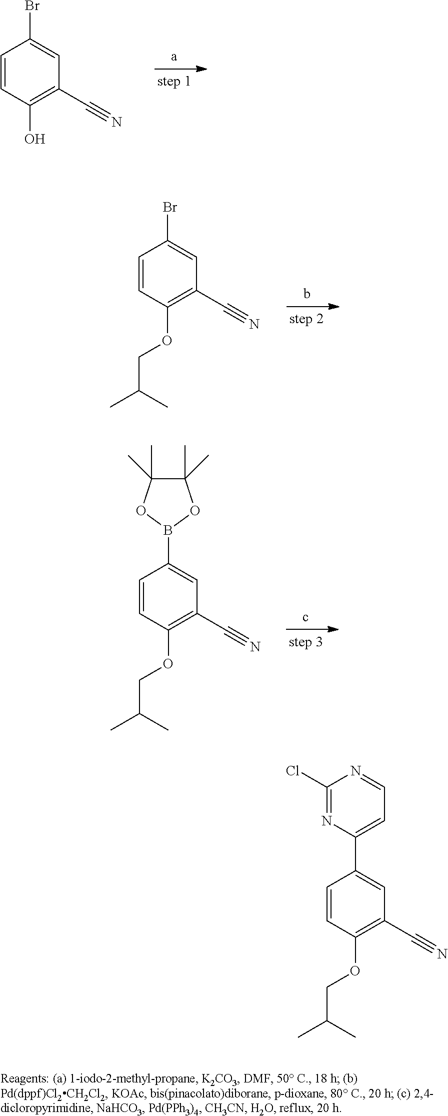

Step 1. 5-Bromo-2-isobutoxy-benzonitrile: To a solution of 5-bromo-2-hydroxy-benzonitrile (0.98 g, 10.0 mmol) in dry DMF (40 mL) was added 1-iodo-2-methyl-propane (3.5 mL, 30 mmol), and K₂CO₃ (6.9 g, 50 mmol). The mixture was heated to 50° C., and stirred for 20 h. After cooling to rt, the reaction mixture was concentrated under reduced pressure. The residue was diluted with chloroform and washed with water (100 mL), then dried (Na₂SO₄) and concentrated under reduced pressure. Purification by column chromatography (SiO₂, EtOAc/Hexanes, 0-80%) afforded the title compound as a colorless oil (2.7 g, 53%).

Step 2. 2-Isobutoxy-5-(4,4,5,5-tetramethyl-1,3,2-dioxaborolan-2-yl)benzonitrile: The compound was prepared according to the method used in Step 2 for Intermediate I-1 using 5-bromo-2-isobutoxy-benzonitrile (2.78 g, 11 mmol) to gave a crude oily residue which was used in the next step without further characterization.

Step 3. 5-(2-Chloropyrimidin-4-yl)-2-isobutoxy-benzonitrile: Treatment of the residue obtained in Step 2 according to the procedure used in Step 3 for Intermediate-I-3 afforded the title compound as a white solid (1.2 g, 42% over two steps).

### Preparation of Intermediate I-6; 5-(2-Chloropyrimidin-4-yl)-2-(cyclopropylmethoxy)benzonitrile

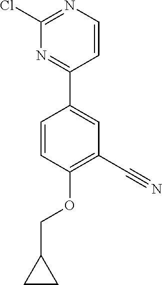 [SMILES: N#Cc1cc(-c2ccnc(Cl)n2)ccc1OCC1CC1]

This compound was prepared according to the procedure described for the preparation of Intermediate I-5 using bromomethylcyclopropane (2.0 g, 15 mmol) to give the title compound as a white solid.

### Preparation of Intermediate I-7; 5-(2-Chloropyrimidin-4-yl)-2-[(3-methyloxetan-3-yl)methoxy]benzonitrile

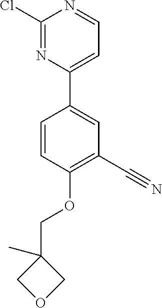 [SMILES: CC1(COc2ccc(-c3ccnc(Cl)n3)cc2C#N)COC1]

This compound was prepared according to the procedure described for the preparation of Intermediate I-3 using (3-methyloxetan-3-yl)methanol (1.2 mL, 12 mmol) and 5-bromo-2-hydroxy-benzonitrile (2.0 g, 10 mmol) to give the title compound as a white solid (1.2 g, 32% over 3 steps).

### Preparation of Intermediate I-8; 2-Amino-5-(2-chloropyrimidin-4-yl)benzonitrile

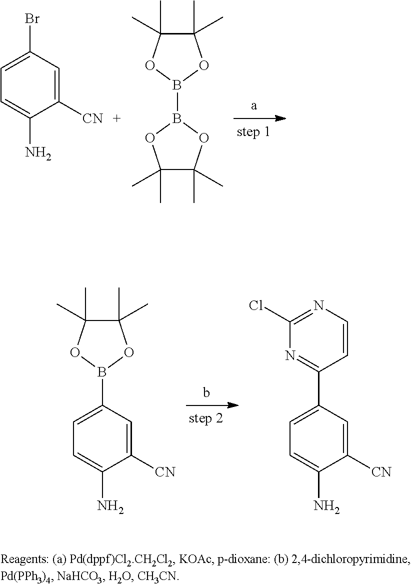

Step 1. 2-Amino-5-(4,4,5,5-tetramethyl-1,3,2-dioxaborolan-2-yl)benzonitrile: To a solution of 2-amino-5-bromobenzonitrile (1.0 g, 5.075 mmol) in p-dioxane (15 mL), bis(pinacolato)diborane (1.95 g, 7.61 mmol), KOAc (1.5 g, 15.23 mmol), and Pd(dppf)Cl₂.CH₂Cl₂ (0.207 g, 0.25 mmol) were added. The resulting mixture was stirred for 16 h at 80° C. The cooled reaction mixture was diluted with 200 mL EtOAc, washed with H₂O and brine, dried (Na₂SO₄), filtered, and concentrated in vacuo. The residue was purified by column chromatography on SiO₂ (Hexanes/EtOAc) to afford the title compound (1.13 g, 91%). GC/MS (EI, M⁺) 244.

Step 2. 2-Amino-5-(2-chloropyrimidin-4-yl)benzonitrile: To a solution of 2-amino-5-(4,4,5,5-tetramethyl-1,3,2-dioxaborolan-2-yl)benzonitrile (1.1 g, 4.5 mmol) in CH₃CN (30 mL) and H₂O (10 mL), 2,4-dichloropyrimidine (0.672 g, 4.5 mmol), NaHCO₃ (1.14 g, 13.5 mmol), and Pd(PPh₃)₄ (0.26 g, 0.225 mmol) were added. The resulting mixture was stirred for 5 h at 80° C. Upon cooling, the desired product precipitates from solution, was washed with 3:1 CH₃CN/H₂O mixture and dried in vacuo to afford the title compound (0.67 g, 65%). LC-MS [M+H]⁺ 231.1.

### Preparation of Intermediate I-9; N-[4-(2-Chloropyrimidin-4-yl)phenyl]-2-methyl-propanamide

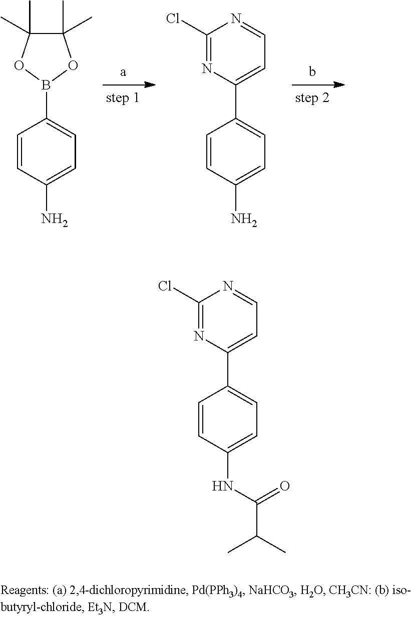

Step 1. 4-(2-Chloropyrimidin-4-yl)aniline: To a solution of 4-(4,4,5,5-tetramethyl-1,3,2-dioxaborolan-2-yl)aniline (1.0 g, 4.56 mmol) in CH₃CN (30 mL) and H₂O (10 mL), 2,4-dichloropyrimidine (0.68 g, 4.56 mmol), NaHCO₃ (1.15 g, 13.68 mmol), and Pd(PPh₃)₄ (0.26 g, 0.225 mmol) were added. The resulting mixture was stirred for 16 h at 80° C. The reaction was cooled, diluted with EtOAc, washed with H₂O, and concentrated onto silica. The residue was purified by column chromatography (SiO₂, EtOAc/Hexanes, 0-100%) to afford the title compound (0.53 g, 56%). GC/MS (EI, M⁺) 205.

Step 2.

N-[4-(2-Chloropyrimidin-4-yl)phenyl]-2-methyl-propanamide: To a solution of 4-(2-chloropyrimidin-4-yl)aniline (0.53 g, 2.58 mmol) in DCM (15 mL) was added iso-butyryl-chloride (0.300 mL, 2.84 mmol), followed by portion-wise addition of Et₃N (0.900 mL, 6.45 mmol). The resulting mixture was stirred for 30 minutes at rt. The reaction was diluted with DCM and washed with saturated aqueous NaHCO₃ and 1N HCl (aq) solution. The residue was dried in vacuo to afford the title compound (0.77 g, 100%). GC/MS (EI, M⁺) 275.

### Preparation of Intermediate I-10; N-[4-(2-Chloropyrimidin-4-yl)-2-cyano-phenyl]cyclopropanecarboxamide

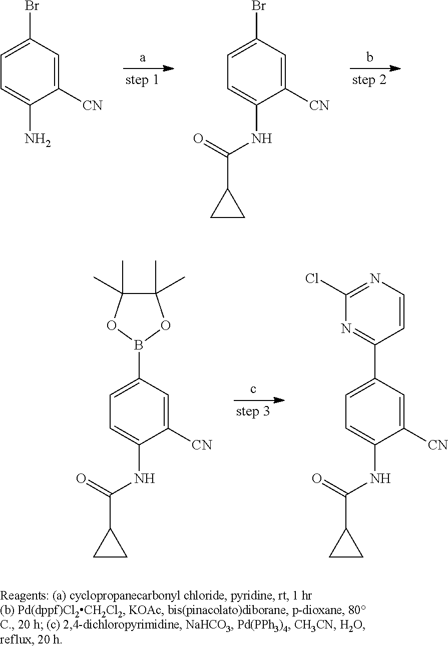 [SMILES: N#Cc1cc(Br)ccc1N ; N#Cc1cc(Br)ccc1NC(=O)C1CC1 ; CC1(C)OB(c2ccc(NC(=O)C3CC3)c(C#N)c2)OC1(C)C ; N#Cc1cc(-c2ccnc(Cl)n2)ccc1NC(=O)C1CC1]

Step 1. N-(4-Bromo-2-cyano-phenyl)cyclopropanecarboxamide. A solution of 2-amino-5-bromo-benzonitrile (5 g, 25 mmol) in pyridine (50 mL) was treated with cyclopropanecarbonyl chloride (2.55 mL, 27.5 mmol), added dropwise over a 30 min period. The reaction was stirred at rt for 1 h, then concentrated under vacuum. The residue was dissolved in EtOAc and washed with H₂O, 10% aqueous HCl, and saturated aqueous NaCl. The organic layer was dried (Na₂SO₄) and concentrated under vacuum to give the crude product. Purification by column chromatography on SiO₂ (Hexanes/EtOAc) gave the title compound (5.94 g, 88%). GC/MS (EI, M+) 265.

Step 2. N-[2-Cyano-4-(4,4,5,5-tetramethyl-1,3,2-dioxaborolan-2-yl)phenyl]cyclopropanecarboxamide. To a solution of N-(4-bromo-2-cyano-phenyl)cyclopropanecarboxamide (5.94 g, 22.4 mmol) in p-dioxane (50 mL), bis(pinacolato)diborane (7.11 g, 28 mmol), KOAc (6.6 g, 67.2 mmol), and Pd(dppf)Cl₂ (0.913 g, 1.12 mmol) were added. The resulting mixture was stirred for 16 h at 80° C. The cooled reaction crude was diluted with 120 mL EtOAc, washed with H₂O and brine, dried (Na₂SO₄), filtered, and concentrated in vacuo. The residue was purified by column chromatography on SiO₂ (Hexanes/EtOAc) to afford the title compound (5.71 g, 82%). GC/MS (EI, M⁺) 312.

Step 3. N-[4-(2-Chloropyrimidin-4-yl)-2-cyano-phenyl]cyclopropanecarboxamide: To a solution of N-[2-cyano-4-(4,4,5,5-tetramethyl-1,3,2-dioxaborolan-2-yl)phenyl]cyclopropanecarboxamide (5.71 g, 18.3 mmol) in CH3CN (100 mL) and H₂O (35 mL), 2,4-dichloropyrimidine (2.7 g, 18.1 mmol), NaHCO₃ (4.61 g, 54 mmol), and Pd(PPh₃)₄ (1.056 g, 1 mmol) were added. The resulting mixture was stirred for 5 h at 90° C. Upon cooling, the product precipitated from solution and was filtered and washed with a 3:1 CH3CN/H₂O mixture, and dried in vacuo to afford the title compound (4.04 g, 76%). GC/MS (EI, M+) 298.

### Preparation of Intermediate I-11; tert-Butyl 4-[4-(2-chloropyrimidin-4-yl)-2-cyano-phenoxy]piperidine-1-carboxylate

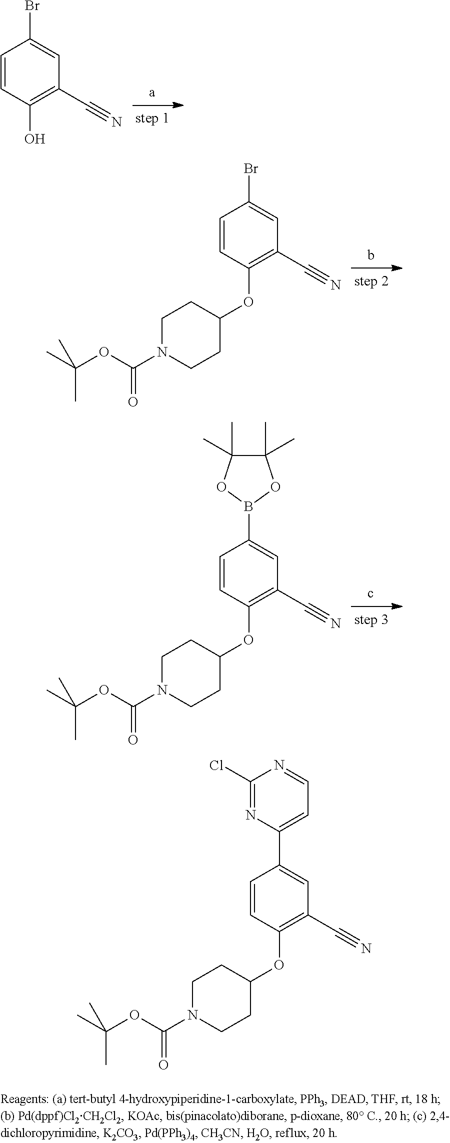 [SMILES: N#Cc1cc(Br)ccc1O ; CC(C)(C)OC(=O)N1CCC(Oc2ccc(Br)cc2C#N)CC1 ; CC(C)(C)OC(=O)N1CCC(Oc2ccc(B3OC(C)(C)C(C)(C)O3)cc2C#N)CC1 ; CC(C)(C)OC(=O)N1CCC(Oc2ccc(-c3ccnc(Cl)n3)cc2C#N)CC1]

Step 1. tert-Butyl 4-(4-bromo-2-cyano-phenoxy)piperidine-1-carboxylate: 5-bromo-2-hydroxy-benzonitrile (1.98 g, 10.0 mmol) in dry THF (40 mL) was combined with tert-butyl 4-hydroxypiperidine-1-carboxylate (2.41 g, 12 mmol), PPh₃ (3.14 g, 12 mmol), followed by addition of DEAD (1.89 mL, 12 mmol) at rt. After stirring at rt for 18 h, the reaction mixture was concentrated under reduced pressure. The residue was purified by column chromatography (SiO₂, EtOAc/Hexanes, 0-80%) to afford the title compound (3.4 g, 89.2%). LC-MS [M+Na]⁺ 404.1.

Step 2. tert-Butyl 4-[2-cyano-4-(4,4,5,5-tetramethyl-1,3,2-dioxaborolan-2-yl)phenoxy]piperidine-1-carboxylate: To a solution of tert-butyl 4-(4-bromo-2-cyanophenoxy)piperidine-1-carboxylate (3.4 g, 8.92 mmol) in p-dioxane (60 mL) was added Pd(dppf)Cl₂.CH₂Cl₂ (0.364 g, 0.446 mmol), bis(pinacolato)diborane (2.5 g, 10 mmol), and KOAc (2.65 g, 27 mmol). After stirring at 80° C. for 20 h, the mixture was filtered to remove KOAc, and the filtrate was concentrated under reduced pressure. The residue was purified by column chromatography (SiO₂, EtOAc/Hexanes, 0-100%) to afford the title compound (3.8 g, 99%). GC/MS (EI, M+) 428.

Step 3. tert-Butyl 4-[4-(2-chloropyrimidin-4-yl)-2-cyanophenoxy]piperidine-1-carboxylate: To a solution of tert-butyl 4-[2-cyano-4-(4,4,5,5-tetramethyl-1,3,2-dioxaborolan-2-yl)phenoxy]piperidine-1-carboxylate (3.8 g, 8.90 mmol) in CH₃CN (50 mL) and H₂O (20 mL) was added 2,4-dichloropyrimidine (1.32 g, 8.9 mmol), K₂CO₃ (4.14 g, 30 mmol) and Pd(PPh₃)₄ (0.58 g, 0.5 mmol). After refluxing for 20 h, the mixture was concentrated and the product was extracted with EtOAc (200 mL). The organic solution was washed with brine (100 mL), dried (MgSO₄), and concentrated under reduced pressure. The residue was purified by column chromatography (SiO₂, EtOAc/Hexanes, 0-100%) to give the title compound (2.6 g, 70.5%); ¹H NMR (CDCl₃) δ 8.66 (d, 1H), 8.36-8.28 (m, 2H), 7.58 (d, 1H), 7.11 (d, 1H), 4.77 (br. s, 1H), 3.72-3.47 (m, 4H), 2.05-1.85 (m, 4H), 1.48 (s, 9H).

### Preparation of Intermediate I-12; tert-Butyl (3R)-3-[4-(2-chloropyrimidin-4-yl)-2-cyano-phenoxy]pyrrolidine-1-carboxylate

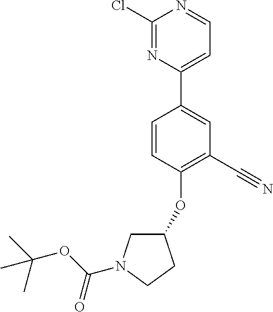 [SMILES: CC(C)(C)OC(=O)N1CC[C@@H](Oc2ccc(-c3ccnc(Cl)n3)cc2C#N)C1]

This compound was prepared according to the procedure described for the preparation of Intermediate I-11 using tert-butyl (3R)-3-hydroxypyrrolidine-1-carboxylate to give the title compound; ¹H NMR (CDCl₃) δ 8.67 (d, 1H), 8.35-8.29 (m, 2H), 7.59 (d, 1H), 7.05 (d, 1H), 5.09 (m, 1H), 3.76-3.57 (m, 4H), 2.36-2.18 (m, 2H), 1.48 (s, 9H).

### Preparation of Intermediate I-13; 2-[(5-Amino-2-pyridyl)-methyl-amino]ethanol

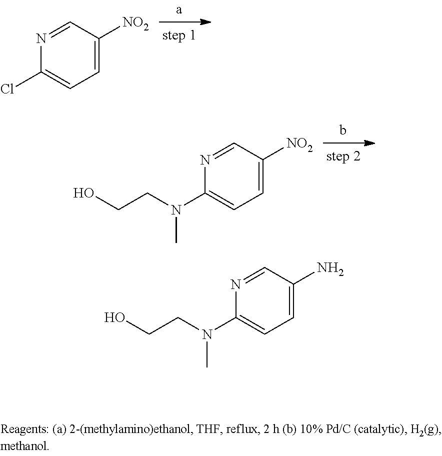

Step 1. 2-[Methyl-(5-nitro-2-pyridyl)amino]ethanol: A solution of 2-chloro-5-nitro-pyridine (0.79 g, 5.0 mmol) and 2-(methylamino)ethanol (1.0 mL, 13.0 mmol) in THF (20 mL) was stirred at reflux for 2 h. After cooling, a yellow precipitate formed and was collected by filtration. The material was carried on without further purification.

Step 2. 2-[(5-Amino-2-pyridyl)-methyl-amino]ethanol. The Standard Method A, Nitro Reduction, was used to prepare the title compound from the material isolated in Step 1.

### Preparation of Intermediate I-14; [(2R)-1-(4-amino-2-pyridyl)pyrrolidin-2-yl]methanol

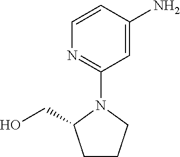 [SMILES: Nc1ccnc(N2CCC[C@@H]2CO)c1]

This compound was prepared according to the procedure described for the preparation of Intermediate I-13 using 2-chloro-4-nitro-pyridine and [(2R)-pyrrolidin-2-yl]methanol as starting materials.

### Preparation of Intermediate I-15; 2-morpholinopyrimidin-5-amine

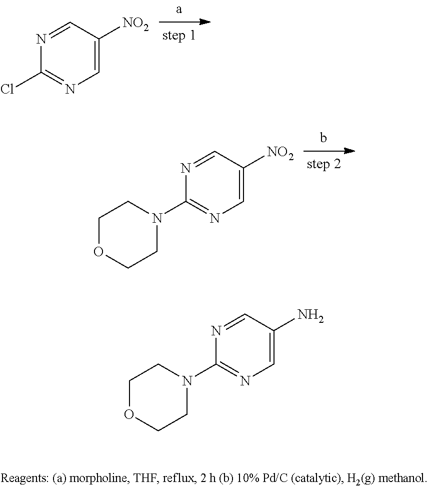 [SMILES: O=[N+]([O-])c1cnc(Cl)nc1 ; O=[N+]([O-])c1cnc(N2CCOCC2)nc1 ; Nc1cnc(N2CCOCC2)nc1]

Step 1. 4-(5-Nitropyrimidin-2-yl)morpholine: A solution of 2-chloro-5-nitro-pyrimidine (1.59 g, 10 mmol) and morpholine (1.3 mL, 15.0 mmol) in THF (20 mL) was stirred at reflux for 2 h. After cooling, hexane was added and the resulting precipitate was collected by filtration. The material was carried on without further purification.

Step 2. 2-Morpholinopyrimidin-5-amine: The Standard Method A, Nitro Reduction, was used to prepare the title compound from the material isolated in Step 1.

### Preparation of Intermediate I-16; 1-(5-Amino-2-pyridyl)azetidin-3-ol

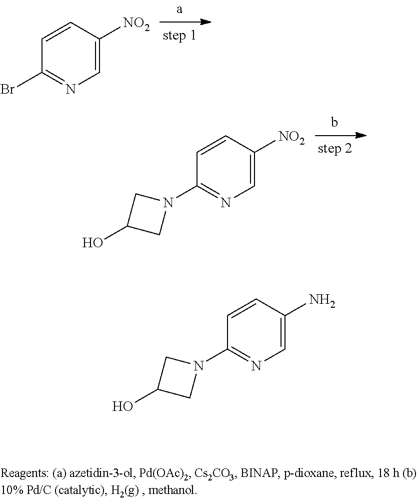 [SMILES: O=[N+]([O-])c1ccc(Br)nc1 ; O=[N+]([O-])c1ccc(N2CC(O)C2)nc1 ; Nc1ccc(N2CC(O)C2)nc1]

Step 1. 1-(5-Nitro-2-pyridyl)azetidin-3-ol: A solution of 2-bromo-5-nitro-pyridine (0.5 g, 2.46 mmol) and azetidin-3-ol (0.404 mL, 3.69 mmol) in p-dioxane (10 mL) was stirred at reflux for 18 h. After cooling, the mixture was filtered through Celite and concentrated under vacuum. The residue was purified by column chromatography (SiO₂, MeOH 20% in CH₂Cl₂ with 0.1% NH₄OH), to give the title compound; ¹H NMR (DMSO-d₆) δ 8.93 (d, 1H), 8.20 (dd, 1H), 6.42 (d, 1H), 5.88 (d, 1H), 4.68-4.59 (m, 1H), 4.38-4.33 (m, 2H), 3.90-3.86 (m, 2H).

Step 2. 1-(5-aAmino-2-pyridyl)azetidin-3-ol: The Standard Method A, Nitro Reduction, was used to prepare the title compound from the material isolated in Step 1. The material was used without purification.

### Preparation of Intermediate I-17; (4-Amino-2-pyridyl)methanol

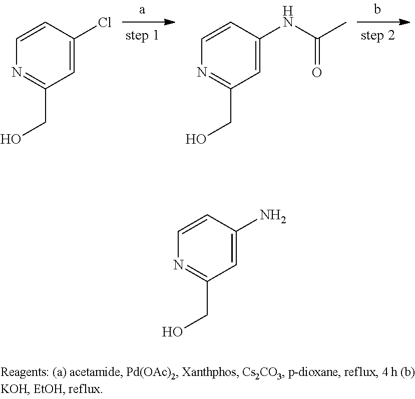

Step 1. N-[2-(Hydroxymethyl)-4-pyridyl]acetamide. A solution of (4-chloro-2-pyridyl)methanol (1.0 g, 6.89 mmol), acetamide (0.61 g, 10.3 mmol), Pd(OAc)₂ (0.075 g, 0.345 mmol), and Xanthphos (0.40 g, 0.689 mmol) in p-dioxane (20 mL) was stirred at reflux for 4 h. After cooling, the mixture was filtered through Celite with the aid of additional DCM, then concentrated under vacuum. The residue was purified by column chromatography (SiO₂, MeOH 20% in CH₂Cl₂ with 0.1% NH₄OH), to give the title compound; ¹H NMR (DMSO-d₆) δ 10.0 (s, 1H), 8.29 (d, 1H), 7.63 (d, 1H), 7.46 (dd, 1H), 5.41 (t, 1H), 4.49 (d, 1H), 2.08 (s, 3H).

Step 2. (4-Amino-2-pyridyl)methanol: N-[2-(Hydroxymethyl)-4-pyridyl]acetamide (0.050 g, 0.30 mmol) was dissolved in EtOH and treated with KOH (0.033 g, 0.60 mmol). The solution was heated at reflux for 2 h. The solution was concentrated under a stream of nitrogen gas and the residue dissolved with DCM. The organic solution was dried (Na₂SO₄) and concentrated to give the title compound. ¹H NMR (DMSO-d₆) δ 7.86 (d, 1H), 6.60 (d, 1H), 6.30 (dd, 1H), 5.97 (s, 2H), 5.22 (s, 1H), 4.34 (s, 2H).

### Preparation of Intermediate I-18; 3-(Methylaminomethyl)aniline

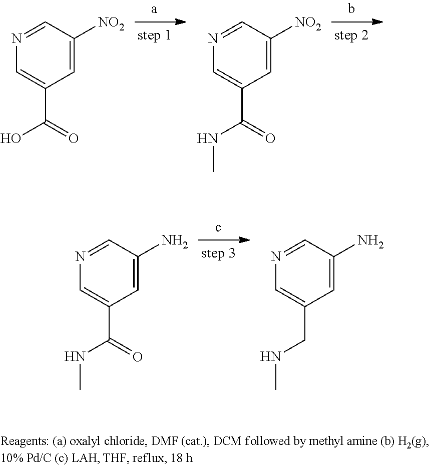

Step 1. N-Methyl-3-nitro-benzamide: 3-Nitrobenzoic acid (1 g, 6 mmol) was dissolved in DCM (15 mL) and treated with oxalyl chloride (3 mL) at rt. The resulting solution was treated with approximately 2 drops of DMF resulting in an exothermic reaction. Stirring continued for 3 h at rt, whereupon the reaction was concentrated under vacuum. A portion of the acid chloride intermediate (⅓) was transferred to a clean flask and fresh DCM was along with 2 M methyl amine solution in THF (4 mmol). The resulting solution was stirred for 3 h at rt, then washed 10% HCl (aq) solution. The organic layer was dried (Na₂SO₄) and concentrated to give the title amide product. The remaining crude acid chloride was used to prepare additional amide products according to the same procedure.

Step 2. 3-Amino-N-methyl-benzamide: Standard Method A; Nitro Reduction, was used to prepare the title compound from the material isolated in Step 1.

Step 3. 3-(Methylaminomethyl)aniline: Standard Method D; LAH Reduction of Amides, was used to prepared the title compound from 3-amino-N-methyl-benzamide (1.5 mmol) in 20 mL of THF, to give the product (89 mg): GC/MS (EI, M+) 137.

### Preparation of Intermediate I-19; 5-Methylpyridin-3-amine

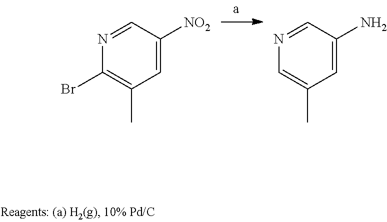

Standard Method A; Nitro Reduction was used to prepare the title compound from 2-bromo-3-methyl-5-nitro-pyridine; GC/MS (EI, M+) 108.

### Preparation of Intermediate I-20; (5-Amino-2-pyridyl)methanol

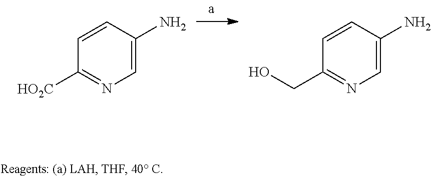 [SMILES: Nc1ccc(C(=O)O)nc1 ; Nc1ccc(CO)nc1]

Standard Method E; LAH Reduction of Carboxylic Acids was used to prepare the title compound from 5-aminopyridine-2-carboxylic acid; ¹H NMR (DMSO-d₆) δ 7.83 (d, 1H), 7.08 (d, 1H), 6.90 (dd, 1H), 5.16 (br s, 3H), 4.36 (s, 2H).

### Preparation of Intermediate I-21; (5-Amino-2-thienyl)-[4-(2-hydroxyethyl)piperazin-1-yl]methanone

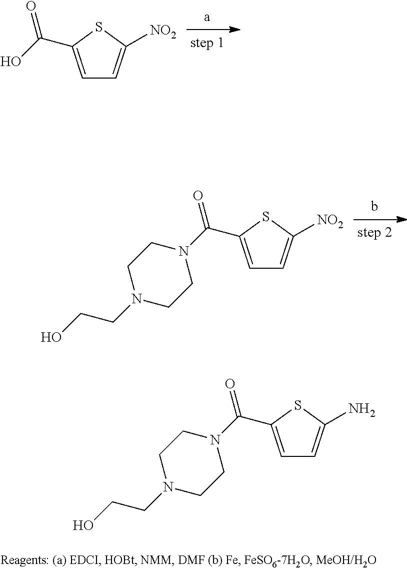 [SMILES: O=C(O)c1ccc([N+](=O)[O-])s1 ; O=C(c1ccc([N+](=O)[O-])s1)N1CCN(CCO)CC1 ; Nc1ccc(C(=O)N2CCN(CCO)CC2)s1]

Step 1. [4-(2-Hydroxyethyl)piperazin-1-yl]-(5-nitro-2-thienyl)methanone. 5-Nitrothiophene-2-carboxylic acid (0.073 g, 0.62 mmol), 2-piperazin-1-ylethanol (0.073 mL, 0.59 mmol), EDCI (0.097 mg, 0.5 mmol), HOBt (0.029 g, 0.211 mmol), and NMM (0.163 mL, 1.47 mmol) were combined with DMF (0.5 mL) and stirred at rt overnight. The reaction was concentrated under vacuum and the residue purified by column chromatography (SiO₂, MeOH 20% in CH₂Cl₂) to give the title compound; LC-MS [M+H]⁺ 286.2.

Step 2. (5-Amino-2-thienyl)-[4-(2-hydroxyethyl)piperazin-1-yl]methanone. [4-(2-Hydroxyethyl)piperazin-1-yl]-(5-nitro-2-thienyl)methanone was dissolved in a mixture of 3:1 MeOH/H₂O (8 mL) and treated with Fe (0.960 g, 1.33 mmol) and FeSO₄-7H₂O (0.370 g, 1.33 mmol). The mixture was heated to 70° C. for 5 h. The reaction was allowed to cool and the mixture was filtered. The filtrate was concentrated under vacuum to give the title compound which was used without further purification; LC-MS [M+H]⁺ 256.0.

### Preparation of Intermediate I-22; (6-Amino-2-pyridyl)-morpholino-methanone

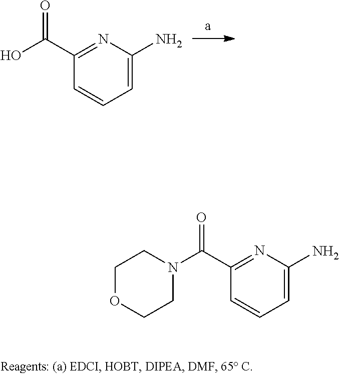 [SMILES: Nc1cccc(C(=O)O)n1 ; Nc1cccc(C(=O)N2CCOCC2)n1]

Standard Method G; EDCI Coupling was used to prepare the title compound from 6-aminopyridine-2-carboxylic acid and morpholine; ¹H NMR (CDCl₃) δ 7.42-7.56 (m, 1H), 6.86 (d, 1H), 6.52 (d, 1H), 3.79 (br s, 2H), 3.55 (d, 4H), 2.93 (d, 4H).

### Preparation of Intermediate I-23; 6-(3-Morpholinopyrrolidin-1-yl)pyridazin-3-amine

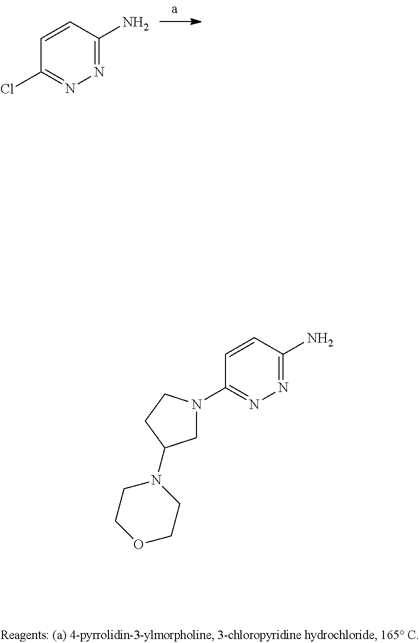 [SMILES: Nc1ccc(Cl)nn1 ; Nc1ccc(N2CCC(N3CCOCC3)C2)nn1]

6-Chloropyridazin-3-amine (0.10 g, 0.77 mmol), 4-pyrrolidin-3-ylmorpholine (0.350, 2.24 mmol), and 3-chloropyridine hydrochloride (0.376 g, 2.51 mmol) as catalyst were combined and heated to 165° C. for 4 h. The residue was purified by column chromatography (SiO₂, MeOH 20% in CH₂Cl₂ with 0.1% NH₄OH), to give the title compound; HPLC ret time: 2.35 min-

The structures and physicochemical characterization of additional synthesized intermediates are provided in Table 1 below. The intermediates were synthesized using the methods outlined above using commercially available starting materials that are well known in the art.

TABLE 1
Additional Intermediates

| | | | Standard |
|---|---|---|---|
| No. | Structure | Analytical Data | Method |
| I-24 | 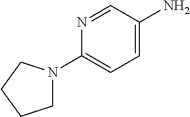 [SMILES: Nc1ccc(N2CCCC2)nc1] | | Intermediate I-13 |
| I-25 | 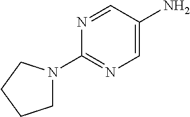 [SMILES: Nc1cnc(N2CCCC2)nc1] | | Intermediate I-13 |
| I-26 | 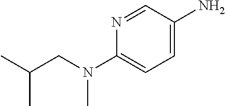 | | Intermediate I-13 |
| I-27 | 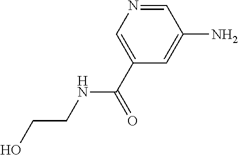 [SMILES: Nc1cncc(C(=O)NCCO)c1] | GC/MS (EI, M⁺) 181 | Intermediate I- 18; Step 1-2 |
| I-28 | 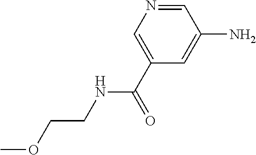 | GC/MS (EI, M⁺) 195 | Intermediate I- 18; Step 1-2 |
| I-29 | 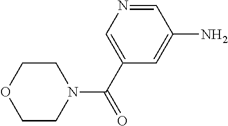 [SMILES: Nc1cncc(C(=O)N2CCOCC2)c1] | GC/MS (EI, M⁺) 207 | Intermediate I- 18; Step 1-2 |
| I-30 | 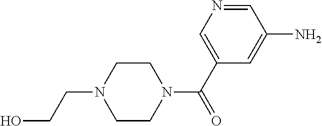 [SMILES: Nc1cncc(C(=O)N2CCN(CCO)CC2)c1] | GC/MS (EI, M⁺) 250 | Intermediate I- 18; Step 1-2 |
| I-31 | 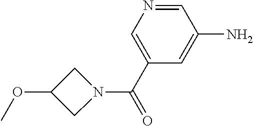 | GC/MS (EI, M⁺) 207 | Intermediate I- 18; Step 1-2 |
| I-32 | 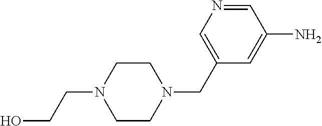 [SMILES: Nc1cncc(CN2CCN(CCO)CC2)c1] | ¹H NMR (CDCl₃) δ 7.86 (d, 1H), 7.74 (d, 1H), 7.08-7.06 (m, 1H), 3.76- 3.74 (m, 2H), 3.47-3.44 (m, 4H), 2.62-2.48 (m, 8H) | Intermediate I-18 |
| I-33 | 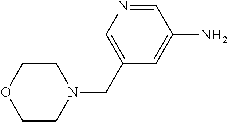 [SMILES: Nc1cncc(CN2CCOCC2)c1] | GC/MS (EI, M⁺) 193 | Intermediate I-18 |
| I-34 | 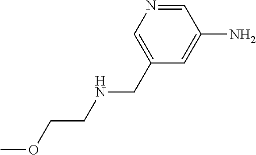 | GC/MS (EI, M⁺) 181 | Intermediate I-18 |
| I-35 | 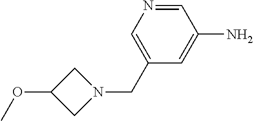 | GC/MS (EI, M⁺) 193 | Intermediate I-18 |
| I-36 | 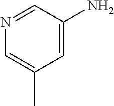 | GC/MS (EI, M⁺) 108 | Standard Method A; Nitro Reduction |
| I-37 | 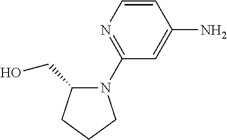 [SMILES: Nc1ccnc(N2CCC[C@@H]2CO)c1] | ¹H NMR (DMSO-d₆) δ 7.53 (d, 1H), 5.85 (dd, 1H), 5.68 (s, 2H), 5.56 (d, 1H), 3.98-3.92 (m, 1H), 3.49 (dd, 1H), 3.30- 3.23 (m, 2H), 3.12-3.06 (m, 1H), 1.96-1.81 (m, 4H). | Intermediate I-14 |
| I-38 | 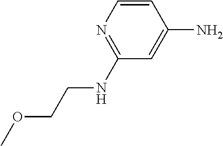 | ¹H NMR (DMSO-d₆) δ 7.47 (d, 1H), 5.78 (dd, 1H), 5.56 (d, 2H), 5.50 (s, 2H), 3.93 (t, 2H), 3.29 (q, 2H), 3.25 (s, 3H). | Intermediate I-14 |
| I-39 | 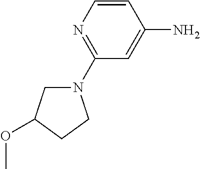 | ¹H NMR (DMSO-d₆) δ 7.55 (d, 1H), 5.83 (dd, 1H), 5.58 (s, 2H), 5.49 (d, 1H), 4.02-3.98 (m, 1H), 3.41-3.31 (m, 3H), 3.27-3.22 (m, 1H), 3.24 (s, 3H), 2.01-1.97 (m, 2H). | Intermediate I-14 |
| I-40 | 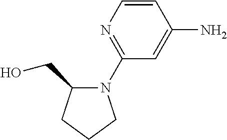 [SMILES: Nc1ccnc(N2CCC[C@H]2CO)c1] | ¹H NMR (DMSO-d₆) δ 7.53 (d, 1H), 5.85 (dd, 1H), 5.68 (s, 2H), 5.56 (d, 1H), 3.98-3.92 (m, 1H), 3.49 (dd, 1H), 3.30- 3.23 (m, 2H), 3.12-3.06 (m, 1H), 1.96-1.81 (m, 4H). | Intermediate I-14 |
| I-41 | 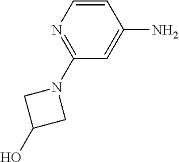 [SMILES: Nc1ccnc(N2CC(O)C2)c1] | ¹H NMR (DMSO-d₆) δ 7.55 (d, 1H), 5.98 (dd, 1H), 5.66 (s, 2H), 5.53 (d, 1H), 5.42 (d, 1H), 4.52-4.47 (m, 1H), 4.00- 3.97 (m, 2H), 3.53-3.49 (m, 2H). | Intermediate I-14 |
| I-42 | 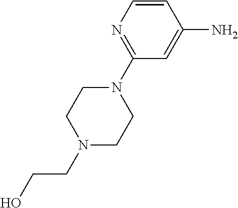 [SMILES: Nc1ccnc(N2CCN(CCO)CC2)c1] | ¹H NMR (CDCl₃) δ 7.89 (d, 1H), 6.03 (dd, 1H), 5.88 (d, 1H), 3.98 (br s, 2H), 3.65 (t, 2H), 3.54- 3.45 (m, 5H), 2.63-2.58 (m, 6H). | Intermediate I-14 |
| I-43 | 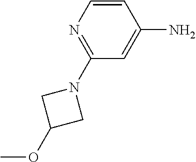 | ¹H NMR (CDCl₃) δ 7.80 (d, 1H), 5.99 (dd, 1H), 5.51 (d, 1H), 4.34-4.49 (m, 1H), 4.21-4.13 (m, 6H), 3.85 (dd, 2H), 3.32 (s, 3H). | Intermediate I-14 |
| I-44 | 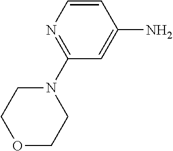 [SMILES: Nc1ccnc(N2CCOCC2)c1] | ¹H NMR (CDCl₃) δ 7.89 (d, 1H), 6.05 (dd, 1H), 5.86 (d, 1H), 4.06 (br s, 2H), 3.81 (t, 4H), 3.44 (t, 4H). | Intermediate I-14 |
| I-45 | 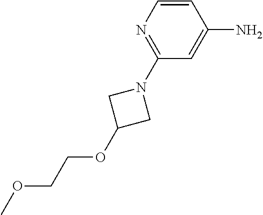 [SMILES: COCCOC1CN(c2cc(N)ccn2)C1] | ¹H NMR (DMSO-d₆) δ 7.58 (d, 1H), 6.21 (br s, 2H), 5.99 (dd, 1H), 5.44 (d, 1H), 4.41-4.37 (m, 1H), 4.05 (t, 2H), 3.63 (dd, 2H), 3.51-3.49 (m, 2H), 3.46-3.43 (m, 2H), 3.25 (s, 3H). | Intermediate I-14 |
| I-46 | 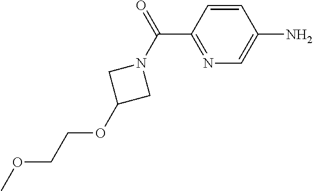 [SMILES: COCCOC1CN(C(=O)c2ccc(N)cn2)C1] | ¹H NMR (DMSO-d₆) δ 7.87 (d, 1H), 7.65 (d, 1H), 6.93 (dd, 1H), 5.93 (s, 2H), 4.70-4.65 (m, 1H), 4.32-4.25 (m, 2H), 4.16 (dd, 1H), 3.51-3.49 (m, 2H), 3.46-3.43 (m, 2H), 3.25 (s, 3H). | Standard Method C; HATU Coupling |
| I-47 | 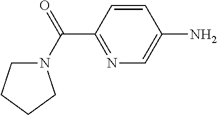 [SMILES: Nc1ccc(C(=O)N2CCCC2)nc1] | ¹H NMR (DMSO-d₆) δ 7.87 (d, 1H), 7.52 (d, 1H), 6.93 (dd, 1H), 5.79 (s, 2H), 3.73 (t, 2H), 3.44 (t, 2H), 1.84-1.77 (m, 4H). | Standard Method C; HATU Coupling |
| I-48 | 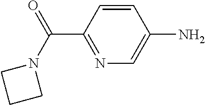 [SMILES: Nc1ccc(C(=O)N2CCC2)nc1] | ¹H NMR (DMSO-d₆) δ 7.86 (d, 1H), 7.64 (d, 1H), 6.92 (dd, 1H), 5.89 (s, 2H), 4.03-3.96 (m, 4H), 1.29-1.22 (m, 2H). | Standard Method C; HATU Coupling |
| I-49 | 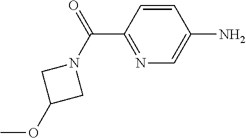 | ¹H NMR (DMSO-d₆) δ 7.87 (d, 1H), 7.65 (d, 1H), 6.93 (dd, 1H), 5.93 (s, 2H), 4.68 (dd, 1H), 4.28 (dd, 1H), 4.20-4.13 (m, 2H), 3.22 (s, 3H). | Standard Method C; HATU Coupling |
| I-50 | 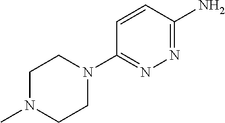 | HPLC ret time 2.35 min | Intermediate I-23 |
| I-51 | 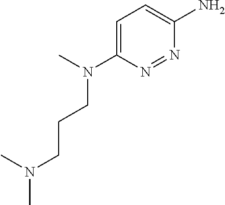 | HPLC ret time 2.35 min | Intermediate I-23 |
| I-52 | 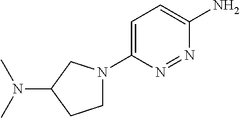 | HPLC ret time 2.35 min | Intermediate I-23 |
| I-53 | 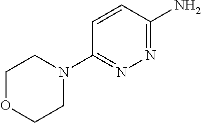 [SMILES: Nc1ccc(N2CCOCC2)nn1] | ¹H NMR (MeOH-d₄) δ 7.20 (d, 1H), 6.91 (d, 1H), 4.91 (s, 2H), 3.71- 3.87 (m. 4H), 3.22-3.40 (m, 4H) | Intermediate I-23 |
| I-54 | 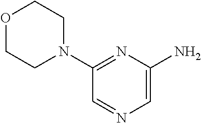 [SMILES: Nc1cncc(N2CCOCC2)n1] | LC/MS [M + H] 181 | Intermediate I-23 |

### Preparation of Specific Example Compounds

### Example Compound 1; 5-[2-({6-[(2-Hydroxyethyl)(methyl)amino]pyridin-3-yl}amino)pyrimidin-4-yl]-2-(tetrahydro-2H-pyran-4-yloxy)benzonitrile

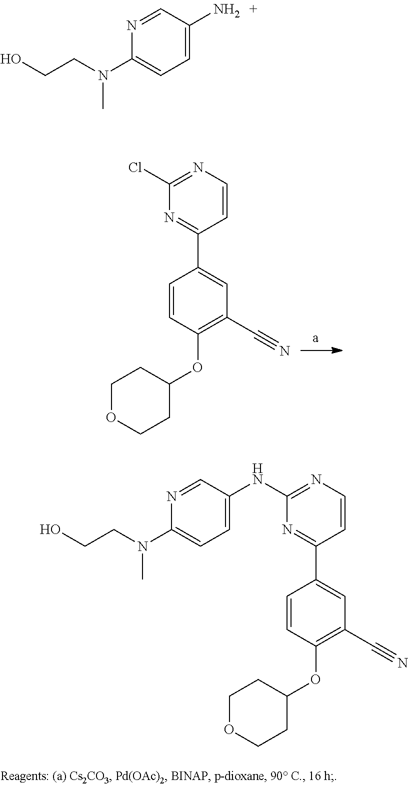 [SMILES: CN(CCO)c1ccc(N)cn1 ; N#Cc1cc(-c2ccnc(Cl)n2)ccc1OC1CCOCC1 ; CN(CCO)c1ccc(Nc2nccc(-c3ccc(OC4CCOCC4)c(C#N)c3)n2)cn1]

5-(2-Chloropyrimidin-4-yl)-2-tetrahydropyran-4-yloxy-benzonitrile (0.16 g, 0.50 mmol), 2-[(5-amino-2-pyridyl)-methyl-amino]ethanol (0.09 g, 0.50 mmol), cesium carbonate (0.31 g, 0.95 mmol), Pd(OAc)₂ (0.020 g, 0.1 mmol) and BINAP (0.10 g, 0.08 mmol) and p-dioxane (10 mL) were added to a flask and the reaction mixture sparged with nitrogen (3 min). The reaction mixture was placed in an oil bath at 90° C. and stirred for 14 h. The reaction was cooled to rt, H₂O (5.0 mL) and 3:1 iPrOH/CHCl₃ (25 mL) were added and the layers separated. The organic layer was dried over sodium sulfate, filtered, and evaporated under reduced pressure to give the crude product. Purification by RP-MPLC (C₁₈, MeOH/H₂O, 0-100%, with 0.1% TFA) provided the title compound. (0.035 g, 16%). ¹H NMR (DMSO-d₆) δ 9.92 (s, 1H), 8.63 (d, 1H), 8.60-8.58 (m, 1H), 8.53 (d, 1H), 8.45-8.42 (m, 1H), 8.17-8.13 (m, 1H), 7.55-7.51 (m, 2H), 7.37 (d, 1H), 4.98-4.92 (m, 1H), 3.91-3.85 (m, 2H), 3.70-3.66 (m, 4H), 3.59-3.53 (m, 2H), 3.21 (s, 3H), 2.07-2.02 (m, 2H), 1.74-1.65 (m, 2H). LC-MS [M+H]⁺ 447.2332.

### Example Compound 2; 5-(2-{[2-(Morpholin-4-yl)pyrimidin-5-yl]amino}pyrimidin-4-yl)-2-(tetrahydro-2H-pyran-4-yloxy)benzonitrile

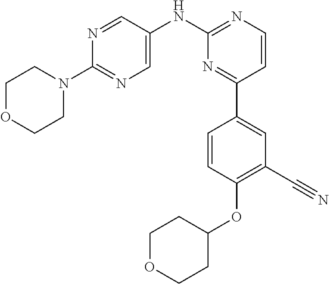 [SMILES: N#Cc1cc(-c2ccnc(Nc3cnc(N4CCOCC4)nc3)n2)ccc1OC1CCOCC1]

This compound was prepared according to the procedure described for the preparation of Example Compound 1, using 2-morpholinopyrimidin-5-amine. The product was purified by column chromatography (SiO₂, MeOH/DCM, 0-40%) to give the title compound; ¹H NMR (DMSO-d₆) δ 9.49 (s, 1H), 8.73 (s, 2H), 8.50-8.48 (m, 2H), 8.40-8.37 (m, 1H), 7.55 (d, 1H), 7.44 (d, 1H), 4.96-4.91 (m, 1H), 3.94-3.84 (m, 2H), 3.72-3.61 (m, 8H), 3.58-3.52 (m, 2H), 2.07-2.01 (m, 2H), 1.73-1.64 (m, 2H). LC-MS [M+H]⁺ 460.2048.

### Example Compound 3; 5-(2-{[2-(Pyrrolidin-1-yl)pyrimidin-5-yl]amino}pyrimidin-4-yl)-2-(tetrahydro-2H-pyran-4-yloxy)benzonitrile

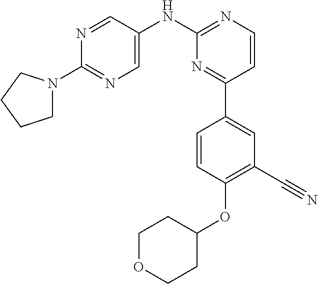 [SMILES: N#Cc1cc(-c2ccnc(Nc3cnc(N4CCCC4)nc3)n2)ccc1OC1CCOCC1]

This compound was prepared according to the procedure described for the preparation of Example Compound 1, using 2-pyrrolidin-1-ylpyrimidin-5-amine. The product was purified by column chromatography (SiO₂, MeOH/DCM, 0-40%) to give the title compound; ¹H NMR (DMSO-d₆) δ 9.37 (s, 1H), 8.64 (s, 2H), 8.48-8.46 (m, 2H), 8.39-8.37 (m, 1H), 7.55 (d, 1H), 7.42 (d, 1H), 4.96-4.91 (m, 1H), 3.90-3.84 (m, 2H), 3.58-3.47 (m, 6H), 2.08-2.00 (m, 2H), 1.96-1.92 (m, 4H), 1.72-1.63 (m, 2H). LC-MS [M+H]⁺ 444.2132.

### Example Compound 4; 5-{2-[(6-Cyclopropylpyridin-3-yl)amino]pyrimidin-4-yl}-2-(tetrahydro-2H-pyran-4-yloxy)benzonitrile

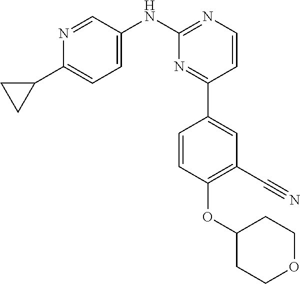 [SMILES: N#Cc1cc(-c2ccnc(Nc3ccc(C4CC4)nc3)n2)ccc1OC1CCOCC1]

This compound was prepared according to the procedure described for the preparation of Example Compound 1, using 6-cyclopropylpyridin-3-amine. Purification by RP-MPLC (C₁₈, MeOH/H₂O, 0-100%, with 0.1% TFA) provided the title compound; ¹H NMR (DMSO-d₆) δ 10.31 (s, 1H), 9.14 (s, 1H), 8.65 (d, 1H), 8.55 (d, 1H), 8.47-8.44 (m, 1H), 8.38-8.35 (m, 1H), 7.62 (d, 1H), 7.55-7.52 (m, 2H), 4.98-4.93 (m, 1H), 3.91-3.85 (m, 2H), 3.59-3.53 (m, 2H), 2.29-2.23 (m, 1H), 2.08-2.02 (M, 2H), 1.74-1.65 (m, 2H), 1.21-1.17 (m, 2H), 1.07-1.02 (m, 2H). LC-MS [M+H]⁺ 414.1897.

### Example Compound 5; 5-(2-{[6-(Pyrrolidin-1-yl)pyridin-3-yl]amino}pyrimidin-4-yl)-2-(tetrahydro-2H-pyran-4-yloxy)benzonitrile

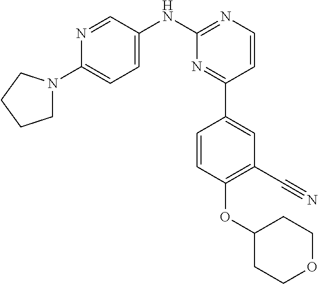 [SMILES: N#Cc1cc(-c2ccnc(Nc3ccc(N4CCCC4)nc3)n2)ccc1OC1CCOCC1]

This compound was prepared according to the procedure described for the preparation of Example Compound 1, using 6-pyrrolidin-1-ylpyridin-3-amine. The product was purified by column chromatography (SiO₂, MeOH/DCM, 0-40%) to give the title compound; ¹H NMR (DMSO-d₆) δ 9.91 (s, 1H), 8.63 (d, 1H), 8.59 (d, 1H), 8.52 (d, 1H), 8.45-8.42 (m, 1H), 8.19-8.16 (m, 1H), 7.56-7.51 (m, 2H), 7.18 (d, 1H), 4.97-4.93 (m, 1H), 3.91-3.83 (m, 2H), 3.59-3.51 (m, 6H), 2.06-2.02 (m, 6H), 1.74-1.65 (m, 2H). LC-MS [M+H]⁺ 443.2236.

### Example Compound 6; 5-(2-{[6-(Morpholin-4-yl)pyridin-3-yl]amino}pyrimidin-4-yl)-2-(tetrahydro-2H-pyran-4-yloxy)benzonitrile

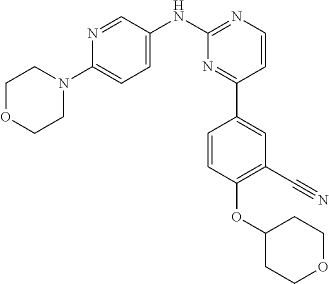 [SMILES: N#Cc1cc(-c2ccnc(Nc3ccc(N4CCOCC4)nc3)n2)ccc1OC1CCOCC1]

This compound was prepared according to the procedure described for the preparation of Example Compound 1, using 6-morpholinopyridin-3-amine. Purification by RP-MPLC (C₁₈, MeOH/H₂O, 0-100%, with 0.1% TFA) provided the title compound; ¹H NMR (DMSO-d₆) δ 9.55 (s, 1H), 8.54 (d, 1H), 8.51-8.49 (m, 2H), 8.43-8.40 (m, 1H), 7.98-7.95 (m, 1H), 7.55 (d, 1H), 7.43 (d, 1H), 6.94 (d, 1H), 4.97-4.92 (m, 1H), 3.90-3.84 (m, 2H), 3.74-3.69 (m, 4H), 3.58-3.52 (m, 2H), 3.41-3.38 (m, 4H), 2.54 (s, 3H), 2.08-2.01 (m, 2H), 1.73-1.65 (m, 2H). LC-MS [M+H]⁺ 459.2102.

### Example Compound 7; 5 5-{2-[(6-Methylpyridin-3-yl)amino]pyrimidin-4-yl}-2-(tetrahydro-2H-pyran-4-yloxy)benzonitrile

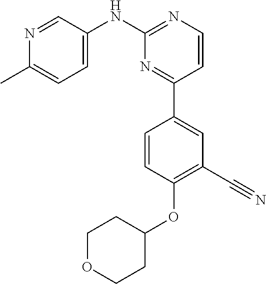 [SMILES: Cc1ccc(Nc2nccc(-c3ccc(OC4CCOCC4)c(C#N)c3)n2)cn1]

This compound was prepared according to the procedure described for the preparation of Example Compound 1, using 6-methylpyridin-3-amine. The product was purified by column chromatography (SiO₂, MeOH/DCM, 0-40%) to give the title compound; ¹H NMR (DMSO-d₆) δ 10.44 (s, 1H), 9.27 (d, 1H), 8.67 (d, 1H), 8.55 (d, 1H), 8.48-8.44 (m, 2H), 7.75 (d, 1H), 7.64 (d, 1H), 7.53 (d, 1H), 4.97-4.92 (m, 1H), 3.91-3.85 (m, 2H), 3.59-3.51 (m, 2H), 2.63 (s, 3H), 2.07-2.03 (m, 2H), 1.74-1.66 (m, 2H). LC-MS [M+H]⁺ 388.1956.

### Example Compound 8; 5-{2-[(6-Ethoxypyridin-3-yl)amino]pyrimidin-4-yl}-2-(tetrahydro-2H-pyran-4-yloxy)benzonitrile

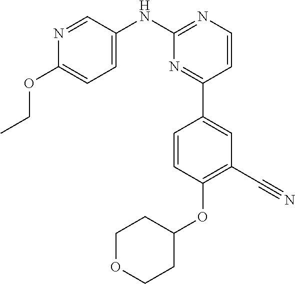 [SMILES: CCOc1ccc(Nc2nccc(-c3ccc(OC4CCOCC4)c(C#N)c3)n2)cn1]

This compound was prepared according to the procedure described for the preparation of Example Compound 1, using 6-ethoxypyridin-3-amine. The product was purified by column chromatography (SiO₂, MeOH/DCM, 0-40%) to give the title compound; ¹H NMR (DMSO-d₆) δ 9.62 (s, 1H), 8.52-8.51 (m, 3H), 8.43-8.40 (m, 1H), 8.04-8.01 (m, 1H), 7.55 (d, 1H), 7.45 (d, 1H), 6.80 (d, 1H), 4.97-4.91 (m, 1H), 4.30-4.24 (m, 2H), 3.90-3.84 (m, 2H), 3.58-3.52 (m, 2H), 2.08-2.01 (m, 2H), 1.73-1.64 (m, 2H), 1.32 (t, 3H). LC-MS [M+H]⁺ 418.2686.

### Example Compound 9; 5-(2-{[6-(Diethylamino)pyridin-3-yl]amino}pyrimidin-4-yl)-2-(tetrahydro-2H-pyran-4-yloxy)benzonitrile

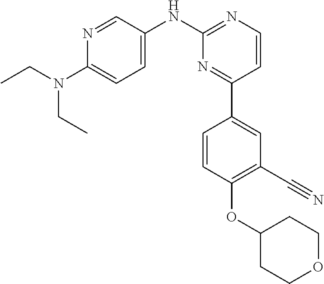 [SMILES: CCN(CC)c1ccc(Nc2nccc(-c3ccc(OC4CCOCC4)c(C#N)c3)n2)cn1]

This compound was prepared according to the procedure described for the preparation of Example Compound 1 using N2,N2-diethylpyridine-2,5-diamine. Purification by RP-MPLC (C₁₈, MeOH/H₂O, 0-100%, with 0.1% TFA) provided the title compound; ¹H NMR (DMSO-d₆) δ 9.88 (s, 1H), 8.62-8.57 (m, 2H), 8.53 (d, 1H), 8.45-8.42 (m, 1H), 8.15 (d, 1H), 7.55-7.49 (m, 2H), 7.28 (br s, 1H), 4.97-4.92 (m, 1H), 3.91-3.85 (m, 2H), 3.61-3.51 (m, 6H), 2.07-2.02 (m, 2H), 1.74-1.65 (m, 2H), 1.19 (t, 6H). LC-MS [M+H]⁺ 445.2336.

### Example Compound 10; 5-(2-{[6-(Dimethylamino)pyridin-3-yl]amino}pyrimidin-4-yl)-2-(tetrahydro-2H-pyran-4-yloxy)benzonitrile

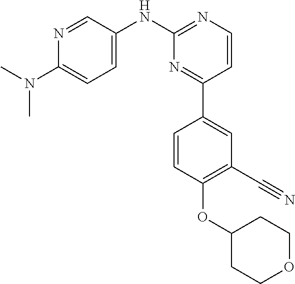 [SMILES: CN(C)c1ccc(Nc2nccc(-c3ccc(OC4CCOCC4)c(C#N)c3)n2)cn1]

This compound was prepared according to the procedure described for the preparation of Example Compound 1 using N2,N2-dimethylpyridine-2,5-diamine. Purification by RP-MPLC (C₁₈, MeOH/H₂O, 0-100%, with 0.1% TFA) provided the title compound; ¹H NMR (DMSO-d₆) δ 9.86 (s, 1H), 8.62 (d, 1H), 8.58 (d, 1H), 8.52 (d, 1H), 8.16-8.12 (m, 1H), 7.55-7.52 (m, 2H), 7.24 (d, 1H), 4.97-4.92 (m, 1H), 3.90-3.85 (m, 2H), 3.59-3.53 (m, 2H), 3.18 (s, 6H), 2.07-2.02 (m, 2H), 1.74-1.65 (m, 2H). LC-MS [M+H]⁺ 417.2021.

### Example Compound 11; 5-[2-(Pyridin-3-ylamino)pyrimidin-4-yl]-2-(tetrahydro-2H-pyran-4-yloxy)benzonitrile

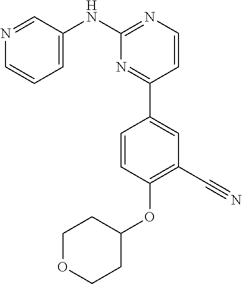 [SMILES: N#Cc1cc(-c2ccnc(Nc3cccnc3)n2)ccc1OC1CCOCC1]

This compound was prepared according to the procedure described for the preparation of Example Compound 1 using pyridin-3-amine. Purification by column chromatography (SiO₂, MeOH/DCM, 0-40%) provided the title compound; ¹H NMR (DMSO-d₆) δ 9.91 (s, 1H), 8.97 (d, 1H), 8.59 (d, 1H), 8.54 (d, 1H), 8.46-8.43 (m, 1H), 8.25-8.19 (m, 2H), 7.57-7.52 (m, 2H), 7.38-7.34 (m, 1H), 4.97-4.91 (m, 1H), 3.91-3.85 (m, 2H), 3.59-3.53 (m, 2H), 2.08-2.02 (m, 2H), 1.74-1.65 (m, 2H). LC-MS [M+H]⁺ 375.1647.

### Example Compound 12; 5-({4-[3-Cyano-4-(tetrahydro-2H-pyran-4-yloxy)phenyl]pyrimidin-2-yl}amino)-N-methylpyridine-3-carboxamide

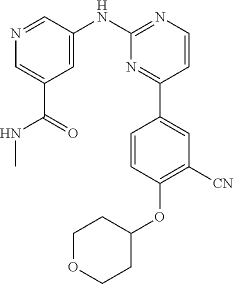 [SMILES: CNC(=O)c1cncc(Nc2nccc(-c3ccc(OC4CCOCC4)c(C#N)c3)n2)c1]

This compound was prepared according to the procedure described for the preparation of Example Compound 1 using 5-amino-N-methyl-pyridine-3-carboxamide. Purification by RP-MPLC (C₁₈, MeOH/H₂O, 0-100%, with 0.1% TFA) provided the title compound; ¹H NMR (CDCl₃) δ 9.02 (d, 1H), 8.67 (s, 1H), 8.57 (d, 1H), 8.50 (d, 1H), 8.35 (dd, 1H), 7.18-7.12 (m, 2H), 4.82-4.77 (m, 1H), 4.07-4.01 (m, 2H), 3.70-3.64 (m, 2H), 3.04 (s, 3H), 2.13-2.08 (m, 2H), 1.95-1.90 (m, 2H). LC-MS [M+H]⁺ 431.1844.

### Example Compound 13; 5-({4-[3-Cyano-4-(tetrahydro-2H-pyran-4-yloxy)phenyl]pyrimidin-2-yl}amino)-N-(2-hydroxyethyl)pyridine-3-carboxamide

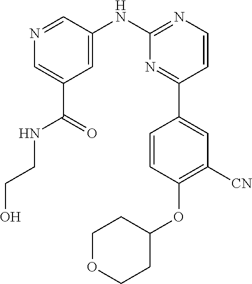 [SMILES: N#Cc1cc(-c2ccnc(Nc3cncc(C(=O)NCCO)c3)n2)ccc1OC1CCOCC1]

This compound was prepared according to the procedure described for the preparation of Example Compound 1 using 5-amino-N-(2-hydroxyethyl)pyridine-3-carboxamide. Purification by RP-MPLC (C₁₈, MeOH/H₂O, 0-100%, with 0.1% TFA) provided the title compound; ¹H NMR (CDCl₃) δ 9.04 (d, 1H), 8.66 (d, 1H), 8.59 (d, 1H), 8.49 (d, 1H), 8.35-8.27 (m, 2H), 7.19-7.16 (m, 2H), 4.82-4.77 (m, 1H), 4.07-4.01 (m, 2H), 3.82 (t, 2H), 3.71-3.66 (m, 2H), 3.62 (t, 2H), 2.13-2.07 (m, 2H), 1.97-1.89 (m, 2H). LC-MS [M+H]⁺ 461.1948.

### Example Compound 14; 5-{2-[(5-{[4-(2-Hydroxyethyl)piperazin-1-yl]carbonyl}pyridin-3-yl)amino]pyrimidin-4-yl}-2-(tetrahydro-2H-pyran-4-yloxy)benzonitrile

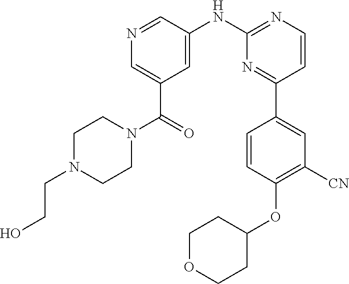 [SMILES: N#Cc1cc(-c2ccnc(Nc3cncc(C(=O)N4CCN(CCO)CC4)c3)n2)ccc1OC1CCOCC1]

This compound was prepared according to the procedure described for the preparation of Example Compound 1 using (5-amino-3-pyridyl)-[4-(2-hydroxyethyl)piperazin-1-yl]methanone. Purification by RP-MPLC (C₁₈, MeOH/H₂O, 0-100%, with 0.1% TFA) provided the title compound; ¹H NMR (CDCl₃) δ 8.83 (d, 1H), 8.52 (d, 1H), 8.43-8.42 (m, 1H), 8.34-8.24 (m, 3H), 7.34 (s, 1H), 7.17 (d, 1H), 7.11 (d, 1H), 4.79-4.75 (m, 1H), 4.07-4.01 (m, 2H), 3.87 (br. s, 2H), 3.70-3.57 (m, 6H), 2.65-2.53 (m, 6H), 2.13-2.06 (m, 2H), 1.97-1.89 (m, 2H). LC-MS [M+H]⁺ 530.2515.

### Example Compound 15; 5-({4-[3-Cyano-4-(tetrahydro-2H-pyran-4-yloxy)phenyl]pyrimidin-2-yl}amino)-N-(2-methoxyethyl)pyridine-3-carboxamide

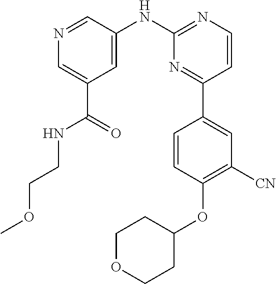 [SMILES: COCCNC(=O)c1cncc(Nc2nccc(-c3ccc(OC4CCOCC4)c(C#N)c3)n2)c1]

This compound was prepared according to the procedure described for the preparation of Example Compound 1 using 5-amino-N-(2-methoxyethyl)pyridine-3-carboxamide. Purification by RP-MPLC (C₁₈, MeOH/H₂O, 0-100%, with 0.1% TFA) provided the title compound; ¹H NMR (CDCl₃) δ 8.99 (d, 1H), 8.74-8.73 (m, 1H), 8.64 (d, 1H), 8.53 (d, 1H), 8.33 (dd, 1H), 8.26 (d, 1H), 7.65 (s, 1H), 7.16 (d, 1H), 7.13 (d, 1H), 6.72-6.69 (m, 1H), 4.78-4.75 (m, 1H), 4.07-4.01 (m, 2H), 3.75-3.60 (m, 6H), 3.40 (s, 3H), 2.13-2.06 (m, 2H), 1.97-1.89 (m, 2H). LC-MS [M+H]⁺ 475.2039.

### Example Compound 16; 5-(2-{[5-(Morpholin-4-ylcarbonyl)pyridin-3-yl]amino}pyrimidin-4-yl)-2-(tetrahydro-2H-pyran-4-yloxy)benzonitrile

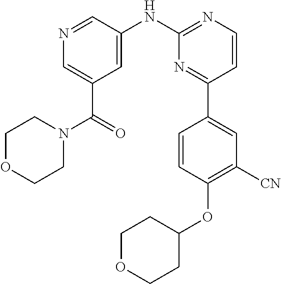 [SMILES: N#Cc1cc(-c2ccnc(Nc3cncc(C(=O)N4CCOCC4)c3)n2)ccc1OC1CCOCC1]

This compound was prepared according to the procedure described for the preparation of Example Compound 1 using (5-amino-3-pyridyl)-morpholino-methanone. Purification by RP-MPLC (C₁₈, MeOH/H₂O, 0-100%, with 0.1% TFA) provided the title compound; ¹H NMR (CDCl₃) δ 8.92 (s, 1H), 8.48 (d, 1H), 8.42 (s, 3H), 8.33 (s, 1H), 8.28-8.26 (m, 3H), 7.17 (d, 1H), 7.11 (d, 1H), 4.79-4.75 (m, 1H), 4.06-4.01 (m, 2H), 3.82-3.48 (m, 10H), 2.13-2.06 (m, 2H), 1.97-1.89 (m, 2H). LC-MS [M+H]⁺ 487.2054.

### Example Compound 17; 5-[2-({5-[(3-Methoxyazetidin-1-yl)carbonyl]pyridin-3-yl}amino)pyrimidin-4-yl]-2-(tetrahydro-2H-pyran-4-yloxy)benzonitrile

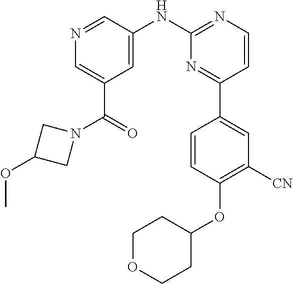 [SMILES: COC1CN(C(=O)c2cncc(Nc3nccc(-c4ccc(OC5CCOCC5)c(C#N)c4)n3)c2)C1]

This compound was prepared according to the procedure described for the preparation of Example Compound 1 using (5-amino-3-pyridyl)-(3-methoxyazetidin-1-yl)methanone. Purification by RP-MPLC (C₁₈, MeOH/H₂O, 0-100%, with 0.1% TFA) provided the title compound; ¹H NMR (CDCl₃) δ 8.92 (d, 1H), 8.68-8.67 (m, 1H), 8.51-8.49 (m, 1H), 8.35-8.32 (m, 1H), 8.26 (d, 1H), 8.10 (s, 1H), 7.17 (d, 1H), 7.14 (d, 1H), 4.79-4.75 (m, 1H), 4.50-4.42 (m, 2H), 4.31-4.25 (m, 2H), 4.15-4.12 (m, 1H), 4.07-4.01 (m, 2H), 3.70-3.64 (m, 2H), 3.32 (s, 3H), 2.13-2.06 (m, 2H), 1.97-1.89 (m, 2H). LC-MS [M+H]⁺ 487.1943.

### Example Compound 18; 5-[2-({5-[(Methylamino)methyl]pyridin-3-yl}amino)pyrimidin-4-yl]-2-(tetrahydro-2H-pyran-4-yloxy)benzonitrile

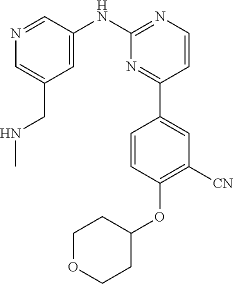 [SMILES: CNCc1cncc(Nc2nccc(-c3ccc(OC4CCOCC4)c(C#N)c3)n2)c1]

This compound was prepared according to the procedure described for the preparation of Example Compound 1 using 5-(methylaminomethyl)pyridin-3-amine. Purification by RP-MPLC (C₁₈, MeOH/H₂O, 0-100%, with 0.1% TFA) provided the title compound; ¹H NMR (CDCl₃) δ 8.73 (d, 1H), 8.51 (d, 1H), 8.36 (d, 1H), 8.31 (s, 1H), 8.28-8.25 (m, 2H), 7.44 (s, 1H), 7.14-7.07 (m, 2H), 4.77-4.74 (m, 1H), 4.07-4.01 (m, 2H), 3.84 (s, 2H), 3.70-3.64 (m, 2H), 2.51 (s, 3H), 2.12-2.06 (m, 2H), 1.97-1.89 (m, 2H). LC-MS [M+H]⁺ 417.1980.

### Example Compound 19; 5-[2-({5-[(Dimethylamino)methyl]pyridin-3-yl}amino)pyrimidin-4-yl]-2-(tetrahydro-2H-pyran-4-yloxy)benzonitrile

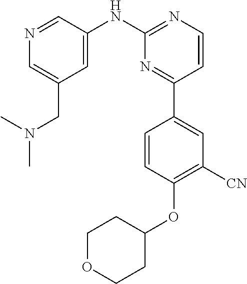 [SMILES: CN(C)Cc1cncc(Nc2nccc(-c3ccc(OC4CCOCC4)c(C#N)c3)n2)c1]

This compound was prepared according to the procedure described for the preparation of Example Compound 1 using 5-(dimethylaminomethyl)pyridin-3-amine. Purification by RP-MPLC (C₁₈, MeOH/H₂O, 0-100%, with 0.1% TFA) provided the title compound; ¹H NMR (CDCl₃) δ 8.80 (d, 1H), 8.51 (d, 1H), 8.31-8.20 (m, 4H), 7.59 (s, 1H), 7.14-7.08 (m, 2H), 4.78-4.74 (m, 1H), 4.07-4.01 (m, 2H), 3.70-3.64 (m, 2H), 3.50 (s, 2H), 2.31 (s, 6H), 2.12-2.06 (m, 2H), 1.97-1.89 (m, 2H). LC-MS [M+H]⁺ 431.2156.

### Example Compound 20; 5-{2-[(5-{[4-(2-Hydroxyethyl)piperazin-1-yl]methyl}pyridin-3-yl)amino]pyrimidin-4-yl}-2-(tetrahydro-2H-pyran-4-yloxy)benzonitrile

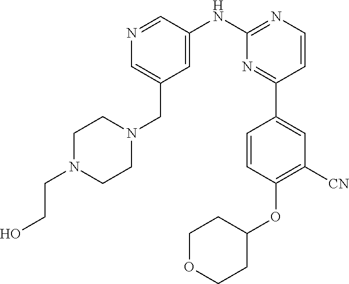 [SMILES: N#Cc1cc(-c2ccnc(Nc3cncc(CN4CCN(CCO)CC4)c3)n2)ccc1OC1CCOCC1]

This compound was prepared according to the procedure described for the preparation of Example Compound 1 using 2-[4-[(5-amino-3-pyridyl)methyl]piperazin-1-yl]ethanol. Purification by RP-MPLC (C₁₈, MeOH/H₂O, 0-100%, with 0.1% TFA) provided the title compound; ¹H NMR (CDCl₃) δ 8.82 (d, 1H), 8.51 (d, 1H), 8.31-8.26 (m, 3H), 8.12-8.10 (m, 1H), 7.21 (s, 1H), 7.14 (d, 1H), 7.09 (d, 1H), 4.78-4.74 (m, 1H), 4.07-4.00 (m, 2H), 3.70-3.62 (m, 2H), 3.61-3.59 (m, 2H), 3.58 (s, 2H), 2.56-2.48 (m, 6H), 2.13-2.05 (m, 2H), 1.97-1.89 (m, 2H), 1.72-1.63 (m, 4H). LC-MS [M+H]⁺ 516.2722.

### Example Compound 21; 5-(2-{[5-(Morpholin-4-ylmethyl)pyridin-3-yl]amino}pyrimidin-4-yl)-2-(tetrahydro-2H-pyran-4-yloxy)benzonitrile

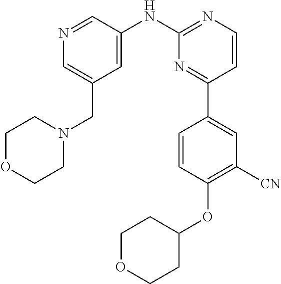 [SMILES: N#Cc1cc(-c2ccnc(Nc3cncc(CN4CCOCC4)c3)n2)ccc1OC1CCOCC1]

This compound was prepared according to the procedure described for the preparation of Example Compound 1 using 5-(morpholinomethyl)pyridin-3-amine. Purification by RP-MPLC (C₁₈, MeOH/H₂O, 0-100%, with 0.1% TFA) provided the title compound; ¹H NMR (CDCl₃) δ 8.83 (d, 1H), 8.51 (d, 1H), 8.31-8.26 (m, 3H), 8.20-8.19 (m, 1H), 7.62 (s, 1H), 7.15 (d, 1H), 7.10 (d, 1H), 4.78-4.74 (m, 1H), 4.07-4.01 (m, 2H), 3.74-3.64 (m, 6H), 3.56 (s, 2H), 2.52-2.50 (m, 4H), 2.13-2.06 (m, 2H), 1.97-1.89 (m, 2H). LC-MS [M+H]⁺ 473.2067.

### Example Compound 22; 5-{2-[(5-{[(2-Methoxyethyl)amino]methyl}pyridin-3-yl)amino]pyrimidin-4-yl}-2-(tetrahydro-2H-pyran-4-yloxy)benzonitrile

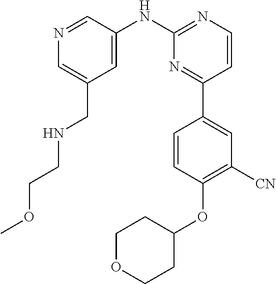 [SMILES: COCCNCc1cncc(Nc2nccc(-c3ccc(OC4CCOCC4)c(C#N)c3)n2)c1]

This compound was prepared according to the procedure described for the preparation of Example Compound 1 using 5-[(2-methoxyethylamino)methyl]pyridin-3-amine. Purification by RP-MPLC (C₁₈, MeOH/H₂O, 0-100%, with 0.1% TFA) provided the title compound; ¹H NMR (CDCl₃) δ 8.78 (d, 1H), 8.50 (d, 1H), 8.31-8.23 (m, 4H), 7.54 (s, 1H), 7.14-7.09 (m, 2H), 4.78-4.74 (m, 1H), 4.07-4.01 (m, 2H), 3.89 (s, 2H), 3.70-3.64 (m, 2H), 3.54 (t, 2H), 3.35 (s, 3H), 2.86 (t, 2H), 2.13-2.06 (m, 2H), 1.97-1.89 (m, 2H). LC-MS [M+H]⁺ 461.2295.

### Example Compound 23; 5-[2-({5-[(3-Methoxyazetidin-1-yl)methyl]pyridin-3-yl}amino)pyrimidin-4-yl]-2-(tetrahydro-2H-pyran-4-yloxy)benzonitrile

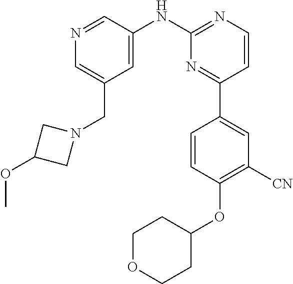 [SMILES: COC1CN(Cc2cncc(Nc3nccc(-c4ccc(OC5CCOCC5)c(C#N)c4)n3)c2)C1]

This compound was prepared according to the procedure described for the preparation of Example Compound 1 using 5-[(3-methoxyazetidin-1-yl)methyl]pyridin-3-amine. Purification by RP-MPLC (C₁₈, MeOH/H₂O, 0-100%, with 0.1% TFA) provided the title compound; ¹H NMR (CDCl₃) δ 8.80 (d, 1H), 8.50 (d, 1H), 8.31-8.28 (m, 2H), 8.22 (d, 1H), 8.16-8.14 (m, 1H), 7.67 (s, 1H), 7.14-7.10 (m, 2H), 4.78-4.74 (m, 1H), 4.10-4.01 (m, 3H), 3.70 (s, 2H), 3.70-3.64 (m, 4H), 3.26 (s, 3H), 3.06-3.02 (m, 2H), 2.13-2.05 (m, 2H), 1.97-1.89 (m, 2H). LC-MS [M+H]⁺ 473.2294.

### Example Compound 24; 5-{2-[(5-Methylpyridin-3-yl)amino]pyrimidin-4-yl}-2-(tetrahydro-2H-pyran-4-yloxy)benzonitrile

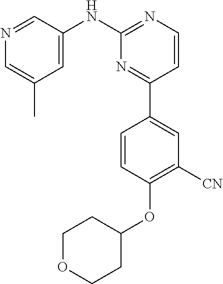 [SMILES: Cc1cncc(Nc2nccc(-c3ccc(OC4CCOCC4)c(C#N)c3)n2)c1]

This compound was prepared according to the procedure described for the preparation of Example Compound 1 using 5-methylpyridin-3-amine. Purification by RP-MPLC (C₁₈, MeOH/H₂O, 0-100%, with 0.1% TFA) provided the title compound; ¹H NMR (CDCl₃) δ 8.67 (d, 1H), 8.51 (d, 1H), 8.30-8.26 (m, 2H), 8.16 (s, 1H), 8.02 (s, 1H), 7.26 (s, 1H), 7.14-7.07 (m, 2H), 4.78-4.74 (m, 1H), 4.07-4.02 (m, 2H), 3.70-3.64 (m, 2H), 2.40 (s, 3H), 2.13-2.07 (m, 2H), 1.97-1.89 (m, 2H). LC-MS [M+H]⁺ 388.1822.

### Example Compound 25; 5-{2-[(5-Fluoropyridin-3-yl)amino]pyrimidin-4-yl}-2-(tetrahydro-2H-pyran-4-yloxy)benzonitrile

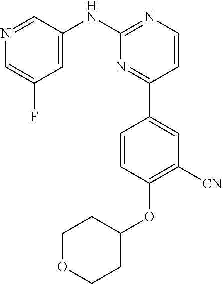 [SMILES: N#Cc1cc(-c2ccnc(Nc3cncc(F)c3)n2)ccc1OC1CCOCC1]

This compound was prepared according to the procedure described for the preparation of Example Compound 1 using 5-fluoropyridin-3-amine. Purification by RP-MPLC (C₁₈, MeOH/H₂O, 0-100%, with 0.1% TFA) provided the title compound; ¹H NMR (CDCl₃) δ 8.56-8.44 (m, 2H), 8.39-8.23 (m, 3H), 8.22-8.12 (m, 1H), 7.70 (s, 1H), 7.19-7.11 (m, 2H), 4.79-4.74 (m, 1H), 4.07-4.01 (m, 2H), 3.70-3.64 (m, 2H), 2.13-2.08 (m, 2H), 1.97-1.89 (m, 2H). LC-MS [M+H]⁺ 392.1521.

### Example Compound 26; 5-{2-[(5-Chloropyridin-3-yl)amino]pyrimidin-4-yl}-2-(tetrahydro-2H-pyran-4-yloxy)benzonitrile

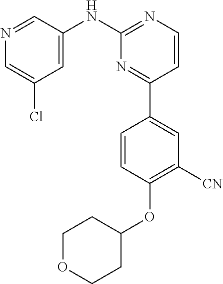 [SMILES: N#Cc1cc(-c2ccnc(Nc3cncc(Cl)c3)n2)ccc1OC1CCOCC1]

This compound was prepared according to the procedure described for the preparation of Example Compound 1 using 5-chloropyridin-3-amine. Purification by RP-MPLC (C₁₈, MeOH/H₂O, 0-100%, with 0.1% TFA) provided the title compound; ¹H NMR (CDCl₃) δ 8.63 (d, 1H), 8.60 (d, 1H), 8.34-8.30 (m, 2H), 8.29-8.23 (m, 1H), 7.23 (d, 1H), 7.14 (d, 2H), 7.00 (dd, 1H), 4.83-4.78 (m, 1H), 4.08-4.03 (m, 2H), 3.72-3.66 (m, 2H), 2.14-2.08 (m, 2H), 1.98-1.90 (m, 2H). LC-MS [M+H]⁺ 408.1198.

### Example Compound 27; 5-{2-[(5-Methoxypyridin-3-yl)amino]pyrimidin-4-yl}-2-(tetrahydro-2H-pyran-4-yloxy)benzonitrile

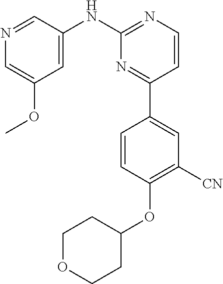 [SMILES: COc1cncc(Nc2nccc(-c3ccc(OC4CCOCC4)c(C#N)c3)n2)c1]

This compound was prepared according to the procedure described for the preparation of Example Compound 1 using 5-methoxypyridin-3-amine. Purification by RP-MPLC (C₁₈, MeOH/H₂O, 0-100%, with 0.1% TFA) provided the title compound; ¹H NMR (CDCl₃) δ 8.51 (d, 1H), 8.34-8.31 (m, 2H), 8.25 (dd, 1H), 8.05 (d, 2H), 7.58 (s, 1H), 7.14-7.08 (m, 2H), 4.76-4.74 (m, 1H), 4.07-4.01 (m, 2H), 3.93 (s, 3H), 3.70-3.64 (m, 2H), 2.12-2.07 (m, 2H), 1.96-1.89 (m, 2H). LC-MS [M+H]⁺ 404.1708.

### Example Compound 28; 5-{2-[(6-{[3-(2-Methoxyethoxy)azetidin-1-yl]carbonyl}pyridin-3-yl)amino]pyrimidin-4-yl}-2-(tetrahydro-2H-pyran-4-yloxy)benzonitrile

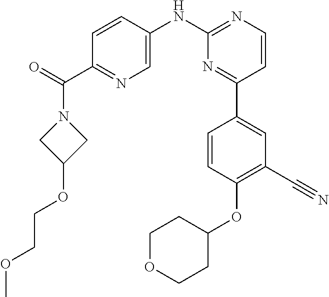 [SMILES: COCCOC1CN(C(=O)c2ccc(Nc3nccc(-c4ccc(OC5CCOCC5)c(C#N)c4)n3)cn2)C1]

This compound was prepared according to the procedure described for the preparation of Example Compound 1 using (5-amino-2-pyridyl)-[3-(2-methoxyethoxy)azetidin-1-yl]methanone as starting material. Purification by RP-MPLC (C₁₈, MeOH/H₂O, 0-100%, with 0.1% TFA) provided the title compound; ¹H NMR (DMSO-d₆) δ 10.2 (s, 1H), 8.95 (d, 1H), 8.64 (d, 1H), 8.57 (d, 1H), 8.48-8.42 (m, 2H), 7.96 (d, 1H), 7.61 (d, 1H), 7.57 (d, 1H), 4.98-4.93 (m, 1H), 4.80-4.75 (m, 1H), 4.39-4.33 (m, 2H), 4.26-4.22 (m, 1H), 3.91-3.83 (m, 3H), 3.59-3.52 (m, 4H), 3.48-3.43 (m, 2H), 3.26 (s, 3H), 2.08-2.02 (m, 2H), 1.74-1.65 (m, 2H). LC-MS [M+H]⁺ 531.2358.

### Example Compound 29; 5-(2-{[6-(Pyrrolidin-1-ylcarbonyl)pyridin-3-yl]amino}pyrimidin-4-yl)-2-(tetrahydro-2H-pyran-4-yloxy)benzonitrile

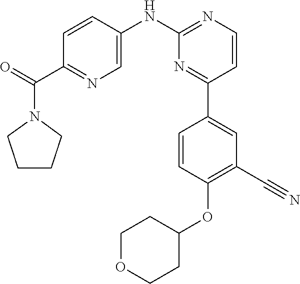 [SMILES: N#Cc1cc(-c2ccnc(Nc3ccc(C(=O)N4CCCC4)nc3)n2)ccc1OC1CCOCC1]

This compound was prepared according to the procedure described for the preparation of Example Compound 1 using (5-amino-2-pyridyl)-pyrrolidin-1-yl-methanone. Purification by RP-MPLC (C₁₈, MeOH/H₂O, 0-100%, with 0.1% TFA) provided the title compound; ¹H NMR (DMSO-d₆) δ 10.2 (s, 1H), 8.96 (s, 1H), 8.64 (d, 1H), 8.58 (d, 1H), 8.47 (dd, 1H), 8.38 (dd, 1H), 7.78 (d, 1H), 7.60 (d, 2H), 4.98-4.92 (m, 1H), 4.00-3.85 (m, 2H), 3.75-3.72 (m, 2H), 3.59-3.53 (m, 2H), 3.52-3.49 (m, 2H), 2.08-2.02 (m, 2H), 1.89-1.81 (m, 4H), 1.73-1.66 (m, 2H). LC-MS [M+H]⁺ 471.2149.

### Example Compound 30; 5-(2-{[6-(Azetidin-1-ylcarbonyl)pyridin-3-yl]amino}pyrimidin-4-yl)-2-(tetrahydro-2H-pyran-4-yloxy)benzonitrile

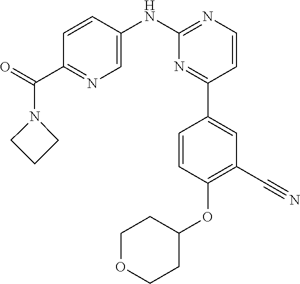 [SMILES: N#Cc1cc(-c2ccnc(Nc3ccc(C(=O)N4CCC4)nc3)n2)ccc1OC1CCOCC1]

This compound was prepared according to the procedure described for the preparation of Example Compound 1 using (5-amino-2-pyridyl)-(azetidin-1-yl)methanone. Purification by RP-MPLC (C₁₈, MeOH/H₂O, 0-100%, with 0.1% TFA) provided the title compound; ¹H NMR (DMSO-d₆) δ 10.2 (s, 1H), 8.94 (s, 1H), 8.64 (d, 1H), 8.56 (d, 1H), 8.48-8.42 (m, 2H), 7.94 (d, 1H), 7.60 (d, 1H), 7.57 (d, 1H), 4.99-4.93 (m, 1H), 4.61 (t, 2H), 4.06 (t, 2H), 3.91-3.85 (m, 2H), 3.59-3.53 (m, 2H), 2.31-2.23 (m, 2H), 2.07-2.03 (m, 2H), 1.73-1.66 (m, 2H). LC-MS [M+H]⁺ 457.1998.

### Example Compound 31; 5-[2-({6-[(3-Methoxyazetidin-1-yl)carbonyl]pyridin-3-yl}amino)pyrimidin-4-yl]-2-(tetrahydro-2H-pyran-4-yloxy)benzonitrile

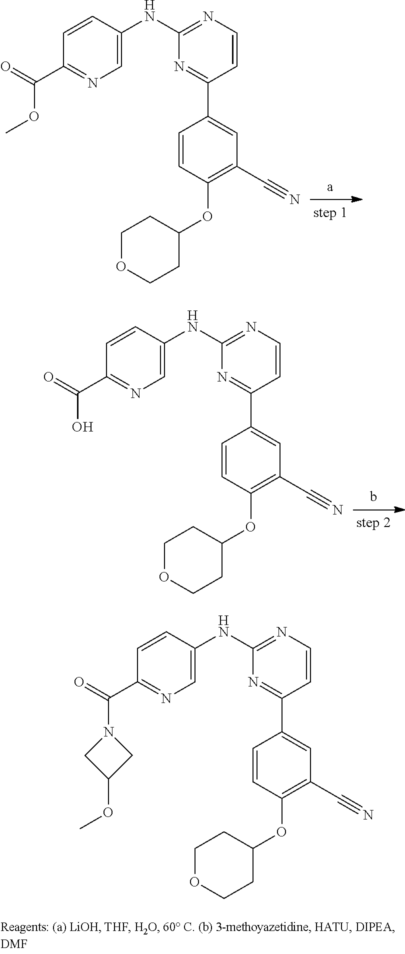

Step 1. 5-[[4-(3-Cyano-4-tetrahydropyran-4-yloxy-phenyl)pyrimidin-2-yl]amino]pyridine-2-carboxylic acid. A solution of methyl 5-({4-[3-cyano-4-(tetrahydro-2H-pyran-4-yloxy)phenyl]pyrimidin-2-yl}amino)pyridine-2-carboxylate (0.50 g, 1.16 mmol) was dissolved in 1:1 THF/H₂O (92 mL) and LiOH (5.0 g, 5.79 mmol) was added. The reaction mixture was heated at reflux overnight, then cooled and acidified with 1 N NH₄Cl to a pH of 4-5. A precipitate formed and the solution was cooled in an ice bath and the mixture filtered. The filtrate was dried under vacuum and used in the next step without further purification.

Step 2. 5-[2-({6-[(3-Methoxyazetidin-1-yl)carbonyl]pyridin-3-yl}amino)pyrimidin-4-yl]-2-(tetrahydro-2H-pyran-4-yloxy)benzonitrile. The title compound was prepared from the material isolated in Step 1 and 3-methoxyazetidine using Standard Method C; HATU Coupling. Purification by RP-MPLC (C₁₈, MeOH/H₂O, 0-100%, with 0.1% TFA) provided the title compound; ¹H NMR (DMSO-d₆) δ 10.3 (s, 1H), 8.96 (s, 1H), 8.65 (d, 1H), 8.58 (d, 1H), 8.49-8.43 (m, 2H), 7.96 (d, 1H), 7.62 (d, 1H), 7.58 (d, 1H), 4.99-4.93 (m, 1H), 4.79-4.75 (m, 1H), 4.38 (dd, 1H), 4.25-4.22 (m, 2H), 3.90-3.82 (m, 2H), 3.59-3.53 (m, 2H), 3.24 (s, 3H), 2.07-2.03 (m, 2H), 1.74-1.65 (m, 2H). LC-MS [M+H]⁺ 487.2080.

### Example Compound 32; 5-[2-({6-[(3-Hydroxyazetidin-1-yl)carbonyl]pyridin-3-yl}amino)pyrimidin-4-yl]-2-(tetrahydro-2H-pyran-4-yloxy)benzonitrile

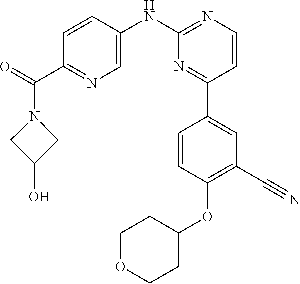 [SMILES: N#Cc1cc(-c2ccnc(Nc3ccc(C(=O)N4CC(O)C4)nc3)n2)ccc1OC1CCOCC1]

This compound was prepared according to the procedure described for the preparation of Example Compound 31 using azetidin-3-ol. Purification by RP-MPLC (C₁₈, MeOH/H₂O, 0-100%, with 0.1% TFA) provided the title compound; ¹H NMR (DMSO-d₆) δ 10.2 (s, 1H), 8.95 (s, 1H), 8.65 (d, 1H), 8.58 (d, 1H), 8.49-8.42 (m, 2H), 7.95 (d, 1H), 7.62 (d, 1H), 7.58 (d, 1H), 5.71 (br s, 1H), 4.98-4.93 (m, 1H), 4.80-4.75 (m, 1H), 4.50 (s, 1H), 4.32-4.23 (m, 2H), 3.90-3.85 (m, 2H), 3.78 (dd, 1H), 3.59-3.53 (m, 2H), 2.07-2.03 (m, 2H), 1.74-1.65 (m, 2H). LC-MS [M+H]⁺ 473.1926.

### Example Compound 33; Methyl 5-({4-[3-cyano-4-(tetrahydro-2H-pyran-4-yloxy)phenyl]pyrimidin-2-yl}amino)pyridine-2-carboxylate

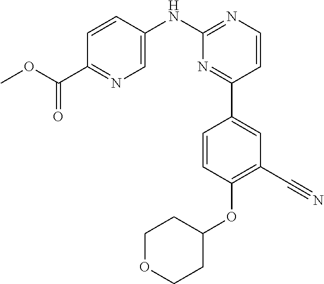 [SMILES: COC(=O)c1ccc(Nc2nccc(-c3ccc(OC4CCOCC4)c(C#N)c3)n2)cn1]

This compound was prepared according to the procedure described for the preparation of Example Compound 1 using methyl 5-aminopyridine-2-carboxylate. Purification by RP-MPLC (C₁₈, MeOH/H₂O, 0-100%, with 0.1% TFA) provided the title compound; ¹H NMR (DMSO-d₆) δ 10.4 (s, 1H), 9.11 (s, 1H), 8.67 (d, 1H), 8.58 (s, 1H), 8.50 (d, 1H), 8.44 (d, 1H), 8.07 (d, 1H), 7.65 (d, 1H), 7.60 (d, 1H), 4.98-4.93 (m, 1H), 3.91-3.86 (m, 2H), 3.86 (s, 3H), 3.58-3.53 (m, 2H), 2.07-1.99 (m, 2H), 1.73-1.66 (m, 2H). LC-MS [M+H]⁺ 432.1660.

### Example Compound 34; 5-[2-(Pyridin-4-ylamino)pyrimidin-4-yl]-2-(tetrahydro-2H-pyran-4-yloxy)benzonitrile

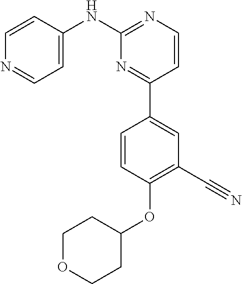 [SMILES: N#Cc1cc(-c2ccnc(Nc3ccncc3)n2)ccc1OC1CCOCC1]

This compound was prepared according to the procedure described for the preparation of Example Compound 1 using pyridin-4-amine. Purification by RP-MPLC (C₁₈, MeOH/H₂O, 0-100%, with 0.1% TFA) provided the title compound; ¹H NMR (DMSO-d₆) δ 10.2 (s, 1H), 8.67 (d, 1H), 8.58 (d, 1H), 8.48 (dd, 1H), 8.39 (d, 2H), 7.81 (d, 2H), 7.63 (d, 1H), 7.58 (d, 1H), 4.99-4.93 (m, 1H), 3.91-3.85 (m, 2H), 3.59-3.53 (m, 2H), 2.08-2.02 (m, 2H), 1.74-1.65 (m, 2H). LC-MS [M+H]⁺ 374.1612.

### Example Compound 35; 5-{2-[(1-Oxidopyridin-4-yl)amino]pyrimidin-4-yl}-2-(tetrahydro-2H-pyran-4-yloxy)benzonitrile

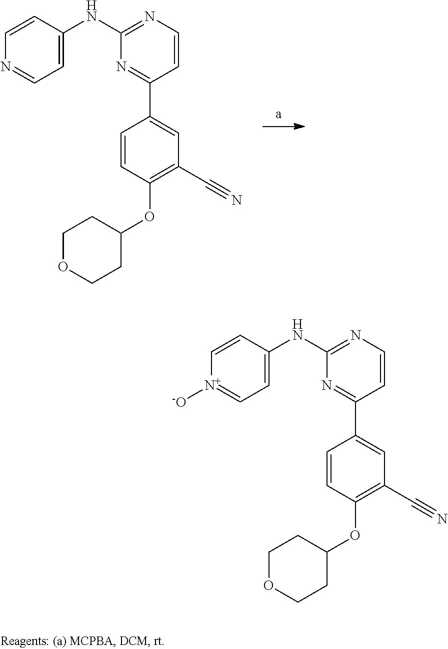

A solution of 5-[2-(pyridin-4-ylamino)pyrimidin-4-yl]-2-(tetrahydro-2H-pyran-4-yloxy)benzonitrile (0.007 g, 0.018 mmol) was treated with 77% MCPBA (0.005 g, 0.02 mmol) and stirred at rt for 4 h. The reaction was quenched by adding a solution of sodium thiosulfate (0.250 g) and a crystal of iodine to detect for the persistence of peracid. After mixing, the layers were separated and the organic layer was washed with saturated NaHCO₃ (aq). The organic layer was dried (Na₂SO₄) and concentrated. The residue was recrystallized from hexanes/EtOAc to give the title compound (0.005 g); LC-MS [M+H]⁺ 390.1542.

### Example Compound 36; Methyl 4-({4-[3-cyano-4-(tetrahydro-2H-pyran-4-yloxy)phenyl]pyrimidin-2-yl}amino)pyridine-2-carboxylate

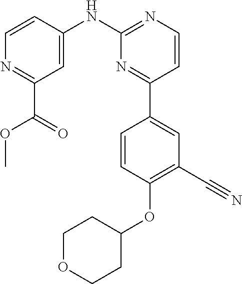 [SMILES: COC(=O)c1cc(Nc2nccc(-c3ccc(OC4CCOCC4)c(C#N)c3)n2)ccn1]

This compound was prepared according to the procedure described for the preparation of Example Compound 1 using methyl 4-aminopyridine-2-carboxylate. The residue was purified by column chromatography (SiO₂, MeOH 20% in CH₂Cl₂ with 0.1% NH₄OH), to give the title compound; ¹H NMR (DMSO-d₆) δ 10.5 (s, 1H), 8.70-8.68 (m, 2H), 8.59 (d, 1H), 8.51 (d, 1H), 8.50 (dd, 1H), 7.96 (d, 1H), 7.67 (d, 1H), 7.56 (d, 1H), 5.00-4.94 (m, 1H), 3.90 (s, 3H), 3.90-3.86 (m, 2H), 3.60-3.55 (m, 2H), 2.07-2.02 (m, 2H), 1.74-1.66 (m, 2H). LC-MS [M+Na]⁺ 538.2167.

### Example Compound 37; 5-[2-({2-[(2R)-2-(Hydroxymethyl)pyrrolidin-1-yl]pyridin-4-yl}amino)pyrimidin-4-yl]-2-(tetrahydro-2H-pyran-4-yloxy)benzonitrile

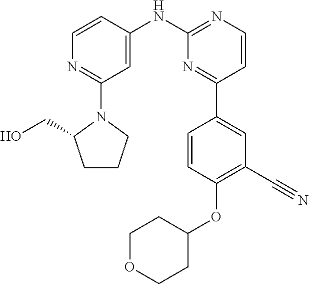 [SMILES: N#Cc1cc(-c2ccnc(Nc3ccnc(N4CCC[C@@H]4CO)c3)n2)ccc1OC1CCOCC1]

This compound was prepared according to the procedure described for the preparation of Example Compound 1 using [(2R)-1-(4-amino-2-pyridyl)pyrrolidin-2-yl]methanol. Purification by RP-MPLC (C₁₈, MeOH/H₂O, 0-100%, with 0.1% TFA) provided the title compound; ¹H NMR (DMSO-d₆) δ 10.8 (br s, 1H), 8.76 (d, 1H), 8.59 (s, 1H), 8.46 (dd, 1H), 7.89 (br s, 1H), 7.85 (d, 1H), 7.78 (d, 1H), 7.57 (d, 1H), 7.06 (d, 1H), 5.00-4.95 (m, 1H), 4.22-4.16 (m, 1H), 3.90-3.85 (m, 2H), 3.64-3.54 (m, 4H), 3.47-3.43 (m, 1H), 3.34-3.24 (m, 2H), 2.51-1.87 (m, 6H), 1.74-1.65 (m, 2H). LC-MS [M+H]⁺ 473.2308.

### Example Compound 38; 5-[2-({2-[(2-Methoxyethyl)amino]pyridin-4-yl}amino)pyrimidin-4-yl]-2-(tetrahydro-2H-pyran-4-yloxy)benzonitrile

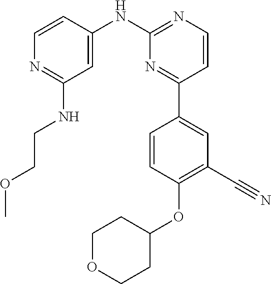 [SMILES: COCCNc1cc(Nc2nccc(-c3ccc(OC4CCOCC4)c(C#N)c3)n2)ccn1]

This compound was prepared according to the procedure described for the preparation of Example Compound 1 using N-(2-methoxyethyl)pyridine-2,4-diamine. Purification by RP-MPLC (C₁₈, MeOH/H₂O, 0-100%, with 0.1% TFA) provided the title compound; ¹H NMR (DMSO-d₆) δ 12.4 (br s, 1H), 10.8 (s, 1H), 8.75 (d, 1H), 8.60 (s, 1H), 8.47 (dd, 1H), 8.39 (br s, 1H), 7.82-7.79 (m, 2H), 7.56 (d, 1H), 7.05 (d, 1H), 5.00-4.94 (m, 1H), 3.90-3.85 (m, 2H), 3.60-3.55 (m, 4H), 3.50-3.46 (m, 2H), 3.30 (s, 3H), 2.08-2.02 (m, 2H), 1.74-1.65 (m, 2H). LC-MS [M+H]⁺ 447.2151.

### Example Compound 39; 5-(2-{[2-(3-Methoxypyrrolidin-1-yl)pyridin-4-yl]amino}pyrimidin-4-yl)-2-(tetrahydro-2H-pyran-4-yloxy)benzonitrile

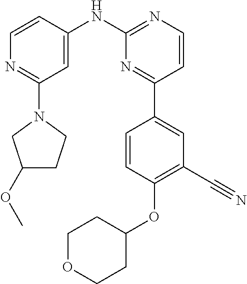 [SMILES: COC1CCN(c2cc(Nc3nccc(-c4ccc(OC5CCOCC5)c(C#N)c4)n3)ccn2)C1]

This compound was prepared according to the procedure described for the preparation of Example Compound 1 using 2-(3-methoxypyrrolidin-1-yl)pyridin-4-amine. Purification by RP-MPLC (C₁₈, MeOH/H₂O, 0-100%, with 0.1% TFA) provided the title compound; ¹H NMR (DMSO-d₆) δ 9.89 (s, 1H), 8.63 (d, 1H), 8.58 (s, 1H), 8.46 (dd, 1H), 7.88 (d, 1H), 7.58-7.54 (m, 2H), 7.31 (s, 1H), 6.83 (dd, 1H), 5.00-4.94 (m, 1H), 4.12-4.07 (m, 1H), 3.90-3.85 (m, 2H), 3.59-3.47 (m, 5H), 3.43-3.36 (m, 1H), 3.26 (s, 3H), 2.13-2.02 (m, 4H), 1.74-1.65 (m, 2H). LC-MS [M+H]⁺ 473.2327.

### Example Compound 40; 5-[2-({2-[(2S)-2-(Hydroxymethyl)pyrrolidin-1-yl]pyridin-4-yl}amino)pyrimidin-4-yl]-2-(tetrahydro-2H-pyran-4-yloxy)benzonitrile

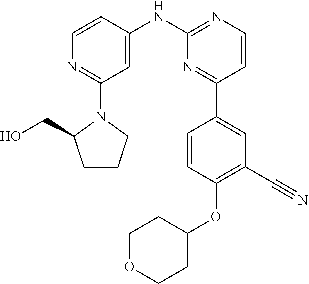 [SMILES: N#Cc1cc(-c2ccnc(Nc3ccnc(N4CCC[C@H]4CO)c3)n2)ccc1OC1CCOCC1]

This compound was prepared according to the procedure described for the preparation of Example Compound 1 using [(2S)-1-(4-amino-2-pyridyl)pyrrolidin-2-yl]methanol. Purification by RP-MPLC (C₁₈, MeOH/H₂O, 0-100%, with 0.1% TFA) provided the title compound; ¹H NMR (DMSO-d₆) δ 9.90 (s, 1H), 8.62 (d, 1H), 8.57 (s, 1H), 8.45 (dd, 1H), 7.87 (d, 1H), 7.58-7.54 (m, 2H), 7.31 (s, 1H), 6.87 (dd, 1H), 5.18 (br s, 1H), 4.99-4.93 (m, 1H), 4.12-4.07 (m, 1H), 3.90-3.85 (m, 2H), 3.62-3.53 (m, 3H), 3.47-3.43 (m, 1H), 3.34-3.24 (m, 2H), 2.51-1.87 (m, 6H), 1.74-1.68 (m, 2H). LC-MS [M+H]⁺ 473.2303.

### Example Compound 41; 5-(2-{[2-(3-Hydroxyazetidin-1-yl)pyridin-4-yl]amino}pyrimidin-4-yl)-2-(tetrahydro-2H-pyran-4-yloxy)benzonitrile

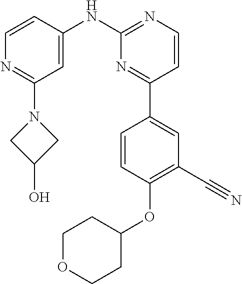 [SMILES: N#Cc1cc(-c2ccnc(Nc3ccnc(N4CC(O)C4)c3)n2)ccc1OC1CCOCC1]

This compound was prepared according to the procedure described for the preparation of Example Compound 1 using 1-(4-amino-2-pyridyl)azetidin-3-ol. Purification by RP-MPLC (C₁₈, MeOH/H₂O, 0-100%, with 0.1% TFA) provided the title compound; ¹H NMR (DMSO-d₆) δ 9.95 (s, 1H), 8.63 (d, 1H), 8.56 (s, 1H), 8.45 (dd, 1H), 7.88 (d, 1H), 7.59-7.56 (m, 2H), 7.14 (s, 1H), 6.94 (dd, 1H), 5.62 (d, 1H), 5.00-4.94 (m, 1H), 4.60-4.54 (m, 1H), 4.16 (t, 2H), 3.90-3.85 (m, 2H), 3.66 (dd, 2H), 3.58-3.53 (m, 2H), 3.34 (s, 3H), 2.08-2.02 (m, 2H), 1.74-1.65 (m, 2H). LC-MS [M+H]⁺ 445.1991.

### Example Compound 42; 5-[2-({2-[4-(2-Hydroxyethyl)piperazin-1-yl]pyridin-4-yl}amino)pyrimidin-4-yl]-2-(tetrahydro-2H-pyran-4-yloxy)benzonitrile

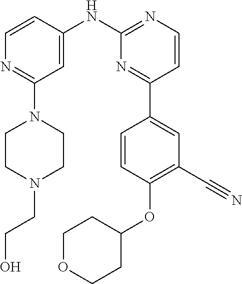 [SMILES: N#Cc1cc(-c2ccnc(Nc3ccnc(N4CCN(CCO)CC4)c3)n2)ccc1OC1CCOCC1]

This compound was prepared according to the procedure described for the preparation of Example Compound 1 using 2-[4-(4-amino-2-pyridyl)piperazin-1-yl]ethanol. Purification by RP-MPLC (C₁₈, MeOH/H₂O, 0-100%, with 0.1% TFA) provided the title compound; ¹H NMR (DMSO-d₆) δ 8.72 (d, 1H), 8.59 (s, 1H), 8.47 (dd, 1H), 8.04 (d, 1H), 7.82-7.73 (m, 1H), 7.72 (d, 1H), 7.57 (d, 1H), 7.24 (d, 1H), 5.00-4.94 (m, 1H), 4.28-4.18 (m, 1H), 3.90-3.85 (m, 2H), 3.90-3.86 (m, 2H), 3.68-3.61 (m, 2H), 3.60-3.53 (m, 2H), 3.48-3.38 (m, 2H), 3.26 (br s, 4H), 2.08-2.02 (m, 2H), 1.74-1.65 (m, 2H). LC-MS [M+H]⁺ 502.2561.

### Example Compound 43; 5-(2-{[2-(3-Methoxyazetidin-1-yl)pyridin-4-yl]amino}pyrimidin-4-yl)-2-(tetrahydro-2H-pyran-4-yloxy)benzonitrile

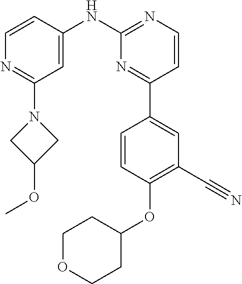 [SMILES: COC1CN(c2cc(Nc3nccc(-c4ccc(OC5CCOCC5)c(C#N)c4)n3)ccn2)C1]

This compound was prepared according to the procedure described for the preparation of Example Compound 1 using 2-(3-methoxyazetidin-1-yl)pyridin-4-amine. Purification by RP-MPLC (C₁₈, MeOH/H₂O, 0-100%, with 0.1% TFA) provided the title compound; ¹H NMR (DMSO-d₆) δ 9.96 (s, 1H), 8.63 (d, 1H), 8.56 (s, 1H), 8.45 (dd, 1H), 7.88 (d, 1H), 7.60-7.56 (m, 2H), 7.23 (s, 1H), 6.88 (dd, 1H), 5.00-4.94 (m, 1H), 4.35-4.30 (m, 1H), 4.15 (t, 2H), 3.90-3.86 (m, 2H), 3.75 (dd, 2H), 3.59-3.53 (m, 2H), 3.26 (s, 3H), 2.08-2.02 (m, 2H), 1.74-1.65 (m, 2H). LC-MS [M+H]⁺ 459.2073.

### Example Compound 44; 5-(2-{[2-(Morpholin-4-yl)pyridin-4-yl]amino}pyrimidin-4-yl)-2-(tetrahydro-2H-pyran-4-yloxy)benzonitrile

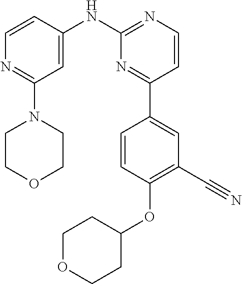 [SMILES: N#Cc1cc(-c2ccnc(Nc3ccnc(N4CCOCC4)c3)n2)ccc1OC1CCOCC1]

This compound was prepared according to the procedure described for the preparation of Example Compound 1 using 2-morpholinopyridin-4-amine. Purification by RP-MPLC (C₁₈, MeOH/H₂O, 0-100%, with 0.1% TFA) provided the title compound; ¹H NMR (DMSO-d₆) δ 9.96 (s, 1H), 8.63 (d, 1H), 8.55 (s, 1H), 8.45 (d, 1H), 7.97 (d, 1H), 7.58 (d, 1H), 7.54 (d, 1H), 7.04 (d, 1H), 5.00-4.94 (m, 1H), 3.90-3.86 (m, 2H), 3.74 (t, 4H), 3.59-3.53 (m, 2H), 3.41 (t, 4H), 2.08-2.02 (m, 2H), 1.74-1.65 (m, 2H). LC-MS [M+H]⁺ 459.2073.

### Example Compound 45; 5-[2-({2-[3-(2-Methoxyethoxy)azetidin-1-yl]pyridin-4-yl}amino)pyrimidin-4-yl]-2-(tetrahydro-2H-pyran-4-yloxy)benzonitrile

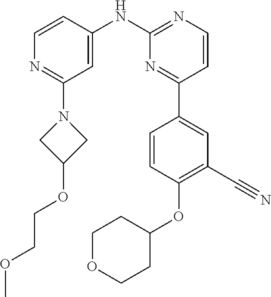 [SMILES: COCCOC1CN(c2cc(Nc3nccc(-c4ccc(OC5CCOCC5)c(C#N)c4)n3)ccn2)C1]

This compound was prepared according to the procedure described for the preparation of Example Compound 1 using 2-[3-(2-methoxyethoxy)azetidin-1-yl]pyridin-4-amine. Purification by RP-MPLC (C₁₈, MeOH/H₂O, 0-100%, with 0.1% TFA) provided the title compound; ¹H NMR (DMSO-d₆) δ 10.0 (s, 1H), 8.64 (d, 1H), 8.56 (d, 1H), 8.46 (dd, 1H), 7.88 (d, 1H), 7.60 (d, 1H), 7.57 (d, 1H), 7.24 (s, 1H), 6.91 (dd, 1H), 5.00-4.93 (m, 1H), 4.46-4.41 (m, 1H), 4.18 (t, 2H), 3.90-3.85 (m, 2H), 3.76 (dd, 2H), 3.59-3.53 (m, 4H), 3.48-3.45 (m, 2H), 3.27 (s, 3H), 2.08-2.02 (m, 2H), 1.74-1.65 (m, 2H). LC-MS [M+H]⁺ 503.2363.

### Example Compound 46; 5-(2-{[6-(3-Hydroxyazetidin-1-yl)pyridin-3-yl]amino}pyrimidin-4-yl)-2-(tetrahydro-2H-pyran-4-yloxy)benzonitrile

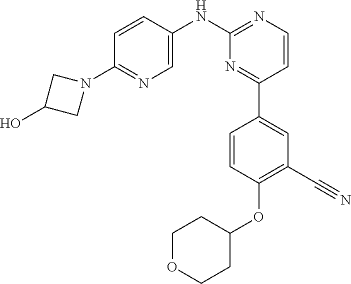 [SMILES: N#Cc1cc(-c2ccnc(Nc3ccc(N4CC(O)C4)nc3)n2)ccc1OC1CCOCC1]

This compound was prepared according to the procedure described for the preparation of Example Compound 1 using 1-(5-amino-2-pyridyl)azetidin-3-ol. Purification by RP-MPLC (C₁₈, MeOH/H₂O, 0-100%, with 0.1% TFA) provided the title compound; ¹H NMR (DMSO-d₆) δ 9.39 (s, 1H), 8.50-8.46 (m, 2H), 8.43-8.39 (m, 2H), 7.85 (dd, 1H), 7.55 (d, 1H), 7.39 (d, 1H), 6.42 (d, 1H), 5.62 (d, 1H), 5.00-4.93 (m, 1H), 4.60-4.54 (m, 1H), 4.12 (t, 2H), 3.89-3.85 (m, 2H), 3.63 (dd, 1H), 3.59-3.53 (m, 2H), 2.08-2.02 (m, 2H), 1.74-1.65 (m, 2H). LC-MS [M+H]⁺ 445.1994.

### Example Compound 47; 5-(2-{[6-(Azetidin-1-ylmethyl)pyridin-3-yl]amino}pyrimidin-4-yl)-2-(tetrahydro-2H-pyran-4-yloxy)benzonitrile

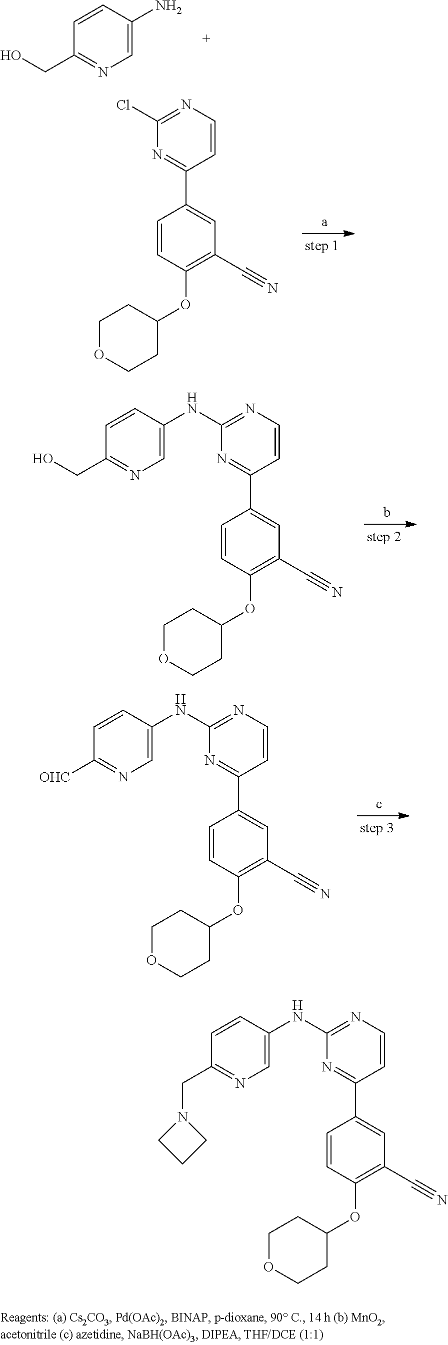

Step 1. 5-[2-[[6-(Hydroxymethyl)-3-pyridyl]amino]pyrimidin-4-yl]-2-tetrahydropyran-4-yloxy-benzonitrile. This compound was prepared according to the procedure described for the preparation of Example Compound 1 using (5-amino-2-pyridyl)methanol. ¹H NMR (DMSO-d₆) δ 9.85 (s, 1H), 8.87 (d, 1H), 8.58 (d, 1H), 8.54 (d, 1H), 8.45 (dd, 1H), 8.20 (dd, 1H), 7.57 (d, 1H), 7.52 (d, 1H), 7.42 (d, 1H), 5.33 (t, 1H), 4.98-4.91 (m, 1H), 4.52 (d, 2H), 3.90-3.85 (m, 2H), 3.58-3.53 (m, 2H), 2.09-2.01 (m, 2H), 1.73-1.65 (m, 2H). LC-MS [M+H]⁺ 387.3.

Step 2. 5-[2-[(6-Formyl-3-pyridyl)amino]pyrimidin-4-yl]-2-tetrahydropyran-4-yloxy-benzonitrile. A solution of 5-[2-[[6-(hydroxymethyl)-3-pyridyl]amino]pyrimidin-4-yl]-2-tetrahydropyran-4-yloxy-benzonitrile (1.0 g, 2.5 mmol) in acetonitrile was treated with MnO₂ (1.0 g, 12.4 mmol) and the mixture stirred at 90° C. for 18 h. The reaction was allowed to cool, then filtered through Celite. The filtrate was concentrated under vacuum to give the title compound (0.9 g). ¹H NMR (DMSO-d₆) δ 10.5 (s, 1H), 9.89 (s, 1H), 9.20 (d, 1H), 8.69 (d, 1H), 8.58 (d, 1H), 8.52-8.48 (m, 2H), 7.96 (d, 1H), 7.68 (d, 1H), 7.59 (d, 1H), 4.99-4.93 (m, 1H), 3.92-3.85 (m, 2H), 3.59-3.53 (m, 2H), 2.09-2.02 (m, 2H), 1.75-1.66 (m, 2H).

Step 3. 5-(2-{[6-(Azetidin-1-ylmethyl)pyridin-3-yl]amino}pyrimidin-4-yl)-2-(tetrahydro-2H-pyran-4-yloxy)benzonitrile. A solution of 5-[2-[(6-formyl-3-pyridyl)amino]pyrimidin-4-yl]-2-tetrahydropyran-4-yloxy-benzonitrile (0.075 g, 0.187 mmol) in 1:1 THF/DCE (2 mL) was treated with sodium triacetoxyborohydride (0.06 g, 6.28 mmol) and DIPEA (0.33 mL, 1.88 mmol) and azetidine (0.13 mL, 1.88 mmol) and stirred at rt overnight. The reaction was quenched with the addition of saturated aqueous NaHCO₃ solution and the product was extracted with DCM. The organic layer was dried over Na₂SO₄ and concentrated under vacuum. Purification by RP-MPLC (C₁₈, MeOH/H₂O, 0-100%, with 0.1% TFA) provided the title compound; ¹H NMR (DMSO-d₆) δ 10.2 (br s, 1H), 10.0 (s, 1H), 9.01 (d, 1H), 8.61 (d, 1H), 8.54 (d, 1H), 8.44 (dd, 1H), 8.32 (dd, 1H), 7.96 (d, 1H), 7.59-7.54 (m, 2H), 7.44 (d, 1H), 4.99-4.93 (m, 1H), 4.48 (d, 2H), 4.11 (q, 4H), 3.91-3.85 (m, 3H), 3.59-3.53 (m, 2H), 2.45-2.33 (m, 2H), 2.10-1.98 (m, 2H), 1.74-1.65 (m, 2H). LC-MS [M+H]⁺ 443.2186.

### Example Compound 48; 5-[2-({6-[(3-Methoxyazetidin-1-yl)methyl]pyridin-3-yl}amino)pyrimidin-4-yl]-2-(tetrahydro-2H-pyran-4-yloxy)benzonitrile

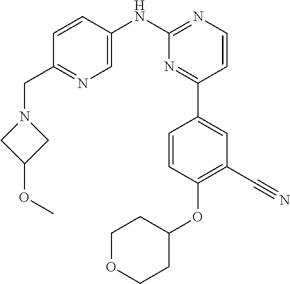 [SMILES: COC1CN(Cc2ccc(Nc3nccc(-c4ccc(OC5CCOCC5)c(C#N)c4)n3)cn2)C1]

This compound was prepared according to the procedure described for the preparation of Example Compound 47 using 3-methoxyazetidine. Purification by RP-MPLC (C₁₈, MeOH/H₂O, 0-100%, with 0.1% TFA) provided the title compound; ¹H NMR (DMSO-d₆) δ 10.1 (s, 1H), 9.01 (d, 1H), 8.62 (d, 1H), 8.55 (d, 1H), 8.44 (dd, 1H), 8.32 (dd, 1H), 7.59-7.54 (m, 2H), 7.45 (d, 1H), 4.99-4.93 (m, 1H), 4.52 (s, 2H), 4.38-4.33 (m, 2H), 4.30-4.26 (m, 1H), 4.07-4.00 (m, 2H), 3.90-3.85 (m, 2H), 3.59-3.54 (m, 2H), 3.25 (s, 3H), 2.10-1.98 (m, 2H), 1.74-1.65 (m, 2H). LC-MS [M+H]⁺ 473.2256.

### Example Compound 49; 5-{2-[(6-{[3-(2-Methoxyethoxy)azetidin-1-yl]methyl}pyridin-3-yl)amino]pyrimidin-4-yl}-2-(tetrahydro-2H-pyran-4-yloxy)benzonitrile

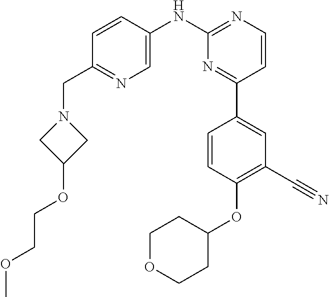 [SMILES: COCCOC1CN(Cc2ccc(Nc3nccc(-c4ccc(OC5CCOCC5)c(C#N)c4)n3)cn2)C1]

This compound was prepared according to the procedure described for the preparation of Example Compound 47 using 3-(2-methoxyethoxy)azetidine. Purification by RP-MPLC (C₁₈, MeOH/H₂O, 0-100%, with 0.1% TFA) provided the title compound; ¹H NMR (DMSO-d₆) δ 10.1 (s, 1H), 9.01 (d, 1H), 8.62 (d, 1H), 8.55 (d, 1H), 8.45 (dd, 1H), 8.32 (dd, 1H), 7.59-7.54 (m, 2H), 7.45 (d, 1H), 4.99-4.93 (m, 1H), 4.52 (d, 2H), 4.41-4.32 (m, 2H), 4.06-4.00 (m, 2H), 3.91-3.85 (m, 2H), 3.59-3.53 (m, 4H), 3.46-3.41 (m, 1H), 3.25 (s, 3H), 2.09-2.00 (m, 2H), 1.74-1.65 (m, 2H). LC-MS [M+H]⁺ 517.2541.

### Example Compound 50; 5-{2-[(6-{[(2-Methoxyethyl)amino]methyl}pyridin-3-yl)amino]pyrimidin-4-yl}-2-(tetrahydro-2H-pyran-4-yloxy)benzonitrile

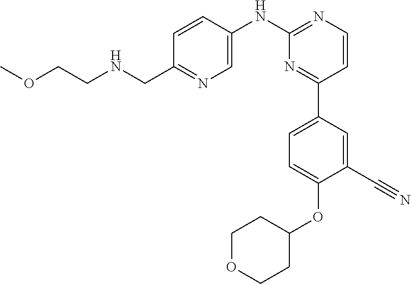 [SMILES: COCCNCc1ccc(Nc2nccc(-c3ccc(OC4CCOCC4)c(C#N)c3)n2)cn1]

This compound was prepared according to the procedure described for the preparation of Example Compound 47 using 2-methoxyethanamine. Purification by RP-MPLC (C₁₈, MeOH/H₂O, 0-100%, with 0.1% TFA) provided the title compound; ¹H NMR (DMSO-d₆) δ 10.1 (s, 1H), 9.02 (d, 1H), 8.61 (d, 1H), 8.55 (d, 1H), 8.45 (dd, 1H), 8.29 (dd, 1H), 7.58-7.55 (m, 2H), 7.47 (d, 1H), 5.00-4.93 (m, 1H), 4.22 (s, 2H), 3.90-3.85 (m, 2H), 3.62-3.53 (m, 2H), 3.35 (s, 3H), 3.13 (t, 2H), 2.09-2.02 (m, 2H), 1.73-1.65 (m, 2H). LC-MS [M+H]⁺ 461.2307.

### Example Compound 51; 5-(2-{[6-(Pyrrolidin-1-ylmethyl)pyridin-3-yl]amino}pyrimidin-4-yl)-2-(tetrahydro-2H-pyran-4-yloxy)benzonitrile

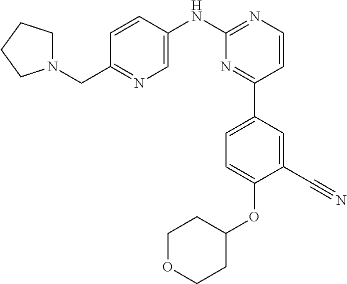 [SMILES: N#Cc1cc(-c2ccnc(Nc3ccc(CN4CCCC4)nc3)n2)ccc1OC1CCOCC1]

This compound was prepared according to the procedure described for the preparation of Example Compound 47 using pyrrolidine. Purification by RP-MPLC (C₁₈, MeOH/H₂O, 0-100%, with 0.1% TFA) provided the title compound; ¹H NMR (DMSO-d₆) δ 10.1 (s, 1H), 9.05 (d, 1H), 8.62 (d, 1H), 8.55 (d, 1H), 8.45 (dd, 1H), 8.33 (dd, 1H), 7.59 (d, 1H), 7.54 (d, 1H), 7.49 (d, 1H), 5.00-4.93 (m, 1H), 4.44 (s, 2H), 3.90-3.85 (m, 2H), 3.59-3.53 (m, 2H), 3.35 (s, 3H), 3.35-3.20 (br m, 4H), 2.08-2.02 (m, 2H), 1.99-1.90 (br m, 4H), 1.73-1.65 (m, 2H). LC-MS [M+H]⁺ 457.2363.

### Example Compound 52; 5-[2-({6-[(Methylamino)methyl]pyridin-3-yl}amino)pyrimidin-4-yl]-2-(tetrahydro-2H-pyran-4-yloxy)benzonitrile

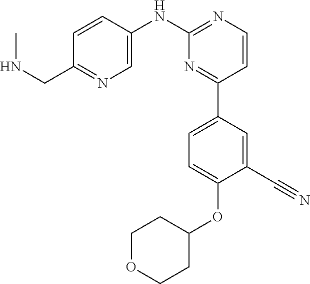 [SMILES: CNCc1ccc(Nc2nccc(-c3ccc(OC4CCOCC4)c(C#N)c3)n2)cn1]

This compound was prepared according to the procedure described for the preparation of Example Compound 47 using a solution of methyl amine in THF. Purification by RP-MPLC (C₁₈, MeOH/H₂O, 0-100%, with 0.1% TFA) provided the title compound; ¹H NMR (DMSO-d₆) δ 9.95 (s, 1H), 8.63 (d, 1H), 8.56 (d, 1H), 8.45 (dd, 1H), 7.88 (d, 1H), 7.59-7.56 (m, 2H), 7.14 (d, 1H), 6.94 (dd, 1H), 4.98-4.92 (m, 1H), 3.90-3.85 (m, 2H), 3.75 (s, 2H), 3.58-3.53 (m, 2H), 2.33 (s, 3H), 2.08-2.02 (m, 2H), 1.73-1.65 (m, 2H). LC-MS [M+H]⁺ 417.2036.

### Example Compound 53; N-{[5-({4-[3-Cyano-4-(tetrahydro-2H-pyran-4-yloxy)phenyl]pyrimidin-2-yl}amino)pyridin-2-yl]methyl}-N-methylacetamide

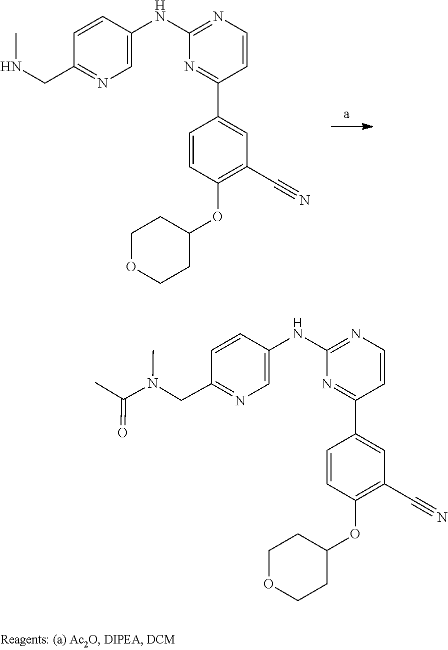 [SMILES: CNCc1ccc(Nc2nccc(-c3ccc(OC4CCOCC4)c(C#N)c3)n2)cn1 ; CC(=O)N(C)Cc1ccc(Nc2nccc(-c3ccc(OC4CCOCC4)c(C#N)c3)n2)cn1]

Standard Method F; Acylation or Sulfonylation was used to prepare the title compound from 5-[2-({6-[(methylamino)methyl]pyridin-3-yl}amino)pyrimidin-4-yl]-2-(tetrahydro-2H-pyran-4-yloxy)benzonitrile and acetic anhydride; Purification by column chromatography (SiO₂, MeOH 20% in CH₂Cl₂ with 0.1% NH₄OH), gave the title compound; ¹H NMR (DMSO-d₆) δ 10.1+10.0 (s, rotamer, 1H), 9.05+8.97 (s, rotamer, 1H), 8.61 (dd, 1H), 8.55 (t, 1H), 8.45 (dd, 1H), 8.33 (dd, 1H), 7.60-7.55 (m, 2H), 7.44+7.33 (d, rotamer, 1H), 4.99-4.92 (m, 1H), 4.61 (d, 2H), 3.90-3.85 (m, 2H), 3.58-3.53 (m, 2H), 3.05 (s, 3H), 2.09 (s, 3H), 2.08-2.02 (m, 2H), 1.73-1.65 (m, 2H). LC-MS [M+H]⁺ 459.2078.

### Example Compound 54; 5-(2-{[6-({Methyl[2-(methylsulfonyl)ethyl]amino}methyl)pyridin-3-yl]amino}pyrimidin-4-yl)-2-(tetrahydro-2H-pyran-4-yloxy)benzonitrile

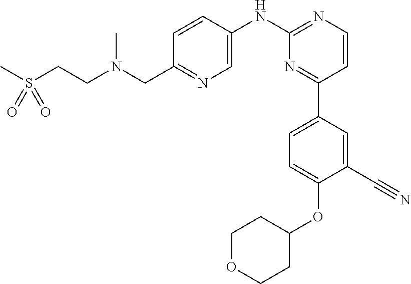 [SMILES: CN(CCS(C)(=O)=O)Cc1ccc(Nc2nccc(-c3ccc(OC4CCOCC4)c(C#N)c3)n2)cn1]

This compound was prepared according to the procedure described for the preparation of Example Compound 53 using 1-methylsulfonylethylene; Purification by column chromatography (SiO₂, MeOH 20% in CH₂Cl₂ with 0.1% NH₄OH), gave the title compound; ¹H NMR (DMSO-d₆) δ 10.1 (s, 1H), 9.08 (d, 1H), 8.63 (d, 1H), 8.55 (d, 1H), 8.46 (dd, 1H), 8.37 (dd, 1H), 7.59 (d, 1H), 7.54 (t, 2H), 4.99-4.92 (m, 1H), 4.45 (s, 2H), 3.90-3.85 (m, 2H), 3.76-3.71 (m, 2H), 3.59-3.53 (m, 4H), 3.13 (s, 3H), 2.79 (s, 3H), 2.08-2.02 (m, 2H), 1.73-1.66 (m, 2H). LC-MS [M+H]⁺ 523.2046.

### Example Compound 55; N-{[5-({4-[3-Cyano-4-(tetrahydro-2H-pyran-4-yloxy)phenyl]pyrimidin-2-yl}amino)pyridin-2-yl]methyl}-N-methylmethanesulfonamide

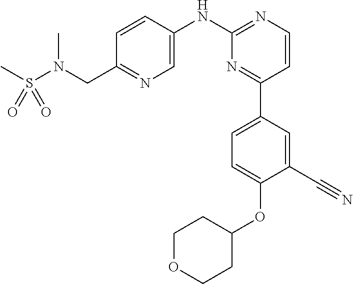 [SMILES: CN(Cc1ccc(Nc2nccc(-c3ccc(OC4CCOCC4)c(C#N)c3)n2)cn1)S(C)(=O)=O]

This compound was prepared according to the procedure described for the preparation of Example Compound 53 using methanesulfonyl chloride; Purification by column chromatography (SiO₂, MeOH 20% in CH₂Cl₂ with 0.1% NH₄OH), gave the title compound; ¹H NMR (DMSO-d₆) δ 10.1 (s, 1H), 9.00 (d, 1H), 8.61 (d, 1H), 8.55 (d, 1H), 8.46 (dd, 1H), 8.34 (dd, 1H), 7.58 (d, 1H), 7.56 (s, 1H), 7.46 (d, 1H), 4.99-4.92 (m, 1H), 4.37 (s, 2H), 3.90-3.85 (m, 2H), 3.58-3.53 (m, 2H), 3.00 (s, 3H), 2.77 (s, 3H), 2.08-2.02 (m, 2H), 1.73-1.66 (m, 2H). LC-MS [M+H]⁺ 495.1741.

### Example Compound 56; N-{[5-({4-[3-Cyano-4-(tetrahydro-2H-pyran-4-yloxy)phenyl]pyrimidin-2-yl}amino)pyridin-2-yl]methyl}-2-hydroxy-N,2-dimethylpropanamide

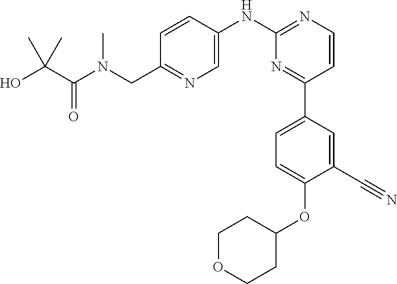 [SMILES: CN(Cc1ccc(Nc2nccc(-c3ccc(OC4CCOCC4)c(C#N)c3)n2)cn1)C(=O)C(C)(C)O]

This compound was prepared according to the procedure described for the preparation of Example Compound 53 using (2-chloro-1,1-dimethyl-2-oxo-ethyl)acetate; Purification by column chromatography (SiO₂, MeOH 20% in CH₂Cl₂ with 0.1% NH₄OH), gave the title compound; ¹H NMR (DMSO-d₆) δ 10.1 (s, 1H), 9.03 (br s, 1H), 8.61 (d, 1H), 8.55 (d, 1H), 8.45 (dd, 1H), 8.36-8.27 (m, 1H), 7.58-7.55 (m, 2H), 7.40-7.30 (m, 1H), 4.99-4.92 (m, 1H), 4.57 (s, 1H), 3.90-3.85 (m, 2H), 3.58-3.53 (m, 2H), 3.34 (s, 3H), 2.08-2.02 (m, 2H), 1.73-1.66 (m, 2H), 1.37 (s, 6H). LC-MS [M+H]⁺ 503.2327.

### Example Compound 57; (2R)—N-{[5-({4-[3-Cyano-4-(tetrahydro-2H-pyran-4-yloxy)phenyl]pyrimidin-2-yl}amino)pyridin-2-yl]methyl}-2-hydroxy-N-methylpropanamide

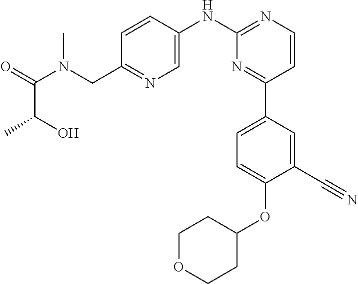 [SMILES: C[C@@H](O)C(=O)N(C)Cc1ccc(Nc2nccc(-c3ccc(OC4CCOCC4)c(C#N)c3)n2)cn1]

Standard Method C; HATU Coupling was used to prepare the title compound from 5-[2-({6-[(methylamino)methyl]pyridin-3-yl}amino)pyrimidin-4-yl]-2-(tetrahydro-2H-pyran-4-yloxy)benzonitrile and (2R)-2-hydroxypropanoic acid; Purification by column chromatography (SiO₂, MeOH 20% in CH₂Cl₂ with 0.1% NH₄OH), gave the title compound; ¹H NMR (DMSO-d₆) δ 9.99 (d, 1H), 8.95 (dd, 1H), 8.59 (d, 1H), 8.55 (d, 1H), 8.45 (dd, 1H), 8.23-8.24 (m, 1H), 7.58-7.54 (m, 2H), 7.34-7.25 (m, 1H), 4.99-4.92 (m, 1H), 4.58 (d, 1H), 4.56-4.50 (m, 1H), 3.90-3.86 (m, 2H), 3.59-3.53 (m, 2H), 3.17 (s, 3H), 2.08-2.02 (m, 2H), 1.73-1.66 (m, 2H), 1.25 (d, 3H). LC-MS [M+H]⁺ 489.2308.

### Example Compound 58; N-{[5-({4-[3-Cyano-4-(tetrahydro-2H-pyran-4-yloxy)phenyl]pyrimidin-2-yl}amino)pyridin-2-yl]methyl}-2-hydroxy-N-methylacetamide

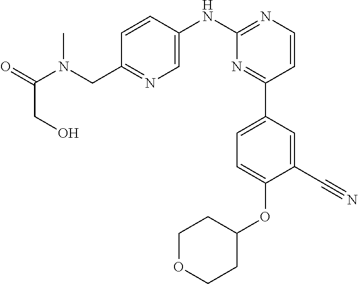 [SMILES: CN(Cc1ccc(Nc2nccc(-c3ccc(OC4CCOCC4)c(C#N)c3)n2)cn1)C(=O)CO]

This compound was prepared according to the procedure described for the preparation of Example Compound 57 using 2-hydroxyacetic acid; Purification by column chromatography (SiO₂, MeOH 20% in CH₂Cl₂ with 0.1% NH₄OH), gave the title compound; ¹H NMR (DMSO-d₆) δ 9.99 (d, 1H), 8.95 (dd, 1H), 8.59 (d, 1H), 8.55 (d, 1H), 8.45 (dd, 1H), 8.23-8.24 (m, 1H), 7.58-7.54 (m, 2H), 7.34-7.25 (m, 1H), 4.99-4.92 (m, 1H), 4.58 (d, 1H), 4.56-4.50 (m, 1H), 3.90-3.86 (m, 2H), 3.59-3.53 (m, 2H), 3.17 (s, 3H), 2.08-2.02 (m, 2H), 1.73-1.66 (m, 2H), 1.25 (d, 3H). LC-MS [M+H]⁺ 489.2308.

### Example Compound 59; N-{[5-({4-[3-Cyano-4-(tetrahydro-2H-pyran-4-yloxy)phenyl]pyrimidin-2-yl}amino)pyridin-2-yl]methyl}-1-hydroxy-N-methylcyclopropanecarboxamide

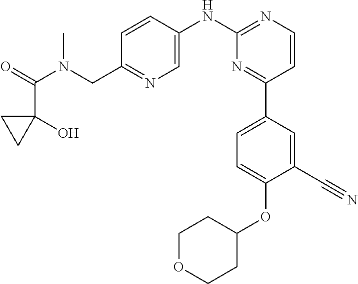 [SMILES: CN(Cc1ccc(Nc2nccc(-c3ccc(OC4CCOCC4)c(C#N)c3)n2)cn1)C(=O)C1(O)CC1]

This compound was prepared according to the procedure described for the preparation of Example Compound 57 using 1-hydroxycyclopropanecarboxylic acid; Purification by column chromatography (SiO₂, MeOH 20% in CH₂Cl₂ with 0.1% NH₄OH), gave the title compound; ¹H NMR (DMSO-d₆) δ 10.1 (s, 1H), 9.03 (br s, 1H), 8.61 (d, 1H), 8.55 (d, 1H), 8.45 (dd, 1H), 8.36-8.29 (m, 1H), 7.58-7.54 (m, 2H), 7.45-7.29 (m, 1H), 4.99-4.92 (m, 1H), 4.59 (s, 2H), 3.90-3.86 (m, 2H), 3.59-3.53 (m, 2H), 3.17 (s, 3H), 2.08-2.02 (m, 2H), 1.73-1.66 (m, 2H), 1.01-0.97 (m, 2H), 0.84-0.80 (m, 2H). LC-MS [M+H]⁺ 501.2245.

### Example Compound 60; 1-Amino-N-{[5-({4-[3-cyano-4-(tetrahydro-2H-pyran-4-yloxy)phenyl]pyrimidin-2-yl}amino)pyridin-2-yl]methyl}-N-methylcyclopropanecarboxamide

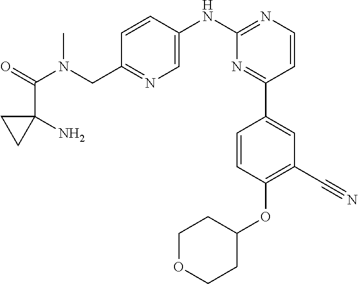 [SMILES: CN(Cc1ccc(Nc2nccc(-c3ccc(OC4CCOCC4)c(C#N)c3)n2)cn1)C(=O)C1(N)CC1]

This compound was prepared according to the procedure described for the preparation of Example Compound 57 using 1-(tert-butoxycarbonylamino)cyclopropanecarboxylic acid, followed by brief treatment of the isolated residue with TFA. The residue was neutralized with sodium bicarbonate solution and extracted with DCM. The solution was dried (Na₂SO₄) and concentrated. RP-MPLC (C¹⁸, MeOH/H₂O, 0-100% w/0.1% TFA) provided the title compound; ¹H NMR (DMSO-d₆) δ 10.0 (s, 1H), 9.01 (br s, 1H), 8.95 (d, 1H), 8.61 (d, 1H), 8.55 (d, 1H), 8.45 (dd, 1H), 8.32 (d, 1H), 7.58-7.54 (m, 2H), 7.45 (d, 1H), 4.99-4.92 (m, 1H), 4.71 (s, 2H), 3.90-3.85 (m, 2H), 3.59-3.53 (m, 2H), 3.17 (s, 3H), 2.08-2.02 (m, 2H), 1.73-1.66 (m, 2H), 1.31-1.28 (m, 4H). LC-MS [M+H]⁺ 500.2464.

### Example Compound 61; 5-(2-{[6-(1-Hydroxyethyl)pyridin-3-yl]amino}pyrimidin-4-yl)-2-(tetrahydro-2H-pyran-4-yloxy)benzonitrile

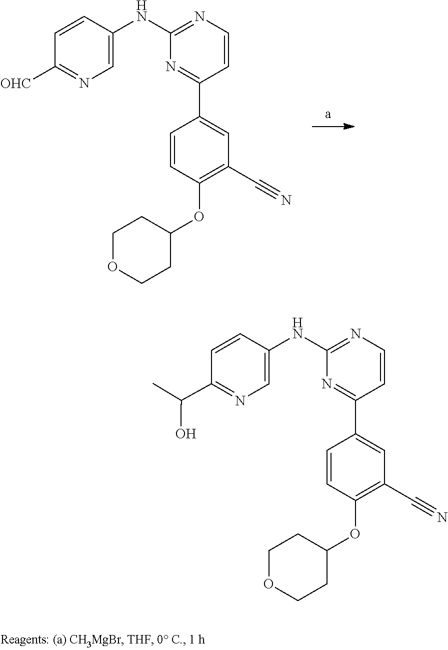 [SMILES: N#Cc1cc(-c2ccnc(Nc3ccc(C=O)nc3)n2)ccc1OC1CCOCC1 ; CC(O)c1ccc(Nc2nccc(-c3ccc(OC4CCOCC4)c(C#N)c3)n2)cn1]

Step 1. 5-[2-[[6-(1-Hydroxyethyl)-3-pyridyl]amino]pyrimidin-4-yl]-2-tetrahydropyran-4-yloxy-benzonitrile. 5-[2-[(6-formyl-3-pyridyl)amino]pyrimidin-4-yl]-2-tetrahydropyran-4-yloxy-benzonitrile (0.50 g, 1.25 mmol) was dissolved in THF (20 mL) and cooled in an ice bath. The solution was treated with a solution of 1.4 M methyl magnesium bromide in THF (1 mL, 1.4 mmol) and allowed to stir at rt for 1 h. The reaction was quenched with a solution of saturated NH₄Cl (aq) and extracted with DCM. The organic layer was dried (Na₂SO₄) and concentrated. Purification by column chromatography (SiO₂, MeOH 20% in CH₂Cl₂ with 0.1% NH₄OH), gave the title compound; ¹H NMR (DMSO-d₆) δ 9.83 (s, 1H), 8.85 (d, 1H), 8.57 (d, 1H), 8.54 (d, 1H), 8.45 (dd, 1H), 8.18 (dd, 1H), 7.57 (d, 1H), 7.52 (d, 1H), 7.45 (d, 1H), 5.25 (d, 2H), 5.00-4.94 (m, 1H), 4.74-4.67 (m, 1H), 3.90-3.86 (m, 2H), 3.59-3.54 (m, 2H), 2.07-2.02 (m, 2H), 1.74-1.66 (m, 2H), 1.37 (d, 3H). LC-MS [M+Na]+440.1704.

### Example Compound 62; 5-{2-[(6-Acetylpyridin-3-yl)amino]pyrimidin-4-yl}-2-(tetrahydro-2H-pyran-4-yloxy)benzonitrile

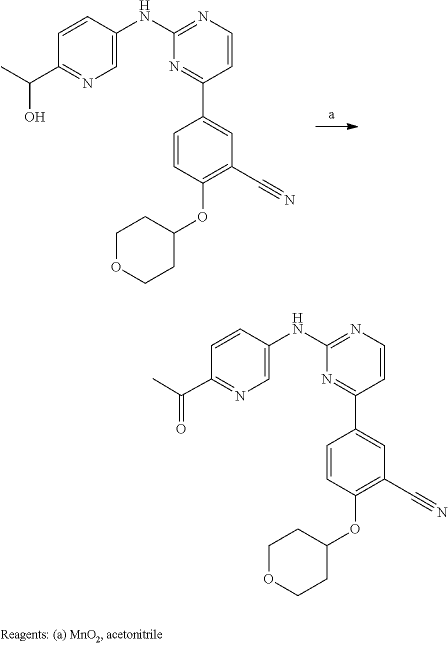 [SMILES: CC(O)c1ccc(Nc2nccc(-c3ccc(OC4CCOCC4)c(C#N)c3)n2)cn1 ; CC(=O)c1ccc(Nc2nccc(-c3ccc(OC4CCOCC4)c(C#N)c3)n2)cn1]

A solution of 5-[2-[[6-(1-hydroxyethyl)-3-pyridyl]amino]pyrimidin-4-yl]-2-tetrahydropyran-4-yloxy-benzonitrile (0.05 g, 0.12 mmol) in acetonitrile was treated with MnO₂ (0.05 g, 0.6 mmol) and the mixture stirred at 90° C. for 12 h. The reaction was allowed to cool, then filtered through Celite. The filtrate was concentrated under vacuum to give the title compound (0.035 g). ¹H NMR (DMSO-d₆) δ 10.4 (s, 1H), 9.08 (d, 1H), 8.67 (d, 1H), 8.58 (d, 1H), 8.50-8.45 (m, 2H), 7.99 (d, 1H), 7.65 (d, 1H), 7.59 (d, 1H), 4.99-4.93 (m, 1H), 3.91-3.85 (m, 2H), 3.59-3.53 (m, 2H), 2.60 (s, 3H), 2.08-2.02 (m, 2H), 1.74-1.65 (m, 2H). LC-MS [M+H]⁺ 416.1732.

### Example Compound 63; 5-[2-({6-[1-(3-Hydroxyazetidin-1-yl)ethyl]pyridin-3-yl}amino)pyrimidin-4-yl]-2-(tetrahydro-2H-pyran-4-yloxy)benzonitrile

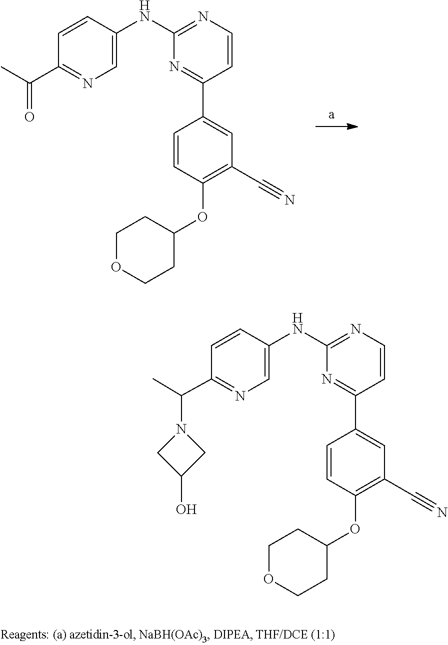 [SMILES: CC(=O)c1ccc(Nc2nccc(-c3ccc(OC4CCOCC4)c(C#N)c3)n2)cn1 ; CC(c1ccc(Nc2nccc(-c3ccc(OC4CCOCC4)c(C#N)c3)n2)cn1)N1CC(O)C1]

A solution of 5-{2-[(6-Acetylpyridin-3-yl)amino]pyrimidin-4-yl}-2-(tetrahydro-2H-pyran-4-yloxy)benzonitrile (0.035 g, 0.084 mmol) in 1:1 THF/DCE (2 mL) was treated with sodium triacetoxyborohydride (0.027 g, 1.26 mmol) and DIPEA (1.5 eq) and azetidin-3-ol hydrochloride (0.014 g, 0.126 mmol) and stirred at rt overnight. The reaction was quenched with the addition of saturated aqueous NaHCO₃ solution and the product was extracted with DCM. The organic layer was dried over Na₂SO₄ and concentrated under vacuum. Purification by RP-MPLC (C₁₈, MeOH/H₂O, 0-100%, with 0.1% TFA) provided the title compound; ¹H NMR (DMSO-d₆) δ 10.1 (s, 1H), 9.02 (d, 1H), 8.62 (d, 1H), 8.55 (d, 1H), 8.45 (dd, 1H), 8.33 (dd, 1H), 7.59-7.54 (m, 2H), 7.49 (d, 1H), 6.23-6.15 (br s, 1H), 5.00-4.93 (m, 1H), 4.64 (t, 1H), 4.51-4.43 (m, 2H), 4.38-4.28 (m, 1H), 4.10-3.91 (m, 2H), 3.91-3.85 (m, 2H), 3.77-3.65 (m, 1H), 3.59-3.53 (m, 2H), 2.08-2.02 (m, 2H), 1.74-1.65 (m, 2H), 1.44 (d, 3H). LC-MS [M+H]⁺ 473.2254.

### Example Compound 64; 5-(2-{[5-(3-Methoxyazetidin-1-yl)pyridin-3-yl]amino}pyrimidin-4-yl)-2-(tetrahydro-2H-pyran-4-yloxy)benzonitrile

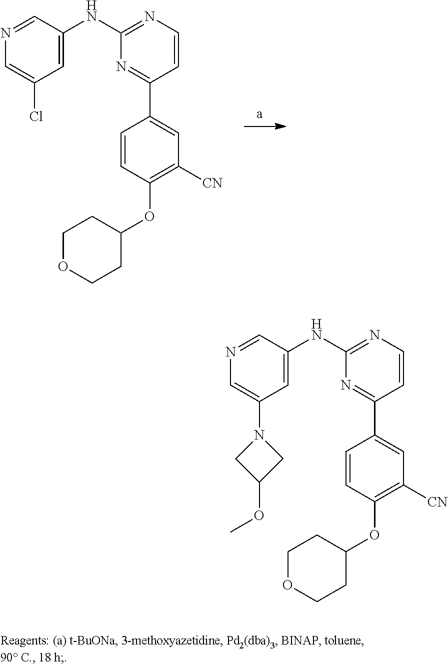 [SMILES: N#Cc1cc(-c2ccnc(Nc3cncc(Cl)c3)n2)ccc1OC1CCOCC1 ; COC1CN(c2cncc(Nc3nccc(-c4ccc(OC5CCOCC5)c(C#N)c4)n3)c2)C1]

5-{2-[(5-Chloropyridin-3-yl)amino]pyrimidin-4-yl}-2-(tetrahydro-2H-pyran-4-yloxy)benzonitrile (0.065 g, 0.16 mmol), 3-methoxyazetidine (0.175 g, 2.0 mmol), sodium t-butoxide (0.1 g), Pd₂(dba)₃ (0.022 g, 0.025 mmol) BINAP (0.09 g, 0.05 mmol), and toluene (10 mL) were added to a flask and the reaction mixture sparged with nitrogen (3 min). The sealed reaction vessel was place in a hot oil bath at 90° C. and stirred for 18 h. Upon cooling, the reaction was adsorbed onto silica by concentrating under vacuum. Column chromatography (SiO₂, MeOH/DCM, 0-40%) gave the title compound; ¹H NMR (CDCl₃) δ 8.54 (d, 1H), 8.45 (d, 1H), 8.39-8.32 (m, 1H), 8.18 (dd, 1H), 7.98 (d, 1H), 7.59 (d, 1H), 7.12-7.07 (m, 2H), 6.04 (dd, 1H), 4.78-4.74 (m, 1H), 4.46-4.41 (m, 1H), 4.29-4.26 (m, 2H), 4.07-4.01 (m, 2H), 3.95-3.92 (m, 2H), 3.70-3.64 (m, 2H), 3.40 (s, 3H), 2.13-2.07 (m, 2H), 1.97-1.89 (m, 2H). LC-MS [M+H]⁺ 459.2178.

### Example Compound 65; 5-(2-{[5-(Morpholin-4-yl)pyridin-3-yl]amino}pyrimidin-4-yl)-2-(tetrahydro-2H-pyran-4-yloxy)benzonitrile

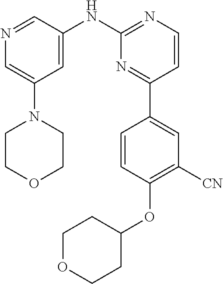 [SMILES: N#Cc1cc(-c2ccnc(Nc3cncc(N4CCOCC4)c3)n2)ccc1OC1CCOCC1]

This compound was prepared according to the procedure described for the preparation of Example Compound 64 using morpholine. Column chromatography (SiO₂, MeOH/DCM, 0-40%) gave the title compound; ¹H NMR (CDCl₃) δ 8.56 (d, 1H), 8.37 (d, 1H), 8.20 (dd, 1H), 8.10 (d, 1H), 8.04 (d, 1H), 7.12 (d, 1H), 7.07 (d, 1H), 6.44 (dd, 1H), 4.78-4.74 (m, 1H), 4.07-4.01 (m, 2H), 3.91-3.89 (m, 4H), 3.41-3.39 (m, 4H), 2.13-2.06 (m, 2H), 1.97-1.89 (m, 2H). LC-MS [M+H]⁺ 459.2269.

### Example Compound 66; 5-(2-{[2-(Hydroxymethyl)pyridin-4-yl]amino}pyrimidin-4-yl)-2-(tetrahydro-2H-pyran-4-yloxy)benzonitrile

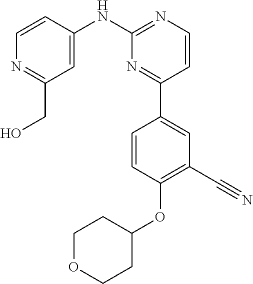 [SMILES: N#Cc1cc(-c2ccnc(Nc3ccnc(CO)c3)n2)ccc1OC1CCOCC1]

This compound was prepared according to the procedure described for the preparation of Example Compound 1 using (4-amino-2-pyridyl)methanol. The residue was purified by column chromatography (SiO₂, MeOH 20% in CH₂Cl₂ with 0.1% NH₄OH), to give the title compound; ¹H NMR (DMSO-d₆) δ 11.4 (s, 1H), 8.82 (d, 1H), 8.64 (d, 1H), 8.54-8.50 (m, 2H), 8.44-8.38 (m, 1H), 8.09-8.01 (m, 1H), 7.88 (d, 1H), 7.57 (d, 1H), 6.21 (br s, 1H), 5.01-4.95 (m, 1H), 4.80 (s, 2H), 3.91-3.84 (m, 2H), 3.60-3.53 (m, 2H), 2.09-2.02 (m, 2H), 1.74-1.68 (m, 2H). LC-MS [M+Na]⁺ 426.1522

### Example Compound 67; N-{[5-({4-[3-Cyano-4-(tetrahydro-2H-pyran-4-yloxy)phenyl]pyrimidin-2-yl}amino)pyridin-2-yl]methyl}morpholine-4-carboxamide

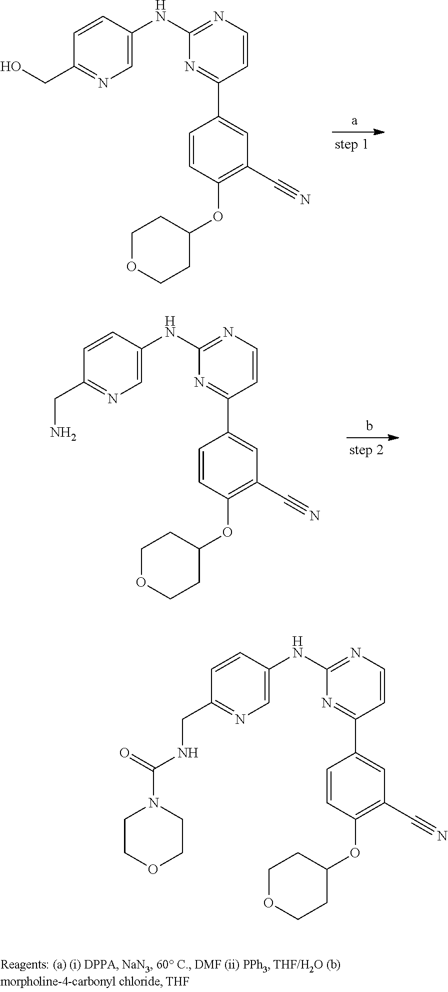 [SMILES: N#Cc1cc(-c2ccnc(Nc3ccc(CO)nc3)n2)ccc1OC1CCOCC1 ; N#Cc1cc(-c2ccnc(Nc3ccc(CN)nc3)n2)ccc1OC1CCOCC1 ; N#Cc1cc(-c2ccnc(Nc3ccc(CNC(=O)N4CCOCC4)nc3)n2)ccc1OC1CCOCC1]

Step 1. 5-[2-[[6-(Aminomethyl)-3-pyridyl]amino]pyrimidin-4-yl]-2-tetrahydropyran-4-yloxy-benzonitrile. A solution of 5-(2-{[2-(hydroxymethyl)pyridin-4-yl]amino}pyrimidin-4-yl)-2-(tetrahydro-2H-pyran-4-yloxy)benzonitrile (0.250 g, 0.62 mmol) in DMF (5 mL) was treated with DPPA (0.4 mL, 1.87 mmol) and stirred at rt for 1 h. Sodium azide (0.120 g, 1.87 mmol) was added and the solution heated to 100° C. and stirred for 4 h. The reaction was cooled to rt and extracted with EtOAc containing 1% MeOH. The combined organics were dried (Na₂SO₄) and concentrated under vacuum. The residue was taken up in THF (10 mL), and water (0.07 mL) and PPh₃ (0.422 g, 1.87 mmol) was added. Heated to reflux and stirred for 3 h. Cooled and concentrated under vacuum. The residue was purified by column chromatography (SiO₂, MeOH 20% in CH₂Cl₂ with 0.1% NH₄OH), to give the title compound; ¹H NMR (DMSO-d₆) δ 10.0 (s, 1H), 9.00 (d, 1H), 8.58 (d, 1H), 8.52 (s, 1H), 8.42 (dd, 1H), 8.29 (br s, 2H), 8.26 (dd, 1H), 7.55-7.51 (m, 2H), 7.43 (d, 1H), 4.98-4.90 (m, 1H), 4.09 (d, 2H), 3.88-3.82 (m, 2H), 3.56-3.51 (m, 2H), 2.07-1.99 (m, 2H), 1.71-1.62 (m, 2H).

Step 2. N-{[5-({4-[3-Cyano-4-(tetrahydro-2H-pyran-4-yloxy)phenyl]pyrimidin-2-yl}amino)pyridin-2-yl]methyl}morpholine-4-carboxamide. A solution of 5-[2-[[6-(Aminomethyl)-3-pyridyl]amino]pyrimidin-4-yl]-2-tetrahydropyran-4-yloxy-benzonitrile (0.05 g, 0.124 mmol) was treated with morpholine-4-carbonyl chloride (0.015 mL, 0.130 mmol) and allowed to stir at rt for 4 h. The residue was purified by column chromatography (SiO₂, MeOH 20% in CH₂Cl₂ with 0.1% NH₄OH), to give the title compound; ¹H NMR (DMSO-d₆) δ 10.2 (s, 1H), 9.06 (s, 1H), 8.63 (d, 1H), 8.55 (d, 1H), 8.45 (dd, 1H), 8.36 (d, 1H), 7.60 (d, 1H), 7.56 (d, 1H), 7.56-7.51 (m, 1H), 7.34-7.30 (m, 1H), 4.99-4.92 (m, 1H), 4.38 (d, 2H), 3.90-3.85 (m, 2H), 3.58-3.53 (m, 6H), 3.32 (t, 4H), 2.08-2.02 (m, 2H), 1.72-1.65 (m, 2H). LC-MS [M+H]⁺ 432.1676

### Example Compound 68; N-{[5-({4-[3-Cyano-4-(tetrahydro-2H-pyran-4-yloxy)phenyl]pyrimidin-2-yl}amino)pyridin-2-yl]methyl}acetamide

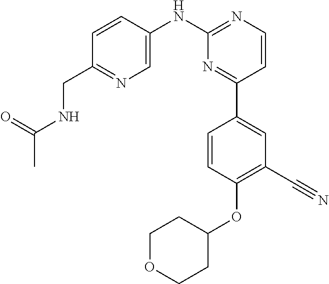 [SMILES: CC(=O)NCc1ccc(Nc2nccc(-c3ccc(OC4CCOCC4)c(C#N)c3)n2)cn1]

This compound was prepared according to the procedure described for the preparation of Example Compound 67 using acetic anhydride and DIPEA in Step 2. The residue was purified by RP-MPLC (C₁₈, MeOH/H₂O, 0-100%, with 0.1% TFA) to give the title compound; ¹H NMR (DMSO-d₆) δ 10.1 (s, 1H), 9.02 (s, 1H), 8.62 (d, 1H), 8.55 (d, 1H), 8.51 (t, 1H), 8.45 (dd, 1H), 8.32 (dd, 1H), 7.59-7.56 (m, 2H), 7.45 (d, 1H), 4.99-4.92 (m, 1H), 4.37 (d, 2H), 3.90-3.85 (m, 2H), 3.58-3.53 (m, 2H), 2.09 (s, 3H), 2.08-2.02 (m, 2H), 1.91 (s, 3H), 1.72-1.65 (m, 2H). LC-MS [M+H]⁺ 445.1992.

### Example Compound 69; 5-[2-({2-[(3-Methoxyazetidin-1-yl)methyl]pyridin-4-yl}amino)pyrimidin-4-yl]-2-(tetrahydro-2H-pyran-4-yloxy)benzonitrile

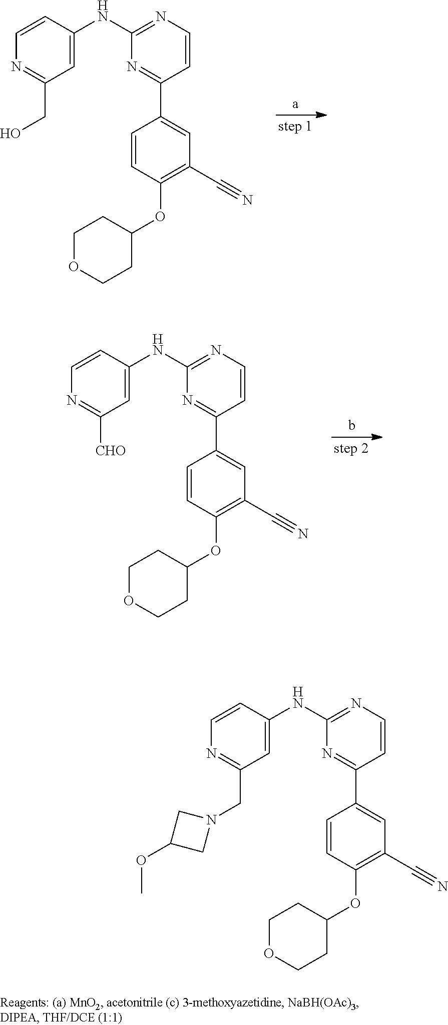 [SMILES: N#Cc1cc(-c2ccnc(Nc3ccnc(CO)c3)n2)ccc1OC1CCOCC1 ; N#Cc1cc(-c2ccnc(Nc3ccnc(C=O)c3)n2)ccc1OC1CCOCC1 ; COC1CN(Cc2cc(Nc3nccc(-c4ccc(OC5CCOCC5)c(C#N)c4)n3)ccn2)C1]

Step 1. 5-[2-[(2-Formyl-4-pyridyl)amino]pyrimidin-4-yl]-2-tetrahydropyran-4-yloxy-benzonitrile. A solution of 5-(2-{[2-(hydroxymethyl)pyridin-4-yl]amino}pyrimidin-4-yl)-2-(tetrahydro-2H-pyran-4-yloxy)benzonitrile (0.6 g, 1.5 mmol) in acetonitrile (50 mL) was treated with MnO₂ (0.6 g, 7.5 mmol) and the mixture stirred at 90° C. for 18 h. The reaction was allowed to cool, then filtered through Celite. The filtrate was concentrated under vacuum to give the title compound (0.9 g). ¹H NMR (DMSO-d₆) δ 10.5 (s, 1H), 9.96 (d, 1H), 8.72 (d, 1H), 8.63 (d, 1H), 8.61 (d, 1H), 8.54 (d, 1H), 8.51 (dd, 1H), 8.00 (dd, 1H), 7.70 (d, 1H), 7.58 (d, 1H), 5.03-4.95 (m, 1H), 3.91-3.85 (m, 2H), 3.60-3.54 (m, 2H), 2.09-2.02 (m, 2H), 1.77-1.66 (m, 2H).

Step 2. 5-[2-({2-[(3-Methoxyazetidin-1-yl)methyl]pyridin-4-yl}amino)pyrimidin-4-yl]-2-(tetrahydro-2H-pyran-4-yloxy)benzonitrile. A solution of 5-[2-[(2-Formyl-4-pyridyl)amino]pyrimidin-4-yl]-2-tetrahydropyran-4-yloxy-benzonitrile (0.050 g, 0.125 mmol) in 1:1 THF/DCE (2 mL) was treated with sodium triacetoxyborohydride (0.4 g, 0.182 mmol) and DIPEA (0.055 mL, 0.312 mmol) and 3-methoxyazetidine (0.023 g, 0.187 mmol) and stirred at rt overnight. The reaction was quenched with the addition of saturated aqueous NaHCO₃ solution and the product was extracted with DCM. The organic layer was dried over Na₂SO₄ and concentrated under vacuum. The residue was purified by column chromatography (SiO₂, MeOH 20% in CH₂Cl₂ with 0.1% NH₄OH), to give the title compound; ¹H NMR (DMSO-d₆) δ 10.5 (s, 1H), 8.70 (d, 1H), 8.60 (d, 1H), 8.55 (d, 1H), 8.48 (dd, 1H), 8.44 (d, 1H), 7.90 (s, 1H), 7.86 (d, 1H), 7.70 (d, 1H), 7.57 (d, 1H), 5.00-4.94 (m, 1H), 4.58 (s, 2H), 4.35-4.34 (m, 2H), 4.31-4.25 (m, 1H), 4.05-4.01 (m, 2H), 3.96-3.86 (m, 2H), 3.60-3.54 (m, 2H), 3.25 (s, 3H), 2.09-2.02 (m, 2H), 1.74-1.65 (m, 2H). LC-MS [M+Na]⁺ 495.2114.

### Example Compound 70; 5-[2-({2-[(3-Hydroxyazetidin-1-yl)methyl]pyridin-4-yl}amino)pyrimidin-4-yl]-2-(tetrahydro-2H-pyran-4-yloxy)benzonitrile

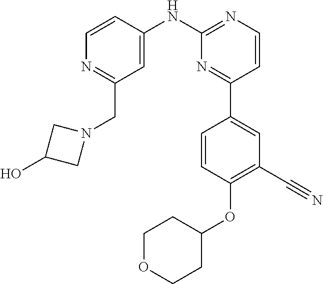 [SMILES: N#Cc1cc(-c2ccnc(Nc3ccnc(CN4CC(O)C4)c3)n2)ccc1OC1CCOCC1]

This compound was prepared according to the procedure described for the preparation of Example Compound 69 using azetidin-3-ol. The residue was purified by column chromatography (SiO₂, MeOH 20% in CH₂Cl₂ with 0.1% NH₄OH), to give the title compound; ¹H NMR (DMSO-d₆) δ 10.5 (s, 1H), 8.70 (d, 1H), 8.60 (d, 1H), 8.48 (dd, 1H), 8.44 (dd, 1H), 7.88 (s, 1H), 7.85 (d, 1H), 7.69 (d, 1H), 7.57 (d, 1H), 5.00-4.94 (m, 1H), 4.59-4.54 (m, 1H), 4.57 (s, 2H), 4.32 (dd, 2H), 3.96-3.85 (m, 4H), 3.60-3.54 (m, 2H), 2.09-2.02 (m, 2H), 1.74-1.65 (m, 2H). LC-MS [M+Na]⁺ 481.1968.

### Example Compound 71; 5-[2-({2-[(3,3-Difluoropyrrolidin-1-yl)methyl]pyridin-4-yl}amino)pyrimidin-4-yl]-2-(tetrahydro-2H-pyran-4-yloxy)benzonitrile

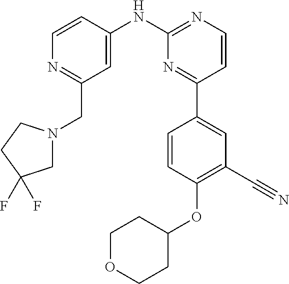 [SMILES: N#Cc1cc(-c2ccnc(Nc3ccnc(CN4CCC(F)(F)C4)c3)n2)ccc1OC1CCOCC1]

This compound was prepared according to the procedure described for the preparation of Example Compound 69 using 3,3-difluoropyrrolidine. The residue was purified by column chromatography (SiO₂, MeOH 20% in CH₂Cl₂ with 0.1% NH₄OH), to give the title compound; ¹H NMR (DMSO-d₆) δ 11.4 (s, 1H), 8.81 (d, 1H), 8.62 (d, 1H), 8.54 (d, 1H), 8.50 (dd, 1H), 8.28 (s, 1H), 8.13 (br s, 1H), 7.88 (d, 1H), 7.58 (d, 1H), 5.01-4.95 (m, 1H), 4.01 (s, 2H), 3.91-3.83 (m, 2H), 3.59-3.53 (m, 2H), 3.12 (t, 2H), 2.91 (t, 2H), 2.34 (sept, 2H), 2.09-2.02 (m, 2H), 1.74-1.65 (m, 2H). LC-MS [M+Na]⁺ 515.1990.

### Example Compound 72; 5-(2-{[6-(Morpholin-4-ylcarbonyl)pyridin-2-yl]amino}pyrimidin-4-yl)-2-(tetrahydro-2H-pyran-4-yloxy)benzonitrile

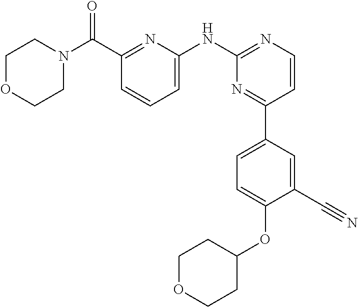 [SMILES: N#Cc1cc(-c2ccnc(Nc3cccc(C(=O)N4CCOCC4)n3)n2)ccc1OC1CCOCC1]

This compound was prepared according to the procedure described for the preparation of Example Compound 1 using (6-amino-2-pyridyl)-morpholino-methanone. Purification by RP-MPLC (C₁₈, MeOH/H₂O, 0-100%, with 0.1% TFA) provided the title compound; ¹H NMR (DMSO-d₆) δ 10.14 (s, 1H), 8.64 (d, 1H), 8.59 (d, 1H), 8.49 (dd, 1H), 8.33 (d, 1H), 7.91 (dd, 1H), 7.63 (d, 1H), 7.57 (d, 1H), 7.20 (dd, 1H), 4.93-4.99 (m, 1H), 3.84-3.91 (m, 2H), 3.62-3.69 (m, 4H), 3.55-3.61 (m, 4H), 3.49-3.54 (m, 2H), 2.01-2.08 (m, 2H), 1.65-1.74 (m, 2H). LC-MS [M+H] 487.2107.

### Example Compound 73; 5-(2-{[6-(2-Methyl-1H-imidazol-1-yl)pyridin-3-yl]amino}pyrimidin-4-yl)-2-(tetrahydro-2H-pyran-4-yloxy)benzonitrile

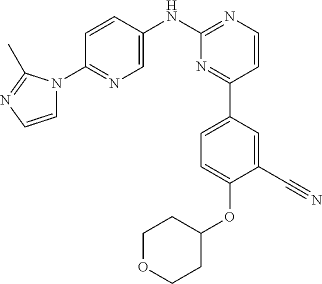 [SMILES: Cc1nccn1-c1ccc(Nc2nccc(-c3ccc(OC4CCOCC4)c(C#N)c3)n2)cn1]

This compound was prepared according to the procedure described for the preparation of Example Compound 1 using 6-(2-methylimidazol-1-yl)pyridin-3-amine. Purification by RP-MPLC (C₁₈, MeOH/H₂O, 0-100%, with 0.1% TFA) provided the title compound; ¹H NMR (DMSO-d₆) δ 10.35 (s, 1H), 9.06 (d, 1H), 8.67 (d, 1H), 8.47 (dd, 1H), 8.05 (d, 1H), 7.80 (d, 1H), 7.77 (d, 1H), 7.64 (d, 1H), 7.57 (d, 1H), 4.96 (m, 1H), 3.91-3.85 (m, 2H), 3.59-3.53 (m, 2H), 2.73 (s, 3H), 2.07-2.03 (m, 2H), 1.74-1.68 (m, 2H). LC-MS [M+H]⁺ 454.2014.

### Example Compound 74; 5-(2-{[5-(Morpholin-4-yl)pyrimidin-2-yl]amino}pyrimidin-4-yl)-2-(tetrahydro-2H-pyran-4-yloxy)benzonitrile

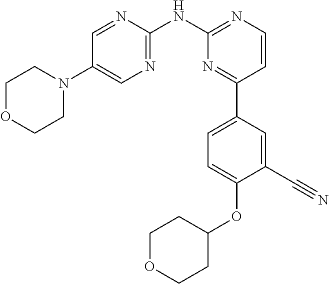 [SMILES: N#Cc1cc(-c2ccnc(Nc3ncc(N4CCOCC4)cn3)n2)ccc1OC1CCOCC1]

This compound was prepared according to the procedure described for the preparation of Example Compound 1 using 5-morpholinopyrimidin-2-amine. Purification by RP-MPLC (C₁₈, MeOH/H₂O, 0-100%, with 0.1% TFA) provided the title compound; ¹H NMR (DMSO-d₆) δ 11.94 (s, 1H), 8.78 (d, 1H), 8.66 (d, 1H), 8.51 (dd, 1H), 8.15 (d, 1H), 7.93 (d, 1H), 7.61 (d, 1H), 6.90 (d, 1H), 4.95-5.02 (m, 1H), 3.92-4.04 (m, 1H), 3.83-3.91 (m, 4H), 3.71-3.83 (m, 3H), 3.57 (ddd, 4H), 2.01-2.09 (m, 2H), 1.65-1.75 (m, 2H). LC-MS [M+H] 460.2089.

### Example Compound 75; 5-(2-{[6-(1H-Imidazol-1-yl)pyridin-3-yl]amino}pyrimidin-4-yl)-2-(tetrahydro-2H-pyran-4-yloxy)benzonitrile

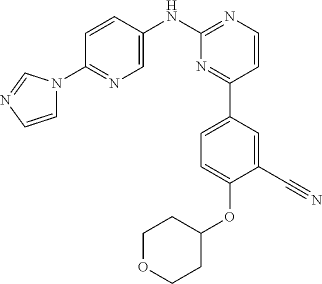 [SMILES: N#Cc1cc(-c2ccnc(Nc3ccc(-n4ccnc4)nc3)n2)ccc1OC1CCOCC1]

This compound was prepared according to the procedure described for the preparation of Example Compound 1 using 6-imidazol-1-ylpyridin-3-amine. Purification by RP-MPLC (C₁₈, MeOH/H₂O, 0-100%, with 0.1% TFA) provided the title compound; ¹H NMR (DMSO-d₆) δ 10.06 (s, 1H), 8.91 (d, 1H), 8.61 (d, 1H), 8.56 (d, 1H), 8.44-8.48 (m, 2H), 8.39 (dd, 1H), 7.91 (s, 1H), 7.79 (d, 1H), 7.55-7.60 (m, 2H), 7.12 (s, 1H), 4.92-4.99 (m, 1H), 3.83-3.91 (m, 2H), 3.56 (ddd, 2H), 2.01-2.08 (m, 2H), 1.65-1.74 (m, 2H). LC-MS [M+H] 440.1826.

### Example Compound 76; 2-(Tetrahydro-2H-pyran-4-yloxy)-5-(2-{[6-(1H-1,2,4-triazol-1-yl)pyridin-3-yl]amino}pyrimidin-4-yl)benzonitrile

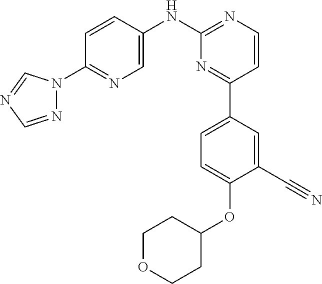 [SMILES: N#Cc1cc(-c2ccnc(Nc3ccc(-n4cncn4)nc3)n2)ccc1OC1CCOCC1]

This compound was prepared according to the procedure described for the preparation of Example Compound 1 using 6-(1,2,4-triazol-1-yl)pyridin-3-amine. Purification by RP-MPLC (C₁₈, MeOH/H₂O, 0-100%, with 0.1% TFA) provided the title compound; ¹H NMR (DMSO-d₆) δ 10.17 (s, 1H), 9.30 (s, 1H), 8.94 (d, 1H), 8.63 (d, 1H), 8.57 (d, 1H), 8.49 (ddd, 2H), 7.87 (d, 1H), 7.56-7.60 (m, 2H), 6.56 (s, 1H), 4.92-4.99 (m, 1H), 3.84-3.91 (m, 2H), 3.56 (ddd, 2H), 2.01-2.09 (m, 2H), 1.65-1.74 (m, 2H). LC-MS [M+H] 441.1849.

### Example Compound 77; 5-(2-{[6-(1-Methyl-1H-pyrazol-4-yl)pyridin-3-yl]amino}pyrimidin-4-yl)-2-(tetrahydro-2H-pyran-4-yloxy)benzonitrile

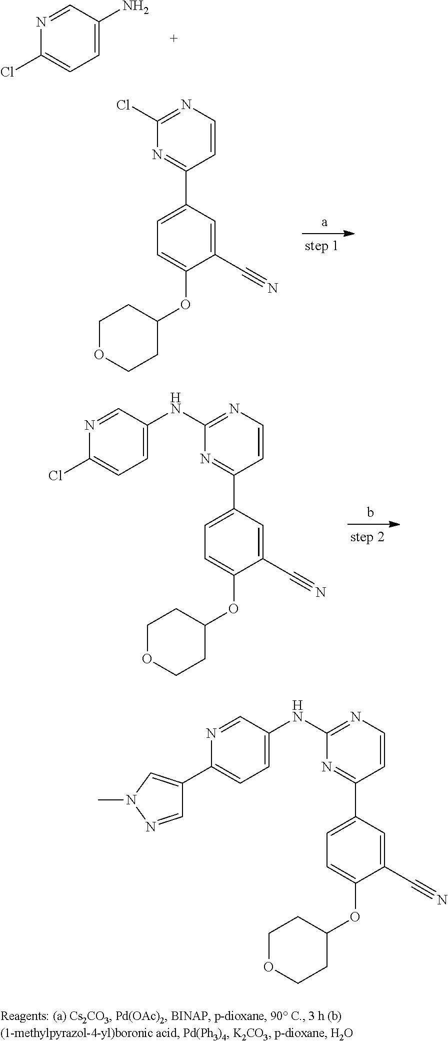

Step 1. 5-[2-[(6-Chloro-3-pyridyl)amino]pyrimidin-4-yl]-2-tetrahydropyran-4-yloxy-benzonitrile. This compound was prepared according to the procedure described for the preparation of Example Compound 1 using 6-chloropyridin-3-amine. The residue was purified by column chromatography (SiO₂, EtOAc/Hexanes, 0-80%) to afford the title compound; LC-MS [M+H]⁺ 408.6.

Step 2. 5-(2-{[6-(1-Methyl-1H-pyrazol-4-yl)pyridin-3-yl]amino}pyrimidin-4-yl)-2-(tetrahydro-2H-pyran-4-yloxy)benzonitrile. Combined 5-[2-[(6-Chloro-3-pyridyl)amino]pyrimidin-4-yl]-2-tetrahydropyran-4-yloxy-benzonitrile (0.070 g, 0.173 mmol), (1-methylpyrazol-4-yl)boronic acid (0.034 g, 0.208 mmol) Pd(PPh₃)₄ (0.010 g, 0.017 mmol), K₂CO₃ (0.048 g, 0.347 mmol), p-dioxane (3 mL), and water (0.3 mL) in a flask and sparged with nitrogen for several minutes before closing the vessel. The reaction was heated to 90° C. for 2 h, then allowed to cool to rt. Diluted the mixture with EtOAc and DCM, then washed with H₂O. The organic layer was dried (MgSO₄) and concentrated under reduced pressure. The residue was purified by column chromatography (SiO₂, EtOAc/Hexanes, 0-80%) to afford the title compound; ¹H NMR (DMSO-d₆) δ 10.07 (br s, 1H), 9.00 (s, 1H), 8.62 (d, 1H), 8.56 (d, 1H), 8.46 (dd, 1H), 8.24-8.33 (m, 2H), 8.01 (s, 1H), 7.77 (d, 1H), 7.54-7.60 (m, 2H), 4.92-4.99 (m, 1H), 3.83-3.92 (m, 5H), 3.52-3.60 (m, 2H), 2.01-2.09 (m, 2H), 1.65-1.74 (m, 2H). LC-MS [M+H] 454.1966.

### Example Compound 78; 5-(2-{[6-(1H-Pyrazol-4-yl)pyridin-3-yl]amino}pyrimidin-4-yl)-2-(tetrahydro-2H-pyran-4-yloxy)benzonitrile

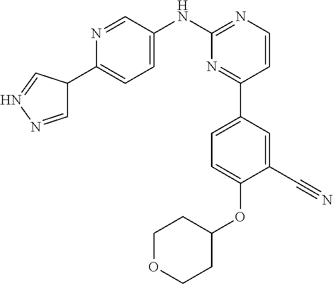 [SMILES: N#Cc1cc(-c2ccnc(Nc3ccc(C4C=NN=C4)nc3)n2)ccc1OC1CCOCC1]

This compound was prepared according to the procedure described for the preparation of Example Compound 77 using tert-butyl 4-(4,4,5,5-tetramethyl-1,3,2-dioxaborolan-2-yl)pyrazole-1-carboxylate, followed by treatment with 4 M HCl in p-dioxane to remove the Boc protecting group. Purification by RP-MPLC (C₁₈, MeOH/H₂O, 0-100%, with 0.1% TFA) provided the title compound; ¹H NMR (DMSO-d₆) δ 10.06 (br s, 1H), 9.01 (s, 1H), 8.62 (d, 1H), 8.56 (d, 1H), 8.46 (dd, 1H), 8.31 (d, 1H), 8.22 (br s, 2H), 7.83 (d, 1H), 7.55-7.65 (m, 3H), 4.92-4.99 (m, 1H), 3.84-3.91 (m, 2H), 3.56 (ddd, 2H), 2.01-2.09 (m, 2H), 1.65-1.74 (m, 2H). LC-MS [M+H] 440.1835.

### Example Compound 79; 5-[2-(2,3′-Bipyridin-5-ylamino)pyrimidin-4-yl]-2-(tetrahydro-2H-pyran-4-yloxy)benzonitrile

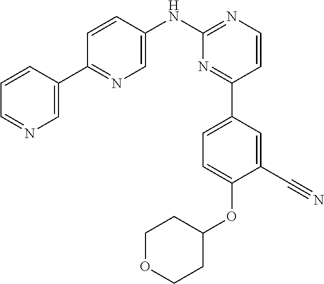 [SMILES: N#Cc1cc(-c2ccnc(Nc3ccc(-c4cccnc4)nc3)n2)ccc1OC1CCOCC1]

This compound was prepared according to the procedure described for the preparation of Example Compound 77 using 3-pyridylboronic acid. Purification by RP-MPLC (C₁₈, MeOH/H₂O, 0-100%, with 0.1% TFA) provided the title compound; ¹H NMR (DMSO-d₆) δ 10.14 (s, 1H), 9.28 (d, 1H), 9.13 (d, 1H), 8.61-8.65 (m, 2H), 8.58 (d, 1H), 8.46-8.52 (m, 2H), 8.40 (dd, 1H), 8.08 (d, 1H), 7.56-7.61 (m, 3H), 4.92-5.00 (m, 1H), 3.84-3.91 (m, 2H), 3.52-3.60 (m, 2H), 2.02-2.09 (m, 2H), 1.65-1.74 (m, 2H). LC-MS [M+H] 451.1855.

### Example Compound 80; 5-{2-[(6-{2-[(2-Methoxyethyl)amino]pyrimidin-5-yl}pyridin-3-yl)amino]pyrimidin-4-yl}-2-(tetrahydro-2H-pyran-4-yloxy)benzonitrile

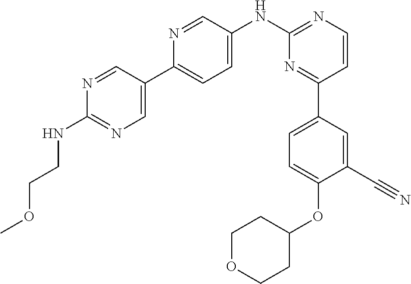 [SMILES: COCCNc1ncc(-c2ccc(Nc3nccc(-c4ccc(OC5CCOCC5)c(C#N)c4)n3)cn2)cn1]

This compound was prepared according to the procedure described for the preparation of Example Compound 77 using N-(2-methoxyethyl)-5-(4,4,5,5-tetramethyl-1,3,2-dioxaborolan-2-yl)pyrimidin-2-amine. Purification by RP-MPLC (C₁₈, MeOH/H₂O, 0-100%, with 0.1% TFA) provided the title compound; ¹H NMR (DMSO-d₆) δ 10.01 (s, 1H), 9.03 (d, 1H), 8.93 (s, 2H), 8.61 (d, 1H), 8.56 (d, 1H), 8.47 (dd, 1H), 8.29 (dd, 1H), 7.87 (d, 1H), 7.54-7.60 (m, 2H), 7.47 (br s, 1H), 4.92-4.99 (m, 1H), 3.83-3.91 (m, 2H), 3.47-3.59 (m, 6H), 3.24-3.29 (m, 3H), 2.01-2.09 (m, 2H), 1.65-1.74 (m, 2H). LC-MS [M+H] 525.2339.

### Example Compound 81; 5-(2-{[6′-(Dimethylamino)-2,3′-bipyridin-5-yl]amino}pyrimidin-4-yl)-2-(tetrahydro-2H-pyran-4-yloxy)benzonitrile

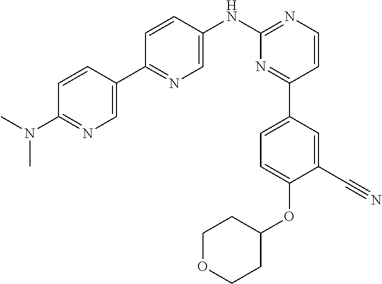 [SMILES: CN(C)c1ccc(-c2ccc(Nc3nccc(-c4ccc(OC5CCOCC5)c(C#N)c4)n3)cn2)cn1]

This compound was prepared according to the procedure described for the preparation of Example Compound 77 using (6-pyrrolidin-1-yl-3-pyridyl)boronic acid. Purification by RP-MPLC (C₁₈, MeOH/H₂O, 0-100%, with 0.1% TFA) provided the title compound; ¹H NMR (DMSO-d₆) δ 9.93 (s, 1H), 8.99 (d, 1H), 8.79 (d, 1H), 8.59 (d, 1H), 8.56 (d, 1H), 8.47 (dd, 1H), 8.24 (dd, 1H), 8.16 (dd, 1H), 7.83 (d, 1H), 7.58 (d, 1H), 7.53 (d, 1H), 6.73 (d, 1H), 4.91-4.99 (m, 1H), 3.84-3.91 (m, 2H), 3.56 (ddd, 2H), 3.06-3.10 (m, 6H), 2.01-2.09 (m, 2H), 1.65-1.74 (m, 2H). LC-MS [M+H] 494.2299.

### Example Compound 82; 5-(2-{[2-(1-Methyl-1H-pyrazol-4-yl)pyridin-4-yl]amino}pyrimidin-4-yl)-2-(tetrahydro-2H-pyran-4-yloxy)benzonitrile

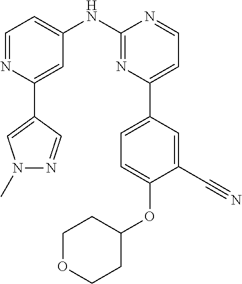 [SMILES: Cn1cc(-c2cc(Nc3nccc(-c4ccc(OC5CCOCC5)c(C#N)c4)n3)ccn2)cn1]

This compound was prepared according to the procedure described for the preparation of Example Compound 77 using 2-chloropyridin-4-amine in Step 1 and (1-methylpyrazol-4-yl)boronic acid hydrochloride and Na₂CO₃ in Step 2. Purification by RP-MPLC (C₁₈, MeOH/H₂O, 0-100%, with 0.1% TFA) provided the title compound; ¹H NMR (MeOH-d₄) δ 8.66 (d, 1H), 8.44-8.40 (m, 2H), 8.37-8.34 (m, 2H), 8.30-8.28 (m, 1H), 8.05 (d, 1H), 7.93 (br. s, 1H), 7.58 (d, 1H), 7.36 (d, 1H), 4.89 (m, 1H), 4.02 (s, 3H), 4.00-3.96 (m, 2H), 3.69-3.64 (m, 2H), 2.13-2.08 (m, 2H), 1.85-1.80 (m, 2H). LC-MS [M+H]⁺ 454.2053.

### Example Compound 83; 5-(2-{[2-(1H-Pyrazol-4-yl)pyridin-4-yl]amino}pyrimidin-4-yl)-2-(tetrahydro-2H-pyran-4-yloxy)benzonitrile

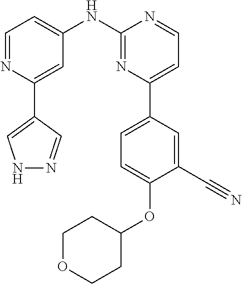 [SMILES: N#Cc1cc(-c2ccnc(Nc3ccnc(-c4cn[nH]c4)c3)n2)ccc1OC1CCOCC1]

This compound was prepared according to the procedure described for the preparation of Example Compound 77 using 2-chloropyridin-4-amine in Step 1 and 1H-pyrazol-4-ylboronic acid and Na₂CO₃ in Step 2. Purification by RP-MPLC (C₁₈, MeOH/H₂O, 0-100%, with 0.1% TFA) provided the title compound; ¹H NMR (MeOH-d₄) δ 8.72 (d, 1H), 8.55-8.41 (m, 3H), 8.35-8.32 (m, 3H), 8.05 (brs, 1H), 7.64 (d, 1H), 7.39 (d, 1H), 4.89 (m, 1H), 4.02-3.97 (m, 2H), 3.70-3.64 (m, 2H), 2.14-2.09 (m, 2H), 1.88-1.80 (m, 2H). LC-MS [M+H]⁺ 440.1883.

### Example Compound 84; 5-[2-({6-[3-(Morpholin-4-yl)pyrrolidin-1-yl]pyridazin-3-yl}amino)pyrimidin-4-yl]-2-(tetrahydro-2H-pyran-4-yloxy)Benzonitrile

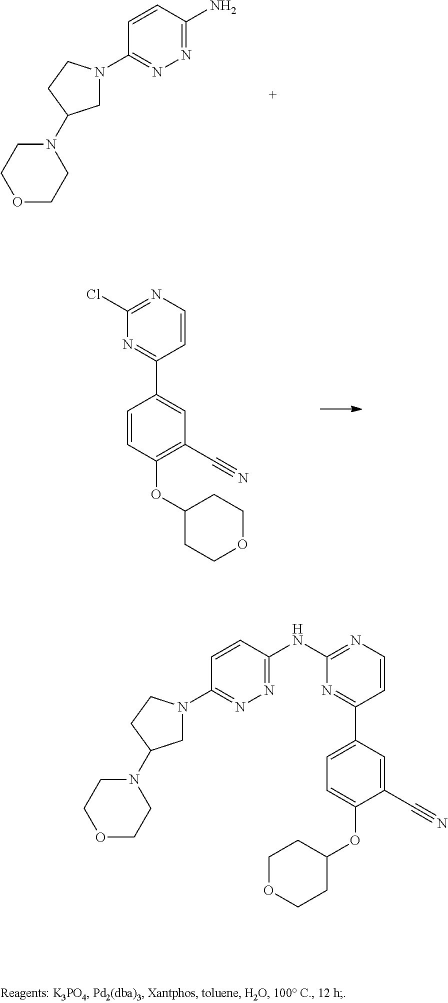 [SMILES: Nc1ccc(N2CCC(N3CCOCC3)C2)nn1 ; N#Cc1cc(-c2ccnc(Cl)n2)ccc1OC1CCOCC1 ; N#Cc1cc(-c2ccnc(Nc3ccc(N4CCC(N5CCOCC5)C4)nn3)n2)ccc1OC1CCOCC1]

5-(2-Chloropyrimidin-4-yl)-2-tetrahydropyran-4-yloxy-benzonitrile (0.05 g, 0.16 mmol), 6-(3-morpholinopyrrolidin-1-yl)pyridazin-3-amine (0.0556 g, 0.223 mmol), potassium phosphate (0.0473 g, 0.223 mmol), Pd₂(dba)₃ (0.0066 g, 0.0064 mmol) and Xantphos (0.0055 g, 0.0096 mmol), toluene (0.7 mL) and H₂O (2.88 μL) were added to a flask and the reaction mixture sparged with argon (3 min). The reaction mixture was placed in an oil bath at 100° C. and stirred for 12 h. The reaction was cooled to rt, diluted with THF and filtered with the aid of additional EtOAc. The filtrate was dried over sodium sulfate, filtered, and evaporated under reduced pressure to give the crude product. Purification by RP-MPLC (C₁₈, MeOH/H₂O, 0-100%, with 0.1% TFA) provided the title compound; ¹H NMR (DMSO-d₆) δ 10.73 (br s, 1H), 8.63 (d, 1H), 8.56 (d, 1H), 8.44 (dd, 1H), 8.23 (d, 1H), 7.67 (d, 1H), 7.55 (d, 1H), 7.48 (br s, 1H), 4.91-5.00 (m, 1H), 3.96 (dd, 2H), 3.83-3.91 (m, 4H), 3.71-3.80 (m, 5H), 3.48-3.60 (m, 8H), 1.99-2.09 (m, 2H), 1.64-1.74 (m, 2H). LC-MS [M+H] 529.2763.

### Example Compound 85; 5-(2-{[6-(4-Methylpiperazin-1-yl)pyridazin-3-yl]amino}pyrimidin-4-yl)-2-(tetrahydro-2H-pyran-4-yloxy)benzonitrile

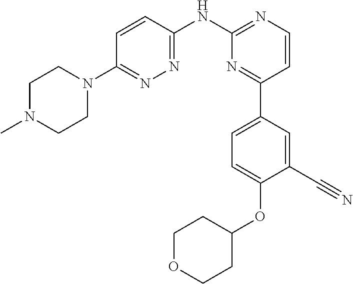 [SMILES: CN1CCN(c2ccc(Nc3nccc(-c4ccc(OC5CCOCC5)c(C#N)c4)n3)nn2)CC1]

This compound was prepared according to the procedure described for the preparation of Example Compound 84 using 6-(4-methylpiperazin-1-yl)pyridazin-3-amine. Purification by RP-MPLC (C₁₈, MeOH/H₂O, 0-100%, with 0.1% TFA) provided the title compound; ¹H NMR (DMSO-d₆) δ 10.44 (br s, 1H), 8.59 (d, 1H), 8.55 (d, 1H), 8.44 (dd, 1H), 8.23 (d, 1H), 7.52-7.63 (m, 3H), 4.90-5.01 (m, 1H), 4.39 (d, 2H), 3.83-3.91 (m, 2H), 3.52-3.60 (m, 4H), 3.13-3.23 (m, 4H), 2.87 (s, 3H), 2.00-2.08 (m, 2H), 1.64-1.73 (m, 2H). LC-MS [M+H] 473.2473.

### Example Compound 86; 5-{2-[(6-{[3-(Dimethylamino)propyl](methyl)amino}pyridazin-3-yl)amino]pyrimidin-4-yl}-2-(tetrahydro-2H-pyran-4-yloxy)benzonitrile

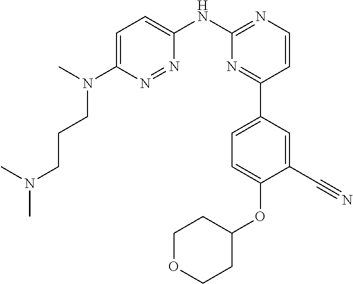 [SMILES: CN(C)CCCN(C)c1ccc(Nc2nccc(-c3ccc(OC4CCOCC4)c(C#N)c3)n2)nn1]

This compound was prepared according to the procedure described for the preparation of Example Compound 84 using N3-[3-(dimethylamino)propyl]-N3-methyl-pyridazine-3,6-diamine. Purification by RP-MPLC (C₁₈, MeOH/H₂O, 0-100%, with 0.1% TFA) provided the title compound; ¹H NMR (DMSO-d₆) δ 9.91 (s, 1H), 8.47-8.58 (m, 2H), 8.38-8.45 (m, 1H), 8.03 (d, 1H), 7.45-7.56 (m, 2H), 7.17 (d, 1H), 4.89-4.99 (m, 1H), 3.80-3.90 (m, 2H), 3.50-3.60 (m, 3H), 3.34 (s, 6H), 3.06 (s, 2H), 2.16 (s, 5H), 2.00-2.10 (m, 2H), 1.64-1.74 (m, 3H). LC-MS [M+H] 489.2746.

### Example Compound 87; 5-[2-({6-[3-(Dimethylamino)pyrrolidin-1-yl]pyridazin-3-yl}amino)pyrimidin-4-yl]-2-(tetrahydro-2H-pyran-4-yloxy)benzonitrile

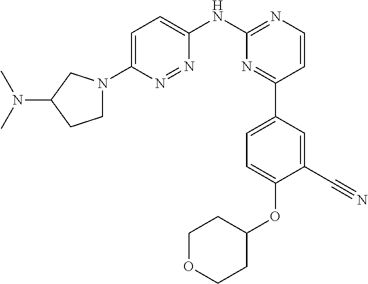 [SMILES: CN(C)C1CCN(c2ccc(Nc3nccc(-c4ccc(OC5CCOCC5)c(C#N)c4)n3)nn2)C1]

This compound was prepared according to the procedure described for the preparation of Example Compound 84 using 6-[3-(dimethylamino)pyrrolidin-1-yl]pyridazin-3-amine. Purification by RP-MPLC (C₁₈, MeOH/H₂O, 0-100%, with 0.1% TFA) provided the title compound; ¹H NMR (DMSO-d₆) δ 8.51 (d, 1H), 8.47 (d, 1H), 8.41 (dd, 1H), 8.24 (d, 1H), 7.39-7.45 (m, 2H), 7.05 (d, 1H), 4.86-4.97 (m, 1H), 3.91-4.00 (m, 2H), 3.82 (dd, 1H), 3.67-3.74 (m, 1H), 3.58-3.66 (m, 2H), 3.42-3.51 (m, 1H), 3.26-3.30 (m, 2H), 2.90-2.99 (m, 1H), 2.32 (s, 6H), 2.05-2.12 (m, 2H), 1.89-1.97 (m, 1H), 1.74-1.83 (m, 2H). LC-MS [M+H] 487.2594.

### Example Compound 88; 5-(2-{[6-(Morpholin-4-yl)pyridazin-3-yl]amino}pyrimidin-4-yl)-2-(tetrahydro-2H-pyran-4-yloxy)benzonitrile

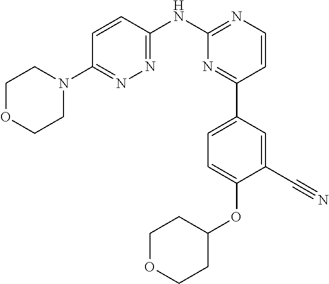 [SMILES: N#Cc1cc(-c2ccnc(Nc3ccc(N4CCOCC4)nn3)n2)ccc1OC1CCOCC1]

This compound was prepared according to the procedure described for the preparation of Example Compound 84 using 6-morpholinopyridazin-3-amine. Purification by RP-MPLC (C₁₈, MeOH/H₂O, 0-100%, with 0.1% TFA) provided the title compound; ¹H NMR (DMSO-d₆) δ 10.12 (s, 1H), 8.49-8.61 (m, 2H), 8.40-8.47 (m, 1H), 8.16-8.23 (m, 1H), 7.49-7.60 (m, 2H), 7.39 (d, 1H), 4.90-5.00 (m, 1H), 3.83-3.92 (m, 2H), 3.70-3.77 (m, 4H), 3.52-3.59 (m, 2H), 3.42-3.52 (m, 4H), 1.99-2.09 (m, 2H), 1.64-1.75 (m, 2H). LC-MS [M+H] 460.2028.

### Example Compound 89; 5-(2-{[6-(Morpholin-4-yl)pyrazin-2-yl]amino}pyrimidin-4-yl)-2-(tetrahydro-2H-pyran-4-yloxy)benzonitrile

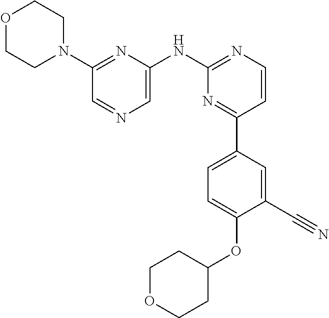 [SMILES: N#Cc1cc(-c2ccnc(Nc3cncc(N4CCOCC4)n3)n2)ccc1OC1CCOCC1]

This compound was prepared according to the procedure described for the preparation of Example Compound 84 using 6-morpholinopyrazin-2-amine. Purification by RP-MPLC (C₁₈, MeOH/H₂O, 0-100%, with 0.1% TFA) provided the title compound; ¹H NMR (DMSO-d₆) δ 8.84-8.90 (m, 1H), 8.56-8.68 (m, 2H), 8.46-8.53 (m, 1H), 7.93 (s, 1H), 7.53-7.64 (m, 2H), 4.92-5.01 (m, 1H), 3.86-3.95 (m, 2H), 3.70-3.78 (m, 4H), 3.53-3.62 (m, 4H), 3.12-3.19 (m, 2H), 2.02-2.12 (m, 2H), 1.68-1.78 (m, 2H). LC-MS [M+H] 460.2104.

### Example Compound 90; 5-[2-(Pyrimidin-2-ylamino)pyrimidin-4-yl]-2-(tetrahydro-2H-pyran-4-yloxy)benzonitrile

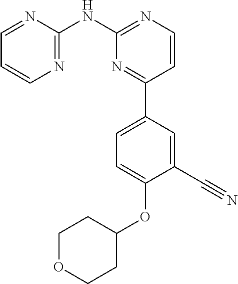 [SMILES: N#Cc1cc(-c2ccnc(Nc3ncccn3)n2)ccc1OC1CCOCC1]

This compound was prepared according to the procedure described for the preparation of Example Compound 84 using pyrimidin-2-amine. Purification by RP-MPLC (C₁₈, MeOH/H₂O, 0-100%, with 0.1% TFA) provided the title compound; ¹H NMR (DMSO-d₆) δ 10.66 (br s, 1H), 8.64-8.72 (m, 3H), 8.57-8.63 (m, 1H), 8.45-8.55 (m, 1H), 7.73 (d, 1H), 7.57 (d, 1H), 7.12 (t, 1H), 4.89-5.00 (m, 1H), 3.82-3.92 (m, 2H), 3.50-3.60 (m, 2H), 2.00-2.10 (m, 2H), 1.62-1.74 (m, 2H). LC-MS [M+H] 375.1586.

### Example Compound 91; 5-[2-(Pyridazin-4-ylamino)pyrimidin-4-yl]-2-(tetrahydro-2H-pyran-4-yloxy)benzonitrile

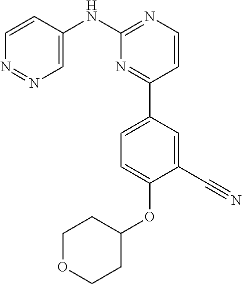 [SMILES: N#Cc1cc(-c2ccnc(Nc3ccnnc3)n2)ccc1OC1CCOCC1]

This compound was prepared according to the procedure described for the preparation of Example Compound 84 using pyridazin-4-amine. Purification by RP-MPLC (C₁₈, MeOH/H₂O, 0-100%, with 0.1% TFA) provided the title compound; ¹H NMR (DMSO-d₆) δ 10.49 (s, 1H), 9.52 (d, 1H), 8.98 (d, 1H), 8.71 (d, 1H), 8.60 (d, 1H), 8.45-8.52 (m, 1H), 8.14-8.25 (m, 1H), 7.72 (d, 1H), 7.59 (d, 1H), 4.92-5.03 (m, 1H), 3.81-3.92 (m, 2H), 3.52-3.60 (m, 2H), 2.00-2.10 (m, 2H), 1.62-1.74 (m, 2H). LC-MS [M+1] 375.1578.

### Example Compound 92; 5-[2-(Pyrazin-2-ylamino)pyrimidin-4-yl]-2-(tetrahydro-2H-pyran-4-yloxy)benzonitrile

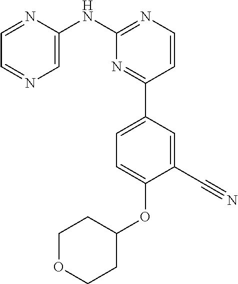 [SMILES: N#Cc1cc(-c2ccnc(Nc3cnccn3)n2)ccc1OC1CCOCC1]

This compound was prepared according to the procedure described for the preparation of Example Compound 84 using pyrazin-2-amine. Purification by RP-MPLC (C₁₈, MeOH/H₂O, 0-100%, with 0.1% TFA) provided the title compound; ¹H NMR (DMSO-d₆) δ 10.38 (s, 1H), 9.54-9.59 (m, 1H), 8.64-8.69 (m, 1H), 8.57-8.61 (m, 1H), 8.46-8.52 (m, 1H), 8.34-8.39 (m, 1H), 8.26 (s, 1H), 7.65-7.70 (m, 1H), 7.57-7.62 (m, 1H), 4.92-5.00 (m, 1H), 3.81-3.91 (m, 2H), 3.51-3.58 (m, 2H), 1.99-2.09 (m, 2H), 1.64-1.75 (m, 2H). LC-MS [M+H] 375.1568.

### Example Compound 93; 5-(2-{[5-(Morpholin-4-yl)pyridin-2-yl]amino}pyrimidin-4-yl)-2-(tetrahydro-2H-pyran-4-yloxy)benzonitrile

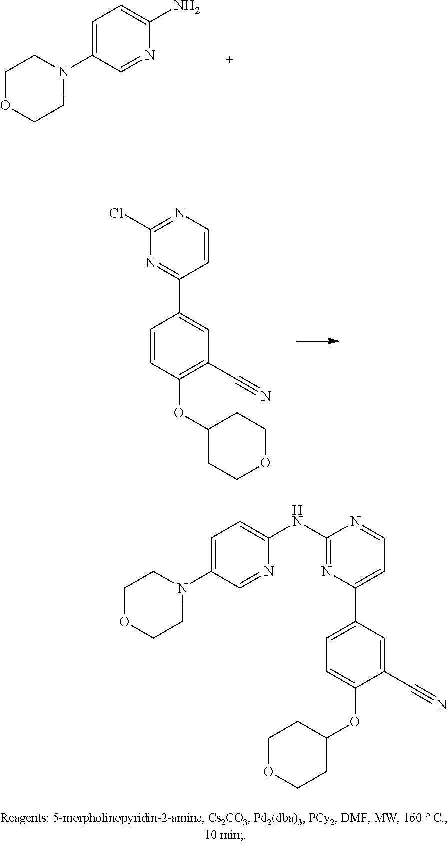

5-(2-Chloropyrimidin-4-yl)-2-tetrahydropyran-4-yloxy-benzonitrile (0.1025 g, 0.323 mmol), 5-morpholinopyridin-2-amine (0.116 g, 0.646 mmol), Cs₂CO₃ (0.263 g, 0.808 mmol), Pd₂(dba)₃ (0.006 g, 0.0065 mmol) and PCy₂ (0.005 g, 0.013 mmol), and DMF (2 mL) were added to a flask and the reaction mixture sparged with nitrogen (3 min). The reaction mixture was irradiated in a microwave reactor at 160° C. for 10 min. The reaction was cooled to rt, diluted with THF and filtered with the aid of additional EtOAc. The filtrate was dried over sodium sulfate, filtered, and evaporated under reduced pressure to give the crude product. Purification by RP-MPLC (C₁₈, MeOH/H₂O, 0-100%, with 0.1% TFA) provided the title compound; LC-MS [M+H]⁺ 458.2075.

### Example Compound 94; 5-[2-({6-[(1E)-3-(Morpholin-4-yl)prop-1-en-1-yl]pyridin-3-yl}amino)pyrimidin-4-yl]-2-(tetrahydro-2H-pyran-4-yloxy)benzonitrile

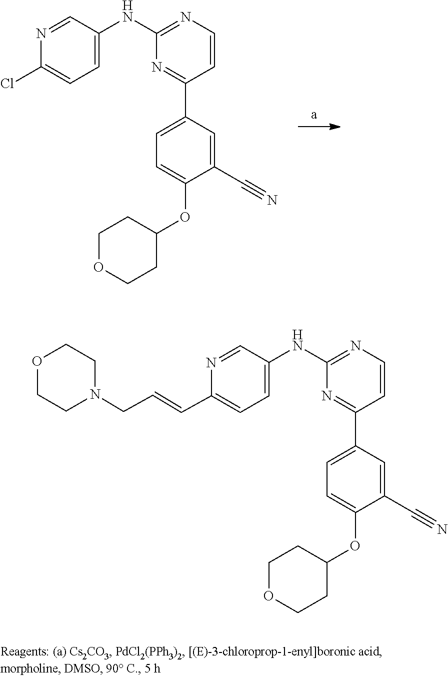 [SMILES: N#Cc1cc(-c2ccnc(Nc3ccc(Cl)nc3)n2)ccc1OC1CCOCC1 ; N#Cc1cc(-c2ccnc(Nc3ccc(/C=C/CN4CCOCC4)nc3)n2)ccc1OC1CCOCC1]

Combined Cs₂CO₃ (0.060 g, 0.490 mmol), morpholine (0.047 mL, 0.490 mmol), 5-[2-[(6-chloro-3-pyridyl)amino]pyrimidin-4-yl]-2-tetrahydropyran-4-yloxy-benzonitrile (0.100 g, 0.245 mmol), [(E)-3-chloroprop-1-enyl]boronic acid, morpholine (0.097 mL, 0.490 mmol), and PdCl₂(PPh₃)₂ (0.0085 g, 0.012 mmol) in a flask with DMSO (2 mL). Heated the mixture to 90° C. and stirred for 5 h. The reaction was allowed to cool and the mixture was diluted with EtOAc and partitioned with water. The layers were separated and the aqueous layer was extracted with DCM. The organic layers were combined, dried (MgSO₄) and concentrated under vacuum. The residue was purified by column chromatography (SiO₂, MeOH 20% in CH₂Cl₂ with 0.1% NH₄OH), to give the title compound; ¹H NMR (DMSO-d₆) δ 10.63 (s, 1H), 9.24-9.31 (m, 1H), 8.71 (d, 1H), 8.55-8.61 (m, 2H), 8.50 (dd, 1H), 8.01 (d, 1H), 7.69 (d, 1H), 7.55-7.64 (m, 1H), 6.94-7.04 (m, 2H), 4.93-5.01 (m, 1H), 3.96-4.06 (m, 4H), 3.83-3.90 (m, 2H), 3.77-3.82 (m, 2H), 3.52-3.61 (m, 2H), 3.37-3.48 (m, 2H), 3.07-3.18 (m, 2H), 2.01-2.09 (m, 2H), 1.65-1.75 (m, 2H). LC-MS [M+H] 499.2462.

### Example Compound 95; 5-{2-[(5-{[4-(2-Hydroxyethyl)piperazin-1-yl]carbonyl}thiophen-2-yl)amino]pyrimidin-4-yl}-2-(tetrahydro-2H-pyran-4-yloxy)benzonitrile

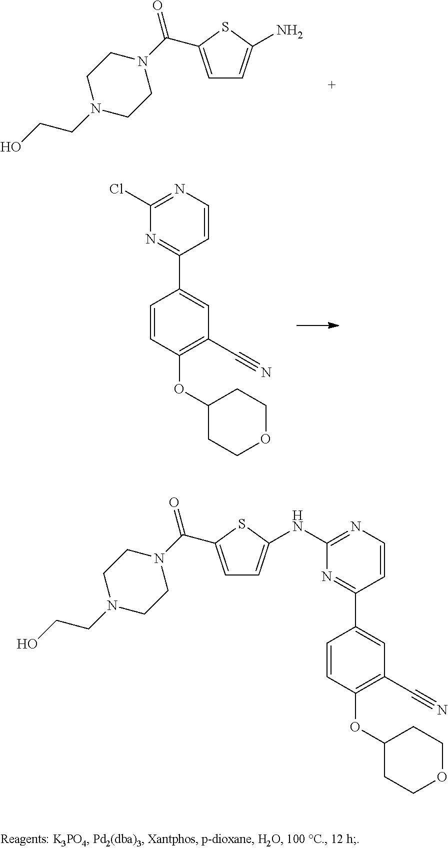 [SMILES: Nc1ccc(C(=O)N2CCN(CCO)CC2)s1 ; N#Cc1cc(-c2ccnc(Cl)n2)ccc1OC1CCOCC1 ; N#Cc1cc(-c2ccnc(Nc3ccc(C(=O)N4CCN(CCO)CC4)s3)n2)ccc1OC1CCOCC1]

5-(2-Chloropyrimidin-4-yl)-2-tetrahydropyran-4-yloxy-benzonitrile (0.08 g, 0.22 mmol), (5-amino-2-thienyl)-[4-(2-hydroxyethyl)piperazin-1-yl]methanone (0.04 g, 0.158 mmol), potassium phosphate (0.075 g, 0.26 mmol), Pd₂(dba)₃ (0.003 g) and Xantphos (0.0058 g) and p-dioxane (0.7 mL) were added to a flask and the reaction mixture sparged with argon (3 min). The reaction mixture was placed in an oil bath at 100° C. and stirred for 12 h. The reaction was cooled to rt, H₂O (5.0 mL) and 3:1 iPrOH/CHCl₃ (25 mL) were added and the layers separated. The organic layer was dried over sodium sulfate, filtered, and evaporated under reduced pressure to give the crude product. Purification by RP-MPLC (C₁₈, MeOH/H₂O, 0-100%, with 0.1% TFA) provided the title compound; ¹H NMR (MeOH-d₄) δ 8.64 (s, 1H), 8.57 (d, 1H), 8.54 (m, 1H), 7.44-7.41 (m, 3H), 6.72 (d, 1H), 4.65 (s, 2H), 4.03-3.98 (m, 2H), 3.93-3.90 (m, 1H), 3.70-3.65 (m, 2H), 3.60 (s, 2H), 2.15-2.09 (m, 2H), 1.88-1.82 (m, 2H). LC-MS [M+H]⁺ 535.2141

### Example Compound 96; 5-{2-[(3-Methyl-1,2-oxazol-5-yl)amino]pyrimidin-4-yl}-2-(tetrahydro-2H-pyran-4-yloxy)benzonitrile

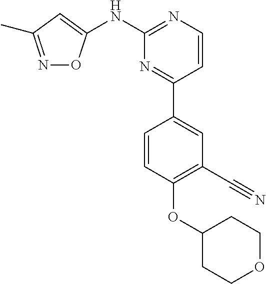 [SMILES: Cc1cc(Nc2nccc(-c3ccc(OC4CCOCC4)c(C#N)c3)n2)on1]

This compound was prepared according to the procedure described for the preparation of Example Compound 95 using 3-methylisoxazol-5-amine. Purification by RP-MPLC (C₁₈, MeOH/H₂O, 0-100%, with 0.1% TFA) provided the title compound; ¹H NMR (MeOH-d₄) δ 8.58 (d, 1H), 8.48 (d, 1H), 8.44 (dd, 1H), 7.47 (d, 1H), 7.41 (d, 1H), 6.36 (s, 1H), 4.93 (m, 1H), 4.03-3.97 (m, 2H), 3.70-3.64 (m, 2H), 2.28 (s, 3H), 2.14-2.10 (m, 2H), 1.89-1.83 (m, 2H). LC-MS [M+H]⁺ 378.1550.

### Example Compound 97; 5-{2-[(1-Methyl-1H-pyrazol-4-yl)amino]pyrimidin-4-yl}-2-(tetrahydro-2H-pyran-4-yloxy)benzonitrile

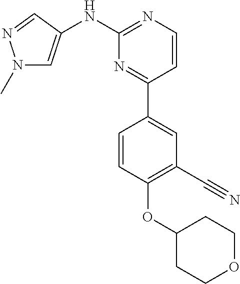 [SMILES: Cn1cc(Nc2nccc(-c3ccc(OC4CCOCC4)c(C#N)c3)n2)cn1]

This compound was prepared according to the procedure described for the preparation of Example Compound 95 using 1-methylpyrazol-4-amine. Purification by RP-MPLC (C₁₈, MeOH/H₂O, 0-100%, with 0.1% TFA) provided the title compound; ¹H NMR (MeOH-d₄) δ 8.44 (d, 1H), 8.41 (d, 1H), 8.36 (dd, 1H), 7.46 (d, 1H), 7.36 (dd, 1H), 6.33 (d, 1H), 4.88 (m, 1H), 4.02-3.97 (m, 2H), 3.76 (s, 3H), 3.71-3.63 (m, 2H), 2.13-2.08 (m, 2H), 1.86-1.81 (m, 2H). LC-MS [M+H]⁺ 377.1721.

### Example Compound 98; 5-[2-(1,3-oxazol-2-ylamino)pyrimidin-4-yl]-2-(tetrahydro-2H-pyran-4-yloxy)benzonitrile

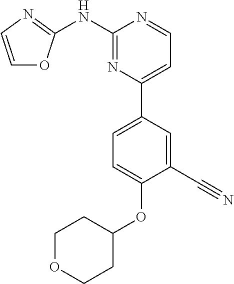 [SMILES: N#Cc1cc(-c2ccnc(Nc3ncco3)n2)ccc1OC1CCOCC1]

This compound was prepared according to the procedure described for the preparation of Example Compound 95 using oxazol-2-amine, and sodium phenoxide as the base. Purification by RP-MPLC (C₁₈, MeOH/H₂O, 0-100%, with 0.1% TFA) provided the title compound; ¹H NMR (DMSO-d₆) 8.62 (d, 1H), 8.58 (d, 1H), 8.48 (dd, 1H), 7.92 (s, 1H), 7.69 (d, 1H), 7.56 (d, 1H), 7.21 (s, 1H), 4.91-4.99 (m, 1H), 3.83-3.91 (m, 2H), 3.55 (ddd, 2H), 2.00-2.09 (m, 2H), 1.64-1.74 (m, 2H); [M+H]⁺ 364.1385.

### Example Compound 99; 5-[2-(1H-Imidazol-4-ylamino)pyrimidin-4-yl]-2-(tetrahydro-2H-pyran-4-yloxy)benzonitrile

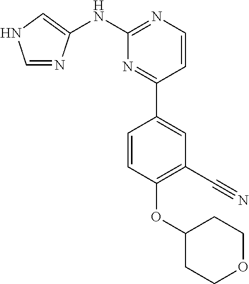 [SMILES: N#Cc1cc(-c2ccnc(Nc3c[nH]cn3)n2)ccc1OC1CCOCC1]

This compound was prepared according to the procedure described for the preparation of Example Compound 95 using 1H-imidazol-4-amine. Purification by RP-MPLC (C₁₈, MeOH/H₂O, 0-100%, with 0.1% TFA) provided the title compound; ¹H NMR (MeOH-d₄) δ 8.71 (d, 1H), 8.69 (d, 1H), 8.58 (d, 1H), 8.53 (d, 1H), 7.73 (d, 1H), 7.41 (d, 1H), 6.06 (m, 1H), 4.95 (m, 1H), 4.03-3.98 (m, 2H), 3.67-3.64 (m, 2H), 2.15-2.09 (m, 2H), 1.89-1.81 (m, 2H). LC-MS [M+H]⁺ 363.1588.

### Example Compound 100; 5-[2-({3-[(3-Methoxyazetidin-1-yl)carbonyl]-1-methyl-1H-pyrazol-5-yl}amino)pyrimidin-4-yl]-2-(tetrahydro-2H-pyran-4-yloxy)benzonitrile

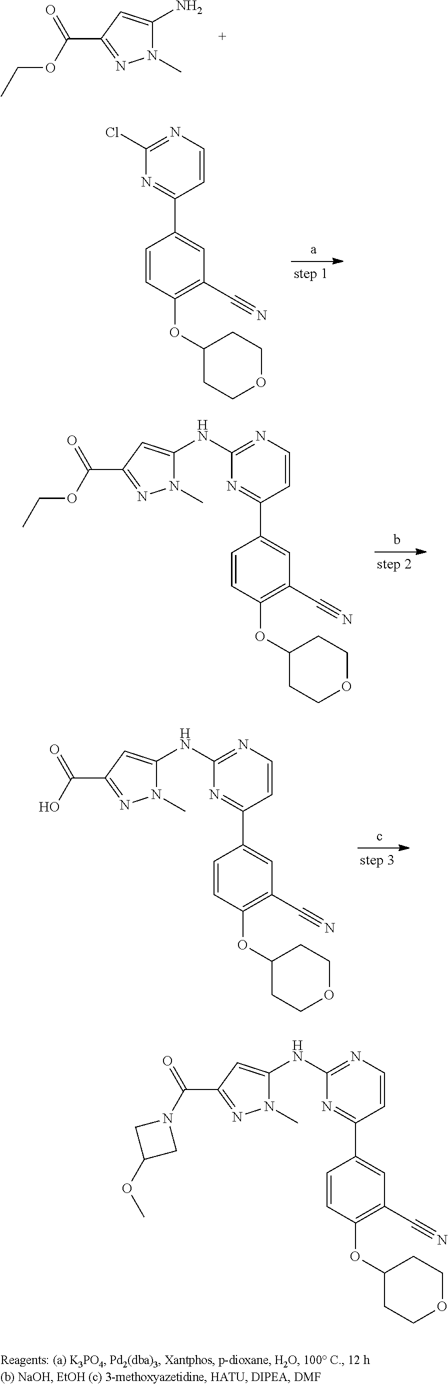

Step 1. Ethyl 5-[[4-(3-cyano-4-tetrahydropyran-4-yloxy-phenyl)pyrimidin-2-yl]amino]-1-methyl-pyrazole-3-carboxylate. This compound was prepared according to the procedure described for the preparation of Example Compound 95 using ethyl 5-amino-1-methyl-pyrazole-3-carboxylate; LC-MS [M+H]⁺ 449.3.

Step 2. 5-[[4-(3-Cyano-4-tetrahydropyran-4-yloxy-phenyl)pyrimidin-2-yl]amino]-1-methyl-pyrazole-3-carboxylic acid. A solution of crude ethyl 5-[[4-(3-cyano-4-tetrahydropyran-4-yloxy-phenyl)pyrimidin-2-yl]amino]-1-methyl-pyrazole-3-carboxylate isolated in Step 1 was dissolved in EtOH (1 mL) and treated with 2N NaOH (aq) solution (1 mL). After stirring several hours at rt, the reaction was concentrated in vacuum and the residue was taken up in 2N HCl (aq) solution resulting in the formation of a precipitate. Filtration of the solid precipitate afforded the title compound; LC-MS [M+H]⁺ 421.3.

Step 3. 5-[2-({3-[(3-Methoxyazetidin-1-yl)carbonyl]-1-methyl-1H-pyrazol-5-yl}amino)pyrimidin-4-yl]-2-(tetrahydro-2H-pyran-4-yloxy)benzonitrile. Standard Method C; HATU Coupling was used to prepare the title compound from 5-[[4-(3-cyano-4-tetrahydropyran-4-yloxy-phenyl)pyrimidin-2-yl]amino]-1-methyl-pyrazole-3-carboxylic acid and 3-methoxyazetidine; RP-MPLC(C¹⁸, MeOH/H₂O, 0-100% w/0.1% TFA) provided the pure product; ¹H NMR (MeOH-d₄) δ 8.45 (d, 1H), 8.41 (d, 1H), 8.37 (d, 1H), 7.38 (d, 1H), 7.37 (d, 1H), 6.75 (s, 1H), 4.89 (m, 1H), 4.83-4.79 (m, 1H), 4.43 (dd, 1H), 4.31-3.36 (m, 2H), 4.02-3.94 (m, 2H), 3.81 (s, 3H), 3.69-3.63 (m, 4H), 2.14-2.07 (m, 2H), 1.87-1.79 (m, 2H). LC-MS [M+H]⁺ 490.2197

### Example Compound 101; 5-[2-({1-Methyl-3-[(4-methylpiperazin-1-yl)carbonyl]-1H-pyrazol-5-yl}amino)pyrimidin-4-yl]-2-(tetrahydro-2H-pyran-4-yloxy)benzonitrile

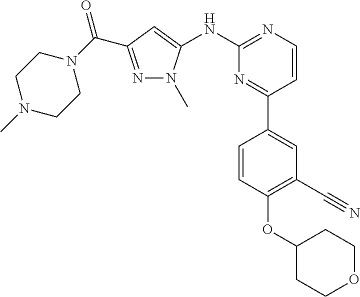 [SMILES: CN1CCN(C(=O)c2cc(Nc3nccc(-c4ccc(OC5CCOCC5)c(C#N)c4)n3)n(C)n2)CC1]

This compound was prepared according to the procedure described for the preparation of Example Compound 100 using 1-methylpiperazine in Step 3. Purification by RP-MPLC (C₁₈, MeOH/H₂O, 0-100%, with 0.1% TFA) provided the title compound; ¹H NMR (MeOH-d₄) δ 8.47 (d, 1H), 8.41 (d, 1H), 8.36 (dd, 1H), 7.40 (d, 1H), 7.37 (d, 1H), 6.77 (s, 1H), 5.40 (s, 1H), 4.90 (m, 1H), 4.02-3.96 (m, 2H), 3.83 (s, 3H), 3.69-3.55 (m, 4H), 3.21 (s, 2H), 2.97 (s, 3H), 2.14-2.07 (m, 2H), 1.89-1.83 (m, 2H). LC-MS [M+H]⁺ 503.2514.

### Example Compound 102; 5-[2-({2-[(3-Methoxyazetidin-1-yl)carbonyl]-1-methyl-1H-imidazol-5-yl}amino)pyrimidin-4-yl]-2-(tetrahydro-2H-pyran-4-yloxy)benzonitrile

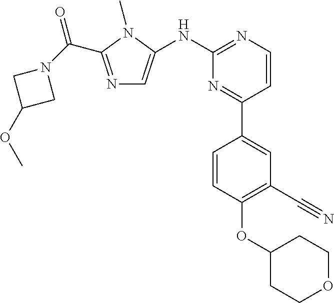 [SMILES: COC1CN(C(=O)c2ncc(Nc3nccc(-c4ccc(OC5CCOCC5)c(C#N)c4)n3)n2C)C1]

This compound was prepared according to the procedure described for the preparation of Example Compound 100 using methyl 5-amino-1-methyl-imidazole-2-carboxylate in Step 1 and 3-methoxyazetidine in Step 3. Purification by RP-MPLC (C₁₈, MeOH/H₂O, 0-100%, with 0.1% TFA) provided the title compound; ¹H NMR (MeOH-d₄) δ 8.50 (s, 1H), 8.42 (dd, 1H), 8.34 (d, 1H), 7.40 (d, 1H), 7.29 (d, 1H), 7.24 (d, 1H), 6.70 (s, 1H), 4.95 (m, 2H), 4.60 (s, 1H), 4.30 (m, 2H), 4.02-3.95 (m, 2H), 3.80 (s, 3H), 3.69-3.64 (m, 2H), 3.31 (s, 3H), 2.14-2.08 (m, 2H), 1.89-1.80 (m, 2H). LC-MS [M+H]⁺ 489.2357.

### Example Compound 103; 5-[2-({1-Methyl-2-[(4-methylpiperazin-1-yl)carbonyl]-1H-imidazol-5-yl}amino)pyrimidin-4-yl]-2-(tetrahydro-2H-pyran-4-yloxy)benzonitrile

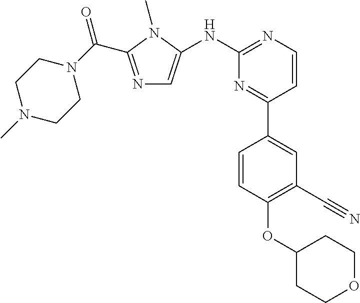 [SMILES: CN1CCN(C(=O)c2ncc(Nc3nccc(-c4ccc(OC5CCOCC5)c(C#N)c4)n3)n2C)CC1]

This compound was prepared according to the procedure described for the preparation of Example Compound 100 using methyl 5-amino-1-methyl-imidazole-2-carboxylate in Step 1 and 1-methylpiperazine in Step 3. Purification by RP-MPLC (C₁₈, MeOH/H₂O, 0-100%, with 0.1% TFA) provided the title compound; ¹H NMR (MeOH-d₄) δ 8.61 (d, 1H), 8.59 (dd, 1H), 8.56 (dd, 1H), 7.50 (d, 1H), 7.43 (d, 1H), 6.49 (s, 1H), 4.50 (dd, 1H), 4.26 (m, 1H), 4.17 (m, 2H), 4.03-3.98 (m, 2H), 3.85 (m, 1H), 3.70-3.64 (m, 2H), 3.56 (s, 2H), 3.31 (s, 3H), 2.15-2.10 (m, 2H), 1.88-1.83 (m, 2H). LC-MS [M+H]⁺ 507.1785.

### Example Compound 104; 5-[2-({4-[(3-Methoxyazetidin-1-yl)carbonyl]-1,3-thiazol-2-yl}amino)pyrimidin-4-yl]-2-(tetrahydro-2H-pyran-4-yloxy)benzonitrile

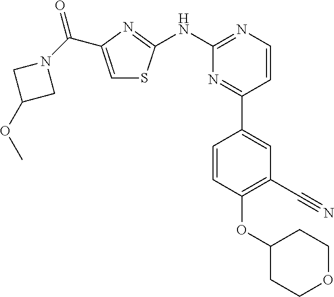 [SMILES: COC1CN(C(=O)c2csc(Nc3nccc(-c4ccc(OC5CCOCC5)c(C#N)c4)n3)n2)C1]

This compound was prepared according to the procedure described for the preparation of Example Compound 100 using methyl 2-aminothiazole-4-carboxylate in Step 1 and 3-methoxyazetidine in Step 3. Purification by RP-MPLC (C₁₈, MeOH/H₂O, 0-100%, with 0.1% TFA) provided the title compound; ¹H NMR (MeOH-d₄) δ 8.63 (d, 1H), 8.56 (s, 1H), 8.55 (dd, 1H), 7.75 (s, 1H), 7.50 (d, 1H), 7.43 (d, 1H), 4.55-4.52 (m, 1H), 4.03-4.96 (m, 3H), 4.03-3.97 (m, 2H), 3.79 (s, 3H), 3.70-3.64 (m, 2H), 2.15-2.09 (m, 2H), 1.89-1.82 (m, 2H). LC-MS [M+H]⁺ 493.1603.

### Example Compound 105; 5-[2-({4-[2-(3-Methoxyazetidin-1-yl)-2-oxoethyl]-1,3-thiazol-2-yl}amino)pyrimidin-4-yl]-2-(tetrahydro-2H-pyran-4-yloxy)benzonitrile

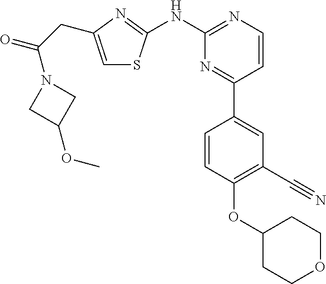 [SMILES: COC1CN(C(=O)Cc2csc(Nc3nccc(-c4ccc(OC5CCOCC5)c(C#N)c4)n3)n2)C1]

This compound was prepared according to the procedure described for the preparation of Example Compound 100 using methyl 2-(2-aminothiazol-4-yl)acetate in Step 1 and 1-methylpiperazine in Step 3. Purification by RP-MPLC (C₁₈, MeOH/H₂O, 0-100%, with 0.1% TFA) provided the title compound; ¹H NMR (MeOH-d₄) δ 8.50 (s, 1H), 8.37 (dd, 2H), 8.34 (d, 1H), 7.40 (d, 1H), 7.26 (d, 1H), 7.24 (d, 1H), 6.66 (s, 1H), 4.70 (s, 2H), 4.03-3.97 (m, 2H), 3.79 (s, 3H), 3.70-3.64 (m, 2H), 3.60 (s, 2H), 2.97 (s, 3H), 3.21 (s, 2H), 2.14-2.09 (m, 2H), 1.86-1.82 (m, 2H). LC-MS [M+H]⁺ 502.2504.

### Example Compound 106; 2-Methoxy-5-{2-[(2-methoxypyridin-4-yl)amino]pyrimidin-4-yl}benzonitrile

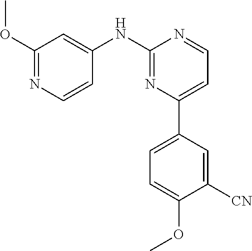 [SMILES: COc1cc(Nc2nccc(-c3ccc(OC)c(C#N)c3)n2)ccn1]

This compound was prepared according to the procedure described for the preparation of Example Compound 1 using 2-methoxypyridin-4-amine and 5-(2-chloropyrimidin-4-yl)-2-methoxybenzonitrile. Purification by RP-MPLC (C₁₈, MeOH/H₂O, 0-100%, with 0.1% TFA) provided the title compound; ¹H NMR (CDCl₃) δ 8.53 (d, 1H), 8.34-8.32 (m, 2H), 8.01 (d, 1H), 7.43 (s, 1H), 7.42-7.14 (m, 3H), 4.04 (s, 3H), 3.96 (s, 3H). LC-MS [M+H]⁺ 334.2067.

### Example Compound 107; 2-Methoxy-5-{2-[(5-methoxypyridin-2-yl)amino]pyrimidin-4-yl}benzonitrile

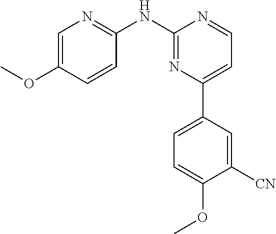 [SMILES: COc1ccc(Nc2nccc(-c3ccc(OC)c(C#N)c3)n2)nc1]

This compound was prepared according to the procedure described for the preparation of Example Compound 106 using 5-methoxypyridin-2-amine. Purification by RP-MPLC (C₁₈, MeOH/H₂O, 0-100%, with 0.1% TFA) provided the title compound; ¹H NMR (CDCl₃) δ 8.52 (d, 1H), 8.34-8.29 (m, 3H), 8.11 (br. s, 1H), 8.02 (d, 1H), 7.34 (dd, 1H), 7.14 (dd, 2H), 4.04 (s, 3H), 3.87 (s, 3H). LC-MS [M+H]⁺ 334.2039.

### Example Compound 108; 2-Methoxy-5-{2-[(6-methoxypyridin-3-yl)amino]pyrimidin-4-yl}benzonitrile

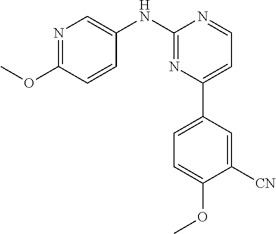 [SMILES: COc1ccc(Nc2nccc(-c3ccc(OC)c(C#N)c3)n2)cn1]

This compound was prepared according to the procedure described for the preparation of Example Compound 106 using 6-methoxypyridin-3-amine. Purification by RP-MPLC (C₁₈, MeOH/H₂O, 0-100%, with 0.1% TFA) provided the title compound; ¹H NMR (CDCl₃) δ 8.44 (d, 1H), 8.36 (d, 1H), 8.28-8.26 (m, 2H), 7.95 (dd, 1H), 7.12-7.06 (m, 3H), 6.80 (d, 1H), 4.02 (s, 3H), 3.95 (s, 3H). LC-MS [M+H]⁺ 334.1843.

### Example Compound 109; 5-[2-({6-[(2-Hydroxyethyl)(methyl)amino]pyridin-3-yl}amino)pyrimidin-4-yl]-2-(2-methylpropoxy)benzonitrile

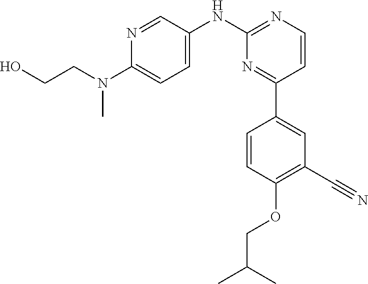 [SMILES: CC(C)COc1ccc(-c2ccnc(Nc3ccc(N(C)CCO)nc3)n2)cc1C#N]

This compound was prepared according to the procedure described for the preparation of Example Compound 1 using 2-[(5-amino-2-pyridyl)-methyl-amino]ethanol and 5-(2-chloropyrimidin-4-yl)-2-isobutoxy-benzonitrile as starting materials. Purification by RP-MPLC (C₁₈, MeOH/H₂O, 0-100%, with 0.1% TFA) provided the title compound; ¹H NMR (DMSO-d₆) δ 9.92 (s, 1H), 8.62 (d, 1H), 8.59 (d, 1H), 8.54-8.51 (m, 1H), 8.16-8.13 (m, 1H), 7.54 (d, 1H), 7.44-4.37 (m, 2H), 4.03 (d, 2H), 3.67 (br s, 4H), 3.20 (s, 3H), 2.13-2.09 (m, 1H), 1.04 (d, 6H). LC-MS [M+H]⁺ 419.2182.

### Example Compound 110; 2-(Cyclopropylmethoxy)-5-{2-[(6-ethoxypyridin-3-yl)amino]pyrimidin-4-yl}benzonitrile

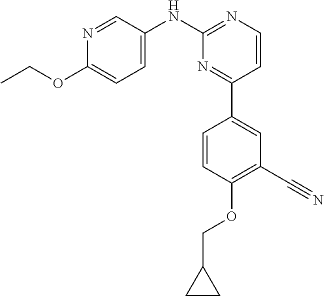 [SMILES: CCOc1ccc(Nc2nccc(-c3ccc(OCC4CC4)c(C#N)c3)n2)cn1]

This compound was prepared according to the procedure described for the preparation of Example Compound 1 using 2-[(5-amino-2-pyridyl)-methyl-amino]ethanol and 5-(2-chloropyrimidin-4-yl)-2-(cyclopropylmethoxy)benzonitrile as starting materials. Purification by column chromatography (SiO₂, MeOH/DCM, 0-40%) provided the title compound; ¹H NMR (DMSO-d₆) δ 9.60 (s, 1H), 8.52-8.50 (m, 3H), 8.43-8.40 (m, 1H), 8.04-8.01 (m, 1H), 7.45 (d, 1H), 7.41 (d, 1H), 6.79 (d, 1H), 4.29-4.24 (m, 2H), 4.10 (d, 2H), 3.34 (s, 3H), 1.32 (t, 3H), 1.28-1.22 (m, 1H), 0.65-0.61 (m, 2H), 0.42-0.38 (m, 2H). LC-MS [M+H]⁺ 388.1803.

### Example Compound 111; 2-(Cyclopropylmethoxy)-5-[2-({6-[(2-hydroxyethyl)(methyl)amino]pyridin-3-yl}amino)pyrimidin-4-yl]benzonitrile

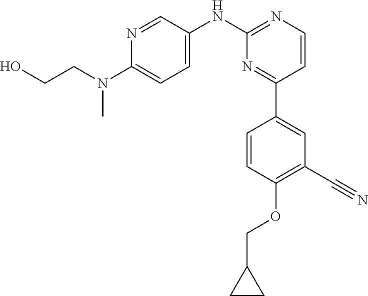 [SMILES: CN(CCO)c1ccc(Nc2nccc(-c3ccc(OCC4CC4)c(C#N)c3)n2)cn1]

This compound was prepared according to the procedure described for the preparation of Example Compound 110 using 2-[(5-amino-2-pyridyl)-methyl-amino]ethanol. Purification by RP-MPLC (C₁₈, MeOH/H₂O, 0-100%, with 0.1% TFA) provided the title compound; ¹H NMR (DMSO-d₆) δ 9.87 (s, 1H), 8.59 (d, 1H), 8.58 (d, 1H), 8.52 (d, 1H), 8.45-8.42 (m, 1H), 8.14-8.10 (m, 1H), 7.53 (d, 1H), 7.40 (d, 1H), 7.32 (d, 1H), 4.12 (d, 2H), 3.67-3.66 (m, 4H), 3.19 (s, 3H), 0.65-0.61 (m, 2H), 0.42-0.38 (m, 2H). LC-MS [M+H]⁺ 417.2021.

### Example Compound 112; 2-(Cyclopropylmethoxy)-5-{2-[(6-methylpyridin-3-yl)amino]pyrimidin-4-yl}benzonitrile

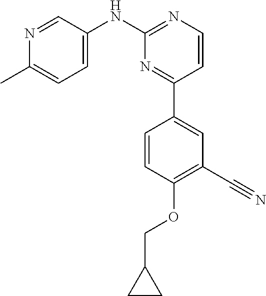 [SMILES: Cc1ccc(Nc2nccc(-c3ccc(OCC4CC4)c(C#N)c3)n2)cn1]

This compound was prepared according to the procedure described for the preparation of Example Compound 110 using 6-methylpyridin-3-amine. Purification by column chromatography (SiO₂, EtOAc/Hexanes, 0-80%) afforded the title compound; ¹H NMR (DMSO-d₆) δ 10.50 (s, 1H), 9.29 (d, 1H), 8.68 (d, 1H), 8.53 (d, 1H), 8.52-8.46 (m, 3H), 7.81 (d, 1H), 7.66 (d, 1H), 7.41 (d, 1H), 4.12 (d, 1H), 2.65 (s, 3H), 1.36-1.26 (m, 1H), 0.67-0.58 (m, 2H), 0.47-0.39 (m, 2H). LC-MS [M+H]⁺ 358.1700.

### Example Compound 113; 2-[(3-Methyloxetan-3-yl)methoxy]-5-{2-[(6-methylpyridin-3-yl)amino]pyrimidin-4-yl}benzonitrile

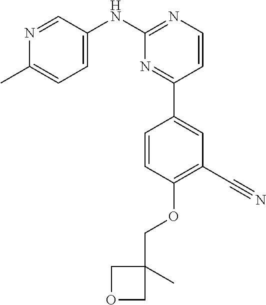 [SMILES: Cc1ccc(Nc2nccc(-c3ccc(OCC4(C)COC4)c(C#N)c3)n2)cn1]

This compound was prepared according to the procedure described for the preparation of Example Compound 1 using 6-methylpyridin-3-amine and 5-(2-chloropyrimidin-4-yl)-2-[(3-methyloxetan-3-yl)methoxy]benzonitrile as starting materials. Purification by column chromatography (SiO₂, MeOH/DCM, 0-40%) provided the title compound; ¹H NMR (DMSO-d₆) δ 9.79 (s, 1H), 8.84 (d, 1H), 8.56 (d, 1H), 8.53 (d, 1H), 8.49-8.46 (m, 1H), 8.11-8.07 (m, 1H), 7.51-7.48 (m, 2H), 7.21 (d, 1H), 4.54 (d, 2H), 4.36-4.31 (m, 4H), 2.43 (s, 3H), 1.42 (s, 3H). LC-MS [M+H]⁺ 388.1813.

### Example Compound 114; 3-Methoxy-5-{2-[(6-methylpyridin-3-yl)amino]pyrimidin-4-yl}-2-(tetrahydro-2H-pyran-4-yloxy)benzonitrile

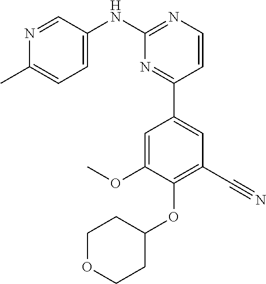 [SMILES: COc1cc(-c2ccnc(Nc3ccc(C)nc3)n2)cc(C#N)c1OC1CCOCC1]

This compound was prepared according to the procedure described for the preparation of Example Compound 1 using 6-methylpyridin-3-amine and 5-(2-chloropyrimidin-4-yl)-3-methoxy-2-tetrahydropyran-4-yloxy-benzonitrile as starting materials. Purification by column chromatography (SiO₂, MeOH/DCM, 0-40%) provided the title compound; ¹H NMR (DMSO-d₆) δ 9.81 (s, 1H), 8.80 (d, 1H), 8.60 (d, 1H), 8.15-8.11 (m, 3H), 7.57 (d, 1H), 7.21 (d, 1H), 4.74-4.67 (m, 1H), 4.00 (s, 3H), 3.95-3.88 (m, 2H), 3.47-3.40 (m, 2H), 2.42 (s, 3H), 1.97-1.92 (m, 2H), 1.74-1.65 (m, 2H). LC-MS [M+H]⁺ 418.1861.

### Example Compound 115; 5-(2-{[6-(Azetidin-1-ylmethyl)pyridin-3-yl]amino}pyrimidin-4-yl)-3-methoxy-2-(tetrahydro-2H-pyran-4-yloxy)benzonitrile

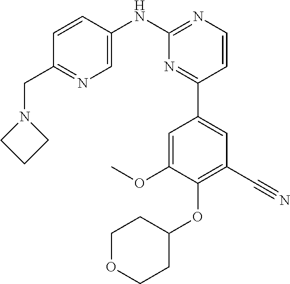 [SMILES: COc1cc(-c2ccnc(Nc3ccc(CN4CCC4)nc3)n2)cc(C#N)c1OC1CCOCC1]

This compound was prepared according to the procedure described for the preparation of Example Compound 47 using 5-(2-chloropyrimidin-4-yl)-3-methoxy-2-tetrahydropyran-4-yloxy-benzonitrile and azetidine as starting materials. Purification by RP-MPLC (C₁₈, MeOH/H₂O, 0-100%, with 0.1% TFA) provided the title compound; ¹H NMR (DMSO-d₆) δ 10.3 (br s, 1H), 10.1 (s, 1H), 8.98 (s, 1H), 8.66 (d, 1H), 8.37 (dd, 1H), 8.14-8.09 (m, 2H), 7.66 (d, 1H), 7.45 (d, 1H), 4.75-4.69 (m, 1H), 4.48 (d, 2H), 4.14-4.09 (m, 4H), 4.01 (s, 3H), 3.93-3.88 (m, 4H), 3.47-3.42 (m, 2H), 2.45-2.33 (m, 2H), 1.98-1.93 (m, 2H), 1.74-1.66 (m, 2H). LC-MS [M+H]⁺ 473.2312.

### Example Compound 116; 3-Methoxy-5-[2-({6-[(3-methoxyazetidin-1-yl)methyl]pyridin-3-yl}amino)pyrimidin-4-yl]-2-(tetrahydro-2H-pyran-4-yloxy)benzonitrile

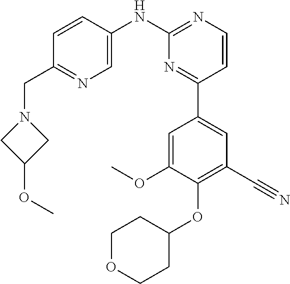 [SMILES: COc1cc(-c2ccnc(Nc3ccc(CN4CC(OC)C4)nc3)n2)cc(C#N)c1OC1CCOCC1]

This compound was prepared according to the procedure described for the preparation of Example Compound 115 using 3-methoxyazetidine. Purification by RP-MPLC (C₁₈, MeOH/H₂O, 0-100%, with 0.1% TFA) provided the title compound; ¹H NMR (DMSO-d₆) δ 10.1 (s, 1H), 8.98 (s, 1H), 8.36 (d, 1H), 8.13 (d, 2H), 7.66 (d, 1H), 7.46 (d, 1H), 4.73-4.69 (m, 1H), 4.52 (s, 2H), 4.38-4.32 (m, 2H), 4.29-4.25 (m, 1H), 4.01 (s, 3H), 3.93-3.88 (m, 4H), 3.44 (t, 2H), 3.25 (s, 3H), 1.99-1.93 (m, 2H), 1.72-1.68 (m, 2H). LC-MS [M+H]⁺ 503.2414.

### Example Compound 117; 5-(2-{[6-(Diethylamino)pyridin-3-yl]amino}pyrimidin-4-yl)-2-{[(3R)-1-(hydroxyacetyl)pyrrolidin-3-yl]oxy}benzonitrile

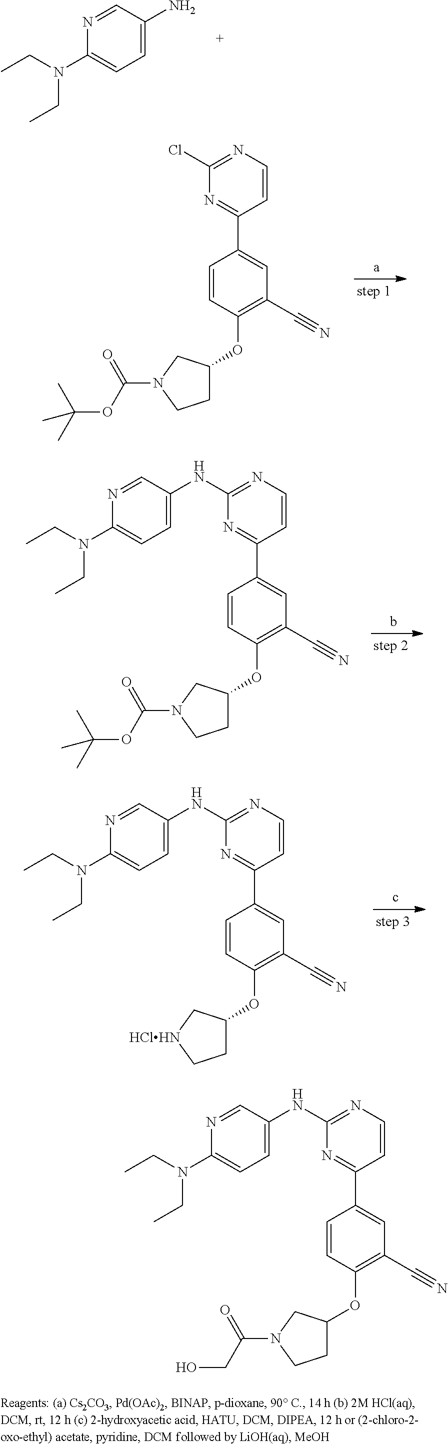

Step 1. tert-butyl (3R)-3-[2-Cyano-4-[2-[[6-(diethylamino)-3-pyridyl]amino]pyrimidin-4-yl]phenoxy]pyrrolidine-1-carboxylate. The procedure used in Step 3 in the preparation of Example Compound 1 was used to prepare the title compound from N2,N2-diethylpyridine-2,5-diamine.

Step 2. 5-[2-[[6-(Diethylamino)-3-pyridyl]amino]pyrimidin-4-yl]-2-[(3R)-pyrrolidin-3-yl]oxy-benzonitrile. Standard Method B, BOC Deprotection, was used to prepare the hydrochloride salt of the title compound from the crude product isolated in Step 1.

Step 3. 5-[2-[[6-(Diethylamino)-3-pyridyl]amino]pyrimidin-4-yl]-2-[(3R)-1-(2-hydroxyacetyl)pyrrolidin-3-yl]oxy-benzonitrile. Standard Method C, HATU Coupling, was used to prepare the title compound starting from the crude product isolated in Step 2. Alternatively, treatment of the product isolated in Step 2 with pyridine and (2-chloro-2-oxo-ethyl)acetate in DCM, followed by treatment of the crude concentrated isolate with LiOH in MeOH also affords the product. Purification by column chromatography (SiO₂, EtOAc/Hexanes, 0-80%) afforded the title compound. ¹H NMR (DMSO-d₆) δ 9.90 (s, 1H), 8.61 (d, 1H), 8.55 (d, 1H), 8.50-8.49 (m, 1H), 8.44-8.41 (m, 1H), 8.16-8.13 (m, 1H), 7.52-7.50 (M, 1H), 7.45 (d, 1H), 7.29 (d, 1H), 5.40-5.30 (m, 1H), 4.09-3.94 (m, 2H), 3.81-3.39 (m, 8H), 2.30-2.10 (m, 2H), 1.16 (t, 6H). LC-MS [M+H]⁺ 488.2421.

### Example Compound 118; 2-{[(3R)-1-(Hydroxyacetyl)pyrrolidin-3-yl]oxy}-5-{2-[(6-methylpyridin-3-yl)amino]pyrimidin-4-yl}benzonitrile

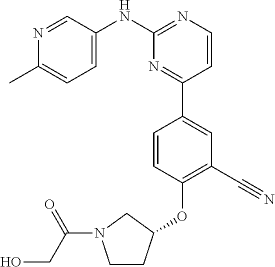 [SMILES: Cc1ccc(Nc2nccc(-c3ccc(O[C@@H]4CCN(C(=O)CO)C4)c(C#N)c3)n2)cn1]

This compound was prepared according to the procedure described for the preparation of Example Compound 117 using 6-methylpyridin-3-amine. Purification by RP-MPLC (C₁₈, MeOH/H₂O, 0-100%, with 0.1% TFA) provided the title compound; ¹H NMR (DMSO-d₆) δ 9.92 (s, 1H), 8.91 (d, 1H), 8.59 (d, 1H), 8.56-8.53 (m, 1H), 8.49-8.46 (m, 1H), 7.55-7.50 (m, 2H), 7.31 (d, 1H), 5.44-5.33 (m, 1H), 4.11-3.96 (m, 2H), 3.84-3.40 (m, 4H), 2.47 (s, 3H), 2.30-2.13 (m, 2H). LC-MS [M+H]⁺ 431.1825.

### Example Compound 119; 2-{[(3R)-1-(Hydroxyacetyl)pyrrolidin-3-yl]oxy}-5-(2-{[6-(morpholin-4-yl)pyridin-3-yl]amino}pyrimidin-4-yl)benzonitrile

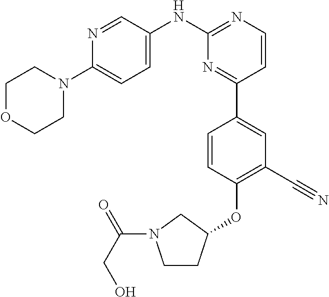 [SMILES: N#Cc1cc(-c2ccnc(Nc3ccc(N4CCOCC4)nc3)n2)ccc1O[C@@H]1CCN(C(=O)CO)C1]

This compound was prepared according to the procedure described for the preparation of Example Compound 117 using 6-morpholinopyridin-3-amine. Purification by RP-MPLC (C₁₈, MeOH/H₂O, 0-100%, with 0.1% TFA) provided the title compound; ¹H NMR (DMSO-d₆) δ 9.49 (s, 1H), 8.51-8.42 (m, 4H), 7.95-7.92 (m, 1H), 7.52 (d, 1H), 7.42 (d, 1H), 6.87 (d, 1H), 5.42-5.32 (m, 1H), 4.69 (s, 1H), 4.07-3.95 (m, 2H), 3.82-3.60 (m, 6H), 3.52-3.36 (m, 6H), 2.27-2.14 (m, 2H). LC-MS [M+H]⁺ 502.2159.

### Example Compound 120; 2-({(3R)-1-[(2S)-2-Hydroxypropanoyl]pyrrolidin-3-yl}oxy)-5-{2-[(6-methylpyridin-3-yl)amino]pyrimidin-4-yl}benzonitrile

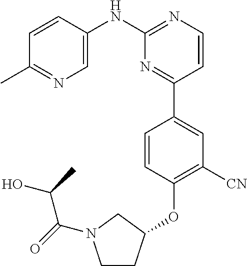 [SMILES: Cc1ccc(Nc2nccc(-c3ccc(O[C@@H]4CCN(C(=O)[C@H](C)O)C4)c(C#N)c3)n2)cn1]

This compound was prepared according to the procedure described for the preparation of Example Compound 117 using 6-methylpyridin-3-amine and (2S)-2-hydroxypropanoic acid as starting materials. Purification by column chromatography (SiO₂, EtOAc/Hexanes, 0-80%) afforded the title compound. ¹H NMR (CDCl₃) δ 8.74 (s, 1H), 8.49 (d, 1H), 8.33-8.27 (m, 2H), 8.05-7.98 (m, 1H), 7.39 (br. s, 1H), 7.18 (d, 1H), 7.11-7.05 (m, 2H), 5.16 (s, 1H), 4.42-4.31 (m, 1H), 3.87-3.75 (m, 4H), 2.55 (s, 3H), 2.50-2.24 (m, 2H), 1.70 (br. s, 1H), 1.45-1.36 (m, 3H). LC-MS [M+H]⁺ 445.2218.

### Example Compound 121; 2-{[(3R)-1-(Hydroxyacetyl)pyrrolidin-3-yl]oxy}-5-{2-[(2-methoxypyridin-4-yl)amino]pyrimidin-4-yl}benzonitrile

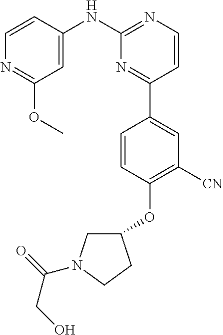 [SMILES: COc1cc(Nc2nccc(-c3ccc(O[C@@H]4CCN(C(=O)CO)C4)c(C#N)c3)n2)ccn1]

This compound was prepared according to the procedure described for the preparation of Example Compound 117 using 2-methoxypyridin-4-amine. Purification by column chromatography (SiO₂, EtOAc/Hexanes, 0-80%) afforded the title compound. ¹H NMR (CDCl₃) δ 8.53 (s, 1H), 8.32-8.28 (m, 2H), 8.06 (d, 1H), 7.75 (s, 1H), 7.35 (s, 1H), 7.18-7.05 (m, 3H), 5.23-5.13 (m, 1H), 4.17-4.10 (m, 2H), 3.93 (s, 3H), 3.91-3.56 (m, 4H), 2.53-2.23 (m, 2H). LC-MS [M+H]⁺ 447.2097.

### Example Compound 122; 2-{[(3R)-1-(Hydroxyacetyl)pyrrolidin-3-yl]oxy}-5-(2-{[2-(trifluoromethyl)pyridin-4-yl]amino}pyrimidin-4-yl)benzonitrile

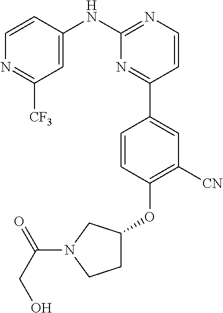 [SMILES: N#Cc1cc(-c2ccnc(Nc3ccnc(C(F)(F)F)c3)n2)ccc1O[C@@H]1CCN(C(=O)CO)C1]

This compound was prepared according to the procedure described for the preparation of Example Compound 117 using 2-(trifluoromethyl)pyridin-4-amine. Purification by column chromatography (SiO₂, EtOAc/Hexanes, 0-80%) afforded the title compound. ¹H NMR (CDCl₃) δ 8.60-8.58 (m, 2H), 8.37-8.26 (m, 3H), 7.92 (s, 1H), 7.71-7.65 (m, 1H), 7.27 (s, 1H), 7.11-7.06 (m, 1H), 5.30-5.19 (m, 1H), 4.21-4.09 (m, 2H), 4.02-3.91 (m, 1H), 3.86-3.57 (m, 4H), 2.55-2.25 (m, 2H). LC-MS [M+H]⁺ 485.1518.

### Example Compound 123; 2-{[(3R)-1-(Hydroxyacetyl)pyrrolidin-3-yl]oxy}-5-{2-[(2-methylpyridin-4-yl)amino]pyrimidin-4-yl}benzonitrile

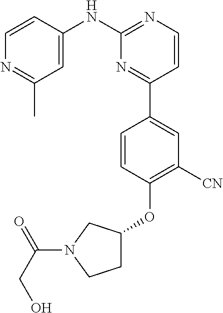 [SMILES: Cc1cc(Nc2nccc(-c3ccc(O[C@@H]4CCN(C(=O)CO)C4)c(C#N)c3)n2)ccn1]

This compound was prepared according to the procedure described for the preparation of Example Compound 117 using 2-methylpyridin-4-amine. Purification by column chromatography (SiO₂, EtOAc/Hexanes, 0-80%) afforded the title compound. ¹H NMR (CDCl₃) δ 8.57 (s, 1H), 8.39 (d, 1H), 8.33-8.26 (m, 2H), 7.70 (s, 1H), 7.52 (s, 2H), 7.19-7.06 (m, 2H), 5.23-5.16 (m, 1H), 4.20-4.12 (m, 2H), 4.08-4.12 (m, 2H), 3.85-3.56 (m, 4H), 2.57 (s, 3H), 2.51-2.32 (m, 2H). LC-MS [M+H]⁺ 431.3445.

### Example Compound 124; 5-(2-{[6-(Dimethylamino)pyridin-3-yl]amino}pyrimidin-4-yl)-2-{[1-(hydroxyacetyl)pyrrolidin-3-yl]oxy}benzonitrile

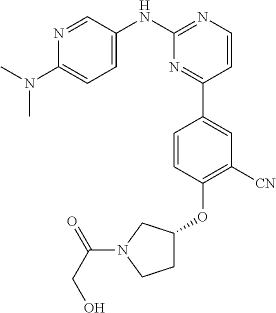 [SMILES: CN(C)c1ccc(Nc2nccc(-c3ccc(O[C@@H]4CCN(C(=O)CO)C4)c(C#N)c3)n2)cn1]

This compound was prepared according to the procedure described for the preparation of Example Compound 117 using N2,N2-dimethylpyridine-2,5-diamine. Purification by column chromatography (SiO₂, EtOAc/Hexanes, 0-80%) afforded the title compound. ¹H NMR (DMSO-d₆) δ 9.95 (s, 1H), 8.65 (d, 1H), 8.61 (d, 1H), 8.54-8.53 (m, 1H), 8.48-8.45 (m, 1H), 8.20-8.17 (m, 1H), 7.57-7.55 (m, 1H), 7.52-7.49 (m, 1H), 7.33 (d, 1H), 5.43-5.35 (m, 1H), 4.08-4.00 (m, 2H), 3.84-3.42 (m, 4H), 3.21 (s, 6H), 2.33-2.14 (m, 2H). LC-MS [M+H]⁺ 460.2111.

### Example Compound 125; 2-{[(3R)-1-(Hydroxyacetyl)pyrrolidin-3-yl]oxy}-5-[2-({6-[(3-methoxyazetidin-1-yl)methyl]pyridin-3-yl}amino)pyrimidin-4-yl]benzonitrile

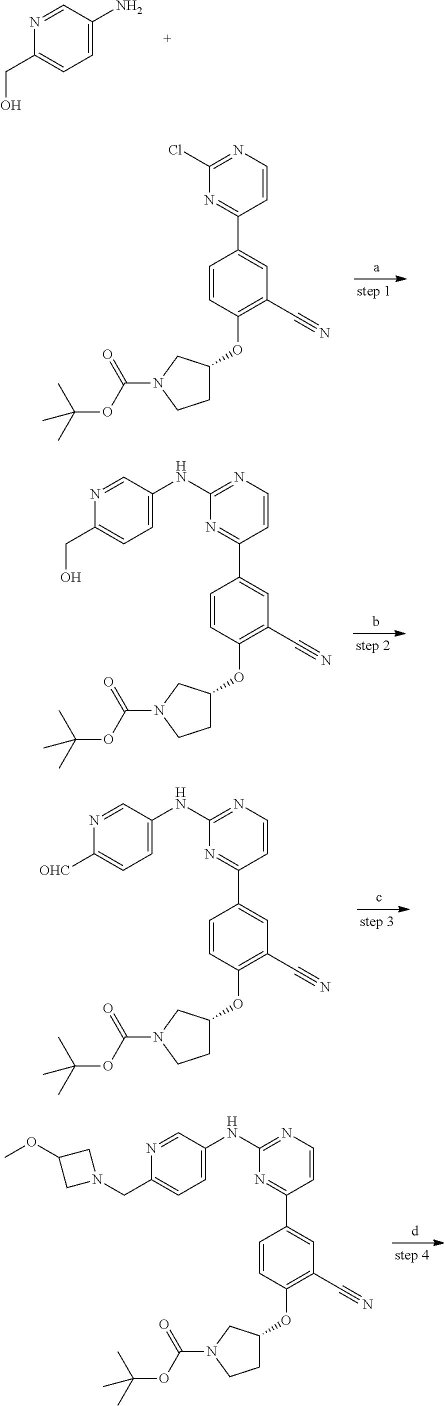 [SMILES: Nc1ccc(CO)nc1 ; CC(C)(C)OC(=O)N1CC[C@@H](Oc2ccc(-c3ccnc(Cl)n3)cc2C#N)C1 ; CC(C)(C)OC(=O)N1CC[C@@H](Oc2ccc(-c3ccnc(Nc4ccc(CO)nc4)n3)cc2C#N)C1 ; CC(C)(C)OC(=O)N1CC[C@@H](Oc2ccc(-c3ccnc(Nc4ccc(C=O)nc4)n3)cc2C#N)C1 ; COC1CN(Cc2ccc(Nc3nccc(-c4ccc(O[C@@H]5CCN(C(=O)OC(C)(C)C)C5)c(C#N)c4)n3)cn2)C1]

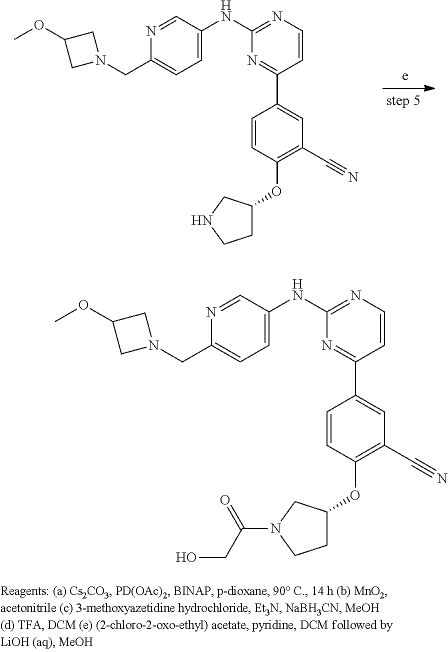 [SMILES: COC1CN(Cc2ccc(Nc3nccc(-c4ccc(O[C@@H]5CCNC5)c(C#N)c4)n3)cn2)C1 ; COC1CN(Cc2ccc(Nc3nccc(-c4ccc(O[C@@H]5CCN(C(=O)CO)C5)c(C#N)c4)n3)cn2)C1]

Step 1. tert-butyl (3R)-3-[2-cyano-4-[2-[[6-(hydroxymethyl)-3-pyridyl]amino]pyrimidin-4-yl]phenoxy]pyrrolidine-1-carboxylate. The procedure used in Step 1 in the preparation of Example Compound 117 was used to prepare the title compound from (5-amino-2-pyridyl)methanol. ¹H NMR (DMSO-d₆) 9.84 (s, 1H), 8.87 (d, 1H), 8.85 (d, 1H), 8.20 (dd, 1H), 7.50-7.55 (m, 2H), 7.42 (d, 1H), 5.29-5.33 (m, 2H), 4.53 (d, 2H), 3.56-3.70 (m, 1H), 3.44-3.52 (m, 1H), 2.18-2.29 (m, 1H), 2.08-2.18 (m, 1H), 1.38-1.44 (m, 9H).

Step 2. tert-Butyl (3R)-3-[2-cyano-4-[2-[(6-formyl-3-pyridyl)amino]pyrimidin-4-yl]phenoxy]pyrrolidine-1-carboxylate. The procedure used in Step 2 for the preparation of Example Compound 47 was used to prepare the title compound from the material isolated in Step 1 above. ¹H NMR (DMSO-d₆) 10.51 (s, 1H), 9.89 (s, 1H), 9.20 (d, 1H), 8.77 (d, 1H), 8.57-8.62 (m, 1H), 8.48-8.56 (m, 2H), 7.97 (d, 1H), 7.68 (d, 1H), 7.51-7.58 (m, 1H), 3.59-3.70 (m, 2H), 3.45-3.52 (m, 2H), 2.23 (d, 1H), 2.12-2.18 (m, 1H), 1.41 (d, 9H).

Step 3. tert-Butyl (3R)-3-[2-cyano-4-[2-[[6-[(3-methoxyazetidin-1-yl)methyl]-3-pyridyl]amino]pyrimidin-4-yl]phenoxy]pyrrolidine-1-carboxylate. Combined the aldehyde isolated in Step 2 above (0.10 g, 0.21 mmol) with 3-methoxyazetidine hydrochloride (0.033 g), triethylamine (0.2 mL), and NaBH₃CN (0.062 g) in MeOH (2 mL) and stirred at rt for several hours. The reaction was diluted with EtOAc and washed with brine, then dried (MgSO₄) and concentrated under vacuum. Purification by column chromatography (SiO₂, MeOH 20% in CH₂Cl₂ with 0.1% NH₄OH), gave the title compound; ¹H NMR (DMSO-d₆) 8.89 (s, 1H), 8.48 (d, 1H), 8.36-8.42 (dd, 1H), 7.36 (d, 1H), 7.32 (d, 2H), 5.23-5.27 (m, 1H), 4.06-4.12 (m, 1H), 3.79 (s, 2H), 3.66-3.73 (m, 3H), 3.64 (s, 1H), 3.51-3.62 (m, 2H), 3.26 (s, 3H), 3.15-3.23 (m, 2H), 2.26 (d, 2H), 1.48 (d, 9H).

Step 4. 5-[2-[[6-[(3-Methoxyazetidin-1-yl)methyl]-3-pyridyl]amino]pyrimidin-4-yl]-2-[(3R)-pyrrolidin-3-yl]oxy-benzonitrile. This compound was prepared using Standard Method B; BOC Deprotection. The residue was purified by column chromatography (SiO₂, MeOH 20% in CH₂Cl₂ with 0.1% NH₄OH), to give the title compound; LC/MS [M+H]⁺ 516.2354

Step 5. 2-{[(3R)-1-(Hydroxyacetyl)pyrrolidin-3-yl]oxy}-5-[2-({6-[(3-methoxyazetidin-1-yl)methyl]pyridin-3-yl}amino)pyrimidin-4-yl]benzonitrile. This compound was prepared using the procedure used in Step 3 in the preparation of Example Compound 117. Purification by column chromatography (SiO₂, MeOH 20% in CH₂Cl₂ with 0.1% NH₄OH), gave the title compound; ¹H NMR (DMSO-d₆) 10.08 (s, 1H), 9.80 (br s, 1H), 9.00 (d, 1H), 8.62 (d, 1H), 8.56 (d, 1H), 8.44-8.52 (m, 1H), 8.31 (dd, 1H), 7.43-7.62 (m, 1H), 5.28-5.49 (m, 1H), 4.38-4.47 (m, 2H), 4.20-4.31 (m, 2H), 3.97-4.09 (m, 2H), 3.92 (s, 1H), 3.60-3.71 (m, 2H), 3.41-3.53 (m, 1H), 3.24 (s, 3H), 3.02-3.11 (m, 4H), 2.29 (s, 1H), 2.17 (s, 1H); LC/MS [M+H]⁺ 516.2354.

### Example Compound 126; 2-{[(3R)-1-(Hydroxyacetyl)pyrrolidin-3-yl]oxy}-5-(2-{[2-(1-methyl-1H-pyrazol-4-yl)pyridin-4-yl]amino}pyrimidin-4-yl)benzonitrile

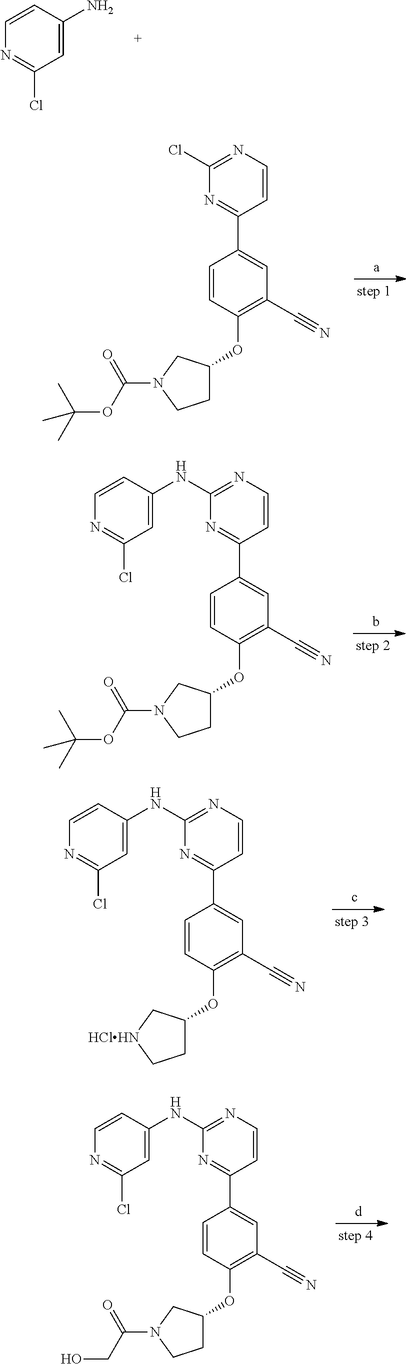

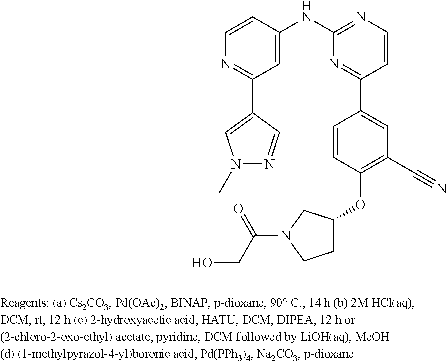 [SMILES: Cn1cc(-c2cc(Nc3nccc(-c4ccc(O[C@@H]5CCN(C(=O)CO)C5)c(C#N)c4)n3)ccn2)cn1]

Step 1. tert-Butyl (3R)-3-[4-[2-[(2-chloro-4-pyridyl)amino]pyrimidin-4-yl]-2-cyano-phenoxy]pyrrolidine-1-carboxylate. The procedure used in Step 3 in the preparation of Example Compound 1 was used to prepare the title compound from 2-chloropyridin-4-amine. LC-MS [M−H]⁻ 491.3

Step 2. 5-[2-[(2-Chloro-4-pyridyl)amino]pyrimidin-4-yl]-2-[(3R)-pyrrolidin-3-yl]oxy-benzonitrile. Standard Method B, BOC Deprotection, was used to prepare the hydrochloride salt of the title compound from the crude product isolated in Step 1. LC-MS [M+H]⁺ 393.3

Step 3. 5-[2-[(2-Chloro-4-pyridyl)amino]pyrimidin-4-yl]-2-[(3R)-1-(2-hydroxyacetyl)pyrrolidin-3-yl]oxy-benzonitrile. Standard Method G, EDCI Coupling was used to prepare the title compound starting from the crude product isolated in Step 2. LC-MS [M+H]⁺ 451.3

Step 4. 2-{[(3R)-1-(Hydroxyacetyl)pyrrolidin-3-yl]oxy}-5-(2-{[2-(1-methyl-1H-pyrazol-4-yl)pyridin-4-yl]amino}pyrimidin-4-yl)benzonitrile This compound was prepared using the procedure for the preparation of Example Compound 82 using the material isolated in Step 3 above. Purification by RP-MPLC (C₁₈, MeOH/H₂O, 0-100%, with 0.1% TFA) provided the title compound; ¹H NMR (DMSO-_{d6}) δ 11.35 (s, 1H), 8.84 (d, 1H), 8.62-8.49 (m, 5H), 8.16 (s, 1H), 7.91-7.87 (m, 2H), 7.56 (dd, 1H), 7.93 (br. s, 1H), 7.58 (d, 1H), 5.41 (d, 1H), 4.12-4.05 (m, 2H), 4.90 (s, 3H), 3.70-3.62 (m, 3H), 3.54-3.43 (m, 2H), 2.13-2.08 (m, 2H), 2.34-2.15 (m, 2H). LC-MS [M+H]⁺ 497.4

### Example Compound 127; 2-({1-[(2S)-2-Hydroxypropanoyl]piperidin-4-yl}oxy)-5-(2-{[6-(morpholin-4-yl)pyridin-3-yl]amino}pyrimidin-4-yl)benzonitrile

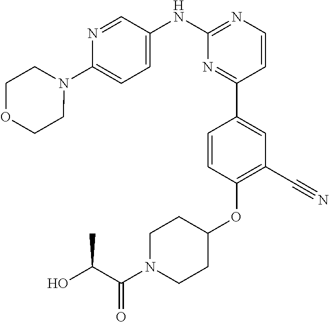 [SMILES: C[C@H](O)C(=O)N1CCC(Oc2ccc(-c3ccnc(Nc4ccc(N5CCOCC5)nc4)n3)cc2C#N)CC1]

This compound was prepared according to the procedure described for the preparation of Example Compound 117 using 6-morpholinopyridin-3-amine and tert-butyl 4-[4-(2-chloropyrimidin-4-yl)-2-cyano-phenoxy]piperidine-1-carboxylate as starting materials. Purification by column chromatography (SiO₂, MeOH/DCM, 0-40%) gave the title compound; ¹H NMR (DMSO-d₆) δ 9.48 (s, 1H), 8.53-8.48 (m, 3H), 8.43-8.41 (m, 1H), 7.95-7.92 (m, 1H), 7.56 (d, 1H), 7.41 (d, 1H), 6.86 (d, 1H), 5.00 (br s, 1H), 4.98-4.95 (m, 1H), 4.50-4.43 (m, 1H), 3.85-3.66 (m, 6H), 3.58-3.46 (m, 2H), 3.39-3.34 (m, 4H), 2.08-1.92 (m, 2H), 1.79-1.62 (m, 2H), 1.20 (d, 3H). LC-MS [M+H]⁺ 530.2411.

### Example Compound 128; 2-({1-[(2S)-2-Hydroxypropanoyl]piperidin-4-yl}oxy)-5-{2-[(6-methylpyridin-3-yl)amino]pyrimidin-4-yl}benzonitrile

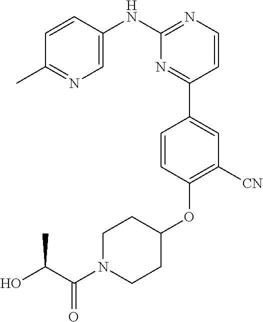 [SMILES: Cc1ccc(Nc2nccc(-c3ccc(OC4CCN(C(=O)[C@H](C)O)CC4)c(C#N)c3)n2)cn1]

This compound was prepared according to the procedure described for the preparation of Example Compound 127 using 6-methylpyridin-3-amine and (2S)-2-hydroxypropanoic acid as starting materials. Purification by column chromatography (SiO₂, MeOH/DCM, 0-40%) gave the title compound; ¹H NMR (DMSO-d₆) δ 9.79 (s, 1H), 8.85 (d, 1H), 8.57 (d, 1H), 8.54 (d, 1H), 8.47-8.44 (m, 1H), 8.09-8.06 (m, 1H), 7.58 (d, 1H), 7.51 (d, 1H), 7.21 (d, 1H), 5.02-4.96 (m, 2H), 4.50-4.44 (m, 1H), 3.84-3.68 (m, 2H), 3.58-3.46 (m, 2H), 2.43 (s, 3H), 2.06-1.94 (m, 2H), 1.78-1.62 (m, 2H), 1.21 (d, 3H). LC-MS [M+H]⁺ 459.2287.

### Example Compound 129; 2-({1-[(2R)-2-Hydroxypropanoyl]piperidin-4-yl}oxy)-5-{2-[(6-methylpyridin-3-yl)amino]pyrimidin-4-yl}benzonitrile

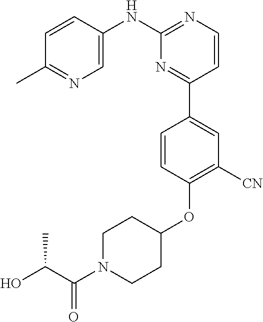 [SMILES: Cc1ccc(Nc2nccc(-c3ccc(OC4CCN(C(=O)[C@@H](C)O)CC4)c(C#N)c3)n2)cn1]

This compound was prepared according to the procedure described for the preparation of Example Compound 127 using 6-methylpyridin-3-amine and the sodium salt of (2R)-2-hydroxypropanoic acid as starting materials. Purification by column chromatography (SiO₂, MeOH/DCM, 0-40%) gave the title compound; ¹H NMR (CDCl₃) δ 8.76 (s, 1H), 8.49 (d, 1H), 8.31-8.29 (m, 2H), 8.02 (d, 1H), 7.45 (s, 1H), 7.18 (d, 1H), 7.11-7.10 (m, 2H), 4.88 (br. s, 1H), 4.53-4.50 (m, 1H), 4.15-3.98 (m, 2H), 3.73-3.47 (m, 3H), 2.56 (s, 3H), 2.05-2.00 (m, 4H), 1.37 (d, 3H). LC-MS [M+H]⁺ 459.2149.

### Example Compound 130; 2-{[1-(Hydroxyacetyl)piperidin-4-yl]oxy}-5-{2-[(6-methylpyridin-3-yl)amino]pyrimidin-4-yl}benzonitrile

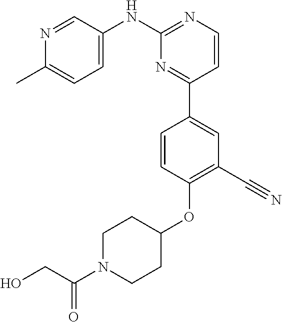 [SMILES: Cc1ccc(Nc2nccc(-c3ccc(OC4CCN(C(=O)CO)CC4)c(C#N)c3)n2)cn1]

This compound was prepared according to the procedure described for the preparation of Example Compound 127 using 6-methylpyridin-3-amine then 2-hydroxyacetic acid as starting materials. Purification by column chromatography (SiO₂, MeOH/DCM, 0-40%) gave the title compound; ¹H NMR (DMSO-d₆) 9.78 (s, 1H), 8.84 (d, 1H), 8.57 (d, 1H), 8.54 (d, 1H), 8.45 (dd, 1H), 8.05-8.10 (m, 1H), 7.58 (d, 1H), 7.51 (d, 1H), 7.21 (d, 1H), 4.96-5.04 (m, 1H), 4.59 (t, 1H), 4.13 (d, 2H), 3.70-3.79 (m, 1H), 3.42-3.61 (m, 2H), 2.43 (s, 3H), 2.29 (s, 1H), 1.94-2.05 (m, 2H), 1.75 (d, 1H), 1.62-1.71 (m, 1H); LC-MS [M+H]⁺ 445.2293.

### Example Compound 131; 2-{[1-(Hydroxyacetyl)piperidin-4-yl]oxy}-5-{2-[(2-methoxypyridin-4-yl)amino]pyrimidin-4-yl}benzonitrile

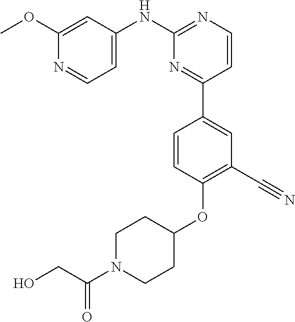 [SMILES: COc1cc(Nc2nccc(-c3ccc(OC4CCN(C(=O)CO)CC4)c(C#N)c3)n2)ccn1]

This compound was prepared according to the procedure described for the preparation of Example Compound 127 using 2-methoxypyridin-4-amine then 2-hydroxyacetic acid as starting materials. Purification by column chromatography (SiO₂, MeOH/DCM, 0-40%) gave the title compound; ¹H NMR (DMSO-d₆) 10.15 (s, 1H), 8.66 (d, 1H), 8.58 (d, 1H), 8.47 (dd, 1H), 7.98 (d, 1H), 7.63 (d, 1H), 7.60 (d, 1H), 7.45 (d, 1H), 7.31 (dd, 1H), 4.99-5.06 (m, 1H), 4.59 (t, 1H), 4.14 (d, 2H), 3.83 (s, 3H), 3.70-3.81 (m, 1H), 3.53-3.65 (m, 1H), 3.41-3.53 (m, 1H), 3.34 (s, 1H), 1.94-2.06 (m, 2H), 1.73-1.78 (m, 1H), 1.66-1.72 (m, 1H); LC-MS [M+H]⁺ 461.3386.

### Example Compound 132; N-[2-Cyano-4-(2-{[2-(pyrrolidin-1-yl)pyrimidin-5-yl]amino}pyrimidin-4-yl)phenyl]-2-methylpropanamide

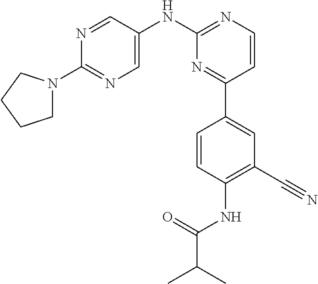 [SMILES: CC(C)C(=O)Nc1ccc(-c2ccnc(Nc3cnc(N4CCCC4)nc3)n2)cc1C#N]

This compound was prepared according to the procedure described for the preparation of Example Compound 138 using 2-pyrrolidin-1-ylpyrimidin-5-amine and N-[4-(2-chloropyrimidin-4-yl)phenyl]-2-methyl-propanamide. The product was purified by column chromatography (SiO₂, MeOH/DCM, 0-40%) to give the title compound; ¹H NMR (DMSO-d₆) δ 10.27 (s, 1H), 9.41 (s, 1H), 8.63 (s, 2H), 8.52-8.50 (m, 2H), 8.40-8.37 (m, 1H), 7.75 (d, 1H), 7.44 (d, 1H), 3.51-3.47 (m, 4H), 2.76-2.68 (m, 1H), 1.96-1.92 (m, 4H), 1.15 (d, 6H). LC-MS [M+H]⁺ 429.2188

### Example Compound 133; N-{2-Cyano-4-[2-({6-[(2-hydroxyethyl)(methyl)amino]pyridin-3-yl}amino)pyrimidin-4-yl]phenyl}-2-methylpropanamide

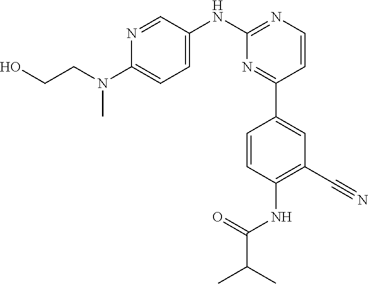 [SMILES: CC(C)C(=O)Nc1ccc(-c2ccnc(Nc3ccc(N(C)CCO)nc3)n2)cc1C#N]

This compound was prepared according to the procedure described for the preparation of Example Compound 132 using 2-[(5-amino-2-pyridyl)-methyl-amino]ethanol. The product was purified by column chromatography (SiO₂, MeOH/DCM, 0-40%) to give the title compound; ¹H NMR (DMSO-d₆) δ 10.33 (s, 1H), 9.96 (s, 1H), 8.64-8.62 (m, 2H), 8.56 (d, 1H), 8.47-8.44 (m, 1H), 8.16-8.12 (m, 1H), 7.79 (d, 1H), 7.60 (d, 1H), 7.36 (d, 1H), 3.69-3.66 (m, 4H), 3.20 (s, 3H), 2.78-2.71 (m, 1H), 1.16 (d, 6H). LC-MS [M+H]⁺ 432.2429.

### Example Compound 134; N-[2-Cyano-4-(2-{[6-(morpholin-4-yl)pyridin-3-yl]amino}pyrimidin-4-yl)phenyl]-2-methylpropanamide

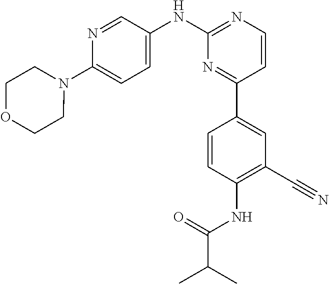 [SMILES: CC(C)C(=O)Nc1ccc(-c2ccnc(Nc3ccc(N4CCOCC4)nc3)n2)cc1C#N]

This compound was prepared according to the procedure described for the preparation of Example Compound 132 using 6-morpholinopyridin-3-amine. The product was purified by column chromatography (SiO₂, MeOH/DCM, 0-40%) to give the title compound; ¹H NMR (DMSO-d₆) δ 10.27 (s, 1H), 9.53 (s, 1H), 8.54-8.49 (m, 3H), 8.43-8.40 (m, 1H), 7.97-7.94 (m, 1H), 7.76 (d, 1H), 7.44 (d, 1H), 6.87 (d, 1H), 3.73-3.70 (m, 4H), 3.38-3.34 (m, 4H), 2.76-2.69 (m, 1H), 1.16 (d, 6H). LC-MS [M+H]⁺ 444.2130.

### Example Compound 135; N-[2-Cyano-4-(2-{[6-(pyrrolidin-1-yl)pyridin-3-yl]amino}pyrimidin-4-yl)phenyl]-2-methylpropanamide

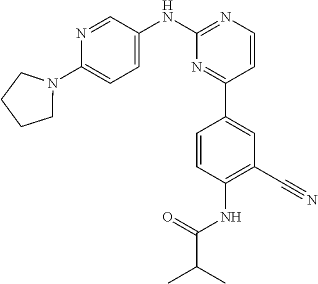 [SMILES: CC(C)C(=O)Nc1ccc(-c2ccnc(Nc3ccc(N4CCCC4)nc3)n2)cc1C#N]

This compound was prepared according to the procedure described for the preparation of Example Compound 132 using 6-pyrrolidin-1-ylpyridin-3-amine. The product was purified by column chromatography (SiO₂, MeOH/DCM, 0-40%) to give the title compound; ¹H NMR (DMSO-d₆) δ 10.34 (s, 1H), 9.92 (s, 1H), 8.63-8.61 (m, 2H), 8.55 (d, 1H), 8.45-8.42 (m, 1H), 8.16-8.13 (m, 1H), 7.79 (d, 1H), 7.57 (d, 1H), 7.13 (d, 1H), 3.53-3.50 (m, 4H), 2.78-2.71 (m, 1H), 2.05-2.01 (m, 4H), 1.16 (d, 6H). LC-MS [M+H]⁺ 428.2162.

### Example Compound 136; N-(2-Cyano-4-{2-[(6-methylpyridin-3-yl)amino]pyrimidin-4-yl}phenyl)-2-methylpropanamide

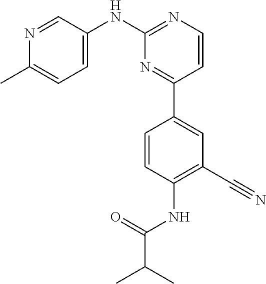 [SMILES: Cc1ccc(Nc2nccc(-c3ccc(NC(=O)C(C)C)c(C#N)c3)n2)cn1]

This compound was prepared according to the procedure described for the preparation of Example Compound 132 using 6-methylpyridin-3-amine. The product was purified by column chromatography (SiO₂, MeOH/DCM, 0-40%) to give the title compound; ¹H NMR (DMSO-d₆) δ 10.30 (s, 1H), 9.83 (s, 1H), 8.83 (d, 1H), 8.60 (d, 1H), 8.57 (d, 1H), 8.46-8.43 (m, 1H), 8.12-8.09 (m, 1H), 7.78 (d, 1H), 7.53 (d, 1H), 7.22 (d, 1H), 2.77-2.70 (m, 1H), 2.43 (s, 3H), 1.16 (d, 6H). LC-MS [M+H]⁺ 373.1718.

### Example Compound 137; N-(2-Cyano-4-{2-[(6-ethoxypyridin-3-yl)amino]pyrimidin-4-yl}phenyl)-2-methylpropanamide

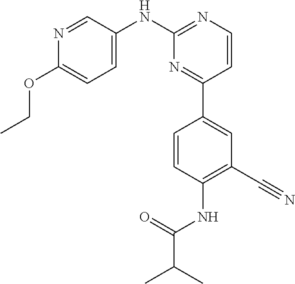 [SMILES: CCOc1ccc(Nc2nccc(-c3ccc(NC(=O)C(C)C)c(C#N)c3)n2)cn1]

This compound was prepared according to the procedure described for the preparation of Example Compound 132 using 6-ethoxypyridin-3-amine. The product was purified by column chromatography (SiO₂, MeOH/DCM, 0-40%) to give the title compound; ¹H NMR (DMSO-d₆) δ 10.29 (s, 1H), 9.66 (s, 1H), 8.57-8.54 (m, 2H), 8.50 (d, 1H), 8.44-8.41 (m, 1H), 8.07-8.03 (m, 1H), 7.77 (d, 1H), 7.48 (d, 1H), 6.80 (d, 1H), 4.30-4.24 (m, 2H), 2.76-2.70 (m, 1H), 1.32 (t, 3H), 1.16 (d, 6H). LC-MS [M+H]⁺ 403.2179.

### Example Compound 138; N-[2-Cyano-4-(2-{[6-(dimethylamino)pyridin-3-yl]amino}pyrimidin-4-yl)phenyl]cyclopropanecarboxamide

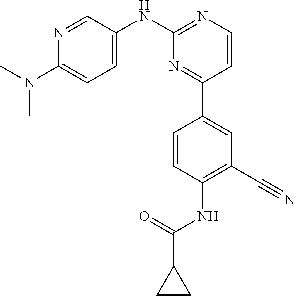 [SMILES: CN(C)c1ccc(Nc2nccc(-c3ccc(NC(=O)C4CC4)c(C#N)c3)n2)cn1]

This compound was prepared according to the procedure described for the preparation of Example Compound 1 using N2,N2-dimethylpyridine-2,5-diamine and N-[4-(2-chloropyrimidin-4-yl)-2-cyano-phenyl]cyclopropanecarboxamide. The product was purified by column chromatography (SiO₂, MeOH/DCM, 0-40%) to give the title compound; ¹H NMR (DMSO-d₆) δ 10.67 (s, 1H), 9.94 (s, 1H), 8.64-8.62 (m, 1H), 8.56 (d, 1H), 8.45-8.41 (m, 1H), 8.17-8.14 (m, 1H), 7.87 (d, 1H), 7.57 (d, 1H), 7.28 (d, 1H), 3.19 (s, 6H), 2.01-1.97 (m, 1H), 0.93-0.86 (m, 4H). LC-MS [M+H]⁺ 400.1877.

### Example Compound 139; N-[2-Cyano-4-(2-{[6-(diethylamino)pyridin-3-yl]amino}pyrimidin-4-yl)phenyl]cyclopropanecarboxamide

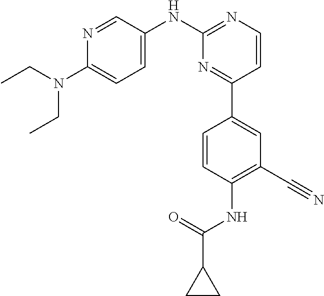 [SMILES: CCN(CC)c1ccc(Nc2nccc(-c3ccc(NC(=O)C4CC4)c(C#N)c3)n2)cn1]

This compound was prepared according to the procedure described for the preparation of Example Compound 138, using N2,N2-diethylpyridine-2,5-diamine. The product was purified by column chromatography (SiO₂, MeOH/DCM, 0-40%) to give the title compound; ¹H NMR (DMSO-d₆) δ 10.62 (s, 1H), 9.32 (s, 1H), 8.52 (d, 1H), 8.48 (d, 1H), 8.41-8.38 (m, 1H), 8.34 (d, 1H), 7.85-7.78 (m, 2H), 7.38 (d, 1H), 6.59 (d, 1H), 3.50-3.44 (m, 4H), 2.52-2.50 (m, 1H), 1.99-1.96 (m, 1H), 1.11 (t, 6H), 0.91-0.88 (m, 4H). LC-MS [M+H]⁺ 428.2125.

### Example Compound 140; N-(2-Cyano-4-{2-[(6-cyclopropylpyridin-3-yl)amino]pyrimidin-4-yl}phenyl)cyclopropanecarboxamide

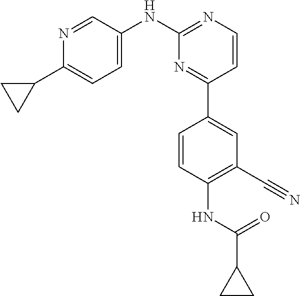 [SMILES: N#Cc1cc(-c2ccnc(Nc3ccc(C4CC4)nc3)n2)ccc1NC(=O)C1CC1]

This compound was prepared according to the procedure described for the preparation of Example Compound 138, using 6-cyclopropylpyridin-3-amine. Purification by RP-MPLC (C₁₈, MeOH/H₂O, 0-100%, with 0.1% TFA) provided the title compound; ¹H NMR (DMSO-d₆) δ 10.69 (s, 1H), 10.37 (s, 1H), 9.15 (d, 1H), 8.68 (d, 1H), 8.58 (d, 1H), 8.47-8.44 (m, 1H), 8.40-8.37 (m, 1H), 7.87 (d, 1H), 7.65 (d, 1H), 7.53 (d, 1H), 2.29-2.23 (m, 1H), 2.04-1.97 (m, 1H), 1.22-1.16 (m, 2H), 1.07-1.03 (m, 2H), 0.94-0.87 (m, 4H). LC-MS [M+H]⁺ 397.1794.

### Example Compound 141; N-[2-Cyano-4-(2-{[6-(morpholin-4-yl)pyridin-3-yl]amino}pyrimidin-4-yl)phenyl]cyclopropanecarboxamide

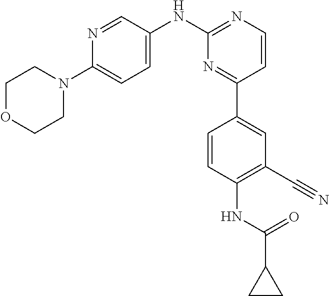 [SMILES: N#Cc1cc(-c2ccnc(Nc3ccc(N4CCOCC4)nc3)n2)ccc1NC(=O)C1CC1]

This compound was prepared according to the procedure described for the preparation of Example Compound 138, using 6-morpholinopyridin-3-amine. Purification by RP-MPLC (C₁₈, MeOH/H₂O, 0-100%, with 0.1% TFA) provided the title compound; ¹H NMR (DMSO-d₆) δ 10.67 (s, 1H), 9.89 (s, 1H), 8.66 (d, 1H), 8.61 (d, 1H), 8.55 (d, 1H), 8.44-8.41 (m, 1H), 8.16-8.12 (m, 1H), 7.86 (d, 1H), 7.55 (d, 1H), 7.28 (d, 1H), 3.78-3.71 (m, 4H), 3.53-3.50 (m, 4H), 2.02-1.96 (m, 1H), 0.93-0.87 (m, 4H). LC-MS [M+H]⁺ 442.2165.

### Example Compound 142; N-[2-Cyano-4-(2-{[2-(3-methoxyazetidin-1-yl)pyridin-4-yl]amino}pyrimidin-4-yl)phenyl]cyclopropanecarboxamide

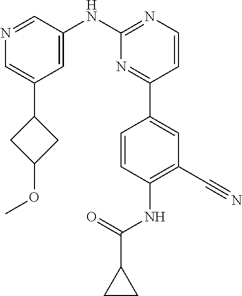 [SMILES: COC1CC(c2cncc(Nc3nccc(-c4ccc(NC(=O)C5CC5)c(C#N)c4)n3)c2)C1]

This compound was prepared according to the procedure described for the preparation of Example Compound 138, using 2-(3-methoxyazetidin-1-yl)pyridin-4-amine. Purification by RP-MPLC (C₁₈, MeOH/H₂O, 0-100%, with 0.1% TFA) provided the title compound; ¹H NMR (CDCl₃) δ 8.52 (d, 1H), 8.46 (d, 1H), 8.39 (d, 1H), 8.23 (dd, 1H), 7.93 (d, 1H), 7.22 (d, 1H), 7.17 (d, 1H), 6.70 (dd, 1H), 4.41-4.38 (m, 1H), 4.31-4.27 (m, 2H), 3.97-3.93 (m, 2H), 3.38 (s, 3H), 1.82-1.78 (m, 1H), 1.18-1.14 (m, 2H), 1.01-0.98 (m, 2H). LC-MS [M+H]⁺ 442.1971.

### Example Compound 143; N-{2-Cyano-4-[2-({2-[4-(2-hydroxyethyl)piperazin-1-yl]pyridin-4-yl}amino)pyrimidin-4-yl]phenyl}cyclopropanecarboxamide

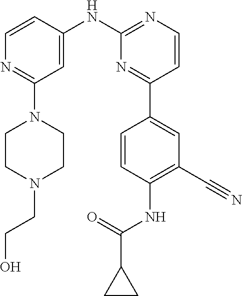 [SMILES: N#Cc1cc(-c2ccnc(Nc3ccnc(N4CCN(CCO)CC4)c3)n2)ccc1NC(=O)C1CC1]

This compound was prepared according to the procedure described for the preparation of Example Compound 138, using 2-[4-(4-amino-2-pyridyl)piperazin-1-yl]ethanol. Purification by RP-MPLC (C₁₈, MeOH/H₂O, 0-100%, with 0.1% TFA) provided the title compound; ¹H NMR (CDCl₃) δ 8.55 (d, 1H), 8.44 (d, 1H), 8.32-8.24 (m, 2H), 8.01 (d, 1H), 7.62 (d, 1H), 7.26 (d, 1H), 6.84 (dd, 1H), 3.76-3.73 (m, 2H), 3.65-3.55 (m, 4H), 2.76-2.68 (m, 4H), 2.66-2.61 (m, 2H), 1.90-1.85 (m, 1H), 1.18-1.12 (m, 2H), 1.02-0.98 (m, 2H). LC-MS [M+H]⁺ 485.2392.

### Example Compound 144; N-[2-Cyano-4-(2-{[6-(hydroxymethyl)pyridin-3-yl]amino}pyrimidin-4-yl)phenyl]cyclopropanecarboxamide

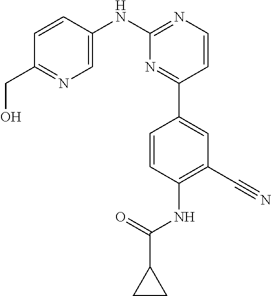 [SMILES: N#Cc1cc(-c2ccnc(Nc3ccc(CO)nc3)n2)ccc1NC(=O)C1CC1]

This compound was prepared according to the procedure described for the preparation of Example Compound 47, Step 1, using Intermediate I-10. Purification by RP-MPLC (C₁₈, MeOH/H₂O, 0-100%, with 0.1% TFA) provided the title compound; ¹H NMR (DMSO-d₆) δ 10.6 (s, 1H), 9.89 (s, 1H), 8.87 (d, 1H), 8.62 (d, 1H), 8.57 (d, 1H), 8.45 (dd, 1H), 8.22 (dd, 1H), 7.86 (d, 1H), 7.55 (d, 1H), 7.43 (d, 1H), 5.33 (t, 1H), 4.52 (d, 2H), 2.00-1.95 (m, 1H), 0.92-0.83 (m, 4H). LC-MS [M+H]⁺ 387.1576.

### Example Compound 145; N-(2-Cyano-4-{2-[(6-{[4-(2-hydroxyethyl)piperazin-1-yl]methyl}pyridin-3-yl)amino]pyrimidin-4-yl}phenyl)cyclopropanecarboxamide

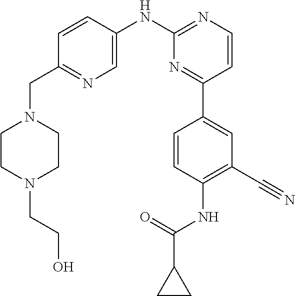 [SMILES: N#Cc1cc(-c2ccnc(Nc3ccc(CN4CCN(CCO)CC4)nc3)n2)ccc1NC(=O)C1CC1]

This compound was prepared according to the procedure described for the preparation of Example Compound 47, using N-[4-(2-chloropyrimidin-4-yl)-2-cyano-phenyl]cyclopropanecarboxamide as a starting material and 2-piperazin-1-ylethanol in Step 3. Purification by RP-MPLC (C₁₈, MeOH/H₂O, 0-100%, with 0.1% TFA) provided the title compound; ¹H NMR (DMSO-d₆) δ 10.7 (s, 1H), 10.1 (s, 1H), 9.03 (s, 1H), 8.65 (d, 1H), 8.59 (s, 1H), 8.45 (d, 1H), 8.33 (d, 1H), 7.87 (d, 1H), 7.61 (d, 1H), 7.52 (d, 1H), 4.03-3.90 (m, 2H), 3.55-3.45 (m, 1H), 3.71 (s, 2H), 3.23-3.05 (m, 8H), 2.55 (t, 2H), 2.46 (t, 2H), 2.00-1.95 (m, 1H), 0.93-0.85 (m, 4H). LC-MS [M+H]⁺ 499.2572.

### Example Compound 146; N-{2-Cyano-4-[2-({6-[(3-methoxyazetidin-1-yl)methyl]pyridin-3-yl}amino)pyrimidin-4-yl]phenyl}cyclopropanecarboxamide

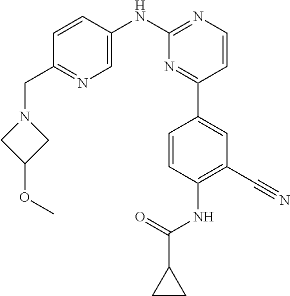 [SMILES: COC1CN(Cc2ccc(Nc3nccc(-c4ccc(NC(=O)C5CC5)c(C#N)c4)n3)cn2)C1]

This compound was prepared according to the procedure described for the preparation of Example Compound 145 using 3-methoxyazetidine hydrochloride. Purification by RP-MPLC (C₁₈, MeOH/H₂O, 0-100%, with 0.1% TFA) provided the title compound; ¹H NMR (DMSO-d₆) δ 10.6 (s, 1H), 10.1 (s, 1H), 9.02 (s, 1H), 8.65 (d, 1H), 8.58 (s, 1H), 8.45 (d, 1H), 8.33 (d, 1H), 7.86 (d, 1H), 7.60 (d, 1H), 7.46 (d, 1H), 4.52 (s, 2H), 4.40-4.30 (m, 2H), 4.29-4.24 (m, 1H), 4.10-4.00 (m, 2H), 3.25 (s, 3H), 2.01-1.95 (m, 1H), 0.92-0.84 (m, 4H). LC-MS [M+H]⁺ 456.2152.

### Example Compound 147; N-(2-Cyano-4-{2-[(6-{[3-(2-methoxyethoxy)azetidin-1-yl]methyl}pyridin-3-yl)amino]pyrimidin-4-yl}phenyl)cyclopropanecarboxamide

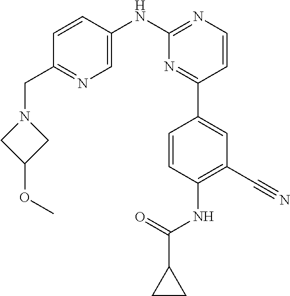 [SMILES: COC1CN(Cc2ccc(Nc3nccc(-c4ccc(NC(=O)C5CC5)c(C#N)c4)n3)cn2)C1]

This compound was prepared according to the procedure described for the preparation of Example Compound 145 using 3-(2-methoxyethoxy)azetidine hydrochloride. Purification by RP-MPLC (C₁₈, MeOH/H₂O, 0-100%, with 0.1% TFA) provided the title compound; LC-MS [M+H]⁺ 500.2419.

### Example Compound 148; N-(2-Cyano-4-{2-[(6-{[(2-methoxyethyl)amino]methyl}pyridin-3-yl)amino]pyrimidin-4-yl}phenyl)cyclopropanecarboxamide

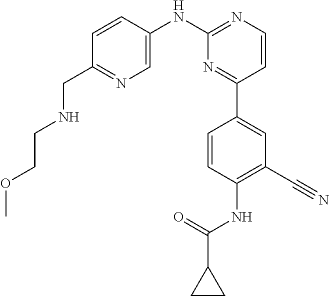 [SMILES: COCCNCc1ccc(Nc2nccc(-c3ccc(NC(=O)C4CC4)c(C#N)c3)n2)cn1]

This compound was prepared according to the procedure described for the preparation of Example Compound 145 using 2-methoxyethanamine. Purification by RP-MPLC (C₁₈, MeOH/H₂O, 0-100%, with 0.1% TFA) provided the title compound; LC-MS [M+H]⁺ 500.2419.

### Example Compound 149; N-(2-Cyano-4-{2-[(6-methylpyridin-3-yl)amino]pyrimidin-4-yl}phenyl)cyclopropanecarboxamide

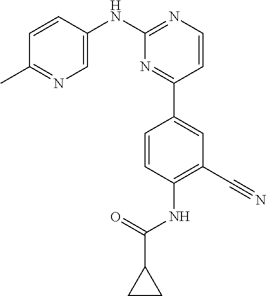 [SMILES: Cc1ccc(Nc2nccc(-c3ccc(NC(=O)C4CC4)c(C#N)c3)n2)cn1]

This compound was prepared according to the procedure described for the preparation of Example Compound 145 using 6-methylpyridin-3-amine. Purification by RP-MPLC (C₁₈, MeOH/H₂O, 0-100%, with 0.1% TFA) provided the title compound; ¹H NMR (DMSO-d₆) δ 10.69 (s, 1H), 10.46 (s, 1H), 9.23 (d, 1H), 8.71 (d, 1H), 8.59 (d, 1H), 8.48-8.44 (m, 2H), 7.87 (d, 1H), 7.74 (d, 1H), 7.68 (d, 1H), 2.62 (s, 3H), 2.02-1.96 (m, 1H), 0.93-0.84 (m, 4H). LC-MS [M+H]⁺ 371.1502

### Example Compound 150; 5-(2-{[3-(Propan-2-yl)-1H-1,2,4-triazol-5-yl]amino}pyrimidin-4-yl)-2-(tetrahydro-2H-pyran-4-yloxy)benzonitrile

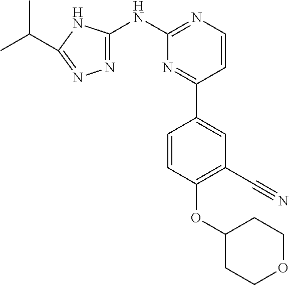 [SMILES: CC(C)c1nnc(Nc2nccc(-c3ccc(OC4CCOCC4)c(C#N)c3)n2)[nH]1]

This compound was prepared according to the procedure described for the preparation of Example Compound 84 using 5-isopropyl-4H-1,2,4-triazol-3-amine. Purification by RP-MPLC (C₁₈, MeOH/H₂O, 0-100%, with 0.1% TFA) provided the title compound; ¹H NMR (MeOH-d₄) δ 8.63 (d, 1H), 8.55 (d, 1H), 8.45 (dd, 1H), 7.57 (d, 1H), 7.41 (d, 1H), 4.92 (m, 1H), 4.01-3.95 (m, 2H), 3.68-3.62 (m, 2H), 3.12-3.05 (m, 1H), 2.61 (m, 1H), 2.14-2.08 (m, 2H), 1.87-1.78 (m, 2H), 1.36 (d, 6H). LC-MS [M+H]⁺ 406.3482.

Example Compounds 151-253 in Table 2 were made by methods similar to those disclosed for Example Compounds 1-150. One of ordinary skill in the art would understand from the disclosed methods of making Example Compounds 1-150 how to make Example Compounds 151-253.

TABLE 2

| Example | | | Molecular |
|---|---|---|---|
| Compound | | | Weight |
| No. | Structure | IUPAC Name | (actual) |
| 151 | 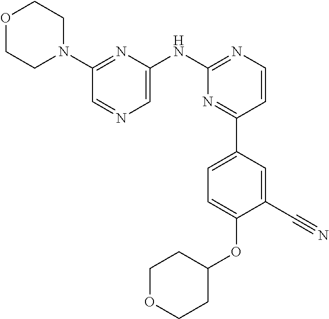 [SMILES: N#Cc1cc(-c2ccnc(Nc3cncc(N4CCOCC4)n3)n2)ccc1OC1CCOCC1] | 5-(2-{[6-(Morpholin-4- yl)pyrazin-2- yl]amino}pyrimidin-4-yl)-2- (tetrahydro-2H-pyran-4- yloxy)benzonitrile | [M + H] 460.2104 |
| 152 | 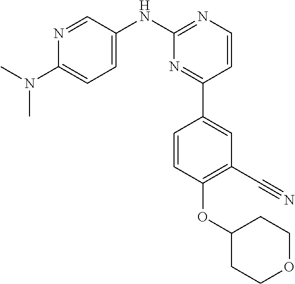 [SMILES: CN(C)c1ccc(Nc2nccc(-c3ccc(OC4CCOCC4)c(C#N)c3)n2)cn1] | 5-(2-{[6- (Dimethylamino)pyridin-3- yl]amino}pyrimidin-4-yl)-2- (tetrahydro-2H-pyran-4- yloxy)benzonitrile | — |
| 153 | 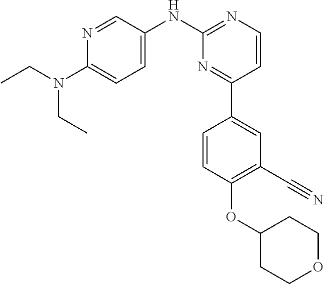 [SMILES: CCN(CC)c1ccc(Nc2nccc(-c3ccc(OC4CCOCC4)c(C#N)c3)n2)cn1] | 5-(2-{[6- (Diethylamino)pyridin- 3-yl]amino}pyrimidin-4-yl)-2- (tetrahydro-2H-pyran-4- yloxy)benzonitrile | — |
| 154 | 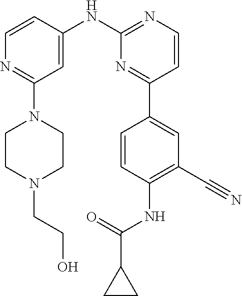 [SMILES: N#Cc1cc(-c2ccnc(Nc3ccnc(N4CCN(CCO)CC4)c3)n2)ccc1NC(=O)C1CC1] | N-{2-Cyano-4-[2-({2-[4-(2- hydroxyethyl)piperazin-1- yl]pyridin-4- yl}amino)pyrimidin-4- yl]phenyl}cyclopropanecarbox- amide | — |
| 155 | 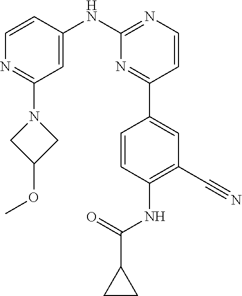 [SMILES: COC1CN(c2cc(Nc3nccc(-c4ccc(NC(=O)C5CC5)c(C#N)c4)n3)ccn2)C1] | N-[2-Cyano-4-(2-{[2-(3- methoxyazetidin-1-yl)pyridin- 4-yl]amino}pyrimidin-4- yl)phenyl]cyclopropanecarbox- amide | [M + H] 442.19489 |
| 156 | 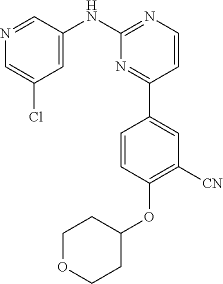 [SMILES: N#Cc1cc(-c2ccnc(Nc3cncc(Cl)c3)n2)ccc1OC1CCOCC1] | 5-{2-[(5-Chloropyridin-3- yl)amino]pyrimidin-4-yl}-2- (tetrahydro-2H-pyran-4- yloxy)benzonitrile | — |
| 157 | 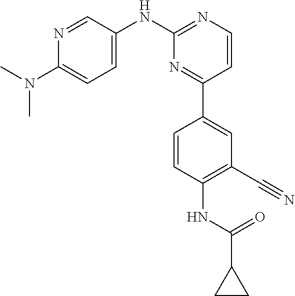 [SMILES: CN(C)c1ccc(Nc2nccc(-c3ccc(NC(=O)C4CC4)c(C#N)c3)n2)cn1] | N-[2-Cyano-4-(2-{[6- (dimethylamino)pyridin-3- yl]amino}pyrimidin-4- yl)phenyl]cyclopropanecarbox- amide | [M + H] 400.1877 |
| 158 | 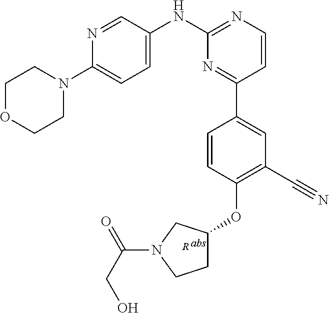 | 2-{[(3R)-1- (Hydroxyacetyl)pyrrolidin-3- yl]oxy}-5-(2-{[6-(morpholin-4- yl)pyridin-3- yl]amino}pyrimidin-4- yl)benzonitrile | [M + H] 502.2159 |
| 159 | 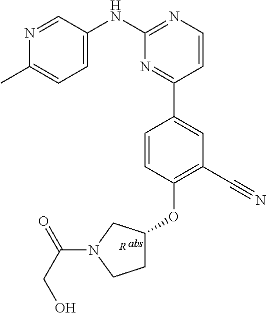 | 2-{[(3R)-1- (Hydroxyacetyl)pyrrolidin-3- yl]oxy}-5-{2-[(6-methylpyridin- 3-yl)amino]pyrimidin-4- yl}benzonitrile | [M + H] 431.1825 |
| 160 | 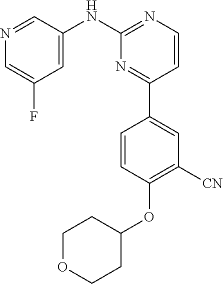 [SMILES: N#Cc1cc(-c2ccnc(Nc3cncc(F)c3)n2)ccc1OC1CCOCC1] | 5-{2-[(5-Fluoropyridin-3- yl)amino]pyrimidin-4-yl}-2- (tetrahydro-2H-pyran-4- yloxy)benzonitrile | — |
| 161 | 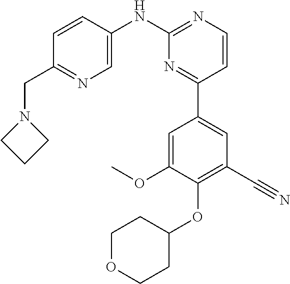 [SMILES: COc1cc(-c2ccnc(Nc3ccc(CN4CCC4)nc3)n2)cc(C#N)c1OC1CCOCC1] | 5-(2-{[6-(Azetidin-1- ylmethyl)pyridin-3- yl]amino}pyrimidin-4-yl)-3- methoxy-2-(tetrahydro-2H- pyran-4-yloxy)benzonitrile | [M + H] 473.2312 |
| 162 | 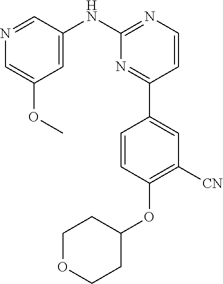 [SMILES: COc1cncc(Nc2nccc(-c3ccc(OC4CCOCC4)c(C#N)c3)n2)c1] | 5-{2-[(5-Methoxypyridin-3- yl)amino]pyrimidin-4-yl}-2- (tetrahydro-2H-pyran-4- yloxy)benzonitrile | — |
| 163 | 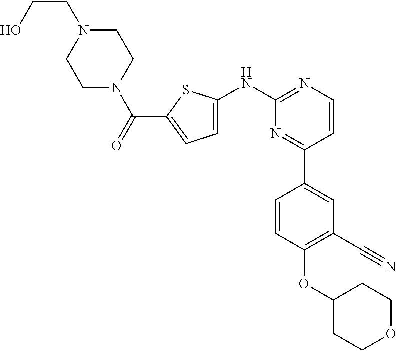 [SMILES: N#Cc1cc(-c2ccnc(Nc3ccc(C(=O)N4CCN(CCO)CC4)s3)n2)ccc1OC1CCOCC1] | 5-{2-[(5-{[4-(2- Hydroxyethyl)piperazin-1- yl]carbonyl}thiophen-2- yl)amino]pyrimidin-4-yl}-2- (tetrahydro-2H-pyran-4- yloxy)benzonitrile | [M + H] 535.2141 |
| 164 | 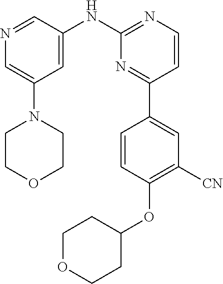 [SMILES: N#Cc1cc(-c2ccnc(Nc3cncc(N4CCOCC4)c3)n2)ccc1OC1CCOCC1] | 5-(2-{[5-(Morpholin-4- yl)pyridin-3- yl]amino}pyrimidin-4-yl)-2- (tetrahydro-2H-pyran-4- yloxy)benzonitrile | — |
| 165 | 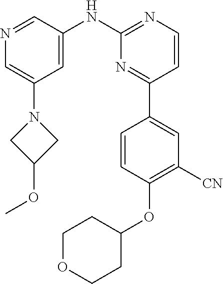 [SMILES: COC1CN(c2cncc(Nc3nccc(-c4ccc(OC5CCOCC5)c(C#N)c4)n3)c2)C1] | 5-(2-{[5-(3-Methoxyazetidin-1- yl)pyridin-3- yl]amino}pyrimidin-4-yl)-2- (tetrahydro-2H-pyran-4- yloxy)benzonitrile | — |
| 166 | 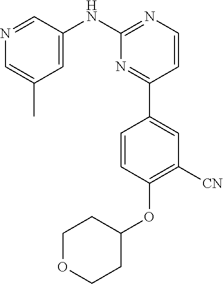 [SMILES: Cc1cncc(Nc2nccc(-c3ccc(OC4CCOCC4)c(C#N)c3)n2)c1] | 5-{2-[(5-Methylpyridin-3- yl)amino]pyrimidin-4-yl}-2- (tetrahydro-2H-pyran-4- yloxy)benzonitrile | — |
| 167 | 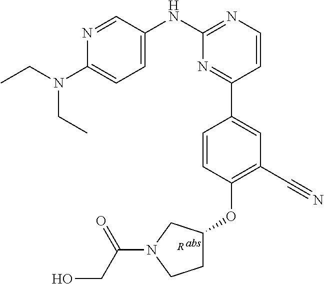 | 5-(2-{[6-(Diethylamino)pyridin- 3-yl]amino}pyrimidin-4-yl)-2- {[(3R)-1- (hydroxyacetyl)pyrrolidin-3- yl]oxy}benzonitrile | — |
| 168 | 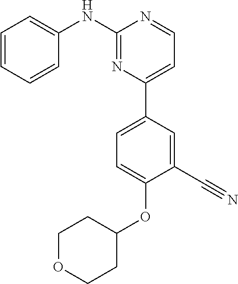 [SMILES: N#Cc1cc(-c2ccnc(Nc3ccccc3)n2)ccc1OC1CCOCC1] | 5-(2-{[2-(1H-Pyrazol-4- yl)pyridin-4- yl]amino}pyrimidin-4-yl)-2- (tetrahydro-2H-pyran-4- yloxy)benzonitrile | [M + H] 440.1883 |
| 169 | 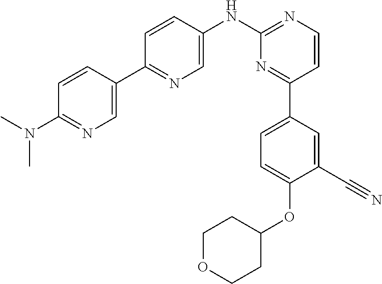 [SMILES: CN(C)c1ccc(-c2ccc(Nc3nccc(-c4ccc(OC5CCOCC5)c(C#N)c4)n3)cn2)cn1] | 5-(2-{[6′-(Dimethylamino)-2,3′- bipyridin-5-yl]amino}pyrimidin- 4-yl)-2-(tetrahydro-2H-pyran-4- yloxy)benzonitrile | [M + H] 494.2299 |
| 170 | 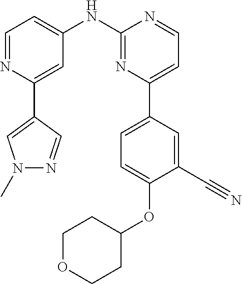 [SMILES: Cn1cc(-c2cc(Nc3nccc(-c4ccc(OC5CCOCC5)c(C#N)c4)n3)ccn2)cn1] | 5-(2-{[2-(1-Methyl-1H-pyrazol- 4-yl)pyridin-4- yl]amino}pyrimidin-4-yl)-2- (tetrahydro-2H-pyran-4- yloxy)benzonitrile | [M + H] 454.2053 |
| 171 | 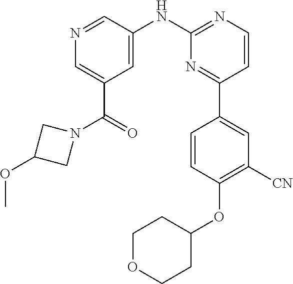 [SMILES: COC1CN(C(=O)c2cncc(Nc3nccc(-c4ccc(OC5CCOCC5)c(C#N)c4)n3)c2)C1] | 5-[2-({5-[(3-Methoxyazetidin- 1-yl)carbonyl]pyridin-3- yl}amino)pyrimidin-4-yl]-2- (tetrahydro-2H-pyran-4- yloxy)benzonitrile | — |
| 172 | 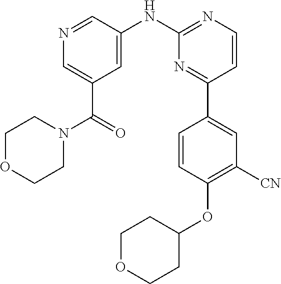 [SMILES: N#Cc1cc(-c2ccnc(Nc3cncc(C(=O)N4CCOCC4)c3)n2)ccc1OC1CCOCC1] | 5-(2-{[5-(Morpholin-4- ylcarbonyl)pyridin-3- yl]amino}pyrimidin-4-yl)-2- (tetrahydro-2H-pyran-4- yloxy)benzonitrile | — |
| 173 | 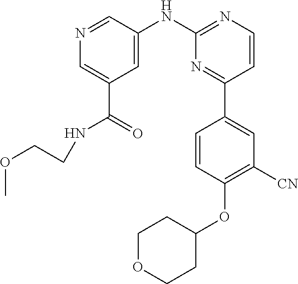 [SMILES: COCCNC(=O)c1cncc(Nc2nccc(-c3ccc(OC4CCOCC4)c(C#N)c3)n2)c1] | 5-({4-[3-Cyano-4-(tetrahydro- 2H-pyran-4- yloxy)phenyl]pyrimidin-2- yl}amino)-N-(2- methoxyethyl)pyridine-3- carboxamide | — |
| 174 | 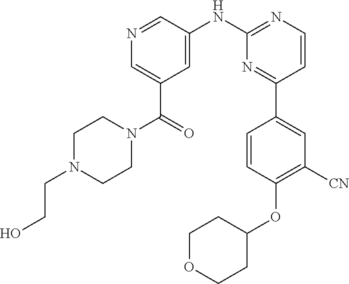 [SMILES: N#Cc1cc(-c2ccnc(Nc3cncc(C(=O)N4CCN(CCO)CC4)c3)n2)ccc1OC1CCOCC1] | 5-{2-[(5-{[4-(2- Hydroxyethyl)piperazin-1- yl]carbonyl}pyridin-3- yl)amino]pyrimidin-4-yl}-2- (tetrahydro-2H-pyran-4- yloxy)benzonitrile | — |
| 175 | 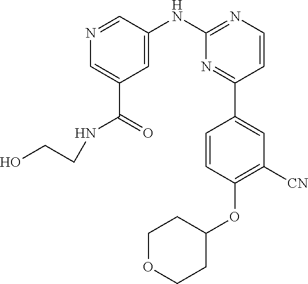 [SMILES: N#Cc1cc(-c2ccnc(Nc3cncc(C(=O)NCCO)c3)n2)ccc1OC1CCOCC1] | 5-({4-[3-Cyano-4-(tetrahydro- 2H-pyran-4- yloxy)phenyl]pyrimidin-2- yl}amino)-N-(2- hydroxyethyl)pyridine-3- carboxamide | — |
| 176 | 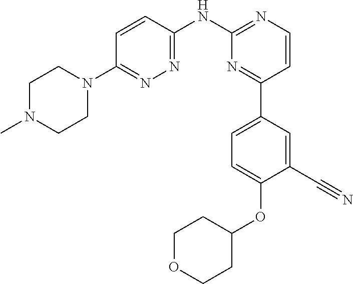 [SMILES: CN1CCN(c2ccc(Nc3nccc(-c4ccc(OC5CCOCC5)c(C#N)c4)n3)nn2)CC1] | 5-(2-{[6-(4-Methylpiperazin-1- yl)pyridazin-3- yl]amino}pyrimidin-4-yl)-2- (tetrahydro-2H-pyran-4- yloxy)benzonitrile | [M + H] 473.2473 |
| 177 | 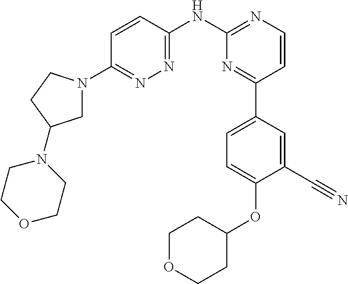 [SMILES: N#Cc1cc(-c2ccnc(Nc3ccc(N4CCC(N5CCOCC5)C4)nn3)n2)ccc1OC1CCOCC1] | 5-[2-({6-[3-(Morpholin-4- yl)pyrrolidin-1-yl]pyridazin-3- yl}amino)pyrimidin-4-yl]-2- (tetrahydro-2H-pyran-4- yloxy)benzonitrile | [M + H] 529.2763 |
| 178 | 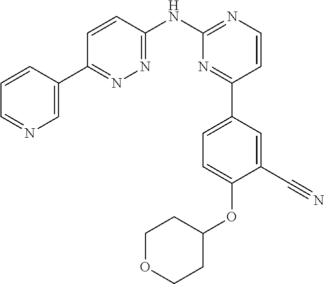 [SMILES: N#Cc1cc(-c2ccnc(Nc3ccc(-c4cccnc4)nn3)n2)ccc1OC1CCOCC1] | 5-[2-(2,3′-Bipyridin-5- ylamino)pyrimidin-4-yl]-2- (tetrahydro-2H-pyran-4- yloxy)benzonitrile | [M + H] 451.1855 |
| 179 | 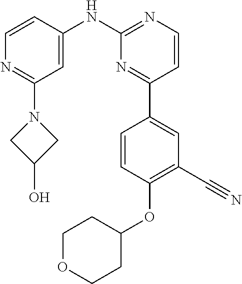 [SMILES: N#Cc1cc(-c2ccnc(Nc3ccnc(N4CC(O)C4)c3)n2)ccc1OC1CCOCC1] | 5-(2-{[2-(3-Hydroxyazetidin-1- yl)pyridin-4- yl]amino}pyrimidin-4-yl)-2- (tetrahydro-2H-pyran-4- yloxy)benzonitrile | [M + H] 445.1991 |
| 180 | 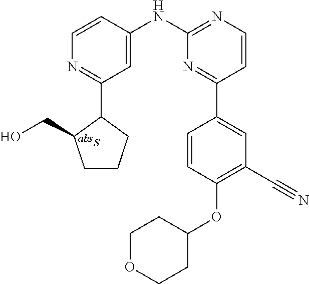 | 5-[2-({2-[(2S)-2- (Hydroxymethyl)pyrrolidin-1- yl]pyridin-4- yl}amino)pyrimidin-4-yl]-2- (tetrahydro-2H-pyran-4- yloxy)benzonitrile | [M + H] 473.2303 |
| 181 | 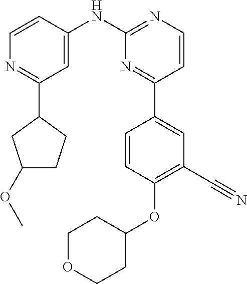 [SMILES: COC1CCC(c2cc(Nc3nccc(-c4ccc(OC5CCOCC5)c(C#N)c4)n3)ccn2)C1] | 5-(2-{[2-(3-Methoxypyrrolidin- 1-yl)pyridin-4- yl]amino}pyrimidin-4-yl)-2- (tetrahydro-2H-pyran-4- yloxy)benzonitrile | [M + H] 473.2327 |
| 182 | 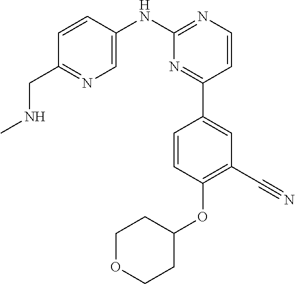 [SMILES: CNCc1ccc(Nc2nccc(-c3ccc(OC4CCOCC4)c(C#N)c3)n2)cn1] | 5-[2-({6- [(Methylamino)methyl]pyridin- 3-yl}amino)pyrimidin-4-yl]-2- (tetrahydro-2H-pyran-4- yloxy)benzonitrile | [M + H] 417.2036 |
| 183 | 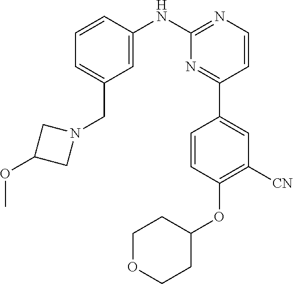 [SMILES: COC1CN(Cc2cccc(Nc3nccc(-c4ccc(OC5CCOCC5)c(C#N)c4)n3)c2)C1] | 5-[2-({5-[(3-Methoxyazetidin-1- yl)methyl]pyridin-3- yl}amino)pyrimidin-4-yl]-2- (tetrahydro-2H-pyran-4- yloxy)benzonitrile | — |
| 184 | 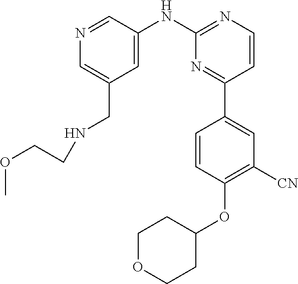 [SMILES: COCCNCc1cncc(Nc2nccc(-c3ccc(OC4CCOCC4)c(C#N)c3)n2)c1] | 5-{2-[(5-{[(2-Methoxy- ethyl)amino]methyl}pyridin- 3-yl)amino]pyrimidin-4- yl}-2-(tetrahydro-2H-pyran-4- yloxy)benzonitrile | — |
| 185 | 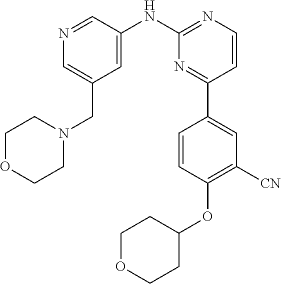 [SMILES: N#Cc1cc(-c2ccnc(Nc3cncc(CN4CCOCC4)c3)n2)ccc1OC1CCOCC1] | 5-(2-{[5-(Morpholin-4- ylmethyl)pyridin-3- yl]amino}pyrimidin-4-yl)-2- (tetrahydro-2H-pyran-4- yloxy)benzonitrile | — |
| 186 | 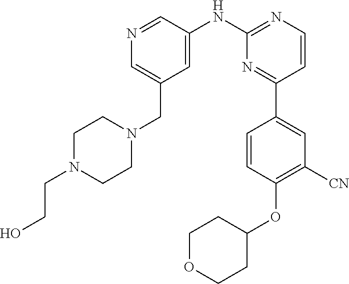 [SMILES: N#Cc1cc(-c2ccnc(Nc3cncc(CN4CCN(CCO)CC4)c3)n2)ccc1OC1CCOCC1] | 5-{2-[(5-{[4-(2- Hydroxyethyl)piperazin-1- yl]methyl}pyridin-3- yl)amino]pyrimidin-4-yl}-2- (tetrahydro-2H-pyran-4- yloxy)benzonitrile | — |
| 187 | 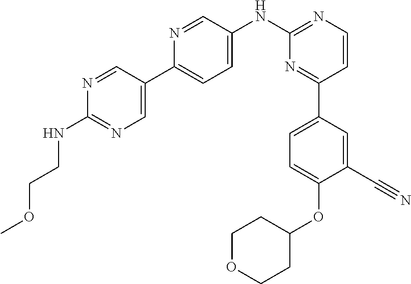 [SMILES: COCCNc1ncc(-c2ccc(Nc3nccc(-c4ccc(OC5CCOCC5)c(C#N)c4)n3)cn2)cn1] | 5-{2-[(6-{2-[(2- Methoxyethyl)amino]pyrimidin- 5-yl}pyridin-3- yl)amino]pyrimidin-4-yl}-2- (tetrahydro-2H-pyran-4- yloxy)benzonitrile | [M + H] 525.2339 |
| 188 | 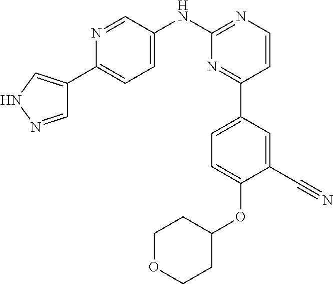 [SMILES: N#Cc1cc(-c2ccnc(Nc3ccc(-c4cn[nH]c4)nc3)n2)ccc1OC1CCOCC1] | 5-(2-{[6-(1H-Pyrazol-4- yl)pyridin-3- yl]amino}pyrimidin-4-yl)-2- (tetrahydro-2H-pyran-4- yloxy)benzonitrile | [M + H] 440.1835 |
| 189 | 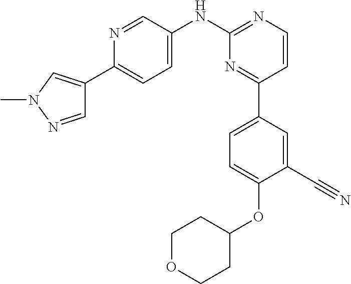 [SMILES: Cn1cc(-c2ccc(Nc3nccc(-c4ccc(OC5CCOCC5)c(C#N)c4)n3)cn2)cn1] | 5-(2-{[6-(1-Methyl-1H-pyrazol- 4-yl)pyridin-3- yl]amino}pyrimidin-4-yl)-2- (tetrahydro-2H-pyran-4- yloxy)benzonitrile | [M + H] 454.1966 |
| 190 | 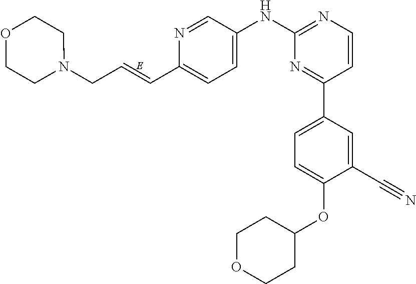 [SMILES: N#Cc1cc(-c2ccnc(Nc3ccc(/C=C/CN4CCOCC4)nc3)n2)ccc1OC1CCOCC1] | 5-[2-({6-[(1E)-3-(Morpholin-4- yl)prop-1-en-1-yl]pyridin-3- yl}amino)pyrimidin-4-yl]-2- (tetrahydro-2H-pyran-4- yloxy)benzonitrile | [M + H] 499.2462 |
| 191 | 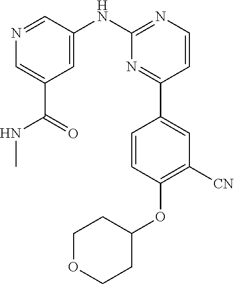 [SMILES: CNC(=O)c1cncc(Nc2nccc(-c3ccc(OC4CCOCC4)c(C#N)c3)n2)c1] | 5-({4-[3-Cyano-4-(tetrahydro- 2H-pyran-4- yloxy)phenyl]pyrimidin-2- yl}amino)-N-methylpyridine-3- carboxamide | — |
| 192 | 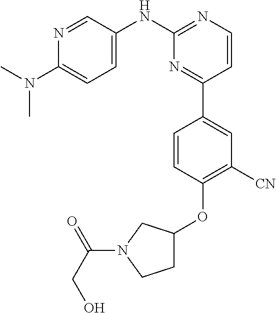 [SMILES: CN(C)c1ccc(Nc2nccc(-c3ccc(OC4CCN(C(=O)CO)C4)c(C#N)c3)n2)cn1] | 5-(2-{[6- (Dimethylamino)pyridin-3- yl]amino}pyrimidin-4-yl)-2-{[1- (hydroxyacetyl)pyrrolidin-3- yl]oxy}benzonitrile | — |
| 193 | 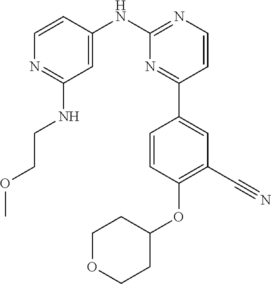 [SMILES: COCCNc1cc(Nc2nccc(-c3ccc(OC4CCOCC4)c(C#N)c3)n2)ccn1] | 5-[2-({2-[(2- Methoxyethyl)amino]pyridin-4- yl}amino)pyrimidin-4-yl]-2- (tetrahydro-2H-pyran-4- yloxy)benzonitrile | [M + H] 447.2151 |
| 194 | 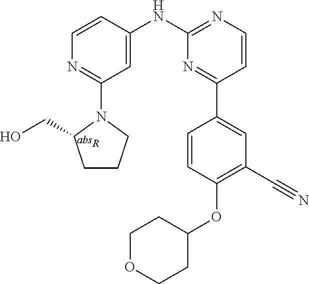 | 5-[2-({2-[(2R)-2- (Hydroxymethyl)pyrrolidin-1- yl]pyridin-4- yl}amino)pyrimidin-4-yl]-2- (tetrahydro-2H-pyran-4- yloxy)benzonitrile | [M + H] 473.2308 |
| 195 | 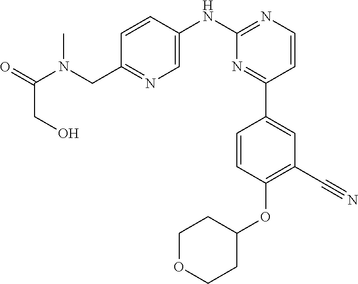 [SMILES: CN(Cc1ccc(Nc2nccc(-c3ccc(OC4CCOCC4)c(C#N)c3)n2)cn1)C(=O)CO] | N-{[5-({4-[3-Cyano-4- (tetrahydro-2H-pyran-4- yloxy)phenyl]pyrimidin-2- yl}amino)pyridin-2-yl]methyl}- 2-hydroxy-N-methylacetamide | [M + H] 475.2146 |
| 196 | 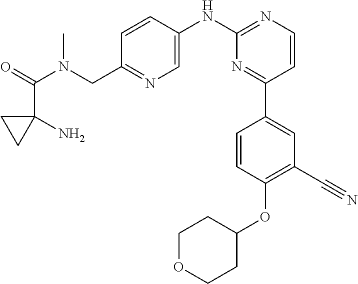 [SMILES: CN(Cc1ccc(Nc2nccc(-c3ccc(OC4CCOCC4)c(C#N)c3)n2)cn1)C(=O)C1(N)CC1] | 1-Amino-N-{[5-({4-[3-cyano-4- (tetrahydro-2H-pyran-4- yloxy)phenyl]pyrimidin-2- yl}amino)pyridin-2-yl]methyl}- N- methylcyclopropanecarboxamide | [M + H] 500.2464 |
| 197 | 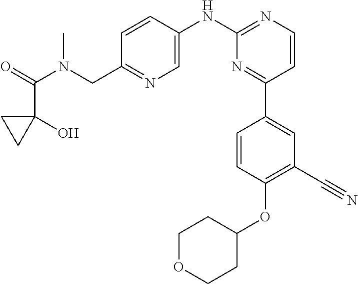 [SMILES: CN(Cc1ccc(Nc2nccc(-c3ccc(OC4CCOCC4)c(C#N)c3)n2)cn1)C(=O)C1(O)CC1] | N-{[5-({4-[3-Cyano-4- (tetrahydro-2H-pyran-4- yloxy)phenyl]pyrimidin-2- yl}amino)pyridin-2-yl]methyl}- 1-hydroxy-N- methylcyclopropanecarboxamide | [M + H] 501.2306 |
| 198 | 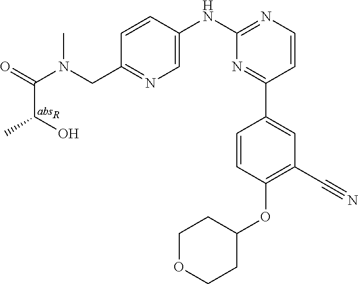 | (2R)-N-{[5-({4-[3-Cyano-4- (tetrahydro-2H-pyran-4- yloxy)phenyl]pyrimidin-2- yl}amino)pyridin-2-yl]methyl}- 2-hydroxy-N- methylpropanamide | [M + H] 489.2308 |
| 199 | 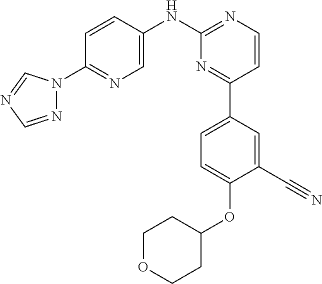 [SMILES: N#Cc1cc(-c2ccnc(Nc3ccc(-n4cncn4)nc3)n2)ccc1OC1CCOCC1] | 2-(Tetrahydro-2H-pyran-4- yloxy)-5-(2-{[6-(1H-1,2,4- triazol-1-yl)pyridin-3- yl]amino}pyrimidin-4- yl)benzonitrile | [M + H] 441.1849 |
| 200 | 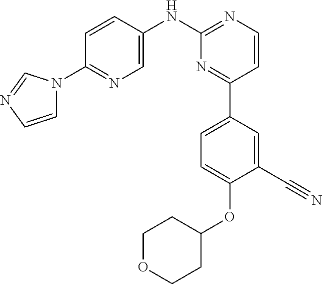 [SMILES: N#Cc1cc(-c2ccnc(Nc3ccc(-n4ccnc4)nc3)n2)ccc1OC1CCOCC1] | 5-(2-{[6-(1H-Imidazol-1- yl)pyridin-3- yl]amino}pyrimidin-4-yl)-2- (tetrahydro-2H-pyran-4- yloxy)benzonitrile | [M + H] 440.1826 |
| 201 | 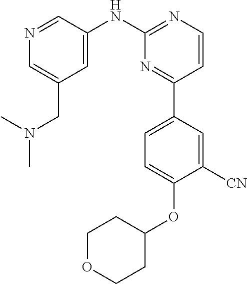 [SMILES: CN(C)Cc1cncc(Nc2nccc(-c3ccc(OC4CCOCC4)c(C#N)c3)n2)c1] | 5-[2-({5- [(Dimethylamino)methyl]pyridin- 3-yl}amino)pyrimidin-4-yl]-2- (tetrahydro-2H-pyran-4- yloxy)benzonitrile | — |
| 202 | 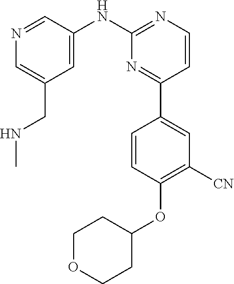 [SMILES: CNCc1cncc(Nc2nccc(-c3ccc(OC4CCOCC4)c(C#N)c3)n2)c1] | 5-[2-({5- [(Methylamino)methyl]pyridin- 3-yl}amino)pyrimidin-4-yl]-2- (tetrahydro-2H-pyran-4- yloxy)benzonitrile | — |
| 203 | 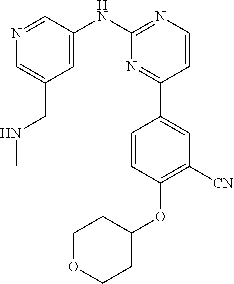 [SMILES: CNCc1cncc(Nc2nccc(-c3ccc(OC4CCOCC4)c(C#N)c3)n2)c1] | 5-[2-({5- [(Methylamino)methyl]pyridin- 3-yl}amino)pyrimidin-4-yl]-2- (tetrahydro-2H-pyran-4- yloxy)benzonitrile | [M + H] 417.2032 |
| 204 | 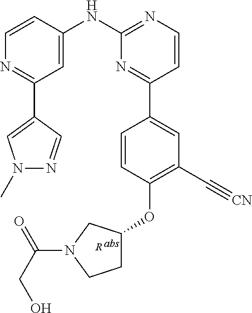 | 2-{[(3R)-1- (Hydroxyacetyl)pyrrolidin-3- yl]oxy}-5-(2-{[2-(1-methyl-1H- pyrazol-4-yl)pyridin-4- yl]amino}pyrimidin-4- yl)benzonitrile | [M + H] 499.2073 |
| 205 | 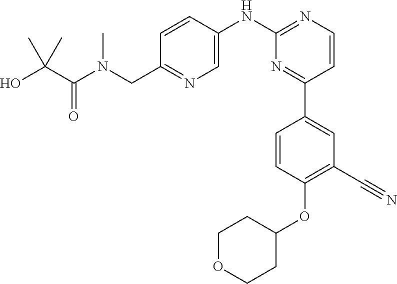 [SMILES: CN(Cc1ccc(Nc2nccc(-c3ccc(OC4CCOCC4)c(C#N)c3)n2)cn1)C(=O)C(C)(C)O] | N-{[5-({4-[3-Cyano-4- (tetrahydro-2H-pyran-4- yloxy)phenyl]pyrimidin-2- yl}amino)pyridin-2-yl]methyl}- 2-hydroxy-N,2- dimethylpropanamide | [M + H] 503.2327 |
| 206 | 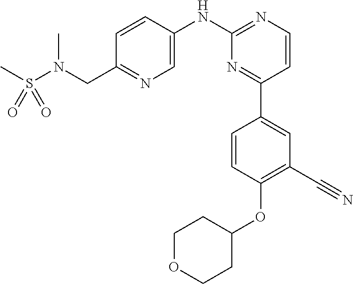 [SMILES: CN(Cc1ccc(Nc2nccc(-c3ccc(OC4CCOCC4)c(C#N)c3)n2)cn1)S(C)(=O)=O] | N-{[5-({4-[3-Cyano-4- (tetrahydro-2H-pyran-4- yloxy)phenyl]pyrimidin-2- yl}amino)pyridin-2-yl]methyl}- N-methylmethanesulfonamide | [M + H] 495.1741 |
| 207 | 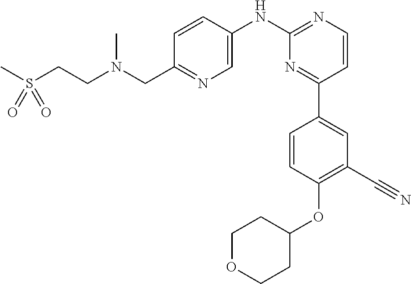 [SMILES: CN(CCS(C)(=O)=O)Cc1ccc(Nc2nccc(-c3ccc(OC4CCOCC4)c(C#N)c3)n2)cn1] | 5-(2-{[6-({Methyl[2- (methylsulfonyl)ethyl]a- mino}methyl)pyridin-3- yl]amino}pyrimidin-4-yl)-2- (tetrahydro-2H-pyran-4- yloxy)benzonitrile | [M + H] 523.2046 |
| 208 | 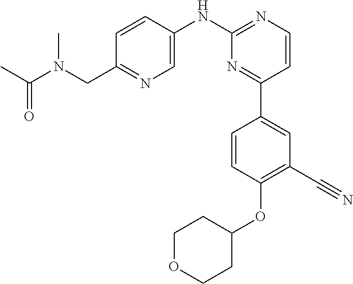 [SMILES: CC(=O)N(C)Cc1ccc(Nc2nccc(-c3ccc(OC4CCOCC4)c(C#N)c3)n2)cn1] | N-{[5-({4-[3-Cyano-4- (tetrahydro-2H-pyran-4- yloxy)phenyl]pyrimidin-2- yl}amino)pyridin-2-yl]methyl}- N-methylacetamide | [M + H] 459.2078 |
| 209 | 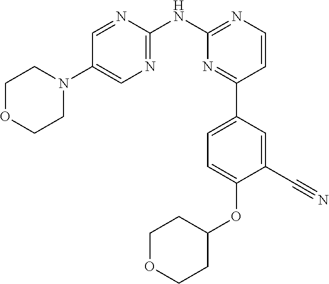 [SMILES: N#Cc1cc(-c2ccnc(Nc3ncc(N4CCOCC4)cn3)n2)ccc1OC1CCOCC1] | 5-(2-{[5-(Morpholin-4- yl)pyrimidin-2- yl]amino}pyrimidin-4-yl)- 2-(tetrahydro-2H-pyran-4- yloxy)benzonitrile | [M + H] 460.2089 |
| 210 | 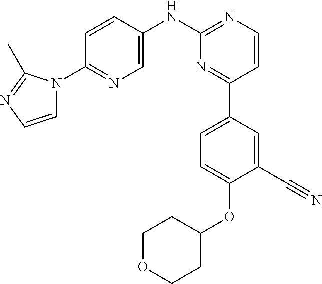 [SMILES: Cc1nccn1-c1ccc(Nc2nccc(-c3ccc(OC4CCOCC4)c(C#N)c3)n2)cn1] | 5-(2-{[6-(2-Methyl-1H- imidazol-1-yl)pyridin-3- yl]amino}pyrimidin-4-yl)- 2-(tetrahydro-2H-pyran-4- yloxy)benzonitrile | [M + H] 454.2014 |
| 211 | 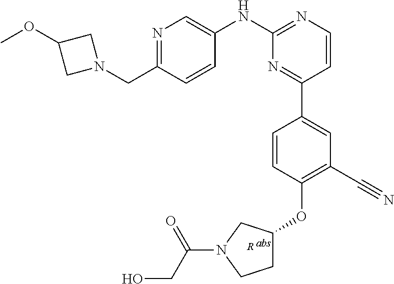 | 2-{[(3R)-1- (Hydroxyacetyl)pyrrolidin-3- yl]oxy}-5-[2-({6-[(3- methoxyazetidin-1- yl)methyl]pyridin-3- yl}amino)pyrimidin-4- yl]benzonitrile | [M + H] 516.2354 |
| 212 | 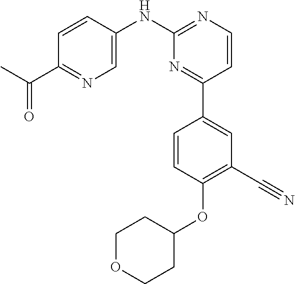 [SMILES: CC(=O)c1ccc(Nc2nccc(-c3ccc(OC4CCOCC4)c(C#N)c3)n2)cn1] | 5-{2-[(6-Acetylpyridin-3- yl)amino]pyrimidin-4-yl}-2- (tetrahydro-2H-pyran-4- yloxy)benzonitrile | [M + H] 416.1732 |
| 213 | 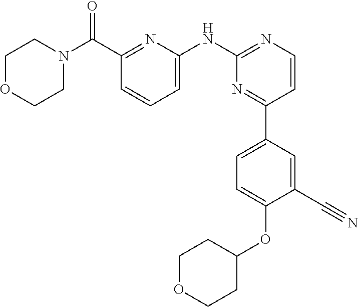 [SMILES: N#Cc1cc(-c2ccnc(Nc3cccc(C(=O)N4CCOCC4)n3)n2)ccc1OC1CCOCC1] | 5-(2-{[6-(Morpholin-4- ylcarbonyl)pyridin-2- yl]amino}pyrimidin-4- yl)-2-(tetrahydro-2H-pyran-4- yloxy)benzonitrile | [M + H] 487.2107 |
| 214 | 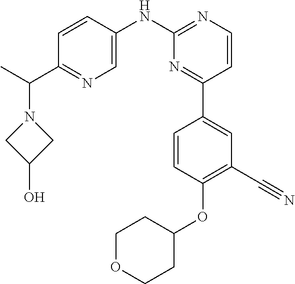 [SMILES: CC(c1ccc(Nc2nccc(-c3ccc(OC4CCOCC4)c(C#N)c3)n2)cn1)N1CC(O)C1] | 5-[2-({6-[1-(3-Hydroxyazetidin- 1-yl)ethyl]pyridin-3- yl}amino)pyrimidin-4-yl]-2- (tetrahydro-2H-pyran-4- yloxy)benzonitrile | [M + H] 473.2254 |
| 215 | 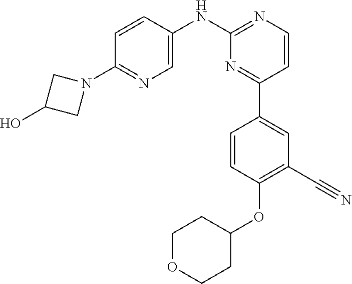 [SMILES: N#Cc1cc(-c2ccnc(Nc3ccc(N4CC(O)C4)nc3)n2)ccc1OC1CCOCC1] | 5-(2-{[6-(3-Hydroxyazetidin-1- yl)pyridin-3- yl]amino}pyrimidin-4-yl)-2- (tetrahydro-2H-pyran-4- yloxy)benzonitrile | [M + H] 445.1994 |
| 216 | 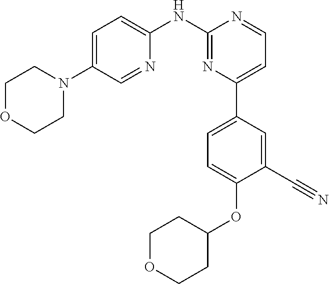 [SMILES: N#Cc1cc(-c2ccnc(Nc3ccc(N4CCOCC4)cn3)n2)ccc1OC1CCOCC1] | 5-(2-{[5-(Morpholin-4- yl)pyridin-2- yl]amino}pyrimidin-4-yl)-2- (tetrahydro-2H-pyran-4- yloxy)benzonitrile | [M + H] 458.2075 |
| 217 | 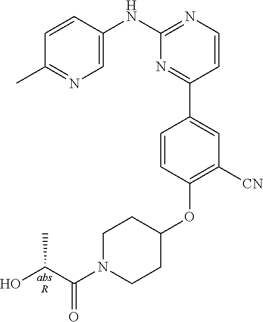 | 2-({1-[(2R)-2- Hydroxypropanoyl]piperidin-4- yl}oxy)-5-{2-[(6-methylpyridin- 3-yl)amino]pyrimidin-4- yl]benzonitrile | [M + H] 459.2149 |
| 218 | 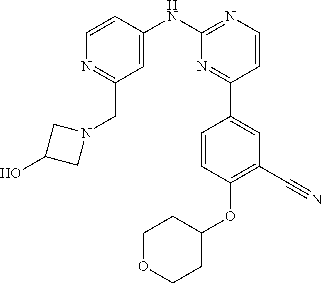 [SMILES: N#Cc1cc(-c2ccnc(Nc3ccnc(CN4CC(O)C4)c3)n2)ccc1OC1CCOCC1] | 5-[2-({2-[(3-Hydroxyazetidin-1- yl)methyl]pyridin-4- yl}amino)pyrimidin-4-yl]-2- (tetrahydro-2H-pyran-4- yloxy)benzonitrile | [M + Na] 481.1968 |
| 219 | 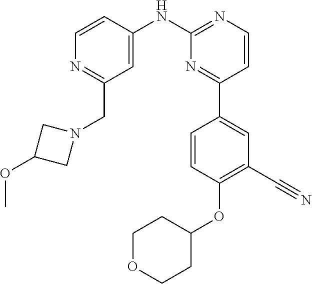 [SMILES: COC1CN(Cc2cc(Nc3nccc(-c4ccc(OC5CCOCC5)c(C#N)c4)n3)ccn2)C1] | 5-[2-({2-[(3-Methoxyazetidin-1- yl)methyl]pyridin-4- yl}amino)pyrimidin-4-yl]-2- (tetrahydro-2H-pyran-4- yloxy)benzonitrile | [M + Na] 495.2114 |
| 220 | 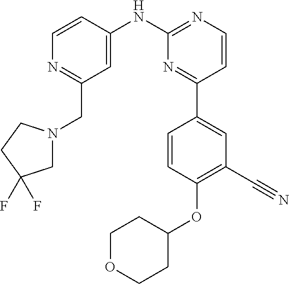 [SMILES: N#Cc1cc(-c2ccnc(Nc3ccnc(CN4CCC(F)(F)C4)c3)n2)ccc1OC1CCOCC1] | 5-[2-({2-[(3,3- Difluoropyrrolidin-1- yl)methyl]pyridin-4- yl}amino)pyrimidin-4-yl]-2- (tetrahydro-2H-pyran-4- yloxy)benzonitrile | [M + Na] 515.1990 |
| 221 | 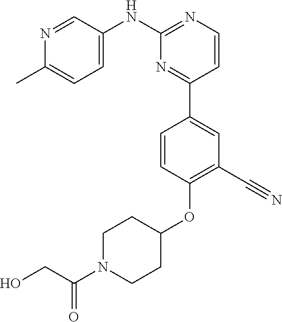 [SMILES: Cc1ccc(Nc2nccc(-c3ccc(OC4CCN(C(=O)CO)CC4)c(C#N)c3)n2)cn1] | 2-{[1-(Hydroxyacetyl)piperidin- 4-yl]oxy}-5-{2-[(6- methylpyridin-3- yl)amino]pyrimidin-4- yl}benzonitrile | [M + H] 445.2293 |
| 222 | 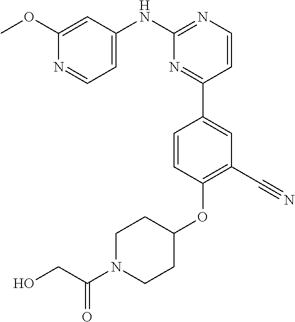 [SMILES: COc1cc(Nc2nccc(-c3ccc(OC4CCN(C(=O)CO)CC4)c(C#N)c3)n2)ccn1] | 2-{[1-(Hydroxyacetyl)piperidin- 4-yl]oxy}-5-{2-[(2- methoxypyridin-4- yl)amino]pyrimidin-4- yl}benzonitrile | [M + H] 461.3386 |
| 223 | 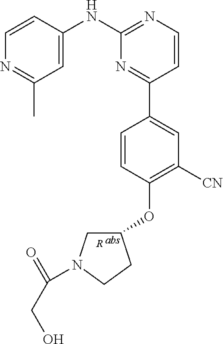 | 2-{[(3R)-1- (Hydroxyacetyl)pyrrolidin-3- yl]oxy}-5-{2-[(2-methylpyridin- 4-yl)amino]pyrimidin-4- yl}benzonitrile | [M + H] 431.3445 |
| 224 | 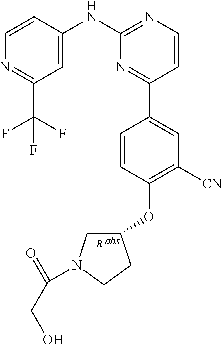 | 2-{[(3R)-1- (Hydroxyacetyl)pyrrolidin-3- yl]oxy}-5-(2-{[2- (trifluoromethyl)pyridin-4- yl]amino}pyrimidin-4- yl)benzonitrile | [M + H] 485.1518 |
| 225 | 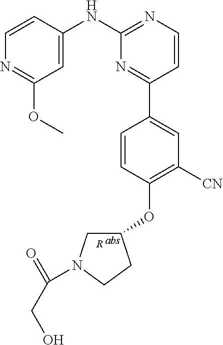 | 2-{[(3R)-1- (Hydroxyacetyl)pyrrolidin-3- yl]oxy}-5-{2-[(2- methoxypyridin-4- yl)amino]pyrimidin-4- yl}benzonitrile | [M + H] 447.2097 |
| 226 | 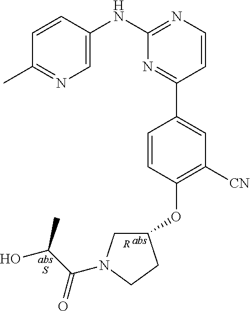 | 2-({(3R)-1-[(2S)-2- Hydroxypropanoyl]pyrrolidin-3- yl}oxy)-5-{2-[(6-methylpyridin- 3-yl)amino]pyrimidin-4- yl}benzonitrile | [M + H] 445.2218 |
| 227 | 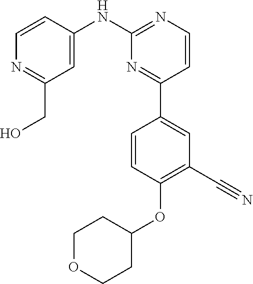 [SMILES: N#Cc1cc(-c2ccnc(Nc3ccnc(CO)c3)n2)ccc1OC1CCOCC1] | 5-(2-{[2- (Hydroxymethyl)pyridin-4- yl]amino}pyrimidin-4-yl)-2- (tetrahydro-2H-pyran-4- yloxy)benzonitrile | [M + H] 426.1522 |
| 228 | 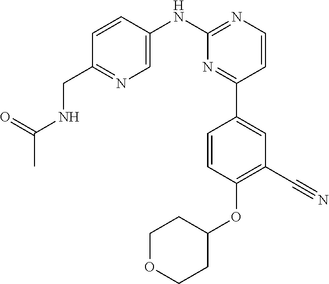 [SMILES: CC(=O)NCc1ccc(Nc2nccc(-c3ccc(OC4CCOCC4)c(C#N)c3)n2)cn1] | N-{[5-({4-[3-Cyano-4- (tetrahydro-2H-pyran-4- yloxy)phenyl]pyrimidin-2- yl}amino)pyridin-2- yl]methyl}acetamide | [M + H] 445.1992 |
| 229 | 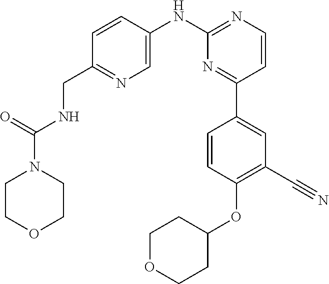 [SMILES: N#Cc1cc(-c2ccnc(Nc3ccc(CNC(=O)N4CCOCC4)nc3)n2)ccc1OC1CCOCC1] | N-{[5-({4-[3-Cyano-4- (tetrahydro-2H-pyran-4- yloxy)phenyl]pyrimidin-2- yl}amino)pyridin-2- yl]methyl}morpholine-4- carboxamide | [M + Na] 538.2167 |
| 230 | 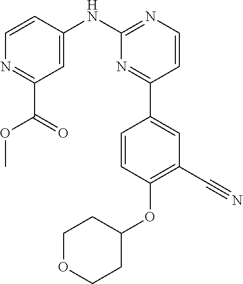 [SMILES: COC(=O)c1cc(Nc2nccc(-c3ccc(OC4CCOCC4)c(C#N)c3)n2)ccn1] | Methyl 4-({4-[3-cyano-4- (tetrahydro-2H-pyran-4- yloxy)phenyl]pyrimidin-2- yl}amino)pyridine-2-carboxylate | [M + H] 432.1676 |
| 231 | 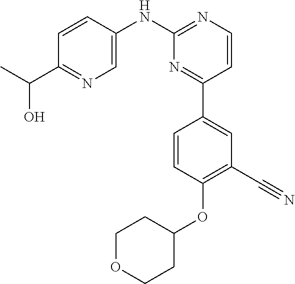 [SMILES: CC(O)c1ccc(Nc2nccc(-c3ccc(OC4CCOCC4)c(C#N)c3)n2)cn1] | 5-(2-{[6-(1- Hydroxyethyl)pyridin-3- yl]amino}pyrimidin-4-yl)-2- (tetrahydro-2H-pyran-4- yloxy)benzonitrile | [M + Na] 440.1704 |
| 232 | 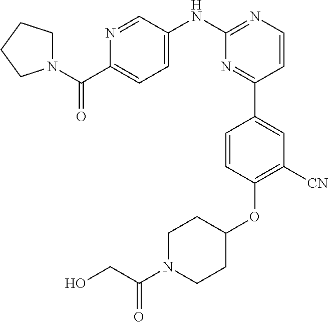 [SMILES: N#Cc1cc(-c2ccnc(Nc3ccc(C(=O)N4CCCC4)nc3)n2)ccc1OC1CCN(C(=O)CO)CC1] | 2-{[1-(Hydroxyacetyl)piperidin- 4-yl]oxy}-5-(2-{[6-(pyrrolidin- 1-ylcarbonyl)pyridin-3- yl]amino}pyrimidin-4- yl)benzonitrile | [M + H] 528.2368 |
| 233 | 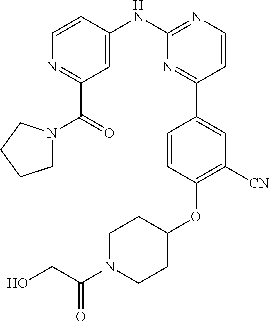 [SMILES: N#Cc1cc(-c2ccnc(Nc3ccnc(C(=O)N4CCCC4)c3)n2)ccc1OC1CCN(C(=O)CO)CC1] | 2-{[1-(Hydroxyacetyl)piperidin- 4-yl]oxy}-5-(2-{[2-(pyrrolidin- 1-ylcarbonyl)pyridin-4- yl]amino}pyrimidin-4- yl)benzonitrile | [M + H] 528.2390 |
| 234 | 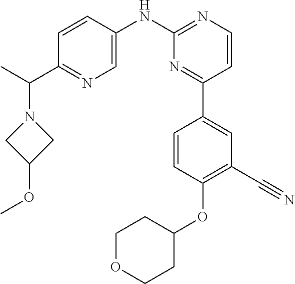 [SMILES: COC1CN(C(C)c2ccc(Nc3nccc(-c4ccc(OC5CCOCC5)c(C#N)c4)n3)cn2)C1] | 5-[2-({6-[1-(3-Methoxyazetidin- 1-yl)ethyl]pyridin-3- yl}amino)pyrimidin-4-yl]-2- (tetrahydro-2H-pyran-4- yloxy)benzonitrile | — |
| 235 | 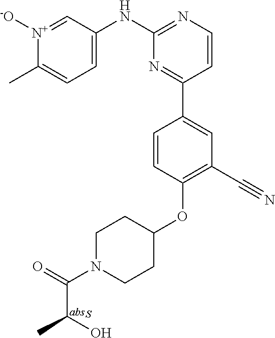 | 2-({1-[(2S)-2- Hydroxypropanoyl]piperidin-4- yl}oxy)-5-{2-[(6-methyl-1- oxidopyridin-3- yl)amino]pyrimidin-4- yl}benzonitrile | [M + H] 475.2093 |
| 236 | 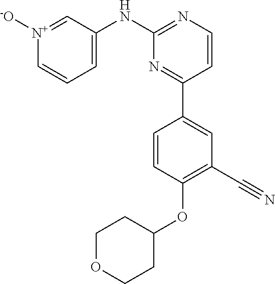 | 5-{2-[(1-Oxidopyridin-3- yl)amino]pyrimidin-4-yl}-2- (tetrahydro-2H-pyran-4- yloxy)benzonitrile | [M + H] 390.1698 |
| 237 | 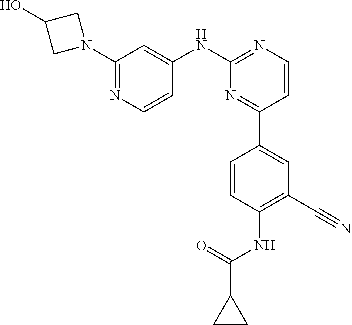 [SMILES: N#Cc1cc(-c2ccnc(Nc3ccnc(N4CC(O)C4)c3)n2)ccc1NC(=O)C1CC1] | N-[2-Cyano-4-(2-{[2-(3- hydroxyazetidin-1-yl)pyridin-4- yl]amino}pyrimidin-4- yl)phenyl]cyclopropanecarbox- amide | [M + H] 428.1842 |
| 238 | 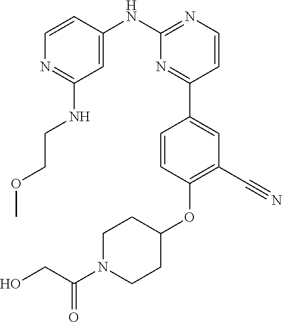 [SMILES: COCCNc1cc(Nc2nccc(-c3ccc(OC4CCN(C(=O)CO)CC4)c(C#N)c3)n2)ccn1] | 2-{[1-(Hydroxyacetyl)piperidin- 4-yl]oxy}-5-[2-({2-[(2- methoxyethyl)amino]pyridin-4- yl}amino)pyrimidin-4- yl]benzonitrile | [M + H] 504.2507 |
| 239 | 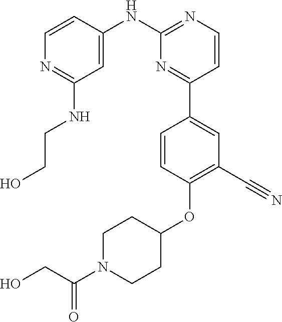 [SMILES: N#Cc1cc(-c2ccnc(Nc3ccnc(NCCO)c3)n2)ccc1OC1CCN(C(=O)CO)CC1] | 2-{[1-(Hydroxyacetyl)piperidin- 4-yl]oxy}-5-[2-({2-[(2- hydroxyethyl)amino]pyridin-4- yl}amino)pyrimidin-4- yl]benzonitrile | [M + H] 490.2223 |
| 240 | 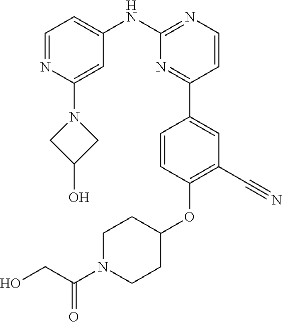 [SMILES: N#Cc1cc(-c2ccnc(Nc3ccnc(N4CC(O)C4)c3)n2)ccc1OC1CCN(C(=O)CO)CC1] | 2-{[1-(Hydroxyacetyl)piperidin- 4-yl]oxy}-5-(2-{[2-(3- hydroxyazetidin-1-yl)pyridin-4- yl]amino}pyrimidin-4- yl)benzonitrile | [M + H] 502.2222 |
| 241 | 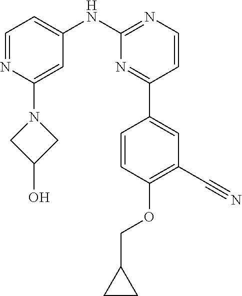 [SMILES: N#Cc1cc(-c2ccnc(Nc3ccnc(N4CC(O)C4)c3)n2)ccc1OCC1CC1] | 2-(Cyclopropylmethoxy)-5-(2- {[2-(3-hydroxyazetidin-1- yl)pyridin-4- yl]amino}pyrimidin-4- yl)benzonitrile | [M + H] 415.1866 |
| 242 | 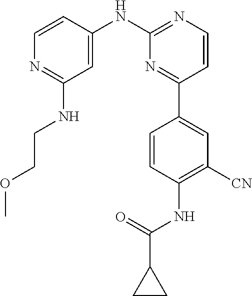 [SMILES: COCCNc1cc(Nc2nccc(-c3ccc(NC(=O)C4CC4)c(C#N)c3)n2)ccn1] | N-{2-Cyano-4-[2-({2-[(2- methoxyethyl)amino]pyridin-4- yl}amino)pyrimidin-4- yl]phenyl}cyclopropanecarbox- amide | [M + H] 430.2000 |
| 243 | 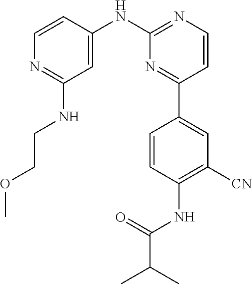 [SMILES: COCCNc1cc(Nc2nccc(-c3ccc(NC(=O)C(C)C)c(C#N)c3)n2)ccn1] | N-{2-Cyano-4-[2-({2-[(2- methoxyethyl)amino]pyridin-4- yl}amino)pyrimidin-4- yl]phenyl}-2- methylpropanamide | [M + H] 432.2160 |
| 244 | 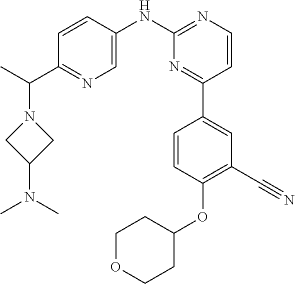 [SMILES: CC(c1ccc(Nc2nccc(-c3ccc(OC4CCOCC4)c(C#N)c3)n2)cn1)N1CC(N(C)C)C1] | 5-{2-[(6-{1-[3- (Dimethylamino)azetidin-1- yl]ethyl}pyridin-3- yl)amino]pyrimidin-4-yl}-2- (tetrahydro-2H-pyran-4- yloxy)benzonitrile | [M + H] 500.2801 |
| 245 | 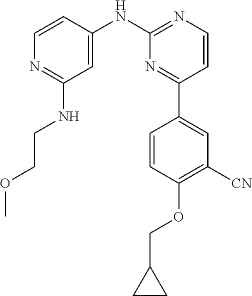 [SMILES: COCCNc1cc(Nc2nccc(-c3ccc(OCC4CC4)c(C#N)c3)n2)ccn1] | 2-(Cyclopropylmethoxy)-5-[2- ({2-[(2- methoxyethyl)amino]pyridin-4- yl}amino)pyrimidin-4- yl]benzonitrile | [M + H] 417.2381 |
| 246 | 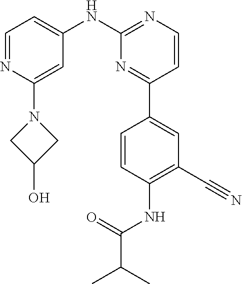 [SMILES: CC(C)C(=O)Nc1ccc(-c2ccnc(Nc3ccnc(N4CC(O)C4)c3)n2)cc1C#N] | N-[2-Cyano-4-(2-{[2-(3- hydroxyazetidin-1-yl)pyridin-4- yl]amino}pyrimidin-4- yl)phenyl]-2-methylpropanamide | [M + H] 430.2192 |
| 247 | 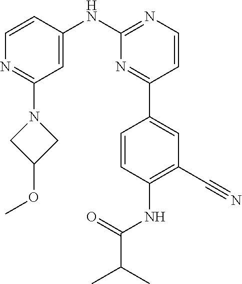 [SMILES: COC1CN(c2cc(Nc3nccc(-c4ccc(NC(=O)C(C)C)c(C#N)c4)n3)ccn2)C1] | N-[2-Cyano-4-(2-{[2-(3- methoxyazetidin-1-yl)pyridin-4- yl]amino}pyrimidin-4- yl)phenyl]-2-methylpropanamide | [M + H] 444.21042 |
| 248 | 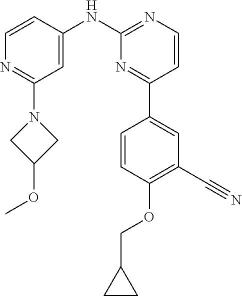 [SMILES: COC1CN(c2cc(Nc3nccc(-c4ccc(OCC5CC5)c(C#N)c4)n3)ccn2)C1] | 2-(Cyclopropylmethoxy)-5-(2- {[2-(3-methoxyazetidin-1- yl)pyridin-4- yl]amino}pyrimidin-4- yl)benzonitrile | [M + H] 429.2000 |
| 249 | 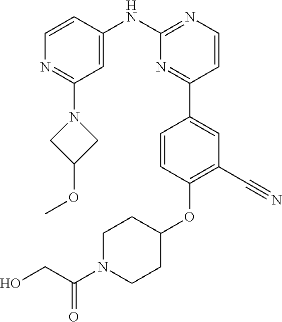 [SMILES: COC1CN(c2cc(Nc3nccc(-c4ccc(OC5CCN(C(=O)CO)CC5)c(C#N)c4)n3)ccn2)C1] | 2-{[1-(Hydroxyacetyl)piperidin- 4-yl]oxy}-5-(2-{[2-(3- methoxyazetidin-1-yl)pyridin-4- yl]amino}pyrimidin-4- yl)benzonitrile | [M + H] 516.2581 |
| 250 | 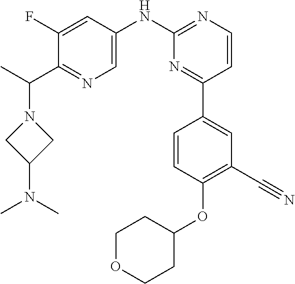 [SMILES: CC(c1ncc(Nc2nccc(-c3ccc(OC4CCOCC4)c(C#N)c3)n2)cc1F)N1CC(N(C)C)C1] | 5-{2-[(6-{1-[3- (Dimethylamino)azetidin-1- yl]ethyl}-5-fluoropyridin-3- yl)amino]pyrimidin-4-yl}-2- (tetrahydro-2H-pyran-4- yloxy)benzonitrile | [M + H] 518.262 |
| 251 | 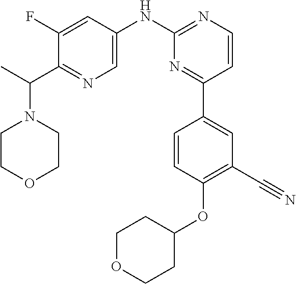 [SMILES: CC(c1ncc(Nc2nccc(-c3ccc(OC4CCOCC4)c(C#N)c3)n2)cc1F)N1CCOCC1] | 5-[2-({5-Fluoro-6-[1- (morpholin-4-yl)ethyl]pyridin-3- yl{amino)pyrimidin-4-yl]-2- (tetrahydro-2H-pyran-4- yloxy)benzonitrile | [M + H] 505.237 |
| 252 | 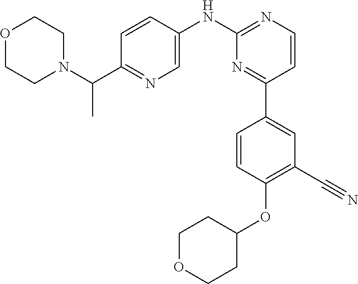 [SMILES: CC(c1ccc(Nc2nccc(-c3ccc(OC4CCOCC4)c(C#N)c3)n2)cn1)N1CCOCC1] | 5-[2-({6-[1-(Morpholin-4- yl)ethyl]pyridin-3- yl}amino)pyrimidin-4-yl]-2- (tetrahydro-2H-pyran-4- yloxy)benzonitrile | [M + H] 487.2491 |
| 253 | 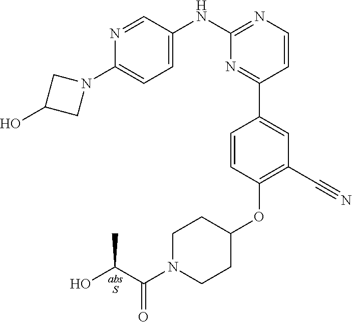 | 5-(2-{[6-(3-Hydroxyazetidin-1- yl)pyridin-3- yl]amino}pyrimidin-4-yl)-2-({1- [(2S)-2- hydroxypropanoyl]piperidin-4- yl}oxy)benzonitrile | [M + H] 516.2479 |

The structures and physicochemical characterization of synthesized Example Compounds are provided in specific examples delineated above. The Example Compounds were synthesized using the methods and intermediates as outlined above using commercially available starting materials that are well known in the art. IUPAC names for the Example Compounds depicted were generated using Advanced Chemistry Development, Inc., (ACD/Labs) (Toronto, Ontario, Canada) ACD/Name IUPAC nomenclature software release 12.00, version 12.01.

The HPLC conditions used to characterize each compound listed above are as follows:

Flow: 1.2 mL/minute

Solvents: A: H₂O+0.01% TFA
B: ACN+0.01% TFA

Gradient: 5% B for 1 minute
5% B to 100% B in 9 minutes
at 100% B for 2.4 minutes
to 0% B in 0.1 minutes
at 0% for 0.5 minutes

Overall time: 13.00 minutes

Column: XTerra MS C₁₈ 3.5 um 4.6×150 mm.

### Biochemical and Biological Examples

### In-Vitro IKKε and TBK1 Kinase Assays

IKKε enzyme was produced as a His-tag fusion in Sf9 cells or purchased as a GST-tag fusion (Invitrogen, Carlsbad, Calif.). TBK1 enzyme was produced as a His-tag fusion in Sf9 cells. Kinase reactions were carried out in reaction buffer using myelin basic protein (Millipore, Ballerica, Mass.), casein or dephosphorylated casein (Sigma, St. Louis, Mo.) as substrate at an ATP concentration equal to twice the K_{m,ATP} value for each enzyme, corresponding to 32 μM ATP for IKKε and 60 μM ATP for TBK1. Radiolabelled [γ³³]ATP (PerkinElmer, Waltham, Mass.) in the amount of 0.3 mCi (IKKe, “normal”) or 0.7 μCi (IKKε, “sensitized”) or 1.25 μCi (TBK1) was added to each assay. Final enzyme concentrations were 0.1 or 0.015 μg/ml (IKKε) and 0.1 or 0.02 μg/ml (TBK1) for the “normal” and “sensitized” assay, respectively, and “sensitized” assays were conducted using only dephosphorylated casein as substrate. Test compounds (or DMSO solvent as a control) were added prior to initiation of the reactions. Reactions were terminated after 30-45 minutes by adding 3% phosphoric acid. Terminated reactions were transferred to P-81 cellulose phosphate filterplates (Whatman, Inc., Piscataway, N.J.) and washed with 1% phosphoric acid on a vacuum apparatus. After air drying, scintillant (PerkinElmer, Waltham, Mass.) was added and the plates were read on a PerkinElmer TopCount NXT instrument. Counts were normalized to DMSO controls after background subtraction.

Using the “sensitized” assay described above for inhibition of IKKε kinase activity, Example Compounds 24, 64, 65, 74, 90, 92, 98, 99, 107, 108, 150, 165, 166, 209, were found to inhibit the kinase activity of IKKε with an IC₅₀ value ranging from greater than 500 nM to about 50 nM;

Example Compounds 1, 2, 3, 6, 14, 16, 17, 18, 19, 20, 25, 26, 28, 31, 35, 47, 49, 50, 51, 72, 73, 84, 85, 86, 91, 93, 96, 97, 100, 101, 103, 105, 106, 110, 112, 113, 114, 115, 116, 118, 120, 122, 124, 125, 129, 132, 133, 134, 135, 136, 137, 140, 144, 145, 146, 147, 148, 156, 159, 160, 161, 164, 171, 172, 174, 176, 177, 186, 191, 192, 199, 201, 202, 203, 210, 211, 213, 216, 217, 224, 226, 233, 234, 235, 236, 242, 243, 246, 247, and 253 were found to inhibit the kinase activity of IKKε with an IC₅₀ value ranging from about 50 nM to about 5 nM; and

Example Compounds 4, 5, 7, 8, 9, 10, 11, 12, 13, 15, 21, 22, 23, 27, 29, 30, 32, 33, 34, 36, 37, 38, 39, 40, 41, 42, 43, 44, 45, 46, 48, 52, 53, 54, 55, 56, 57, 58, 59, 60, 61, 62, 63, 66, 67, 68, 69, 70, 71, 75, 76, 77, 78, 79, 80, 81, 82, 83, 87, 88, 89, 94, 95, 102, 104, 109, 111, 117, 119, 121, 123, 126, 127, 128, 130, 131, 138, 139, 141, 142, 143, 149, 151, 152, 153, 154, 155, 157, 158, 162, 167, 169, 173, 175, 178, 179, 180, 181, 182, 183, 184, 185, 187, 188, 189, 190, 193, 194, 195, 196, 197, 198, 200, 204, 205, 206, 207, 208, 212, 214, 215, 218, 219, 220, 221, 222, 223, 225, 227, 228, 229, 230, 231, 232, 237, 238, 239, 240, 241, 244, 245, 248, 249, 250, 251, and 252 were found to inhibit the kinase activity of IKKε with an IC₅₀ value of less than about 5 nM.

Table 3, below, shows the specific IKKε kinase inhibitory activity as determined for a subset of compounds according to Formulae I and/or II.

Generally, compounds found to inhibit the kinase activity of IKKε would also be expected to inhibit the kinase activity of TBK1, given the high degree of similarity of the amino acid sequences encoding these two closely-related kinases, and particularly those sequences encoding the kinase domains of these enzymes.

### Assays to Detect the In-Situ Phosphorylation of IRF3 (and IRF7)

HEK293T cells were cotransfected in a 10-cm dish with IRF3 and IKKε expression plasmids using Lipofectamine 2000 (Invitrogen, Carlsbad, Calif.). The following day, cells were replated at 20,000 per well in 96-well plates and treated with test compounds (compounds according to Formulae I and/or II) for 20 hours. Cell lysates were prepared and analyzed using an ELISA for phospho-Ser396 (anti-IRF3 capture antibody, Santa Cruz Biotechnology, Inc., Santa Cruz, Calif.; anti-p-Ser396 IRF3 detection antibody, Cell Signaling, Danvers, Mass.). pIRF3 levels in compound treated cells were normalized to DMSO treated cells (no compound). Cell viability was assayed in a parallel set of plates to monitor cytotoxic effects of the test compounds (CellTiter-Glo, Promega, Inc., Madison, Wis.). TBK1 activity was tested by Western blotting using a phospho-specific IRF7 antibody. Similar to above, HEK293T cells were transfected with IRF7 and TBK1 expression plasmids. Cells were seeded in 12-well plates at 150,000 per well and treated overnight with test compounds. Protein lysates were prepared and processed for Western blotting followed by detection using a phosphor-Ser477/Ser479 IRF7 antibody (BD Biosciences, San Jose, Calif.)

Using the assay described above, Example Compounds 12, 16, 20, 28, 39, 84, 97, 105, 121, 126, 128, 129, 130, 136, 169, 172, 174, 175, 177, 181, 186, 191, 204, 216, 217, 219, 221, 225, 233, 235, 236, 237, 239, 240, 246, and 253 were found to inhibit the in-situ IKKε-mediated phosphorylation of IRF3 with an IC₅₀ value ranging from about 5 μM to about 1 μM;

Example Compounds 8, 15, 17, 18, 21, 23, 32, 34, 40, 47, 48, 49, 50, 52, 57, 59, 70, 79, 85, 87, 88, 115, 117, 118, 122, 123, 139, 159, 161, 167, 171, 173, 176, 178, 180, 182, 183, 185, 192, 197, 198, 203, 207, 218, 223, 224, 228, 238, 242, and 247 were found to inhibit the in-situ IKKε-mediated phosphorylation of IRF3 with an IC₅₀ value ranging from about 1 μM to about 250 nM;

Example Compounds 5, 6, 7, 9, 10, 19, 22, 29, 31, 37, 41, 45, 53, 54, 58, 60, 63, 76, 83, 94, 102, 109, 112, 116, 119, 124, 131, 152, 153, 158, 163, 168, 179, 184, 190, 194, 195, 196, 199, 201, 202, 208, 214, 222, 229, 230, 231, 232, 234, 250, and 251 were found to inhibit the in-situ IKKε-mediated phosphorylation of IRF3 with an IC₅₀ value ranging from about 250 nM to about 100 nM; and

Example Compounds 4, 38, 42, 43, 44, 46, 55, 56, 62, 71, 73, 75, 77, 78, 80, 82, 95, 103, 138, 140, 141, 143, 154, 157, 170, 187, 188, 189, 193, 200, 205, 206, 210, 212, 215, 220, 227, 244, 249, and 252 were found to inhibit the in-situ IKKε-mediated phosphorylation of IRF3 with an IC₅₀ value of less than about 100 nM.

Table 3, below, shows the specific in-situ IRF3 phosphorylation inhibitory activity of a subset of compounds according to Formulae I and/or II, as determined using the assay described above.

### ELISA to Detect Secreted RANTES

Prostate cancer DU145 cells were seeded at 20,000 cells/well in a 96-well tissue culture plate. The following day media was removed and replaced with complete media containing IKKε/TBK1 inhibitor (starting concentration 25 μM, 1:3 dilutions, final DMSO 0.05%). Cells were incubated for 20 hours and culture supernatant used to determine secreted RANTES levels using a commercially available ELISA kit (R & D Systems, Minneapolis, Minn.).

An alternative method was also developed to monitor Poly(I:C) (Sigma-Aldrich, St. Louis, Mo.) induced RANTES production in human fibroblast cells, MALME-3 (American Type Tissue Collection, Manassas, Va.). Cells were seeded at 2500 per well in a 96-well plate and the following day media was removed and replaced with complete media containing various concentrations of compound. One hour post-compound addition cells were treated with 100 ug/ml Poly(I:C) and the following day supernatant was collected and analyzed using the human RANTES ELISA kit as described above.

Using the assay described above for prostate cancer DU145 cells, Example Compounds 2, 11, 28 and 146 were found to inhibit the secretion of RANTES with an IC₅₀ ranging from about 60 nM to about 125 nM; and

Example Compounds 47, 48, 49, 111, and 141 were found to inhibito the secretion of RANTES with an IC₅₀ ranging from about 20 nM to about 60 nM.

Table 3, below, shows the specific in-situ RANTES secretion inhibitory activity of a subset of compounds according to Formulae I and/or II, as determined using the assay described for prostate cancer DU145 cells, above.

TABLE 3
Activities of a Subset of Compounds According to
Formulae I and/or II in Inhibiting the Kinase Activity of
IKKε In Vitro, IKKε-mediated Phosporylation of
IRF3 In Situ (i.e., In HEK293T Cells in Culture), and
RANTES Secretion by for prostate cancer DU145
cells in Culture.

| Example | | | RANTES |
|---|---|---|---|
| Compound | | pIRF3 ELISA | Secretion IC50 |
| No. | IKKε IC50 (nM) | IC50 (nM) | (nM) |
| 47 | 6.37 | 265 | 39.5 |
| 48 | 2.74 | 315 | 52.2 |
| 49 | 6.11 | 363 | 55.8 |
| 141 | 1.91 | 32.6 | 21.0 |

### Inhibition of RANTES and IP-10 Production by Human Fibroblast-Like Synoviocytes from Patients with Rheumatoid Arthritis

### Introduction

Rheumatoid arthritis (RA) synovial cells have upregulated IKKe, IRF3, RANTES, and IP-10 levels. IKKε knockout mice have moderately reduced arthritis and reduced levels of the above mentioned proteins. Treatment of human fibroblast like synoviocyte (HFLS) cells isolated from RA patients with Poly(I:C) mimics the diseased state of RA cells. If pretreatment of HFLS cells with compounds according to Formulae I and/or II were to inhibit production of RANTES and IP-10 chemokines in response to Poly(I:C) stimulation, such compounds would have therapeutic potential in treating patients with RA.

### Protocol:

HFLS cells (HFLS-RA) isolated from patients with rheumatoid arthritis are to be obtained from Cell Applications, Inc. (San Diego, Calif.). Cells are seeded in synoviocyte growth medium (Cell Applications, Inc., San Diego, Calif.) and are allowed to grow overnight. The following day, media is replaced and cells are treated with varying concentrations of selected compounds according to Formulae I and/or II (e.g., Example Compound 5) (0.1% final DMSO concentration). Two hours later, cells are induced with 50 μg/mL Poly(I:C) (Sigma-Aldrich, St. Louis, Mo.). Supernatants are collected 20 hours post-induction and used to monitor RANTES and IP-10 levels using DuoSet ELISA kits (Human CXCL10/IP-10 DuoSet & Human CCL5/RANTES DuoSet; R&D Systems, Inc., Minneapolis, Minn.).

### Results:

It is expected that pretreatment of HFLS cells with a compound according to Formulae I and/or II will inhibit production of RANTES and IP-10 chemokines from these cells using this assay.

### Identification of Genes Modulated by IKKε/TBK1 Inhibition in HFLS-RA Cells

### Introduction

IKKε and TBK1 play important roles in modulating several innate/adaptive immune and interferon-regulated genes in response to bacterial and viral infections. To identify genes that are under the control of IKKε and TBK1 kinase activity HFLS-RA cells (Cell Applications, Inc., San Diego, Calif.) can be pretreated with a compound according to Formulae I and/or II, and then treated with the TLR3 agonist Poly(I:C). A focused RT-PCR array containing either 84 innate/adaptive immune-regulated or 84 IFNα/β-regulated genes can probed by qRT-PCR using mRNA isolated from the treated cells, as well as from untreated control cells, according to the following protocol.

### Protocol:

HFLS cells isolated from patients with RA are to be obtained from Cell Applications, Inc. (HFLS-RA, Cell Applications, Inc., San Diego, Calif.). Cells are seeded in synoviocyte growth medium (Cell Applications, Inc., San Diego, Calif.) and allowed to grow overnight. The following day, media is replaced and cells were treated with 500 nM of a Compound according to Formulae I and/or II. Two hours later, cells are induced with 50 μg/mL Poly(I:C) (Sigma-Aldrich, St. Louis, Mo.). Cells are harvested 5 hours later and total RNA is isolated and processed using the RNeasy Mini Kit, QIAshredder and RNase-Free DNase Set (all from Qiagen, Inc., Valencia, Calif.). RNA is quantified using Quant-iT™ RiboGreen® RNA Assay Kit (Invitrogen, Inc., Carlsbad, Calif.). First strand cDNA is synthesized using RT² First Strand Kit (SABiosciences, Frederick, Md.). Real time PCR-based gene expression analysis is performed on the Human Innate & Adaptive Immune Responses (SABiosciences, Frederick, Md.) and the Human Interferon α/β Response Arrays (SABiosciences, Frederick, Md.) using the 7300 Real-Time PCR System (Applied Biosystems, Foster City, Calif.). To confirm gene modulation, TaqMan Gene Expression Assay probes CASP-1, IFN-β, IRF1, TLR3, MYD88, and GAPDH can be purchased from Applied Biosystems, Inc. (Foster City, Calif.) and run on the ABI-7300 Real-Time PCR System (Applied Biosystems, Inc., Foster City, Calif.).

### Conclusion:

It is expected that the induction of genes normally induced by Poly(I:C) treatment will be potently inhibited by pre-treatment with a compound according to Formulae I and/or II. If so, such inhibition of proinflammatory cytokine and chemokine production would suggest that the compounds according to Formulae I and/or II can used to treat, or lessen the symptoms of rheumatoid arthritis.

### Cell Growth Inhibition Assays

DU4475, COLO205, and OPM2 cells are to be plated in 96-well plates at 5000 cells/well. The following day test compounds (compounds according to Formulae I and/or II) are added, maintaining the final DMSO solvent concentration at 0.4%. After the desired incubation time (3-5 days), cell number are assayed using the CellTiter-Glo luminescent cell viability assay (Promega, Inc., Madison, Wis.). Viability is expressed as percent DMSO control after background subtraction.

Using the assays described above compounds according to Formulae I and/or II may be found to inhibit the growth of DU4475 cells.

### Glucose Uptake Assay Using Differentiated 3T3-L1 Adipocytes

Studies have demonstrated that IKKε knockout mice exhibit reduced weight gain and less complications associated with diabetes compared to wild type mice under high-fat diet conditions (Chiang et al.; The protein kinase IKKε regulates energy balance in obese mice; Cell, 138:961-975, 2009). To determine if IKKε/TBK1 inhibitors prevent fatty acid induced insulin resistance in 3T3-L1 adipocytes, insulin-stimulated glucose uptake in the presence of compounds according to Formulae I and/or II can be monitored.

Murine 3T3-L1 cells can be differentiated to adipocytes in 96-well plates by incubating for 2 days in adipogenic cocktail (10 ug/ml insulin, 115 ug/ml isobutylmethylxanthine, 1 uM dexamethasone) followed by incubation in insulin-supplemented medium for 2 days and complete media for an additional 5-10 days. Adipocytes can be treated with BSA-complexed palmitic acid and a compound according to Formulae I and/or II for 48 hours. Following free fatty acid treatment, adipoctyes were insulin-deprived in serum-free media for 2 hours. Subsequently, the media is replaced with KRH buffer containing a compound according to Formulae I and/or II and 300 nM insulin for 15-20 minutes. [¹⁴C]-labeled 2-deoxyglucose is then added for 15 minutes. Cells are then thoroughly washed with ice-cold PBS, and intracellular [¹⁴C]-2-deoxyglucose is measured in cell lysates by scintillation.

It is expected that in this cell culture model of obesity-induced insulin resistance, compounds according to Formulae I and/or II will be found to reverse the inhibitory effects of free fatty acid on insulin-stimulated glucose uptake. If so, these results would suggest that compounds according to Formulae I and/or II have the potential to alleviate obesity-mediated insulin resistance.

### Evaluation of Compounds According to Formulae I and/or II in a Collagen-Induced Arthritis Model in Mice

### Protocol

Male DBA/1 mice are injected with 150 μL of 2 mg/kg bovine type II collagen in Freund's complete adjuvant on days 0 and 21. On days 18 through 34, 100 mg/kg or 150 mg/kg of a compound according to Formulae I and/or II is to be administered orally each day. Also on days 18 through 34, all mouse paws are given a clinical score on a scale of 0-5, based upon the severity of erythema and swelling. Body weights are measured every other day beginning on day 18. Mice are euthanized on day 34, livers re weighed and paws frozen in preparation for subsequent histopathology evaluation.

### Results

In vehicle-treated, immunized mice, symptoms of arthritis have previously been shown to first appear on day 23 and can be present in all mice by day 27. It is expected that in mice treated with a compound according to Formulae I and/or II, that appearance of symptoms will be delayed. This drug-related delay should also be evident in the rate of increase in clinical score. Histopathological analysis of joints can also be conducted to confirm the activity of the compounds.

### Conclusions

It is expected that Compounds according to Formulae I and/or II will show significant, dose-dependent effects in reducing the collagen-induced arthritis in this mouse model. Both the rate of disease progression and magnitude of disease severity may inhibited. It is also expected that mice administered compounds according to Formulae I and/or II will loose less weight, consistent with a decreased severity of disease. Anti-type II collagen antibody titers can be measured in order to determine the extent to which the activity of a compound according to Formulae I and/or II is due to effects on inflamed joint tissues directly, or through possible reduction in antibody titer.

### IKKε/TBK1 Inhibition in RAW264.7 Mouse Cells Prevents Induction of RANTES and IFN-β after Treatment with Nucleic Acid Agonists

### Introduction

Mouse RAW264.7 macrophage-like cells can provide a model for macrophage function in tissue culture. To investigate the efficacy of compounds according to Formulae I and/or II in inhibiting nucleic acid cytosolic receptor pathways RAW264.7 cells can be pretreated with a compound according to Formulae I and/or II and can then exposed to various single stranded and double stranded RNA and DNA agonists introduced into the cell. To track IKKε/TBK1 signaling pathway activation, RANTES or IFN-β protein secretion can be monitored by ELISA-based assays (R & D systems), such as those described above.

### Protocol:

RAW264.7 cells are seeded in 96-well culture plates and allowed to grow overnight. The following day, media is replaced and cells are pretreated with a compound according to Formulae I and/or II (0.1% final DMSO concentration). After one hour cells are transfected with Lipofectime LTX reagent (Invitrogen, Carlsbad, Calif.) and one of the following agonists: low molecular weight Poly(I:C) (InvivoGen, San Diego, Calif.) at 10 μg/ml to activate RIG-I; high molecular weight Poly(I:C) (InvivoGen, San Diego, Calif.) at 10 μg/ml to activate MDA5; Poly(dA:dT) (InvivoGen, San Diego, Calif.) at 1 ug/ml; 45-basepair double stranded interferon stimulatory DNA oligo (ISD) at 10 μg/ml (Stetson and Medzhitov; Recognition of cytosolic DNA activates an IRF3-dependent innate immune response; Immunity, 24:93-103, 2006); ssDNA at 10 μg/ml (InvivoGen, San Diego, Calif.), ssRNA at 0.5 μg/ml (InvivoGen, San Diego, Calif.), or salmon sperm genomic DNA (gDNA) (InvivoGen, San Diego, Calif.) at 10 ug/ml to activate DAI, IFI16, and other cytosolic nucleic acid receptors. RANTES and IFN-β secretion are quantified using ELISA kits (Mouse CCL5/RANTES, R&D Systems, Inc., Minneapolis, Minn. and Mouse IFN-β, Thermo Fisher Scientific, Rockford, Ill.).

### Results:

The low molecular weight and high molecular weight poly(I:C) are expected to induce both RANTES and IFN-β protein secretion and that induction of secretion may be inhibited by treatment with a compound according to Formulae I and/or II. The double and single stranded DNA agonists; ISD, ssDNA, poly(dA:dT), and gDNA, can all potently induced RANTES and IFN-β secretion, and that induction of secretion may be inhibited by treatment with a compound according to Formulae I and/or II. The ssRNA agonist may also be expected to induce RANTES secretion, and that induction of secretion may potently inhibited by a compound according to Formulae I and/or II.

### Conclusion:

It is expected that the inhibition of IKKε and/or TBK1 with small molecule inhibitors will potently reduce secreted levels of IFN-β and RANTES after transfection of single or double stranded RNA and DNA molecules Inhibition of secretion of key proinflammatory cytokines, such as IFN-β and RANTES may be useful for the treatment of various autoimmune diseases as described above.

### Modulation of Agonist Induced Genes in Normal and SLE PBMCs

To determine if inhibition of IKKε and/or TBK1 can modulate nucleic acid agonist induced gene expression, high molecular weight poly(I:C) (MDA5 agonist) and low weight poly(I:C) (RIG-I agonist) can be electroporated into human peripheral blood mononuclear cells (PBMCs), that can be obtained from normal donors, or low molecular weight Poly(I:C) can be electroporated into PBMCs from donors that have Systemic Lupus Erythematosus (SLE). Induction of IFN-α2, IFN-β, and BLyS mRNA production can be monitored by qRT-PCR.

### Protocol

Human PBMCs are collected from healthy donors using routine laboratory procedures. PBMCs from SLE patients can be purchased from Astarte Biologics (Redmond, Wash.). The PBMCs can be electroporated using Nucleofector® Kit V (Lonza, Walkersville, Md.) with 0.4 ug/mL of high molecular weight poly (I:C) (InvivoGen, San Diego, Calif.) or 0.4 ug/mL low molecular weight poly (I:C) (InvivoGen, San Diego, Calif.) and seeded into wells containing serial dilutions of a compound according to Formulae I and/or II (0.1% final DMSO concentration). Cells are then harvested 4 hours post-electroporation and total RNA can be isolated and processed using RNeasy Mini Kit, QIAshredder, and RNase-Free DNase Set (all from Qiagen, Germantown, Md.). RNA can be quantitated using Quant-iT™ RiboGreen® RNA Assay Kit (Invitrogen, Carlsbad, Calif.). Reverse transcription and real-time PCR can be performed using the QuantiTect Probe RT-PCR Kit (Qiagen, Germantown, Md.) and the 7300 Real-Time PCR System (Applied Biosystems, Foster City, Calif.). Probe sets, IFN-α2, IFN-β1, BLyS, and GAPDH can be used for normalization, and can all purchased from Applied Biosystems, Inc (Carlsbad, Calif.).

### CONCLUSION

It is expected that PBMC samples from both normal and SLE patients would show robust induction of IFN-α2, IFN-β1, and BLyS mRNAs after LMW poly(I:C) agonist treatment. It is also expected that induction of IFN-α2, IFN-β1, and BLyS mRNAs would be potently inhibited by a compound according to Formulae I and/or II in a dose-dependent manner. Treatment of normal PBMCs with HMW poly(I:C) would be expected to show a similar response to the LMW studies. These results would suggest that activation of RIG-I and MDA5 receptors and IKKε/TBK1 pathway dependent induction of type I interferons (IFN-α2 and IFN-β1), as well as downstream interferon-signature genes (e.g. BLyS), can be dramatically reduced by treatment with a compound according to Formulae I and/or II. If so, these results would further suggest that compounds according to Formulae I and/or II can be used to limit flare ups and other complications in SLE patients arising from elevations in nucleic acid agonists.

All publications and patent applications mentioned in the specification are indicative of the level of those skilled in the art to which the present invention pertains. The mere mentioning of the publications and patent applications does not necessarily constitute an admission that they are prior art to the instant application.

Although the foregoing invention has been described in some detail by way of illustration and example for purposes of clarity of understanding, it will be clear to the skilled artisan that certain changes and modifications may be practiced within the scope of the appended claims.

What is claimed is:

1. A compound having a structure according to Formula I: 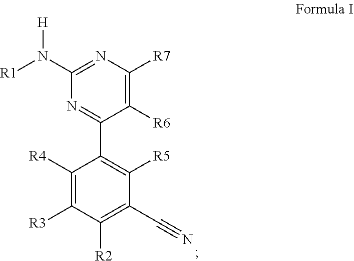
and pharmaceutically acceptable salts thereof, wherein:
R1 is optionally-substituted heteroaryl, optionally-substituted heterocyclyl; optionally-substituted heteroarylalkylene, optionally-substituted heterocycloalkylene, optionally-substituted heteroarylalkenylene, optionally-substituted heterocycloalkenylene, optionally-substituted heteroarylalkynylene, or optionally-substituted heterocycloalkynylene;
R2 is chosen from alkyl, alkenyl, alkynyl, carbocycle, cycloalkyl, cycloalkenyl, heterocyclyl, aryl, heteroaryl, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, alkyl-N-amido, cycloalkyl-N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide, wherein any of the foregoing groups are optionally substituted one or more times with alkyl, alkenyl, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide; and
R3, R4, R5, R6, and R7 are each independently chosen from alkyl, alkenyl, alkynyl, carbocycle, cycloalkyl, cyclo alkenyl, heterocyclyl, aryl, heteroaryl, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, alkyl-N-amido, cycloalkyl-N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide, wherein any of the foregoing groups are optionally substituted one or more times with alkyl, alkenyl, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide;
with the proviso that when R3, R4, R5, R6, and R7 are all hydro, then R2 is not heterocyclyl bonded to the phenyl ring through a nitrogen atom of the heterocyclyl; and
with the proviso that the compound is NOT:
Benzonitrile, 5-[2-(1H-benzimidazol-6-ylamino)-4-pyrimidinyl]-2-[(tetrahydro-2H-pyran-4-yl)oxy]-;
Benzonitrile, 5-[2-(1,3-benzodioxol-5-ylamino)-4-pyrimidinyl]-2-[(tetrahydro-2H-pyran-4-yl)oxy]-;
Benzonitrile, 5-[2-(6-benzothiazolylamino)-4-pyrimidinyl]-2-[(tetrahydro-2H-pyran-4-yl)oxy]-; or
Benzonitrile, 5-[2-(5-benzothiazolylamino)-4-pyrimidinyl]-2-[(tetrahydro-2H-pyran-4-yl)oxy]-.

2. The compound according to claim 1, wherein R1 is selected from heteroaryl, heterocyclo, heteroarylalkylene, heterocycloalkylene, heteroarylalkenylene, heterocycloalkenylene, heteroarylalkynylene, and heterocycloalkynylene, wherein any of the foregoing groups are optionally substituted one or more times with alkyl, alkenyl, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide.

3. The compound according to claim 1 or 2, wherein R1 is selected from heteroaryl and heterocyclyl; wherein either of the foregoing groups is optionally substituted one or more times with alkyl, alkenyl, alkynyl, carbocycle, cycloalkyl, cycloalkenyl, heterocycle, aryl, heteroaryl, halo, hydro, hydroxyl, alkoxy, alkynyloxy, cycloalkyloxy, heterocycloxy, aryloxy, heteroaryloxy, arylalkoxy, heteroarylalkoxy, mercapto, alkylthio, arylthio, arylalkyl, heteroarylalkyl, heteroarylalkenyl, arylalkynyl, haloalkyl, aldehyde, thiocarbonyl, heterocyclonoyl, O-carboxy, C-carboxy, carboxylic acid, ester, C-carboxy salt, carboxyalkyl, carboxyalkenylene, carboxyalkyl salt, carboxyalkoxy, carboxyalkoxyalkanoyl, amino, aminoalkyl, nitro, O-carbamyl, N-carbamyl, O-thiocarbamyl, N-thiocarbamyl, C-amido, N-amido, aminothiocarbonyl, hydroxyaminocarbonyl, alkoxyaminocarbonyl, cyano, nitrile, cyanato, isocyanato, thiocyanato, isothiocyanato, sulfinyl, sulfonyl, sulfonamide, aminosulfonyl, aminosulfonyloxy, sulfonamidecarbonyl, alkanoylaminosulfonyl, trihalomethylsulfonyl, or trihalomethylsulfonamide.

4. The compound according to any one of claims 1-3, wherein R3, R4, R5, R6, and R7 are each independently selected from hydro, halo, C₁₋₅ alkyl, nitro, cyano, C₁₋₅ alkoxy, C-amido, N-amido, C-carboxy, O-carboxy, sulfonamide, amino, hydroxyl, mercapto, alkylthio, sulfonyl, and sulfinyl.

5. The compound according to any one of claims 1-4, wherein R4, R5, R6, and R7 are each hydro.

6. The compound according to any one of claims 1-5, wherein R3 is hydro or methoxy.

7. A compound having a structure according to Formula II: 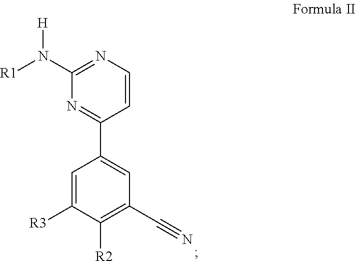
and pharmaceutically acceptable salts thereof, wherein
R1 is an optionally-substituted 5 or 6-membered heteroaryl group comprising from one to three heteroatoms independently chosen from nitrogen (N), oxygen (O), and sulfur (S);
R2 is chosen from an optionally substituted C₁₋₄ alkoxyl, heterocycloxyl, cycloalkylalkoxyl, heterocycloalkoxyl, C₁₋₄ alkyl-N-amido, or cycloalkyl-N-amido; and
R3 is hydro or methoxy.

8. The compound according to any one of claims 1-7, wherein R1 is an optionally substituted six-membered heteroaryl group comprising one or two nitrogens.

9. The compound according to claim 8, wherein the optionally substituted six-membered heteroaryl group is chosen from pyridyl, pyridazinyl, pyrimidinyl, or pyrazinyl.

10. The compound according to claim 8 or 9, wherein the optionally substituted six-membered heteroaryl group is 2-pyridyl, 3-pyridyl, 4-pyridyl, 3-pyridazinyl, 4-pyridazinyl, 2-pyrimidinyl, 4-pyrimidinyl, 5-pyrimidinyl, or 2-pyrazinyl.

11. The compound according to any one of claims 1-7, wherein R1 is an optionally substituted five-membered heteroaryl group comprising one, two, or three nitrogens.

12. The compound according to claim 11, wherein the optionally substituted five-membered heteroaryl group is chosen from pyrazolyl, imidazolyl, thienyl, oxazolyl, isoxazolyl, thiozolyl or triazolyl.

13. The compound according to claim 11 or 12, wherein the optionally substituted five-membered heteroaryl group is 4-pyrazolyl, 5-pyrazolyl, 4-imidazolyl, 5-imidazolyl, or 3-triazolyl.

14. The compound according to any one of claims 1-7, wherein R1 is an optionally substituted five-membered heteroaryl group comprising one, two, or three heteroatoms independently chosen from N, O and S.

15. The compound according to claim 14, wherein the optionally substituted five-membered heteroaryl group is chosen from thienyl, oxazolyl, isoxazolyl, or thiozolyl.

16. The compound according to claim 14 or 15, wherein the optionally substituted five-membered heteroaryl group is 2-thienyl, 2-oxazolyl, 5-isoxazolyl, or 2-thiozolyl.

17. The compound according to any one of claims 1-16, as applicable, wherein when the heteroaryl group or heterocyclyl group of R1 is substituted, then the substituent is chosen from: halo, methoxyl, ethoxyl, trihalomethyl, hydroxyl, hydroxylalkyl, C₁-C₄ alkyl, C-carboxyl, carbocyclyl, 4-6 membered heterocyclyl, heterocyclylalkyl, heterocyclylalkenyl, heterocyclonoyl, heterocyclonoylalkyl, heteroaryl, amino, aminoalkyl, N-amido, N-amidoalkyl, sulfamoylalkyl, C-amido, and N-amidoalkyl; wherein, where applicable, said substituent is optionally further substituted with halo, hydroxyl, hydroxylalkyl, methoxyl, ethoxyl, alkoxyalkoxyl, C₁-C₄ alkyl, hydroylated C₁-C₄ alkyl, amino, alkoxyamino, heterocyclyl, sulfonyl, hydroylated heterocyclyl, or aminated heterocyclyl group.

18. The compound according to any one of claims 1-17, wherein R1 is chosen from

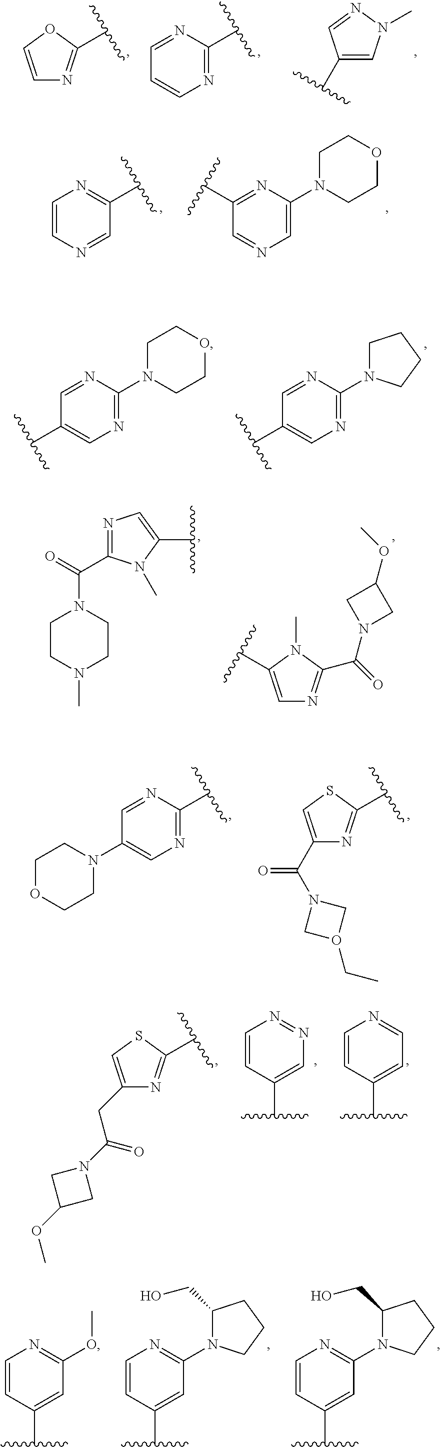

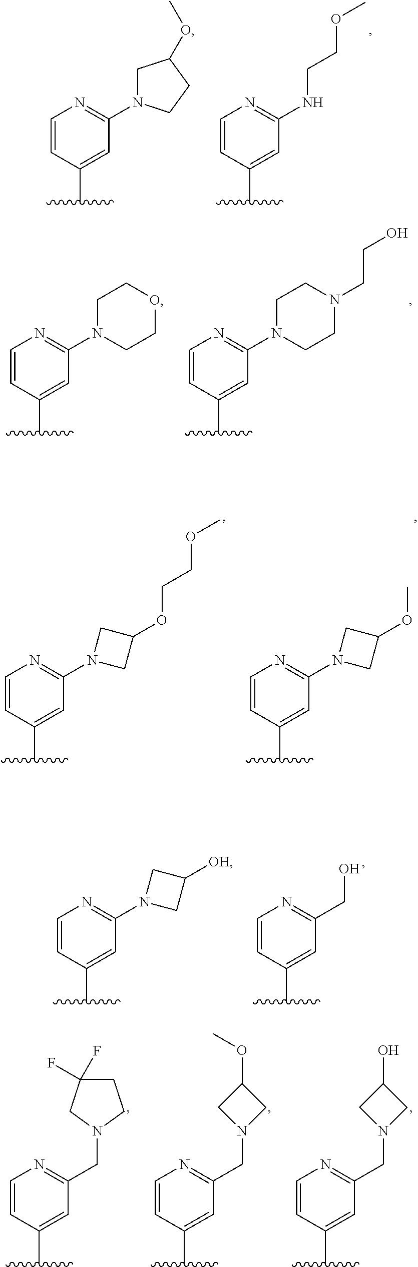

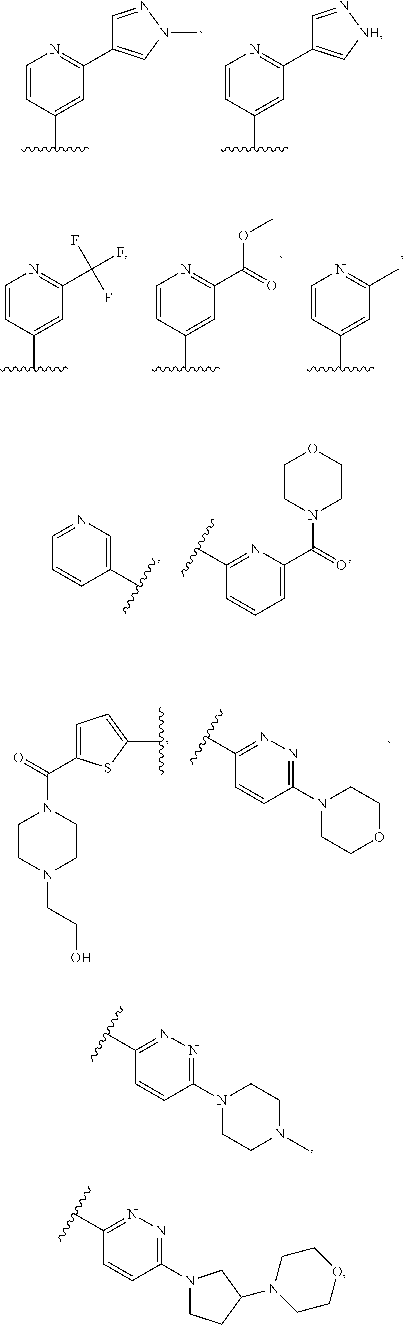

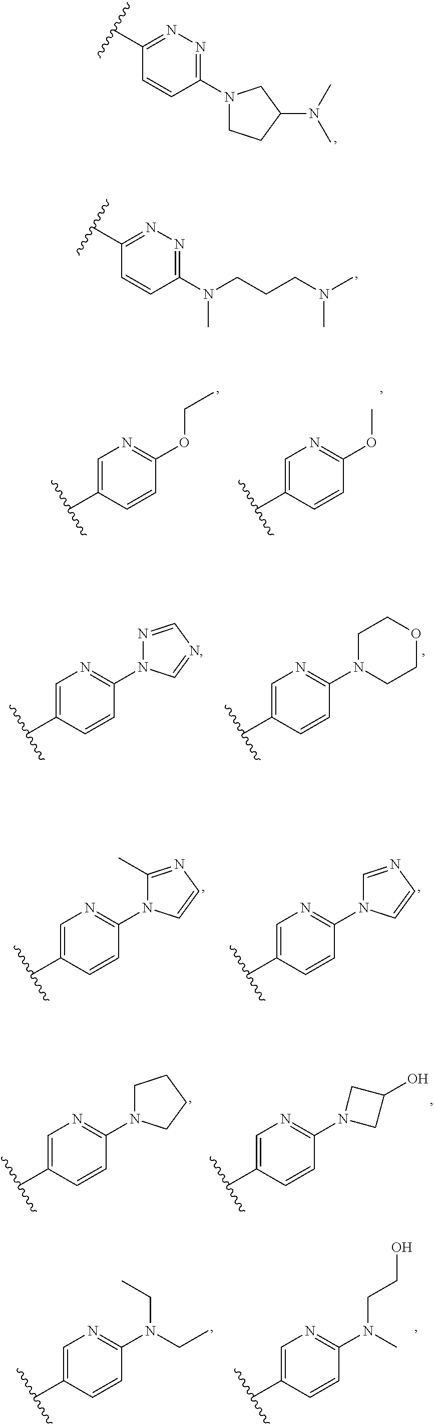

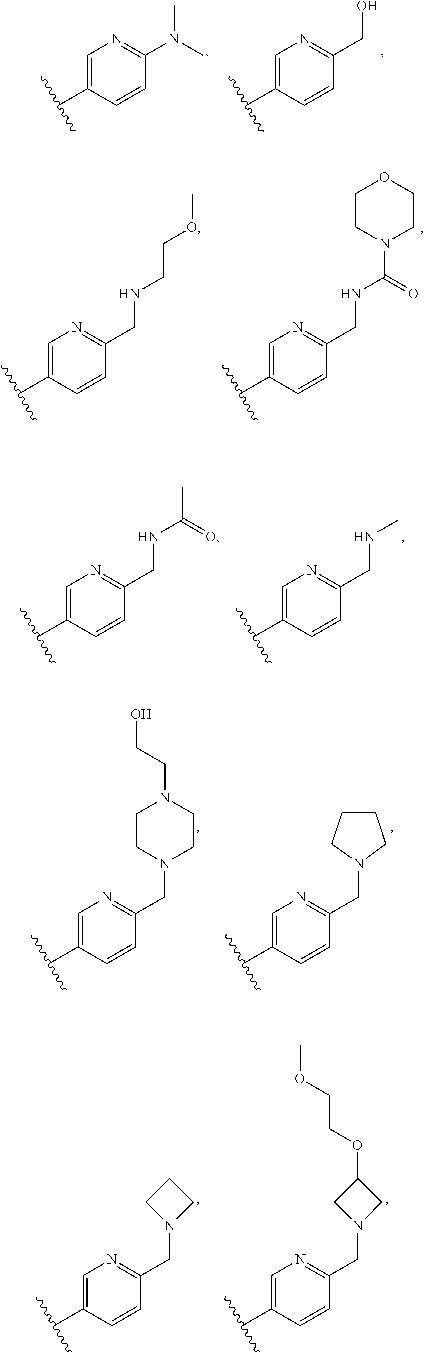

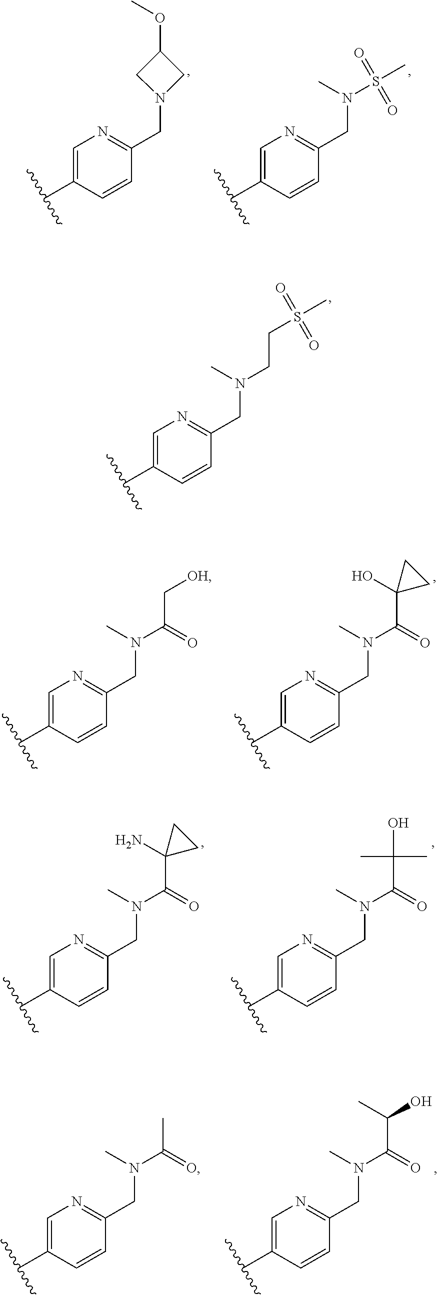

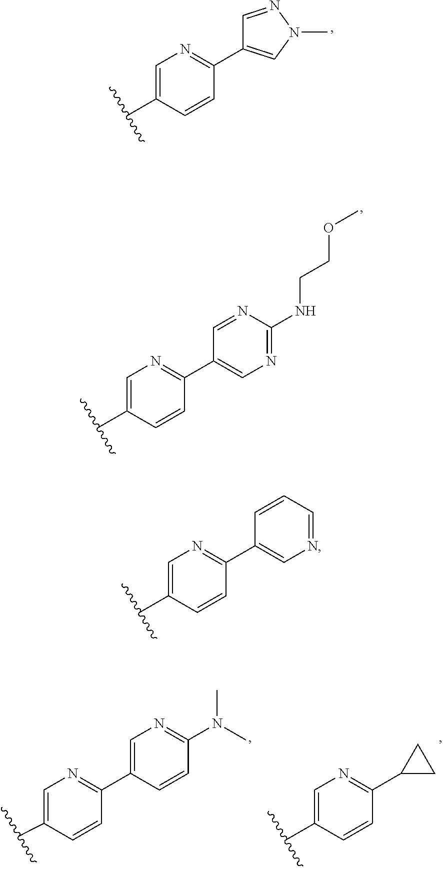 [SMILES: *c1ccc(-c2cnn(C)c2)nc1 ; *c1ccc(-c2cnc(NCCOC)nc2)nc1 ; *c1ccc(-c2cccnc2)nc1 ; *c1ccc(-c2ccc(N(C)C)nc2)nc1 ; *c1ccc(C2CC2)nc1]

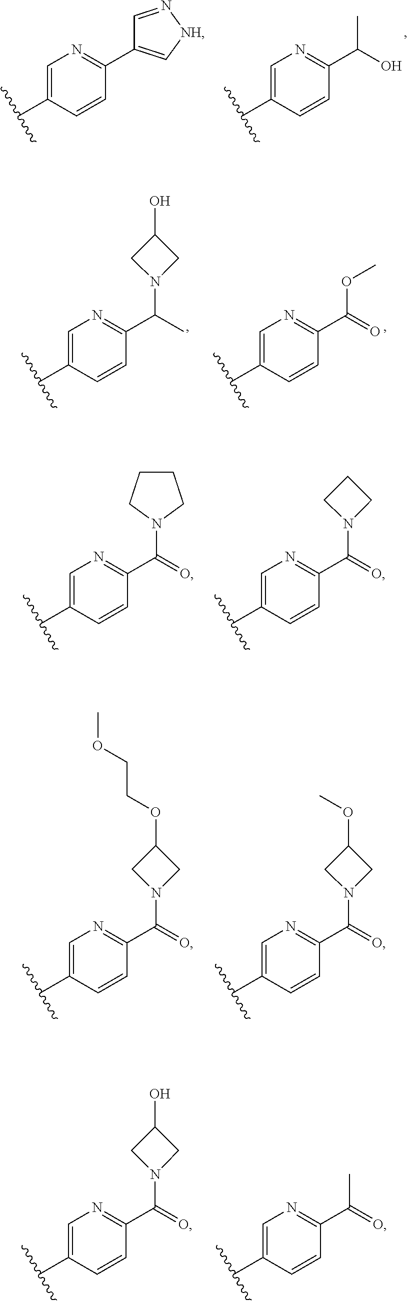

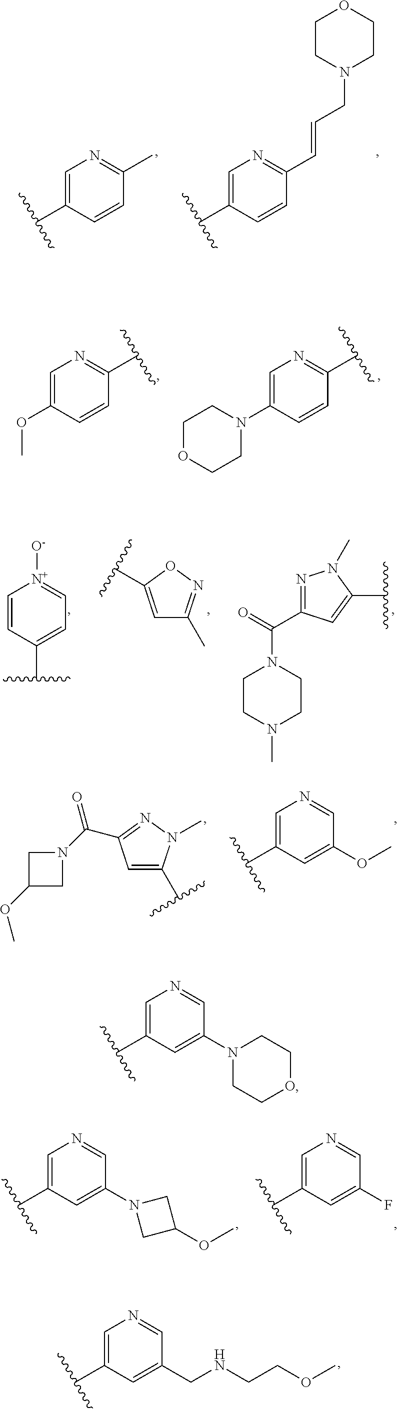

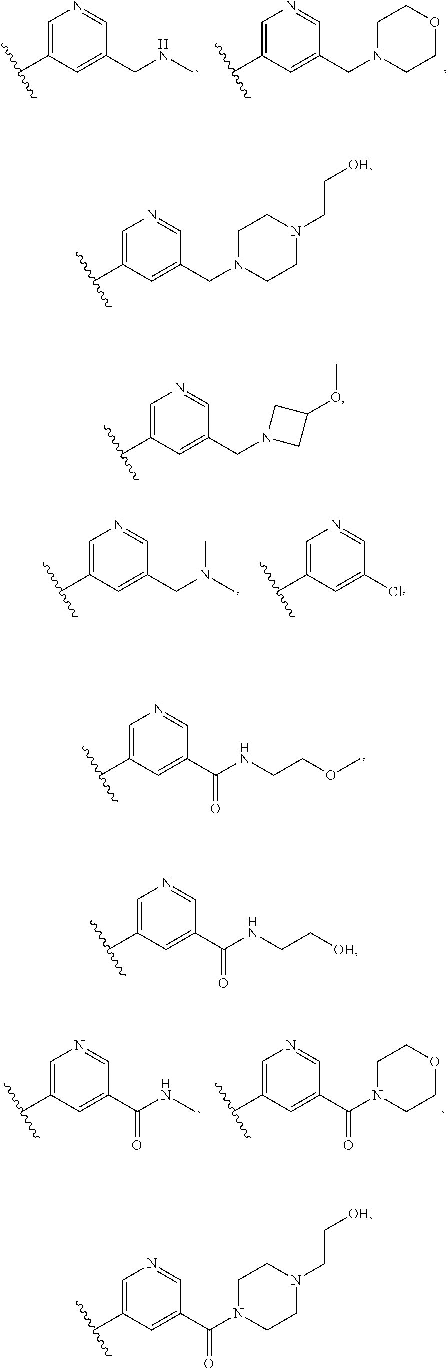

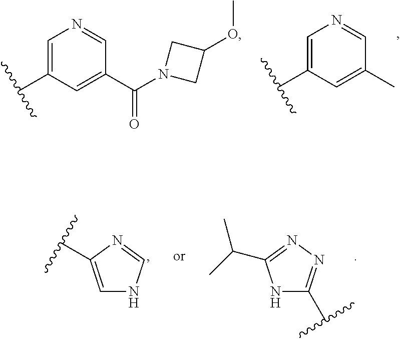

19. The compound according to any one of claims 1-18, wherein R2 is an optionally substituted tetrahydropyran-4-yloxyl, cyclopropanecarbonylamino, pyrrolidin-3-yloxyl, 2-methylpropanoylamino, 4-piperidyloxyl, cyclopropylmethoxyl, methoxyl, (3-methyloxetan-3-yl)methoxyl, isobutoxyl, or methyl group.

20. The compound according to any one of claims 1-19, wherein R2 is substituted pyrrolidin-3-yloxyl, or 4-piperidyloxyl, and the substitutent is 2-hydroxyethanoyl (2-hydroxyacetyl) or 2-hydroxypropanoyl, including stereoisomers (2R)-2-hydroxypropanoyl, and (2S)-2-hydroxypropanoyl.

21. The compound according to any one of claims 1-20, wherein R2 is chosen from

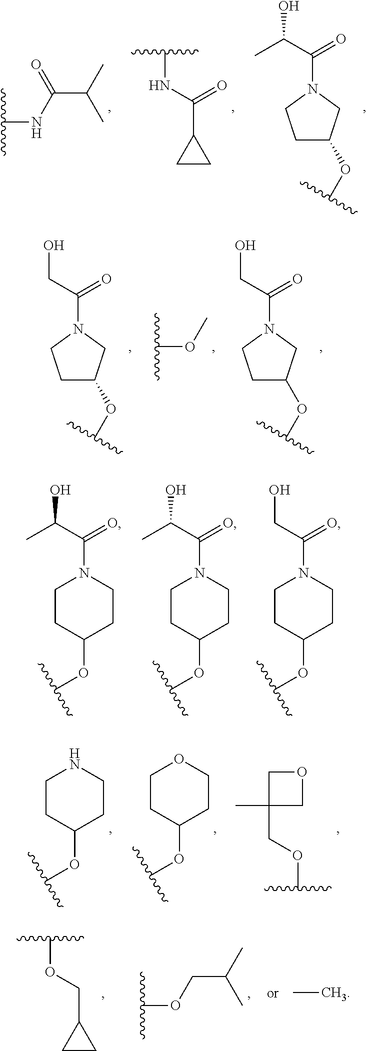

22. The compound according to any one of claims 1-21, wherein R3 is hydro.

23. The compound according to claim 1, wherein the compound according to Formula I is chosen from Table 2 or from:
5-[2-({6-[(2-Hydroxyethyl)(methyl)amino]pyridin-3-yl}amino)pyrimidin-4-yl]-2-(tetrahydro-2H-pyran-4-yloxy)benzonitrile;
5-(2-{[2-(Morpholin-4-yl)pyrimidin-5-yl]amino}pyrimidin-4-yl)-2-(tetrahydro-2H-pyran-4-yloxy)benzonitrile;
5-(2-{[2-(Pyrrolidin-1-yl)pyrimidin-5-yl]amino}pyrimidin-4-yl)-2-(tetrahydro-2H-pyran-4-yloxy)benzonitrile;
5-{2-[(6-Cyclopropylpyridin-3-yl)amino]pyrimidin-4-yl}-2-(tetrahydro-2H-pyran-4-yloxy)benzonitrile;
5-(2-{[6-(Pyrrolidin-1-yl)pyridin-3-yl]amino}pyrimidin-4-yl)-2-(tetrahydro-2H-pyran-4-yloxy)benzonitrile;
5-(2-{[6-(Morpholin-4-yl)pyridin-3-yl]amino}pyrimidin-4-yl)-2-(tetrahydro-2H-pyran-4-yloxy)benzonitrile;
5-{2-[(6-Methylpyridin-3-yl)amino]pyrimidin-4-yl}-2-(tetrahydro-2H-pyran-4-yloxy)benzonitrile;
5-{2-[(6-Ethoxypyridin-3-yl)amino]pyrimidin-4-yl}-2-(tetrahydro-2H-pyran-4-yloxy)benzonitrile;
5-(2-{[6-(Diethylamino)pyridin-3-yl]amino}pyrimidin-4-yl)-2-(tetrahydro-2H-pyran-4-yloxy)benzonitrile;
5-(2-{[6-(Dimethylamino)pyridin-3-yl]amino}pyrimidin-4-yl)-2-(tetrahydro-2H-pyran-4-yloxy)benzonitrile;
5-[2-(Pyridin-3-ylamino)pyrimidin-4-yl]-2-(tetrahydro-2H-pyran-4-yloxy)benzonitrile;
5-({-4-[3-Cyano-4-(tetrahydro-2H-pyran-4-yloxy)phenyl]pyrimidin-2-yl}amino)-N-methylpyridine-3-carboxamide;
5-({4-[3-Cyano-4-(tetrahydro-2H-pyran-4-yloxy)phenyl]pyrimidin-2-yl}amino)-N-(2-hydroxyethyl)pyridine-3-carboxamide;
5-{2-[(5-{[4-(2-Hydroxyethyl)piperazin-1-yl]carbonyl}pyridin-3-yl)amino]pyrimidin-4-yl}-2-(tetrahydro-2H-pyran-4-yloxy)benzonitrile;
5-({4-[3-Cyano-4-(tetrahydro-2H-pyran-4-yloxy)phenyl]pyrimidin-2-yl}amino)-N-(2-methoxyethyl)pyridine-3-carboxamide;
5-(2-{[5-(Morpholin-4-ylcarbonyl)pyridin-3-yl]amino}pyrimidin-4-yl)-2-(tetrahydro-2H-pyran-4-yloxy)benzonitrile;
5-[2-({5-[(3-Methoxyazetidin-1-yl)carbonyl]pyridin-3-yl}amino)pyrimidin-4-yl]-2-(tetrahydro-2H-pyran-4-yloxy)benzonitrile;
5-[2-({5-[(Methylamino)methyl]pyridin-3-yl}amino)pyrimidin-4-yl]-2-(tetrahydro-2H-pyran-4-yloxy)benzonitrile;
5-[2-({5-[(Dimethylamino)methyl]pyridin-3-yl}amino)pyrimidin-4-yl]-2-(tetrahydro-2H-pyran-4-yloxy)benzonitrile;
5-{2-[(5-{[4-(2-Hydroxyethyl)piperazin-1-yl]methyl}pyridin-3-yl)amino]pyrimidin-4-yl}-2-(tetrahydro-2H-pyran-4-yloxy)benzonitrile;
5-(2-{[5-(Morpholin-4-ylmethyl)pyridin-3-yl]amino}pyrimidin-4-yl)-2-(tetrahydro-2H-pyran-4-yloxy)benzonitrile;
5-{2-[(5-{[(2-Methoxyethyl)amino]methyl}pyridin-3-yl)amino]pyrimidin-4-yl}-2-(tetrahydro-2H-pyran-4-yloxy)benzonitrile;
5-[2-({5-[(3-Methoxyazetidin-1-yl)methyl]pyridin-3-yl}amino)pyrimidin-4-yl]-2-(tetrahydro-2H-pyran-4-yloxy)benzonitrile;
5-{2-[(5-Methylpyridin-3-yl)amino]pyrimidin-4-yl}-2-(tetrahydro-2H-pyran-4-yloxy)benzonitrile;
5-{2-[(5-Fluoropyridin-3-yl)amino]pyrimidin-4-yl}-2-(tetrahydro-2H-pyran-4-yloxy)benzonitrile;
5-{2-[(5-Chloropyridin-3-yl)amino]pyrimidin-4-yl}-2-(tetrahydro-2H-pyran-4-yloxy)benzonitrile;
5-{2-[(5-Methoxypyridin-3-yl)amino]pyrimidin-4-yl}-2-(tetrahydro-2H-pyran-4-yloxy)benzonitrile;
5-{2-[(6-{[3-(2-Methoxyethoxy)azetidin-1-yl]carbonyl}pyridin-3-yl)amino]pyrimidin-4-yl}-2-(tetrahydro-2H-pyran-4-yloxy)benzonitrile;
5-(2-{[6-(Pyrrolidin-1-ylcarbonyl)pyridin-3-yl]amino}pyrimidin-4-yl)-2-(tetrahydro-2H-pyran-4-yloxy)benzonitrile;
5-(2-{[6-(Azetidin-1-ylcarbonyl)pyridin-3-yl]amino}pyrimidin-4-yl)-2-(tetrahydro-2H-pyran-4-yloxy)benzonitrile;
5-[2-({6-[(3-Methoxyazetidin-1-yl)carbonyl]pyridin-3-yl}amino)pyrimidin-4-yl]-2-(tetrahydro-2H-pyran-4-yloxy)benzonitrile;
5-[2-({6-[(3-Hydroxyazetidin-1-yl)carbonyl]pyridin-3-yl}amino)pyrimidin-4-yl]-2-(tetrahydro-2H-pyran-4-yloxy)benzonitrile;
Methyl 5-({4-[3-cyano-4-(tetrahydro-2H-pyran-4-yloxy)phenyl]pyrimidin-2-yl}amino)pyridine-2-carboxylate;
5-[2-(Pyridin-4-ylamino)pyrimidin-4-yl]-2-(tetrahydro-2H-pyran-4-yloxy)benzonitrile;
5-{2-[(1-Oxidopyridin-4-yl)amino]pyrimidin-4-yl}-2-(tetrahydro-2H-pyran-4-yloxy)benzonitrile;
Methyl 4-({4-[3-cyano-4-(tetrahydro-2H-pyran-4-yloxy)phenyl]pyrimidin-2-yl}amino)pyridine-2-carboxylate;
5-[2-({2[(2R)-2-(Hydroxymethyl)pyrrolidin-1-yl]pyridin-4-yl}amino)pyrimidin-4-yl]-2-(tetrahydro-2H-pyran-4-yloxy)benzonitrile;
5-[2-({2-[(2-Methoxyethyl)amino]pyridin-4-yl}amino)pyrimidin-4-yl]-2-(tetrahydro-2H-pyran-4-yloxy)benzonitrile;
5-(2-{[2-(3-Methoxypyrrolidin-1-yl)pyridin-4-yl]amino}pyrimidin-4-yl)-2-(tetrahydro-2H-pyran-4-yloxy)benzonitrile;
5-[2-({2-[(2S)-2-(Hydroxymethyl)pyrrolidin-1-yl]pyridin-4-yl}amino)pyrimidin-4-yl]-2-(tetrahydro-2H-pyran-4-yloxy)benzonitrile;
5-(2-{[2-(3-Hydroxyazetidin-1-yl)pyridin-4-yl]amino}pyrimidin-4-yl)-2-(tetrahydro-2H-pyran-4-yloxy)benzonitrile;
5-[2-({2-[4-(2-Hydroxyethyl)piperazin-1-yl]pyridin-4-yl}amino)pyrimidin-4-yl]-2-(tetrahydro-2H-pyran-4-yloxy)benzonitrile;
5-(2-{[2-(3-Methoxyazetidin-1-yl)pyridin-4-yl]amino}pyrimidin-4-yl)-2-(tetrahydro-2H-pyran-4-yloxy)benzonitrile;
5-(2-{[2-(Morpholin-4-yl)pyridin-4-yl]amino}pyrimidin-4-yl)-2-(tetrahydro-2H-pyran-4-yloxy)benzonitrile;
5-[2-({2-[3-(2-Methoxyethoxy)azetidin-1-yl]pyridin-4-yl}amino)pyrimidin-4-yl]-2-(tetrahydro-2H-pyran-4-yloxy)benzonitrile;
5-(2-{[6-(3-Hydroxyazetidin-1-yl)pyridin-3-yl]amino}pyrimidin-4-yl)-2-(tetrahydro-2H-pyran-4-yloxy)benzonitrile;
5-(2-{[6-(Azetidin-1-ylmethyl)pyridin-3-yl]amino}pyrimidin-4-yl)-2-(tetrahydro-2H-pyran-4-yloxy)benzonitrile;
5-[2-({6-[(3-Methoxyazetidin-1-yl)methyl]pyridin-3-yl}amino)pyrimidin-4-yl]-2-(tetrahydro-2H-pyran-4-yloxy)benzonitrile;
5-{2-[(6-{[3-(2-Methoxyethoxy)azetidin-1-yl]methyl}pyridin-3-yl)amino]pyrimidin-4-yl}-2-(tetrahydro-2H-pyran-4-yloxy)benzonitrile;
5-{2-[(6-{[(2-Methoxyethyl)amino]methyl}pyridin-3-yl)amino]pyrimidin-4-yl}-2-(tetrahydro-2H-pyran-4-yloxy)benzonitrile;
5-(2-{[6-(Pyrrolidin-1-ylmethyl)pyridin-3-yl]amino}pyrimidin-4-yl)-2-(tetrahydro-2H-pyran-4-yloxy)benzonitrile;
5-[2-({6-[(Methylamino)methyl]pyridin-3-yl}amino)pyrimidin-4-yl]-2-(tetrahydro-2H-pyran-4-yloxy)benzonitrile;
N-{[5-({4-[3-Cyano-4-(tetrahydro-2H-pyran-4-yloxy)phenyl]pyrimidin-2-yl}amino)pyridin-2-yl]methyl}-N-methylacetamide;
5-(2-{[6-({Methyl[2-(methylsulfonyl)ethyl]amino}methyl)pyridin-3-yl]amino}pyrimidin-4-yl)-2-(tetrahydro-2H-pyran-4-yloxy)benzonitrile;
N-{[5-({4-[3-Cyano-4-(tetrahydro-2H-pyran-4-yloxy)phenyl]pyrimidin-2-yl}amino)pyridin-2-yl]methyl}-N-methylmethanesulfonamide;
N-{[5-({4-[3-Cyano-4-(tetrahydro-2H-pyran-4-yloxy)phenyl]pyrimidin-2-yl}amino)pyridin-2-yl]methyl}-2-hydroxy-N,2-dimethylpropanamide;
(2R)—N-{[5-({4-[3-Cyano-4-(tetrahydro-2H-pyran-4-yloxy)phenyl]pyrimidin-2-yl}amino)pyridin-2-yl]methyl}-2-hydroxy-N-methylpropanamide;
N-{[5-({4-[3-Cyano-4-(tetrahydro-2H-pyran-4-yloxy)phenyl]pyrimidin-2-yl}amino)pyridin-2-yl]methyl}-2-hydroxy-N-methylacetamide;
N-{[5-({4-[3-Cyano-4-(tetrahydro-2H-pyran-4-yloxy)phenyl]pyrimidin-2-yl}amino)pyridin-2-yl]methyl}-1-hydroxy-N-methylcyclopropanecarboxamide;
1-Amino-N-{[5-({4-[3-cyano-4-(tetrahydro-2H-pyran-4-yloxy)phenyl]pyrimidin-2-yl}amino)pyridin-2-yl]methyl}-N-methylcyclopropanecarboxamide;
5-(2-{[6-(1-Hydroxyethyl)pyridin-3-yl]amino}pyrimidin-4-yl)-2-(tetrahydro-2H-pyran-4-yloxy)benzonitrile;
5-{2-[(6-Acetylpyridin-3-yl)amino]pyrimidin-4-yl}-2-(tetrahydro-2H-pyran-4-yloxy)benzonitrile;
5-[2-({6-[1-(3-Hydroxyazetidin-1-yl)ethyl]pyridin-3-yl}amino)pyrimidin-4-yl]-2-(tetrahydro-2H-pyran-4-yloxy)benzonitrile;
5-(2-{[5-(3-Methoxyazetidin-1-yl)pyridin-3-yl]amino}pyrimidin-4-yl)-2-(tetrahydro-2H-pyran-4-yloxy)benzonitrile;
5-(2-{[5-(Morpholin-4-yl)pyridin-3-yl]amino}pyrimidin-4-yl)-2-(tetrahydro-2H-pyran-4-yloxy)benzonitrile;
5-(2-{[2-(Hydroxymethyl)pyridin-4-yl]amino}pyrimidin-4-yl)-2-(tetrahydro-2H-pyran-4-yloxy)benzonitrile;
N-{[5-({4-[3-Cyano-4-(tetrahydro-2H-pyran-4-yloxy)phenyl]pyrimidin-2-yl}amino)pyridin-2-yl]methyl}morpholine-4-carboxamide;
N-{[5-({4-[3-Cyano-4-(tetrahydro-2H-pyran-4-yloxy)phenyl]pyrimidin-2-yl}amino)pyridin-2-yl]methyl}acetamide;
5-[2-({2-[(3-Methoxyazetidin-1-yl)methyl]pyridin-4-yl}amino)pyrimidin-4-yl]-2-(tetrahydro-2H-pyran-4-yloxy)benzonitrile;
5-[2-({2-[(3-Hydroxyazetidin-1-yl)methyl]pyridin-4-yl}amino)pyrimidin-4-yl]-2-(tetrahydro-2H-pyran-4-yloxy)benzonitrile;
5-[2-({-[(3,3-Difluoropyrrolidin-1-yl)methyl]pyridin-4-yl}amino)pyrimidin-4-yl]-2-(tetrahydro-2H-pyran-4-yloxy)benzonitrile;
5-(2-{[6-(Morpholin-4-ylcarbonyl)pyridin-2-yl]amino}pyrimidin-4-yl)-2-(tetrahydro-2H-pyran-4-yloxy)benzonitrile;
5-(2-{[6-(2-Methyl-1H-imidazol-1-yl)pyridin-3-yl]amino}pyrimidin-4-yl)-2-(tetrahydro-2H-pyran-4-yloxy)benzonitrile;
5-(2-{[5-(Morpholin-4-yl)pyrimidin-2-yl]amino}pyrimidin-4-yl)-2-(tetrahydro-2H-pyran-4-yloxy)benzonitrile;
5-(2-{[6-(1H-Imidazol-1-yl)pyridin-3-yl]amino}pyrimidin-4-yl)-2-(tetrahydro-2H-pyran-4-yloxy)benzonitrile;
2-(Tetrahydro-2H-pyran-4-yloxy)-5-(2-{[6-(1H-1,2,4-triazol-1-yl)pyridin-3-yl]amino}pyrimidin-4-yl)benzonitrile;
5-(2-{[6-(1-Methyl-1H-pyrazol-4-yl)pyridin-3-yl]amino}pyrimidin-4-yl)-2-(tetrahydro-2H-pyran-4-yloxy)benzonitrile;
5-(2-{[6-(1H-Pyrazol-4-yl)pyridin-3-yl]amino}pyrimidin-4-yl)-2-(tetrahydro-2H-pyran-4-yloxy)benzonitrile;
5-[2-(2,3′-Bipyridin-5-ylamino)pyrimidin-4-yl]-2-(tetrahydro-2H-pyran-4-yloxy)benzonitrile;
5-{2-[(6-{2-[(2-Methoxyethyl)amino]pyrimidin-5-yl}pyridin-3-yl)amino]pyrimidin-4-yl}-2-(tetrahydro-2H-pyran-4-yloxy)benzonitrile;
5-(2-{[6′-(Dimethylamino)-2,3′-bipyridin-5-yl]amino}pyrimidin-4-yl)-2-(tetrahydro-2H-pyran-4-yloxy)benzonitrile;
5-(2-{[2-(1-Methyl-1H-pyrazol-4-yl)pyridin-4-yl]amino}pyrimidin-4-yl)-2-(tetrahydro-2H-pyran-4-yloxy)benzonitrile;
5-(2-{[2-(1H-Pyrazol-4-yl)pyridin-4-yl]amino}pyrimidin-4-yl)-2-(tetrahydro-2H-pyran-4-yloxy)benzonitrile;
5-[2-({6-[3-(Morpholin-4-yl)pyrrolidin-1-yl]pyridazin-3-yl}amino)pyrimidin-4-yl]-2-(tetrahydro-2H-pyran-4-yloxy)benzonitrile;
5-(2-{[6-(4-Methylpiperazin-1-yl)pyridazin-3-yl]amino}pyrimidin-4-yl)-2-(tetrahydro-2H-pyran-4-yloxy)benzonitrile;
5-{2-[(6-{[3-(Dimethylamino)propyl](methyl)amino}pyridazin-3-yl)amino]pyrimidin-4-yl}-2-(tetrahydro-2H-pyran-4-yloxy)benzonitrile;
5-[2-({6-[3-(Dimethylamino)pyrrolidin-1-yl]pyridazin-3-yl}amino)pyrimidin-4-yl]-2-(tetrahydro-2H-pyran-4-yloxy)benzonitrile;
5-(2-{[6-(Morpholin-4-yl)pyridazin-3-yl]amino}pyrimidin-4-yl)-2-(tetrahydro-2H-pyran-4-yloxy)benzonitrile;
5-(2-{[6-(Morpholin-4-yl)pyrazin-2-yl]amino}pyrimidin-4-yl)-2-(tetrahydro-2H-pyran-4-yloxy)benzonitrile;
5-[2-(Pyrimidin-2-ylamino)pyrimidin-4-yl]-2-(tetrahydro-2H-pyran-4-yloxy)benzonitrile;
5-[2-(Pyridazin-4-ylamino)pyrimidin-4-yl]-2-(tetrahydro-2H-pyran-4-yloxy)benzonitrile;
5-[2-(Pyrazin-2-ylamino)pyrimidin-4-yl]-2-(tetrahydro-2H-pyran-4-yloxy)benzonitrile;
5-(2-{[5-(Morpholin-4-yl)pyridin-2-yl]amino}pyrimidin-4-yl)-2-(tetrahydro-2H-pyran-4-yloxy)benzonitrile;
5-[2-({6-[(1E)-3-(Morpholin-4-yl)prop-1-en-1-yl]pyridin-3-yl}amino)pyrimidin-4-yl]-2-(tetrahydro-2H-pyran-4-yloxy)benzonitrile;
5-{2-[(5-{[4-(2-Hydroxyethyl)piperazin-1-yl]carbonyl}thiophen-2-yl)amino]pyrimidin-4-yl}-2-(tetrahydro-2H-pyran-4-yloxy)benzonitrile;
5-{2-[(3-Methyl-1,2-oxazol-5-yl)amino]pyrimidin-4-yl}-2-(tetrahydro-2H-pyran-4-yloxy)benzonitrile;
5-{2-[(1-Methyl-1H-pyrazol-4-yl)amino]pyrimidin-4-yl}-2-(tetrahydro-2H-pyran-4-yloxy)benzonitrile;
5-[2-(1,3-oxazol-2-ylamino)pyrimidin-4-yl]-2-(tetrahydro-2H-pyran-4-yloxy)benzonitrile;
5-[2-(1H-Imidazol-4-ylamino)pyrimidin-4-yl]-2-(tetrahydro-2H-pyran-4-yloxy)benzonitrile;
5-[2-({3-[(3-Methoxyazetidin-1-yl)carbonyl]-1-methyl-1H-pyrazol-5-yl}amino)pyrimidin-4-yl]-2-(tetrahydro-2H-pyran-4-yloxy)benzonitrile;
5-[2-({1-Methyl-3-[(4-methylpiperazin-1-yl)carbonyl]-1H-pyrazol-5-yl}amino)pyrimidin-4-yl]-2-(tetrahydro-2H-pyran-4-yloxy)benzonitrile;
5-[2-({2-[(3-Methoxyazetidin-1-yl)carbonyl]-1-methyl-1H-imidazol-5-yl}amino)pyrimidin-4-yl]-2-(tetrahydro-2H-pyran-4-yloxy)benzonitrile;
5-[2-({1-Methyl-2-[(4-methylpiperazin-1-yl)carbonyl]-1H-imidazol-5-yl}amino)pyrimidin-4-yl]-2-(tetrahydro-2H-pyran-4-yloxy)benzonitrile;
5-[2-({4-[(3-Methoxyazetidin-1-yl)carbonyl]-1,3-thiazol-2-yl}amino)pyrimidin-4-yl]-2-(tetrahydro-2H-pyran-4-yloxy)benzonitrile;
5-[2-({4-[2-(3-Methoxyazetidin-1-yl)-2-oxoethyl]-1,3-thiazol-2-yl}amino)pyrimidin-4-yl]-2-(tetrahydro-2H-pyran-4-yloxy)benzonitrile;
2-Methoxy-5-{2-[(2-methoxypyridin-4-yl)amino]pyrimidin-4-yl}benzonitrile;
2-Methoxy-5-{2-[(5-methoxypyridin-2-yl)amino]pyrimidin-4-yl}benzonitrile;
2-Methoxy-5-{2-[(6-methoxypyridin-3-yl)amino]pyrimidin-4-yl}benzonitrile;
5-[2-({6-[(2-Hydroxyethyl)(methyl)amino]pyridin-3-yl}amino)pyrimidin-4-yl]-2-(2-methylpropoxy)benzonitrile;
2-(Cyclopropylmethoxy)-5-{2-[(6-ethoxypyridin-3-yl)amino]pyrimidin-4-yl}benzonitrile;
2-(Cyclopropylmethoxy)-5-[2-({6-[(2-hydroxyethyl)(methyl)amino]pyridin-3-yl}amino)pyrimidin-4-yl]benzonitrile;
2-(Cyclopropylmethoxy)-5-{2-[(6-methylpyridin-3-yl)amino]pyrimidin-4-yl}benzonitrile;
2-[(3-Methyloxetan-3-yl)methoxy]-5-{2-[(6-methylpyridin-3-yl)amino]pyrimidin-4-yl}benzonitrile;
3-Methoxy-5-{2-[(6-methylpyridin-3-yl)amino]pyrimidin-4-yl}-2-(tetrahydro-2H-pyran-4-yloxy)benzonitrile;
5-(2-{[6-(Azetidin-1-ylmethyl)pyridin-3-yl]amino}pyrimidin-4-yl)-3-methoxy-2-(tetrahydro-2H-pyran-4-yloxy)benzonitrile;
3-Methoxy-5-[2-({6-[(3-methoxyazetidin-1-yl)methyl]pyridin-3-yl}amino)pyrimidin-4-yl]-2-(tetrahydro-2H-pyran-4-yloxy)benzonitrile;
5-(2-{[6-(Diethylamino)pyridin-3-yl]amino}pyrimidin-4-yl)-2-{[(3R)-1-(hydroxyacetyl)pyrrolidin-3-yl]oxy}benzonitrile;
2-{[(3R)-1-(Hydroxyacetyl)pyrrolidin-3-yl]oxy}-5-{2-[(6-methylpyridin-3-yl)amino]pyrimidin-4-yl}benzonitrile;
2-{[(3R)-1-(Hydroxyacetyl)pyrrolidin-3-yl]oxy}-5-(2-{[6-(morpholin-4-yl)pyridin-3-yl]amino}pyrimidin-4-yl)benzonitrile;
2-({(3R)-1-[(2S)-2-Hydroxypropanoyl]pyrrolidin-3-yl}oxy)-5-{2-[(6-methylpyridin-3-yl)amino]pyrimidin-4-yl}benzonitrile;
2-{[(3R)-1-(Hydroxyacetyl)pyrrolidin-3-yl]oxy}-5-{2-[(2-methoxypyridin-4-yl)amino]pyrimidin-4-yl}benzonitrile;
2-{[(3R)-1-(Hydroxyacetyl)pyrrolidin-3-yl]oxy}-5-(2-{[2-(trifluoromethyl)pyridin-4-yl]amino}pyrimidin-4-yl)benzonitrile;
2-{[(3R)-1-(Hydroxyacetyl)pyrrolidin-3-yl]oxy}-5-{2-[(2-methylpyridin-4-yl)amino]pyrimidin-4-yl}benzonitrile;
5-(2-{[6-(Dimethylamino)pyridin-3-yl]amino}pyrimidin-4-yl)-2-{[1-(hydroxyacetyl)pyrrolidin-3-yl]oxy}benzonitrile;
2-{[(3R)-1-(Hydroxyacetyl)pyrrolidin-3-yl]oxy}-5-[2-({6-[(3-methoxyazetidin-1-yl)methyl]pyridin-3-yl}amino)pyrimidin-4-yl]benzonitrile;
2-{[(3R)-1-(Hydroxyacetyl)pyrrolidin-3-yl]oxy}-5-(2-{[2-(1-methyl-1H-pyrazol-4-yl)pyridin-4-yl]amino}pyrimidin-4-yl)benzonitrile;
2-({1-[(2S)-2-Hydroxypropanoyl]piperidin-4-yl}oxy)-5-(2-{[6-(morpholin-4-yl)pyridin-3-yl]amino}pyrimidin-4-yl)benzonitrile;
2-({1-[(2S)-2-Hydroxypropanoyl]piperidin-4-yl}oxy)-5-{2-[(6-methylpyridin-3-yl)amino]pyrimidin-4-yl}benzonitrile;
2-({1-[(2R)-2-Hydroxypropanoyl]piperidin-4-yl}oxy)-5-{2-[(6-methylpyridin-3-yl)amino]pyrimidin-4-yl}benzonitrile;
2-{[1-(Hydroxyacetyl)piperidin-4-yl]oxy}-5-{2-[(6-methylpyridin-3-yl)amino]pyrimidin-4-yl}benzonitrile;
2-{[1-(Hydroxyacetyl)piperidin-4-yl]oxy}-5-{2-[(2-methoxypyridin-4-yl)amino]pyrimidin-4-yl}benzonitrile;
N-[2-Cyano-4-(2-{[2-(pyrrolidin-1-yl)pyrimidin-5-yl]amino}pyrimidin-4-yl)phenyl]-2-methylpropanamide;
N-{2-Cyano-4-[2-({6-[(2-hydroxyethyl)(methyl)amino]pyridin-3-yl}amino)pyrimidin-4-yl]phenyl}-2-methylpropanamide;
N-[2-Cyano-4-(2-{[6-(morpholin-4-yl)pyridin-3-yl]amino}pyrimidin-4-yl)phenyl]-2-methylpropanamide;
N-[2-Cyano-4-(2-{[6-(pyrrolidin-1-yl)pyridin-3-yl]amino}pyrimidin-4-yl)phenyl]-2-methylpropanamide;
N-(2-Cyano-4-{2-[(6-methylpyridin-3-yl)amino]pyrimidin-4-yl}phenyl)-2-methylpropanamide;
N-(2-Cyano-4-{2-[(6-ethoxypyridin-3-yl)amino]pyrimidin-4-yl}phenyl)-2-methylpropanamide;
N-[2-Cyano-4-(2-{[6-(dimethylamino)pyridin-3-yl]amino}pyrimidin-4-yl)phenyl]cyclopropanecarboxamide;
N-[2-Cyano-4-(2-{[6-(diethylamino)pyridin-3-yl]amino}pyrimidin-4-yl)phenyl]cyclopropanecarboxamide;
N-(2-Cyano-4-{2-[(6-cyclopropylpyridin-3-yl)amino]pyrimidin-4-yl}phenyl)cyclopropanecarboxamide;
N-[2-Cyano-4-(2-{[6-(morpholin-4-yl)pyridin-3-yl]amino}pyrimidin-4-yl)phenyl]cyclopropanecarboxamide;
N-[2-Cyano-4-(2-{[2-(3-methoxyazetidin-1-yl)pyridin-4-yl]amino}pyrimidin-4-yl)phenyl]cyclopropanecarboxamide;
N-{2-Cyano-4-[2-({2-[4-(2-hydroxyethyl)piperazin-1-yl]pyridin-4-yl}amino)pyrimidin-4-yl]phenyl}cyclopropanecarboxamide;
N-[2-Cyano-4-(2-{[6-(hydroxymethyl)pyridin-3-yl]amino}pyrimidin-4-yl)phenyl]cyclopropanecarboxamide;
N-(2-Cyano-4-{2-[(6-{[4-(2-hydroxyethyl)piperazin-1-yl]methyl}pyridin-3-yl)amino]pyrimidin-4-yl}phenyl)cyclopropanecarboxamide;
N-{2-Cyano-4-[2-({6-[(3-methoxyazetidin-1-yl)methyl]pyridin-3-yl}amino)pyrimidin-4-yl]phenyl}cyclopropanecarboxamide;
N-(2-Cyano-4-{2-[(6-{[3-(2-methoxyethoxy)azetidin-1-yl]methyl}pyridin-3-yl)amino]pyrimidin-4-yl}phenyl)cyclopropanecarboxamide;
N-(2-Cyano-4-{2-[(6-{[(2-methoxyethyl)amino]methyl}pyridin-3-yl)amino]pyrimidin-4-yl}phenyl)cyclopropanecarboxamide;
N-(2-Cyano-4-{2-[(6-methylpyridin-3-yl)amino]pyrimidin-4-yl}phenyl)cyclopropanecarboxamide; or
5-(2-{[3-(Propan-2-yl)-1H-1,2,4-triazol-5-yl]amino}pyrimidin-4-yl)-2-(tetrahydro-2H-pyran-4-yloxy)benzonitrile.

24. A pharmaceutical composition comprising at least one compound of any one of claims 1-23 and a pharmaceutically acceptable vehicle.

25. A method of treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, in a human patient, comprising administering a therapeutically effective amount of a compound of claims 1-23, or pharmaceutical composition of claim 24, to said patient.

26. The method of claim 25, further comprising identifying a patient in need of such treatment prior to the administering step.

27. A method of treating inflammation, comprising administering a therapeutically effective amount of a compound of any one of claims 1-23, or pharmaceutical composition of claim 24, to said patient.

28. A method of treating RA, administering a therapeutically effective amount of a compound of any one of claims 1-23, or pharmaceutical composition of claim 24, to said patient.

29. A method of treating SLE, administering a therapeutically effective amount of a compound of any one of claims 1-23, or pharmaceutical composition of claim 24, to said patient.

30. A method of treating a disease associated with aberrant accumulation of cytosolic nucleic acids, comprising administering a therapeutically effective amount of a compound of any one of claims 1-23, or pharmaceutical composition of claim 24, to said patient.

31. A method of treating Sjögrens syndrome, comprising administering a therapeutically effective amount of a compound of any one of claims 1-23, or pharmaceutical composition of claim 24, to said patient.

32. A method of treating Aicardi-Goutières syndrome, comprising administering a therapeutically effective amount of a compound of any one of claims 1-23, or pharmaceutical composition of claim 24, to said patient.

33. A method of treating a subtype of lupus associated with aberrant accumulation of cytosolic nucleic acids, comprising administering a therapeutically effective amount of a compound of any one of claims 1-23, or pharmaceutical composition of claim 24, to said patient.

34. A method of treating chilblain lupus, comprising administering a therapeutically effective amount of a compound of any one of claims 1-23, or pharmaceutical composition of claim 24, to said patient.

35. A method of treating RVCL, comprising administering a therapeutically effective amount of a compound of any one of claims 1-23, or pharmaceutical composition of claim 24, to said patient.

36. A method of treating systemic sclerosis, comprising administering a therapeutically effective amount of a compound of any one of claims 1-23, or pharmaceutical composition of claim 24, to said patient.

37. A method of treating myositis, comprising administering a therapeutically effective amount of a compound of any one of claims 1-23, or pharmaceutical composition of claim 24, to said patient.

38. A method of treating dermatomyositis, comprising administering a therapeutically effective amount of a compound of any one of claims 1-23, or pharmaceutical composition of claim 24, to said patient.

39. A method of treating polymyositis, comprising administering a therapeutically effective amount of a compound of any one of claims 1-23, or pharmaceutical composition of claim 24, to said patient.

40. A method of treating psoriasis, comprising administering a therapeutically effective amount of a compound of any one of claims 1-23, or pharmaceutical composition of claim 24, to said patient.

41. A method of treating COPD, comprising administering a therapeutically effective amount of a compound of any one of claims 1-23, or pharmaceutical composition of claim 24, to said patient.

42. A method of treating IBD, comprising administering a therapeutically effective amount of a compound of any one of claims 1-23, or pharmaceutical composition of claim 24, to said patient.

43. A method of treating obesity, comprising administering a therapeutically effective amount of a compound of any one of claims 1-23, or pharmaceutical composition of claim 24, to said patient.

44. A method of treating insulin resistance, comprising administering a therapeutically effective amount of a compound of any one of claims 1-23, or pharmaceutical composition of claim 24, to said patient.

45. A method of treating NIDDM, comprising administering a therapeutically effective amount of a compound of any one of claims 1-23, or pharmaceutical composition of claim 24, to said patient.

46. A method of treating metabolic syndrome, comprising administering a therapeutically effective amount of a compound of any one of claims 1-23, or pharmaceutical composition of claim 24, to said patient.

47. A method of treating cancer, comprising identifying a human patient having cancer and administering a therapeutically effective amount of a compound of any one of claims 1-23, or pharmaceutical composition of claim 24, to said patient.

48. A method of delaying the onset, or reducing the severity of, one or more symptoms of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, in a human patient, comprising administering a therapeutically effective amount of a compound of any one of claims 1-23, or pharmaceutical composition of claim 24, to said patient.

49. The method of claim 48, further comprising identifying a patient in need of such treatment prior the administering step.

50. A method of making a compound of any one of claims 1-23, comprising following one of the synthetic schemes disclosed herein.

51. The use of a compound of any one of claims 1-23 for the manufacture of a medicament useful for human therapy.

52. The use of claim 51, wherein said therapy comprises therapy for the treatment of inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, in a human patient.

53. The use of claim 52, wherein said therapy comprises therapy for the delaying the onset of, or reducing the symptoms of, inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, in a human patient.

54. A composition for treating inflammation, RA, SLE, diseases associated with aberrant accumulation of cytosolic nucleic acids (including Sjögrens syndrome, Aicardi-Goutières syndrome, subtypes of SLE, chilblain lupus, and RVCL), systemic sclerosis, myositis (including dermatomyositis and polymyositis), psoriasis, COPD, IBD, obesity, insulin resistance, NIDDM, metabolic syndrome and cancer, and complications associated with these diseases and disorders, in a human patient, said composition comprising a compound of any one of claims 1-23.

55. A method of inhibiting the kinase activity of IKKε, TBK1, or both IKKε and TBK1 in human cells comprising, contacting said cells with a compound of any one of claims 1-23 or a pharmaceutical composition of claim 24.

56. The method of claim 55 wherein said cells are within the body of a human patient.

57. The method of claim 55 or 56, wherein said method consists of inhibiting the kinase activity of IKKε.

58. The method of claim 55 or 56, wherein said method consists of inhibiting the kinase activity of TBK1.

59. The method of claim 55 or 56, wherein said method consists of inhibiting the kinase activity of IKKε and TBK1.
